(19) United States
(12) Patent Application Publication
(10) Pub. No.: US 2015/0132339 A1
(43) Pub. Date: May 14, 2015

(54) ADJUVANTED FORMULATIONS OF STREPTOCOCCUS PNEUMONIAE ANTIGENS

(71) Applicant: NOVARTIS AG, Basel (CH)

(72) Inventors: Simone Bufali, Siena (IT); Paolo Costantino, Colle di val D'Elsa (IT); Michele Pallaoro, Siena (IT); Derek O'hagan, Winchester, MA (US); Rino Rappuoli, Castelnuovo Berardenga (IT); Manmohan Singh, Cary, NC (US)

(21) Appl. No.: 14/381,090

(22) PCT Filed: Mar. 7, 2013
(86) PCT No.: PCT/EP2013/054545

§ 371 Date: Aug. 26, 2014

Related U.S. Application Data
(60) Provisional application No. 61/607,987, filed on Mar. 7, 2012.
(60) Provisional application No. 61/608,013, filed on Mar. 7, 2012.

(51) Int. Cl. A61K 39/09; A61K 33/08; A61K 31/4745; A61K 39/39

(52) U.S. Cl. CPC A61K 39/092; A61K 39/39; A61K 33/08; A61K 31/4745; A61K 2039/55505
USPC 424/197.11; 424/244.1

(57) ABSTRACT

The efficacy of S. pneumoniae vaccines can be enhanced by adjuvanting S. pneumoniae saccharide and/or protein antigens with a mixture of a TLR agonist (preferably a TLR7 agonist) and an insoluble metal salt (preferably an aluminium salt). The TLR agonist is typically adsorbed to the metal salt. The S. pneumoniae antigen can also be adsorbed to the metal salt.

This application claims the benefit of U.S. provisional applications 61/607,987 and 61/608,013 (both filed Mar. 7, 2012), the complete contents of both of which are hereby incorporated herein by reference for all purposes.

### TECHNICAL FIELD

The invention is in the field of adjuvanting antigens (particularly protein or saccharide antigens) from Streptococcus pneumoniae to increase their immunogenicity.

### BACKGROUND ART

There are currently two main types of pneumococcal vaccine. Pneumococcal conjugate vaccine (PCV) is given to all children under two years of age as part of the childhood vaccination programme, and pneumococcal polysaccharide vaccine (PPV) is given to people who are 65 years of age or over, and people at high risk. A 7-valent conjugate vaccine (PCV7, Prevnar, Pfizer) was licensed in 2000 and contains saccharides from serotypes 4, 6B, 9V, 14, 18C, 19F, 23F, which are the serotypes most commonly causing invasive pneumococcal disease among young children in North America. PCV10 (Synflorix, GlaxoSmithKline) was licensed for use in Canada, Australia, and

Europe in 2008/09 and contains PCV7 serotypes plus serotypes 1, 5, and 7F. PCV13 (Prevnarl3, Pfizer) added serotypes 3, 6A, and 19A to the PCV10 serotypes and was licensed in Chile and European in 2009. PPV23 (Pneumovax, Merck) is a 23-valent formulation of polysaccharide serotypes 1, 2, 3, 4, 5, 6B, 7F, 8, 9N, 9V, 10A, 11A, 12F, 14, 15B, 17F, 18C, 19A, 19F, 20, 22F, 23F, and 33F. These vaccines induce anti-capsular antibodies to the specific pneumococcal serotype included in the formulation and have been shown to be protective against invasive disease, especially septicemia and meningitis. PPV23 is unadjuvanted, but all of the conjugated vaccines include an aluminium salt adjuvant.

In addition to these saccharide-based compositions, vaccines comprising S. pneumoniae protein antigens are known in the art. References 1 and 231 describe protective immunogenic compositions comprising S. pneumoniae pilus proteins adjuvanted with an aluminium salt.

It is an object of the invention to provide further adjuvanted immunogenic compositions for protecting against S. pneumoniae, and in particular to provide compositions which are superior to those adjuvanted with aluminium salts.

### DISCLOSURE OF THE INVENTION

The inventors have found that the efficacy of S. pneumoniae vaccines can be enhanced by adjuvanting S. pneumoniae antigens with a mixture of a TLR agonist (preferably a TLR7 agonist, such as compound ‘K2’ identified below) and an insoluble metal salt (preferably an aluminium salt). The TLR agonist is typically adsorbed to the metal salt, as disclosed in reference 2. A S. pneumoniae antigen can also be adsorbed to the metal salt. In some embodiments the S. pneumoniae antigen is a S. pneumoniae saccharide antigen; in other embodiments the S. pneumoniae antigen is a S. pneumoniae protein antigen.

In a first aspect, the invention provides an immunogenic composition comprising (i) a TLR agonist (ii) an insoluble metal salt and (iii) one or more S. pneumoniae saccharide antigens, wherein the TLR agonist is an agonist of human TLR7.

In a second aspect, the invention provides an immunogenic composition comprising (i) a TLR agonist (ii) an insoluble metal salt and (iii) one or more S. pneumoniae saccharide antigens, wherein the insoluble metal salt is an aluminium salt.

In a third aspect, the invention provides an immunogenic composition comprising (i) a TLR agonist (ii) an insoluble metal salt, (iii) a buffer and (iv) one or more S. pneumoniae saccharide antigens.

In a fourth aspect, the invention provides an immunogenic composition comprising (i) a TLR agonist (ii) an insoluble metal salt and (iii) one or more S. pneumoniae saccharide antigens, wherein the pH of the composition is between 6 and 8, preferably between 6 and 7.

In a fifth aspect, the invention provides an immunogenic composition comprising (i) a TLR agonist (ii) an insoluble metal salt and (iii) one or more S. pneumoniae saccharide antigens, wherein at least one of the one or more S. pneumoniae saccharide antigens is conjugated to CRM197, and optionally wherein the composition does not include diphtheria toxoid, tetanus toxoid and pertussis toxoid.

In a sixth aspect, the invention provides an immunogenic composition comprising (i) a TLR agonist (ii) an insoluble metal salt and (iii) one or more S. pneumoniae saccharide antigens selected from 2-10 different serotypes, or 12 or more different serotypes; and optionally wherein the composition does not include diphtheria toxoid, tetanus toxoid and pertussis toxoid.

In a seventh aspect, the invention provides an immunogenic composition comprising (i) a TLR agonist (ii) an insoluble metal salt and (iii) S. pneumoniae saccharide antigens from precisely 11 different serotypes, provided that the 11 different serotypes are not serotypes 1, 3, 4, 5, 6B, 7F, 9V, 14, 18C, 19F and 23F.

In an eighth aspect, the invention provides an immunogenic composition comprising (i) a TLR agonist (ii) an insoluble metal salt and (iii) one or more S. pneumoniae saccharide antigens, wherein at least one of said one or more S. pneumoniae saccharide antigens is conjugated directly to a carrier, preferably by reductive amination.

In a ninth aspect, the invention provides an immunogenic composition comprising (i) a TLR agonist (ii) an insoluble metal salt and (iii) one or more S. pneumoniae saccharide antigens, wherein at least one of said one or more S. pneumoniae saccharide antigens is conjugated to a carrier via a linker.

In a tenth aspect, the invention provides an immunogenic composition comprising: (i) an aluminium hydroxide adjuvant; (ii) a TLR7 agonist of formula (K); (iii) S. pneumoniae saccharide antigens from serotypes 1, 5, 6B, 14, and 23F, in which each saccharide is conjugated to CRM197; in which the TLR7 agonist and/or at least one of the saccharides is/are adsorbed to the aluminium hydroxide adjuvant.

In an eleventh aspect, the invention provides an immunogenic composition comprising: (i) an aluminium hydroxide adjuvant; (ii) a TLR7 agonist of formula (K); (iii) S. pneumoniae saccharide antigen from only serotype 5 conjugated to CRM197; in which the TLR7 agonist and/or at least one of the saccharides is/are adsorbed to the aluminium hydroxide adjuvant.

In a twelfth aspect, the invention provides an immunogenic composition comprising: (a) an adjuvant complex comprising a first TLR agonist adsorbed to an insoluble metal salt; (b) an adjuvant complex comprising a second TLR agonist adsorbed to an insoluble metal salt; and (c) at least one S. pneumoniae saccharide antigen, wherein the saccharide antigen(s) are preferably adsorbed to the metal salt(s).

In a thirteenth aspect, the invention provides a process for preparing an immunogenic composition, wherein the process comprises mixing a TLR agonist, an insoluble metal salt, and S. pneumoniae saccharide antigen(s).

In a fourteenth aspect, the invention provides a process for preparing an immunogenic composition, comprising one of: (i) combining a S. pneumoniae saccharide antigen with a mixture comprising a TLR agonist and an insoluble metal salt; (ii) combining an insoluble metal salt with a mixture comprising a TLR agonist and a S. pneumoniae saccharide antigen; or (iii) combining a TLR agonist with a mixture comprising an insoluble metal salt and a S. pneumoniae saccharide antigen.

In a fifteenth aspect, the invention provides a process for preparing an immunogenic composition, comprising steps of (i) preparing an aqueous mixture of a TLR agonist and a soluble aluminium salt; then (ii) adding a non-aluminium salt to the aqueous mixture in order to form a precipitated aluminium salt to which the TLR agonist is adsorbed; and (iii) adding S. pneumoniae saccharide antigen(s) in step (i), step (ii), and/or a third step. The invention also provides an immunogenic composition obtained or obtainable by this process.

In a sixteenth aspect, the invention provides a process for preparing an immunogenic composition, comprising a step of mixing (i) an aqueous mixture of a TLR agonist and a soluble aluminium salt with (ii) a buffered aqueous mixture of a S. pneumoniae saccharide immunogen, wherein the mixing step causes precipitation of an aluminium salt to which the TLR agonist and the immunogen are adsorbed. The invention also provides an immunogenic composition obtained or obtainable by this process.

In a seventeenth aspect, the invention provides a process for preparing a sterile immunogenic composition, comprising steps of combining (i) a S. pneumoniae saccharide immunogen with (ii) a sterile complex of a TLR agonist and an insoluble metal salt. This process may comprise e.g. the steps of (a) mixing a TLR agonist and an insoluble metal salt such that the TLR agonist adsorbs to the insoluble metal salt to form the complex; and (b) sterilising the complex. As an alternative, this process may comprise e.g. the steps of (a) sterilising a solution or suspension of a TLR agonist and (b) combining the sterilised solution or suspension with a sterile insoluble metal salt; or by (a) sterilising an insoluble metal salt and (b) combining the sterilised insoluble metal salt with a sterile solution or suspension of a TLR agonist; or by combining (a) a sterile solution or suspension of a TLR agonist with (b) a sterile insoluble metal salt. Sterilisation of the TLR agonist solution/suspension may be achieved by sterile filtration. Sterilisation of the insoluble metal salt may be achieved by autoclaving.

In an 18th aspect, the invention provides an immunogenic composition comprising (i) a TLR agonist (ii) an insoluble metal salt and (iii) one or more S. pneumoniae protein antigens, wherein the TLR agonist is an agonist of human TLR7.

In a 19th aspect, the invention provides an immunogenic composition comprising (i) a TLR agonist (ii) an insoluble metal salt and (iii) one or more S. pneumoniae protein antigens, wherein the insoluble metal salt is an aluminium salt.

In a 20th aspect, the invention provides an immunogenic composition comprising (i) a TLR agonist (ii) an insoluble metal salt, (iii) a buffer and (iv) one or more S. pneumoniae protein antigens.

In a 21st aspect, the invention provides an immunogenic composition comprising (i) a TLR agonist (ii) an insoluble metal salt and (iii) one or more S. pneumoniae protein antigens, wherein the pH of the composition is between 6 and 8, preferably between 6 and 7.

In a 22nd aspect, the invention provides an immunogenic composition comprising (i) a TLR agonist (ii) an insoluble metal salt and (iii) at least two of:

(a) a first polypeptide comprising a first amino acid sequence, where the first amino acid sequence comprises an amino acid sequence (i) having at least 90% sequence identity to SEQ ID NO: 236 and/or (ii) consisting of a fragment of at least 7 contiguous amino acids from SEQ ID NO: 236;

(b) a second polypeptide, comprising a second amino acid sequence, where the second amino acid sequence comprises an amino acid sequence (i) having at least 90% sequence identity to SEQ ID NO: 237 and/or (ii) consisting of a fragment of at least 7 contiguous amino acids from SEQ ID NO:237; and/or

(c) a third polypeptide, comprising a third amino acid sequence, where the third amino acid sequence comprises an amino acid sequence (i) having at least 90% sequence identity to SEQ ID NO: 238 and/or (ii) consisting of a fragment of at least 7 contiguous amino acids from SEQ ID NO: 238.

In a 23rd aspect, the invention provides an immunogenic composition comprising (i) a TLR agonist (ii) an insoluble metal salt, and (iii) a polypeptide comprising amino acid sequence:

A-{1-X-L-}ₙ-B

wherein: each X is an amino acid sequence of first polypeptide, second polypeptide or third polypeptide as defined in the 22nd aspect; L is an optional linker amino acid sequence; A is an optional N terminal amino acid sequence; B is an optional C terminal amino acid sequence; n is an integer of 2 or more.

In a 24th aspect, the invention provides an immunogenic composition comprising (i) a TLR agonist (ii) an insoluble metal salt and (iii) a protein antigencomprising the amino acid sequence of: SEQ ID NOs: 246, 248, 250, 252, 254 or 256.

In a 25th aspect, the invention provides an immunogenic composition comprising (i) a TLR agonist (ii) an insoluble metal salt and (iii) a polypeptide comprising amino acid sequence SEQ ID NO: 318.

In a 26th aspect, the invention provides an immunogenic composition comprising (i) a TLR agonist (ii) an insoluble metal salt and (iii) an immunogenic composition comprising: (a) a first polypeptide comprising a first amino acid sequence, where the first amino acid sequence comprises or consists of: SEQ ID NO.335, or an amino acid sequence having at least 80% sequence identity to SEQ ID NO: 335, or an amino acid sequence that competes with SEQ ID NO: 335 for binding to an antibody raised against SEQ ID NO: 335, or a fragment of at least 7 amino acids of SEQ ID NO: 335; and/or (b) a second polypeptide, comprising a second amino acid sequence, where the second amino acid sequence comprises or consists of: SEQ ID NO: 336, or an amino acid sequence having at least 80% sequence identity to SEQ ID NO: 336, or an amino acid sequence that competes with SEQ ID NO: 336 for binding to an antibody raised against SEQ ID NO: 336, or a fragment of at least 7 amino acids of SEQ ID NO: 336; and/or (c) a third polypeptide, comprising a third amino acid sequence, where the third amino acid sequence comprises or consists of: SEQ ID NO: 337, or an amino acid sequence having at least 80% sequence identity to SEQ ID NO: 337, or an amino acid sequence that competes with SEQ ID NO: 337 for binding to an antibody raised against SEQ ID NO: 337, or a fragment of at least 7 amino acids of SEQ ID NO: 337; and/or (d) a fourth polypeptide, comprising a fourth amino acid sequence, where the fourth amino acid sequence comprises or consists of: SEQ ID NO: 338, or an amino acid sequence having at least 80% sequence identity to SEQ ID NO: 338, or an amino acid sequence that competes with SEQ ID NO: 338 for binding to an antibody raised against SEQ ID NO: 338, or a fragment of at least 7 amino acids of SEQ ID NO: 338; and/or (e) a fifth polypeptide, comprising a fifth amino acid sequence, where the fifth amino acid sequence comprises or consists of: SEQ ID NO: 339, or an amino acid sequence having at least 80% sequence identity to SEQ ID NO: 339, or an amino acid sequence that competes with SEQ ID NO: 339 for binding to an antibody raised against SEQ ID NO: 339, or a fragment of at least 7 amino acids of SEQ ID NO: 339; and/or (f) a sixth polypeptide, comprising a sixth amino acid sequence, where the sixth amino acid sequence comprises or consists of: SEQ ID NO: 340, or an amino acid sequence having at least 80% sequence identity to SEQ ID NO: 340, or an amino acid sequence that competes with SEQ ID NO: 340 for binding to an antibody raised against SEQ ID NO: 340, or a fragment of at least 7 amino acids of SEQ ID NO: 340. Preferably, the first, second, third, fourth, fifth and/or sixth polypeptide contains 50 or fewer, 45 or fewer, 40 or fewer, 35 or fewer, 34 or fewer, 33 or fewer, 30 or fewer, or 25 or fewer amino acid residues.

In a 27th aspect, the invention provides an immunogenic composition comprising (i) a TLR agonist (ii) an insoluble metal salt and (iii) a polypeptide comprising amino acid sequence:

A-{-X-L-}ₙ-B

wherein: each X is an amino acid sequence of first amino acid sequence, second amino acid sequence, third amino acid sequence, fourth amino acid sequence, fifth amino acid sequence or sixth amino acid sequence as defined in the 26th aspect; L is an optional linker amino acid sequence; A is an optional N terminal amino acid sequence; B is an optional C terminal amino acid sequence; n is an integer of 2 or more. For instance, n can provide 2, 3, 4, 5 or 6 different amino acid sequences, and ideally comprising amino acid sequences from two or three different RrgB clades.

Immunogenic compositions of the 26th and/or 27th aspects preferably comprise two, three, four, five or six different amino acid sequences, more preferably from two or three different RrgB clades e.g. comprising at least one amino acid sequence selected from two or more of the following groups defined in the 26th aspect: (a) the first and second amino acid sequence; (b) the third and fourth amino acid sequence; and (c) the fifth and sixth amino acid sequence.

In an 28th aspect, the invention provides an immunogenic composition comprising: (i) an aluminium hydroxide adjuvant; (ii) a TLR7 agonist of formula (K); (iii) RrgB321; in which the TLR7 agonist and/or RrgB321 is/are adsorbed to the aluminium hydroxide adjuvant.

In a 29th aspect, the invention provides an immunogenic composition comprising: (a) an adjuvant complex comprising a first TLR agonist adsorbed to an insoluble metal salt; (b) an adjuvant complex comprising a second TLR agonist adsorbed to an insoluble metal salt; and (c) at least one S. pneumoniae protein antigen, wherein preferably, the protein antigen(s) are preferably adsorbed to the metal salt(s).

In a 30th aspect, the invention provides a process for preparing an immunogenic composition, wherein the process comprises mixing a TLR agonist, an insoluble metal salt, and S. pneumoniae protein antigen(s).

In a 31st aspect, the invention provides a process for preparing an immunogenic composition, comprising one of: (i) combining a S. pneumoniae protein antigen with a mixture comprising a TLR agonist and an insoluble metal salt; (ii) combining an insoluble metal salt with a mixture comprising a TLR agonist and a S. pneumoniae protein antigen; or (iii) combining a TLR agonist with a mixture comprising an insoluble metal salt and a S. pneumoniae protein antigen.

In a 32nd aspect, the invention provides a process for preparing an immunogenic composition, comprising steps of (i) preparing an aqueous mixture of a TLR agonist and a soluble aluminium salt; then (ii) adding a non-aluminium salt to the aqueous mixture in order to form a precipitated aluminium salt to which the TLR agonist is adsorbed; and (iii) adding S. pneumoniae protein antigen(s) in step (i), step (ii), and/or a third step. In one aspect, the invention provides an immunogenic composition obtained or obtainable by the method of the 32nd aspect.

In a 33rd aspect, the invention provides a process for preparing an immunogenic composition, comprising a step of mixing (i) an aqueous mixture of a TLR agonist and a soluble aluminium salt with (ii) a buffered aqueous mixture of a S. pneumoniae protein immunogen, wherein the mixing step causes precipitation of an aluminium salt to which the TLR agonist and the immunogen are adsorbed.

The invention also provides an immunogenic composition obtained or obtainable by the process of the 33rd aspect.

In a 34th aspect, the invention provides a process for preparing a sterile immunogenic composition, comprising steps of combining (i) a S. pneumoniae protein immunogen with (ii) a sterile complex of a TLR agonist and an insoluble metal salt. Preferably, the process comprises the steps of (a) mixing a TLR agonist and an insoluble metal salt such that the TLR agonist adsorbs to the insoluble metal salt to form the complex; and (b) sterilising the complex. As an alternative, the process comprises the steps of (a) sterilising a solution or suspension of a TLR agonist and (b) combining the sterilised solution or suspension with a sterile insoluble metal salt; or by (a) sterilising an insoluble metal salt and (b) combining the sterilised insoluble metal salt with a sterile solution or suspension of a TLR agonist; or by combining (a) a sterile solution or suspension of a TLR agonist with (b) a sterile insoluble metal salt. Sterilisation of the TLR agonist solution/suspension is preferably achieved by sterile filtration, and/or sterilisation of the insoluble metal salt is achieved by autoclaving.

In a 35th aspect, the invention provides a method of raising an immune response in a subject, comprising the step of administering to the subject the composition of any aspect. Preferably, the method comprises administering to the subject two or more doses of the composition of any aspect.

In some embodiments, the invention provides a method of raising an immune response in a subject, comprising administering to the subject two or more doses of the composition of any preceding aspect.

In some embodiments, the TLR agonist is an agonist of human TLR7. Preferably, the TLR agonist includes at least one adsorptive moiety which allows it to adsorb to insoluble metal salts; more preferably, the adsorptive moiety is a phosphate or a phosphonate.

In some embodiments the TLR agonist has formula (C), (D), (E), (F), (G), (H), (I), (II), (J) or (K) as defined in the description. In some embodiments, the TLR agonist is one of compounds 1 to 102 as defined in reference 2, or a pharmaceutically acceptable salt thereof. In some embodiments, the TLR agonist is compound K2, or a pharmaceutically acceptable salt thereof.

In some embodiments, the insoluble metal salt is an aluminium salt, preferably an aluminium hydroxide. In some embodiments, the aluminium salt has an Al⁺⁺⁻ concentration between 10-500 μg/ml.

In some embodiments, >80% of the TLR agonist is adsorbed to the insoluble metal salt.

In some embodiments, the one or more S. pneumoniae saccharide antigen(s) is selected from serotypes 1, 2, 3, 4, 5, 6A, 6B, 7F, 8, 9N, 9V, 10A, 11A, 12F, 14, 15B, 17F, 18C, 19A, 19F, 20, 22F, 23F, and/or 33F.

In some embodiments, the composition or process comprises a 5 valent combination of serotypes e.g. from serotypes 1, 5, 6B, 14, and 23F.

In some embodiments, the composition or process comprises a 7 valent combination of serotypes e.g. from serotypes 4, 6B, 9V, 14, 18C, 19F, and 23F.

In some embodiments, the composition or process comprises a 9 valent combination of serotypes e.g. from serotypes 1, 4, 5, 6B, 9V, 14, 18C, 19F and 23F.

In some embodiments, the composition or process comprises a 10 valent combination of serotypes e.g. from serotypes 1, 4, 5, 6B, 7F, 9V, 14, 18C, 19F and 23F.

In some embodiments, the composition or process comprises a combination of 11 serotypes.

In some embodiments, the composition or process comprises a 12 valent combination of serotypes, e.g. from serotypes 1, 4, 5, 6B, 7F, 9V, 14, 18C, 19F and 23F, preferably further comprising serotypes 6A and 19A; 6A and 22F; 19A and 22F; 6A and 15B; 19A and 15B; or 22F and 15B.

In some embodiments, the composition or process comprises a 13 valent combination e.g. from serotypes 1, 3, 4, 5, 6B, 7F, 9V, 14, 18C, 19F and 23F, in addition to serotypes 19A and 22F; 8 and 12F; 8 and 15B; 8 and 19A; 8 and 22F; 12F and 15B; 12F and 19A; 12F and 22F; 15B and 19A; 15B and 22F; or 6A and 19A. In some embodiments, the combination of 13 serotypes comprises serotypes 1, 3, 4, 5, 6A, 6B, 7F, 9V, 14, 18C, 19, 19F and 23F, or 1, 3, 4, 5, 6A, 6B, 7F, 9V, 14, 18C, 19A, 19F and 23F.

In some embodiments, the weight of each saccharide is 0.01-500 μg/ml for each serotype. In some embodiments, wherein there are saccharides from two or more serotypes, there is a 1:1 weight ratios of saccharides.

In some embodiments, the immunogenic composition comprises S. pneumoniae saccharide antigen from one serotype, preferably selected from serotypes 1, 2, 3, 4, 5, 6A, 6B, 7F, 8, 9N, 9V, 10A, 11A, 12F, 14, 15B, 17F, 18C, 19A, 19F, 20, 22F, 23F, and/or 33F, more preferably from serotype 1, 5, 6B, 14, or 23F.

In some embodiments, one or more S. pneumoniae saccharide antigen(s) is conjugated to a carrier protein. In some embodiments, the carrier protein is a bacterial toxin, toxoid, or mutant thereof, preferably selected from diphtheria, tetanus or Hinfluenzae, preferably diphtheria. A preferred carrier is CRM197, which is preferably at a concentration of 55-60μg/ml.

In some embodiments, there are S. pneumoniae saccharide antigens from two or more different serotypes, and the two or more saccharide antigens are conjugated to the same type of carrier protein. In other embodiments, there are S. pneumoniae saccharide antigens from two or more different serotypes, and the two or more saccharide antigens are conjugated different types of carrier protein.

In some embodiments, the carrier is conjugated directly to the saccharide, preferably by reductive amination between the saccharide and the carrier. In some embodiments, the carrier is conjugated to the saccharide via a linker, preferably an adipic acid linker, a carbonyl linker, a β-propionamido linker, nitrophenyl-ethylamine linker, haloacyl halide linker, glycosidic linker, 6-aminocaproic acid linker, ADH linker, or a C4 to C12 linker.

In some embodiments, the composition or process comprises a buffer, preferably a histidine buffer. Preferably, the histidine buffer is at a concentration of less than 50 mM histidine buffer.

In some embodiments, the composition or process has a pH between 6 and 8, preferably between pH 6 and 7.

In some embodiments, a composition or process which comprises a S. pneumoniae saccharide antigen further comprises a S. pneumoniae protein antigen. In some embodiments, a composition or process which comprises a S. pneumoniae protein antigen further comprises a S. pneumoniae saccharide antigen.

TLR Agonists

Compositions of the invention include a TLR agonist i.e. a compound which can agonise a Toll-like receptor. Most preferably, a TLR agonist is an agonist of a human TLR. The TLR agonist can activate any of TLR1, TLR2, TLR3, TLR4, TLR5, TLR6, TLR7, TLR8, TLR9 or TLR11; preferably it can activate human TLR7.

Agonist activity of a compound against any particular Toll-like receptor can be determined by standard assays. Companies such as Imgenex and Invivogen supply cell lines which are stably co-transfected with human TLR genes and NFKB, plus suitable reporter genes, for measuring TLR activation pathways. They are designed for sensitivity, broad working range dynamics and can be used for high-throughput screening. Constitutive expression of one or two specific TLRs is typical in such cell lines. See also reference 3. Many TLR agonists are known in the art e.g. reference 4 describes certain lipopeptide molecules that are TLR2 agonists, references 5 to 8 each describe classes of small molecule agonists of TLR7, and references 9 & 10 describe TLR7 and TLR8 agonists for treatment of diseases.

A TLR agonist used with the invention ideally includes at least one adsorptive moiety. The inclusion of such moieties in TLR agonists allows them to adsorb to insoluble metal salts (e.g. by ligand exchange or any other suitable mechanism) and improves their immunological behaviour (see reference 2). Phosphorus-containing adsorptive moieties are particularly useful, and so an adsorptive moiety may comprise a phosphate, a phosphonate, a phosphinate, a phosphonite, a phosphinite, etc.

Preferably the TLR agonist includes at least one phosphonate group.

Thus, in preferred embodiments, a composition of the invention includes a TLR7 agonist which includes a phosphonate group. This phosphonate group can allow adsorption of the agonist to an insoluble metal salt, such as to an aluminium salt.

TLR agonists useful with the invention may include a single adsorptive moiety, or may include more than one e.g. between 2 and 15 adsorptive moieties. Typically a compound will include 1, 2 or 3 adsorptive moieties.

Phosphorus-containing TLR agonists useful with the invention can be represented by fomula (A1):

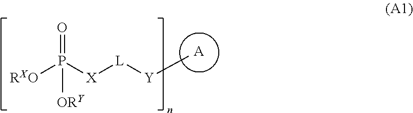

wherein:
R^{X} and RY are independently selected from H and C₁-C₆ alkyl;
X is selected from a covalent bond, O and NH;
Y is selected from a covalent bond, O, C(O), S and NH;
L is a linker e.g. selected from, C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂;
each p is independently selected from 1, 2, 3, 4, 5 and 6;
q is selected from 1, 2, 3 and 4;
n is selected from 1, 2 and 3; and
A is a TLR agonist moiety.

In one embodiment, the TLR agonist according to formula (A1) is as follows: R^{X} and R^{Y} are H; X is O; L is selected from C₁-C₆ alkylene and —((CH₂)ₚO)_{q}(CH₂)ₚ— each optionally substituted with 1 to 2 halogen atoms; p is selected from 1, 2 and 3; q is selected from 1 and 2; and n is 1. Thus in these embodiments the adsorptive moiety comprises a phosphate group.

In other embodiments, the TLR agonist according to formula (A1) is as follows: R^{X} and R^{Y} are H; X is a covalent bond; L is selected from C₁-C₆ alkylene and —((CH₂)ₚO)_{q}(CH₂)ₚ— each optionally substituted with 1 to 2 halogen atoms; p is selected from 1, 2 or 3; q is selected from 1 or 2; and n is 1. Thus in these embodiments the adsorptive moiety comprises a phosphonate group.

Useful ‘A’ moieties for fomula (A1) include, but are not limited to, radicals of any of the following compounds, defined herein or as disclosed in references 4-10 and 213-231:

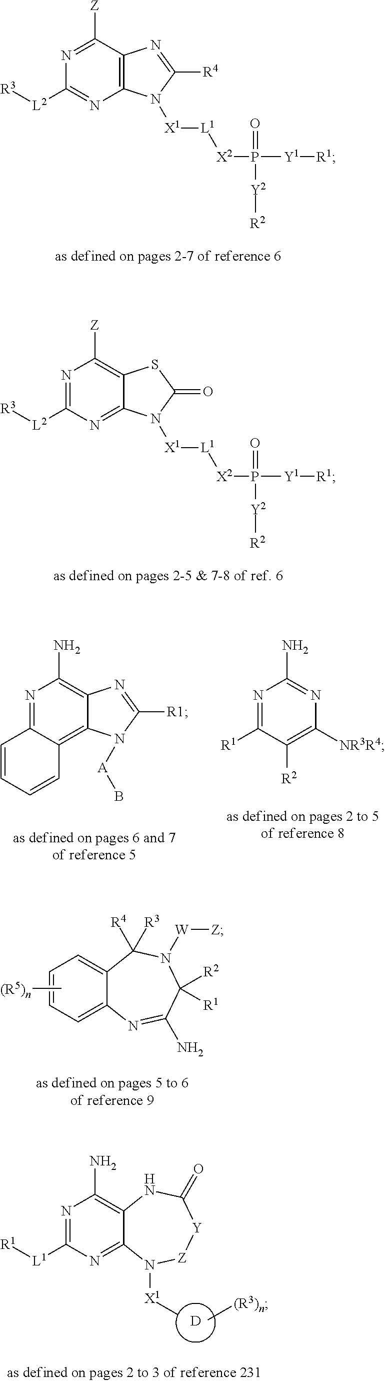

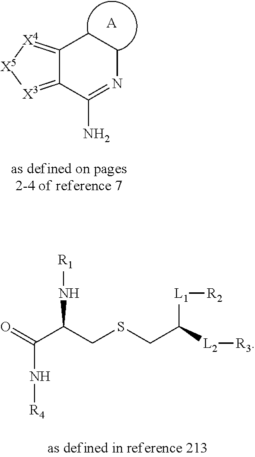

In some embodiments, the TLR agonist moiety ‘A’ has a molecular weight of less than 1000 Da. In some embodiments, the TLR agonist of fomula (A1) has a molecular weight of less than 1000 Da.

Preferred TLR agonists are water-soluble. Thus they can form a homogenous solution when mixed in an aqueous buffer with water at pH 7 at 25° C. and 1 atmosphere pressure to give a solution which has a concentration of at least 50 μg/ml. The term “water-soluble” thus excludes substances that are only sparingly soluble under these conditions.

Useful TLR agonists include those having formula (C), (D), (E), (F), (G), (H), (I), (II), (J) or (K) as described in more detail below. Other useful TLR agonists are compounds 1 to 102 as defined in reference 2. Preferred TLR7 agonists have formula (K), such as ‘K2’. These can be used as salts e.g. the arginine salt of K2.

Preferred TLR4 agonists are analogs of monophosphoryl lipid A (MPL). For instance, a useful TLR4 agonist is a 3d-MPL (i.e. 3-O-deacylated monophosphoryl lipid A; also known as 3-de-O-acylated monophosphoryl lipid A or 3-O-desacyl-4′-monophosphoryl lipid A). The name indicates that position 3 of the reducing end glucosamine in monophosphoryl lipid A is de-acylated. It has been prepared from a heptoseless mutant of Salmonella minnesota, and is chemically similar to lipid A but lacks an acid-labile phosphoryl group and a base-labile acyl group. It activates cells of the monocyte/macrophage lineage and stimulates release of several cytokines, including IL-1, IL-12, TNF-α and GM-CSF. Preparation of 3d-MPL was originally described in reference 11, and the product has been manufactured and sold by Corixa Corporation. It is present in the AS04 adjuvant used by GlaxoSmithKline. Further details can be found in references 12 to 15.

Typical compositions include 3d-MPL at a concentration of between 25 μg/ml and 200 μg/ml e.g. in the range 50-150 μg/ml, 75-125 μg/ml, 90-110 μg/ml, or about 100 μg/ml. It is usual to administer between 25-75 μg of 3d-MPL per dose e.g. between 45-55 μg, or about 50 μg 3d-MPL per dose.

3d-MPL can take the form of a mixture of related molecules, varying by their acylation (e.g. having 3, 4, 5 or 6 acyl chains, which may be of different lengths). The two glucosamine (also known as 2-deoxy-2-amino-glucose) monosaccharides are N-acylated at their 2-position carbons (i.e. at positions 2 and 2′), and there is also O-acylation at the 3′ position. The group attached to carbon 2 has formula —NH—CO—CH₂—CR¹R^{1′}. The group attached to carbon 2′ has formula —NH—CO—CH₂—CR²R²′. The group attached to carbon 3′ has formula —O—CO—CH₂—CR³R^{3′}. A representative structure is:

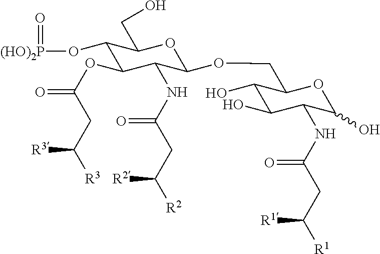

Groups R¹, R² and R³ are each independently —(CH₂)ₙ—CH₃. The value of n is preferably between 8 and 16, more preferably between 9 and 12, and is most preferably 10.

Groups R^{1′}, R^{2′} and R^{3′} can each independently be: (a) —H; (b) —OH; or (c) —O—CO—R⁴,where R⁴ is either —H or —(CH₂)ₘ—CH₃, wherein the value of m is preferably between 8 and 16, and is more preferably 10, 12 or 14. At the 2 position, m is preferably 14. At the 2′ position, m is preferably 10.

At the 3′ position, m is preferably 12. Groups R^{1′}, R^{2′} and R^{3′} are thus preferably —O-acyl groups from dodecanoic acid, tetradecanoic acid or hexadecanoic acid.

When all of R^{1′}, R^{2′} and R^{3′} are H then the 3d-MPL has only 3 acyl chains (one on each of positions 2, 2′ and 3′). When only two of R^{1′}, R^{2′} and R^{3′} are H then the 3d-MPL can have 4 acyl chains. When only one of R^{1′}, R^{2′} and R^{3′} is H then the 3d-MPL can have 5 acyl chains. When none of R^{1′}, R^{2′} and R^{3′} is —H then the 3d-MPL can have 6 acyl chains. The 3d-MPL used according to the invention can be a mixture of these forms, with from 3 to 6 acyl chains, but it is preferred to include 3d-MPL with 6 acyl chains in the mixture, and in particular to ensure that the 6 acyl chain form makes up at least 10% by weight of the total 3d-MPL e.g. ≧20%, ≧30%, ≧40%, ≧50% or more. 3d-MPL with 6 acyl chains has been found to be the most adjuvant-active form.

Thus the most preferred form of 3d-MPL for use with the invention is:

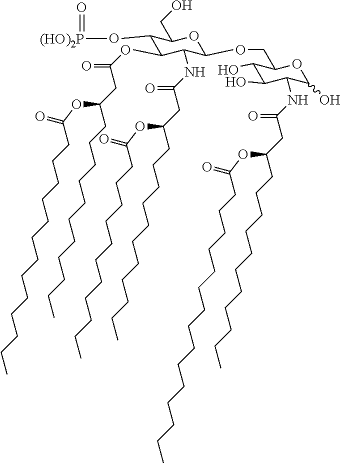

Where 3d-MPL is used in the form of a mixture then references to amounts or concentrations of 3d-MPL in compositions of the invention refer to the combined 3d-MPL species in the mixture.

In aqueous conditions, 3d-MPL can form micellar aggregates or particles with different sizes e.g. with a diameter <150 nm or >500 nm. Either or both of these can be used with the invention, and the better particles can be selected by routine assay. Smaller particles (e.g. small enough to give a clear aqueous suspension of 3d-MPL) are preferred for use according to the invention because of their superior activity [16]. Preferred particles have a mean diameter less than 150 nm, more preferably less than 120 nm, and can even have a mean diameter less than 100 nm. In most cases, however, the mean diameter will not be lower than 50 nm. Where 3d-MPL is adsorbed to aluminum phosphate then it may not be possible to measure the 3D-MPL particle size directly, but particle size can be measured before adsorption takes place. Particle diameter can be assessed by the routine technique of dynamic light scattering, which reveals a mean particle diameter. Where a particle is said to have a diameter of x nm, there will generally be a distribution of particles about this mean, but at least 50% by number (e.g. ≧60%, ≧70%, ≧80%, ≧90%, or more) of the particles will have a diameter within the range x±25%.

A composition of the invention can include more than one TLR agonist. These two agonists are different from each other and they can target the same TLR or different TLRs. Both agonists can be adsorbed to a metal salt.

Insoluble Metal Salts

TLR agonists can adsorb to insoluble metal salts to form an adsorbed complex for adjuvanting S. pneumoniae antigens. For instance, they can be adsorbed to insoluble calcium salts (e.g. calcium phosphate) or, preferably, to insoluble aluminium salts. Such aluminium salts have a long history of use in vaccines.

Useful aluminium salts include, but are not limited to, aluminium hydroxide and aluminium phosphate adjuvants. Such salts are described e.g. in chapters 8 & 9 of reference 17. Aluminium salts which include hydroxide ions are the preferred insoluble metal salts for use with the present invention as these hydroxide ions can readily undergo ligand exchange. Thus preferred salts for adsorption of TLR agonists are aluminium hydroxide and/or aluminium hydroxyphosphate. These have surface hydroxyl moieties which can readily undergo ligand exchange with phosphorus-containing groups (e.g. phosphates, phosphonates) to provide stable adsorption.

The adjuvants commonly known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-3100 cm⁻¹ (chapter 9 of ref. 17). The degree of crystallinity of an aluminium hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants e.g. with needle-like particles with diameters about 2 nm. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

The adjuvants commonly known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 1.2. Hydroxyphosphates can be distinguished from strict AlPO₄ by the presence of hydroxyl groups. For example, an IR spectrum band at 3164 cm⁻¹ (e.g. when heated to 200° C.) indicates the presence of structural hydroxyls (chapter 9 of reference 17).

The PO₄/Al³⁺ molar ratio of an aluminium phosphate adjuvant will generally be between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs, with primary particles in the range of 50 nm). Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

In solution both aluminium phosphate and hydroxide adjuvants tend to form stable porous aggregates 1-10 μm in diameter [18].

A composition including an TLR agonist of the invention adsorbed to a metal salt can also include a buffer (e.g. a phosphate or a histidine or a Tris buffer). When such a composition includes a phosphate buffer, however, it is preferred that the concentration of phosphate ions in the buffer should be less than 50 mM e.g. <40 mM, <30 mM, <20 mM, <10 mM, or <5 mM, or between 1-15 mM. A histidine buffer is preferred e.g. between 1-50 mM, between 5-25 mM, or about 10 mM.

Because of the insolubility of adsorptive metal salts which are useful with the invention, compositions containing adsorbed immunopotentiators will generally be suspensions having a cloudy appearance. This can mask contaminating bacterial growth and so a composition of the invention may include a preservative such as thiomersal or 2-phenoxyethanol. It is preferred that a composition should be substantially free from (e.g. <10 μg/ml) mercurial material e.g. thiomersal-free. Vaccines containing no mercury are more preferred.

A composition can include a mixture of both an aluminium oxyhydroxide and an aluminium hydroxyphosphate, and a TLR agonist may be adsorbed to one or both of these salts.

The concentration of Al^{30 ++} in a composition for administration to a patient is preferably less than 10 mg/ml e.g. ≦5 mg/ml, ≦4 mg/ml, ≦3 mg/ml, ≦2 mg/ml, ≦1 mg/ml, etc. A preferred range of Al⁺⁺⁺ in a composition of the invention is between 0.3 and 1 mg/ml or between 0.3-0.5 mg/ml. A maximum of 0.85 mg/dose is preferred. Because the inclusion of a TLR agonist can improve the adjuvant effect of aluminium salts then the invention advantageously permits lower amounts of Al⁺⁺⁺ per dose, and so a composition of the invention can usefully include between 10 and 250 μg of Al⁺⁺⁺ per unit dose. Current pediatric vaccines typically include at least 300 μg Al⁺⁺⁺. In concentration terms, a composition of the invention may have an Al′ concentration between 10 and 500 μg/ml e.g. between 10-300 μg/ml, between 10-200 μg/ml, or between 10-100 μg/ml.

In general, when a composition includes both a TLR agonist and an aluminium salt, the weight ratio of agonist to Al⁺⁺⁺ will be less than 5:1 e.g. less than 4:1, less than 3:1, less than 2:1, or less than 1:1. Thus, for example, with an Al⁺⁺⁺ concentration of 0.5 mg/ml the maximum concentration of TLR agonist would be 1.5mg/ml. But higher or lower levels can be used.

Where a composition includes a TLR agonist and an insoluble metal salt, it is preferred that at least 50% (by mass) of the agonist in the composition is adsorbed to the metal salt e.g. ≧60%, ≧70%, ≧80%, ≧85%, ≧90%, ≧92%, ≧94%, ≧95%, ≧96%, ≧97%, ≧98%, ≧99%, or even 100%.

S. pneumoniae Antigens

Both saccharide and polypeptide antigens are known for S. pneumoniae. In some compositions the S. pneumoniae antigen(s) is/are saccharide antigen(s); in some embodiments of such compositions, the composition does not include a S. pneumoniae protein antigen. In other compositions the S. pneumoniae antigen(s) is/are protein antigen(s); in some embodiments of such compositions, the composition does not include a S. pneumoniae saccharide antigen. In still other embodiments, compositions include both S. pneumoniae protein antigen(s) and S. pneumoniae saccharide antigen(s).

Saccharide Antigens

S. pneumoniae causes bacterial meningitis and existing vaccines are based on capsular saccharides. Thus compositions of the invention can include at least one pneumococcal capsular saccharide conjugated to a carrier protein.

The invention can include capsular saccharide from one or more different pneumococcal serotypes. Where a composition includes saccharide antigens from more than one serotype, these are preferably prepared separately, conjugated separately, and then combined. Methods for purifying pneumococcal capsular saccharides are known in the art (e.g. see reference 19) and vaccines based on purified saccharides from 23 different serotypes have been known for many years. Improvements to these methods have also been described e.g. for serotype 3 as described in reference 20, or for serotypes 1, 4, 5, 6A, 6B, 7F and 19A as described in reference 21.Approximately 91 serotypes of capsular pneumococci have been identified. Examples of saccharides from these serotypes are provided in references 22-24 and chapters 22 & 23 of ref. 25. Serotypes 1, 2, 3, 4, 5, 6A 6B, 7F, 8, 9N, 9V, 10A, 11A, 12F, 14, 15B, 17F, 18C, 19A, 19F, 20, 22F, 23F, and 33F, are thought to account for 85-90% of invasive pneumococcal disease. The repeating units for the main S. pneumoniae serotypes are described in FIG. 1 and refs 26 and 27. As discussed above, existing pneumococcal vaccines are known to induce anti-capsular antibodies to the specific pneumococcal serotype included in the formulation, and so compositions of the invention ideally include saccharides from one or more of these main S. pneumoniae serotypes.

The saccharide is from the capsular saccharide of a pneumococcus. The saccharide may be a polysaccharide having the size that arises during purification of the saccharide from bacteria, or it may be an oligosaccharide achieved by fragmentation of such a polysaccharide. In the 7-valent PREVNAR™ product for instance, 6 of the saccharides are presented as intact polysaccharides while one (the 18C serotype) is presented as an oligosaccharide.

A composition may include a capsular saccharide from one or more of the following pneumococcal serotypes: 1, 2, 3, 4, 5, 6A, 6B, 7F, 8, 9N, 9V, 10A, 11A, 12F, 14, 15B, 17F, 18C, 19A, 19F, 20, 22F, 23F and/or 33F. A composition may include multiple serotypes e.g. 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more serotypes. A composition may include 2-10 or at least 12 different serotypes e.g. 2, 3, 4, 5, 6, 7, 8, 9, 10, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more serotypes.

Compositions which include 6B are useful. 7-valent, 9-valent, 10-valent, 11-valent and 13-valent conjugate combinations are already known in the art, as is a 23-valent unconjugated combination.

Preferably, saccharides from at least serotypes 6B, 14and 23F are used e.g. saccharides from serotypes 1, 5, 6B, 14, and 23F are used, or saccharides from serotypes 6B, 14, 19F and 23F are used. Further serotypes are preferably selected from one or more of serotypes 1, 3, 4, 5, 7F, 9V and 18C and/or serotypes 3, 6A and 19A. In some embodiments, 1 or more saccharides are omitted from these lists e.g. 1, 2, 3 etc. saccharides may be omitted.

A useful combination of serotypes is a 7 valent combination e.g. including capsular saccharide from each of serotypes 4, 6B, 9V, 14, 18C, 19F, and 23F. Another useful combination is a 9 valent combination e.g. including capsular saccharide from each of serotypes 1, 4, 5, 6B, 9V, 14, 18C, 19F and 23F. Another useful combination is a 10 valent combination e.g. including capsular saccharide from each of serotypes 1, 4, 5, 6B, 7F, 9V, 14, 18C, 19F and 23F. Another useful combination is a 10-valent combination may include saccharide from serotypes 1, 4, 5, 6B, 7F, 9V, 14, 18C, 19F and 23F. An 11-valent combination may further include saccharide from serotype 3. In some embodiments, there are not saccharides from 11 different serotypes. Where there are saccharides from 11 different serotypes, the saccharides are preferably not from serotypes 1, 3, 4, 5, 6B, 7F, 9V, 14, 18C, 19F and 23F.

A 12-valent combination may add to the 10-valent mixture: serotypes 6A and 19A; 6A and 22F; 19A and 22F; 6A and 15B; 19A and 15B;o r 22F and 15B; A 13-valent combination may add to the 11-valent mixture: serotypes 19A and 22F; 8 and 12F; 8 and 15B; 8 and 19A; 8 and 22F; 12F and 15B; 12F and 19A; 12F and 22F; 15B and 19A; 15B and 22F; 6A and 19A, etc. Thus, a useful 13-valent combination includes capsular saccharide from serotypes 1, 3, 4, 5, 6A, 6B, 7F, 9V, 14, 18C, 19 (or 19A), 19F and 23F e.g. prepared as disclosed in references 28 to 31. One such combination includes serotype 6B saccharide at about 8 μg/ml and the other 12 saccharides at concentrations of about 4 μg/ml each. Another such combination includes serotype 6A and 6B saccharides at about 8 μg/ml each and the other 11 saccharides at about 4 μg/ml each.

If saccharides are enclosed then it is preferred to include 1, 2 or 3 of serotypes 1, 5 and 14.

The composition optionally does not include diphtheria toxoid, tetanus toxoid and pertussis toxoid.

Suitable carrier proteins for conjugates include bacterial toxins, such as diphtheria or tetanus toxins, or toxoids or mutants thereof. These are commonly used in conjugate vaccines. For example, the CRM197 diphtheria toxin mutant is useful [32]. Other suitable carrier proteins include synthetic peptides [33,34], heat shock proteins [35,36], pertussis proteins [37,38], cytokines [39], lymphokines [39], hormones [39], growth factors [39], artificial proteins comprising multiple human CD4′ T cell epitopes from various pathogen-derived antigens [40] such as N19 [41], protein D from H.influenzae [42-44], pneumolysin [45] or its non-toxic derivatives [46], pneumococcal surface protein PspA [47], iron-uptake proteins [48], toxin A or B from C.difficile [49], recombinant Pseudomonas aeruginosa exoprotein A (rEPA) [50], etc.

Particularly useful carrier proteins for pneumococcal conjugate vaccines are CRM197, tetanus toxoid, diphtheria toxoid and H. influenzae protein D. CRM197 is used in PREVNAR™. A 13-valent mixture may use CRM197 as the carrier protein for each of the 13 conjugates, and CRM197 may be present at about 55-60μg/ml.

Where a composition includes conjugates from more than one pneumococcal serotype, it is possible to use the same carrier protein for each separate conjugate, or to use different carrier proteins. In both cases, though, a mixture of different conjugates will usually be formed by preparing each serotype conjugate separately, and then mixing them to form a mixture of separate conjugates. Reference 51 describes potential advantages when using different carrier proteins in multivalent pneumococcal conjugate vaccines, but the PREVNAR™ products successfully use the same carrier for each of seven different serotypes.

In some embodiments, a single conjugate may carry saccharides from multiple serotypes [52]. Usually, however, an individual conjugate will include saccharide from a single serotype.

Conjugates may have excess carrier (w/w) or excess saccharide (w/w). In some embodiments, a conjugate may include equal weights of each.

The carrier molecule may be covalently conjugated to the saccharide directly or via a linker. Various linkers are known. Direct linkages to the protein may be achieved by, for instance, reductive amination between the saccharide and the carrier, as described in, for example, references 53 and 54. The saccharide may first need to be activated e.g. by oxidation e.g. with periodate to introduce an aldehyde group, which can then form a direct covalent linkage to a carrier protein via reductive amination e.g. to the c amino group of a lysine. If the saccharide includes multiple aldehyde groups per molecule then this linkage technique can lead to a cross-linked product, where multiple aldehydes react with multiple carrier amines. This cross-linking conjugation technique is particularly useful for at least pneumococcal serotypes 4, 6B, 9V, 14, 18C, 19F and 23F. Linkages via a linker group may be made using any known procedure, for example, the procedures described in references 55 and 56. A preferred type of linkage is an adipic acid linker, which may be formed by coupling a free NH₂ group (e.g. introduced by amination) with adipic acid (using, for example, diimide activation), and then coupling a protein to the resulting saccharide-adipic acid intermediate [57,58]. Another preferred type of linkage is a carbonyl linker, which may be formed by reaction of a free hydroxyl group of a saccharide CDI [59, 60] followed by reaction with a protein to form a carbamate linkage. Other linkers include β-propionamido [61], nitrophenyl-ethylamine [62], haloacyl halides [63], glycosidic linkages [64], 6-aminocaproic acid [65], ADH [66], C₄ to C₁₂ moieties [67], etc. Carbodiimide condensation can also be used [68].

A pneumococcal saccharide may comprise a full length intact saccharide as prepared from pneumococcus, and/or may comprise fragments of full length saccharides i.e. the saccharides may be shorter than the native capsular saccharides seen in bacteria. The saccharides may thus be depolymerised, with depolymerisation occurring during or after saccharide purification but before conjugation. Depolymerisation reduces the chain length of the saccharides. Depolymerisation can be used in order to provide an optimum chain length for immunogenicity and/or to reduce chain length for physical manageability of the saccharides. Where more than one pneumococcal serotype is used then it is possible to use intact saccharides for each serotype, fragments for each serotype, or to use intact saccharides for some serotypes and fragments for other serotypes.

Where a composition includes saccharide from any of serotypes 4, 6B, 9V, 14, 19F and 23F, these saccharides are preferably intact. In contrast, where a composition includes saccharide from serotype 18C, this saccharide is preferably depolymerised.

A serotype 3 saccharide may also be depolymerised, For instance, a serotype 3 saccharide can be subjected to acid hydrolysis for depolymerisation [58] e.g. using acetic acid. The resulting fragments may then be oxidised for activation (e.g. periodate oxidation, maybe in the presence of bivalent cations e.g. with MgCl₂), conjugated to a carrier (e.g. CRM197) under reducing conditions (e.g. using sodium cyanoborohydride), and then (optionally) any unreacted aldehydes in the saccharide can be capped (e.g. using sodium borohydride) [58]. Conjugation may be performed on lyophilized material e.g. after co lyophilizing activated saccharide and carrier.

A serotype 1 saccharide may be at least partially de-O-acetylated e.g. achieved by alkaline pH buffer treatment [59] such as by using a bicarbonate/carbonate buffer. Such (partially) de O acetylated saccharides can be oxidised for activation (e.g. periodate oxidation), conjugated to a carrier (e.g. CRM197) under reducing conditions (e.g. using sodium cyanoborohydride), and then (optionally) any unreacted aldehydes in the saccharide can be capped (e.g. using sodium borohydride) [59]. Conjugation may be performed on lyophilized material e.g. after co lyophilizing activated saccharide and carrier.

A serotype 19A saccharide may be oxidised for activation (e.g. periodate oxidation), conjugated to a carrier (e.g. CRM197) in DMSO under reducing conditions, and then (optionally) any unreacted aldehydes in the saccharide can be capped (e.g. using sodium borohydride) [ ]. Conjugation may be performed on lyophilized material e.g. after co lyophilizing activated saccharide and carrier.

One or more pneumococcal capsular saccharide conjugates may be present in lyophilised form.

Pneumococcal conjugates can ideally elicit anticapsular antibodies that bind to the relevant saccharide e.g. elicit an anti-saccharide antibody level >0.20 μg/mL [ ]. The antibodies may be evaluated by enzyme immunoassay (EIA) and/or measurement of opsonophagocytic activity (OPA). The EIA method has been extensively validated and there is a link between antibody concentration and vaccine efficacy.

The concentration of a pneumococcal conjugate, measured as saccharide, is typically between 0.01 and 50 μg/ml for each serotype; preferably between 0.1 and 40 μg/ml; more preferably between 0.5 and 30 μg/ml; most preferably between 1 and 25 μg/ml, such as between 2 and 20 μg/ml for each serotype e.g. 2, 3, 4, 5, 6, 7, 8, 9, 10, 11, 12, 13, 14, 15, 16, 17, 18, 19 or 20 μg/ml for each serotype. In compositions comprising a mixture of saccharides from different serotypes, the amount of each saccharide in the mixture is typically approximately the same. As an alternative, a composition comprising a mixture of saccharides from different serotypes comprises different amounts of each saccharide e.g. a higher amount of saccharide(s) may be used if it is less immunogenic than other saccharides in the mixture.

Pneumococcal saccharide antigen(s) described herein may be combined with one or more pneumococcal protein antigens. Thus the invention provides an immunogenic composition comprising (i) a TLR agonist; (ii) an insoluble metal salt; (iii) one or more S. pneumoniae protein antigen(s), preferably as a mixture or hybrid; and (iv) one or more pneumococcal capsular saccharides described herein.

Pilus Antigens

When a composition includes one or more S. pneumoniae protein antigen(s), the preferred protein antigen(s) is/are a pilus antigen. Many strains of S. pneumoniae possess a pilus, encoded within a pathogenicity islet (rlrA). The islet encodes three surface proteins (RrgA, RrgB, and RrgC) and three sortase enzymes. In some embodiments of the invention, a composition will include, in addition to an antigen from one of the groups of the invention, one or more of: RrgA; RrgB; RrgC; SrtB; SrtC; and/or SrtD. Of these six proteins, including one or more of RrgA, RrgB and/or RrgC is preferred. RrgB is the most preferred pilus protein to be included.

Some strains possess a different pilus type [69], TI-2′. The PI-2 operon encodes PitA, SipA, PitB, SrtG1, and SrtG2. In some embodiments of the invention, a composition will include, in addition to an antigen from one of the groups of the invention, one or more of: PitA, SipA, PitB, SrtG1, and/or SrtG2.

RrgA is one of the surface subunits of the pneumococcal pilus [70,71] and is an important adhesin [72].There are at least two allelic forms of RrgA and, for reference purposes, their amino acid sequences are SEQ ID NOs: 172 and 179 herein. The two alleles are well conserved at their N- and C-termini but deviate in between.

Preferred RrgA polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 172; and/or (b) comprising a fragment of at least ‘n’ consecutive amino acids of SEQ ID NO: 172, wherein ‘n’ is 7 or more (e.g. 8, 10, 12, 14, 16, 18, 20, 25, 30, 35, 40, 50, 60, 70, 80, 90, 100, 150, 200, 250 or more). These RrgA proteins include variants of SEQ ID NO: 172. Preferred fragments of (b) comprise an epitope from SEQ ID NO: 172. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 172 while retaining at least one epitope of SEQ ID NO: 172. Other fragments omit one or more protein domains. One suitable fragment is SEQ ID NO: 192, which omits the natural leader peptide and sortase recognition sequences.

Other preferred RrgA polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 179; and/or (b) comprising a fragment of at least ‘n’ consecutive amino acids of SEQ ID NO: 179, wherein ‘n’ is 7 or more (e.g. 8, 10, 12, 14, 16, 18, 20, 25, 30, 35, 40, 50, 60, 70, 80, 90, 100, 150, 200, 250 or more). These RrgA proteins include variants of SEQ ID NO: 179. Preferred fragments of (b) comprise an epitope from SEQ ID NO: 179. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 179 while retaining at least one epitope of SEQ ID NO: 179. Other fragments omit one or more protein domains. One suitable fragment is SEQ ID NO: 191, which omits the natural leader peptide and sortase recognition sequences.

RrgB is one of the surface subunits of the pneumococcal pilus [70]. There are at least three allelic forms of RrgB and, for reference purposes, their amino acid sequences are SEQ ID NOs: 236, 237 and 238 herein. The three alleles are well conserved at their N- and C-termini but deviate in between.

Preferred RrgB polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 236; and/or (b) comprising a fragment of at least ‘n’ consecutive amino acids of SEQ ID NO: 236, wherein ‘n’ is 7 or more (e.g. 8, 10, 12, 14, 16, 18, 20, 25, 30, 35, 40, 50, 60, 70, 80, 90, 100, 150, 200, 250 or more). These RrgB proteins include variants of SEQ ID NO: 236. Preferred fragments of (b) comprise an epitope from SEQ ID NO: 236. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 236 while retaining at least one epitope of SEQ ID NO: 236. Other fragments omit one or more protein domains.

Other preferred RrgB polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 237; and/or (b) comprising a fragment of at least ‘n’ consecutive amino acids of SEQ ID NO: 237, wherein ‘n’ is 7 or more (e.g. 8, 10, 12, 14, 16, 18, 20, 25, 30, 35, 40, 50, 60, 70, 80, 90, 100, 150, 200, 250 or more). These RrgB proteins include variants of SEQ ID NO: 237. Preferred fragments of (b) comprise an epitope from SEQ ID NO: 237. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 237 while retaining at least one epitope of SEQ ID NO: 237. Other fragments omit one or more protein domains.

Other preferred RrgB polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 238; and/or (b) comprising a fragment of at least ‘n’ consecutive amino acids of SEQ ID NO: 238, wherein ‘n’ is 7 or more (e.g. 8, 10, 12, 14, 16, 18, 20, 25, 30, 35, 40, 50, 60, 70, 80, 90, 100, 150, 200, 250 or more). These RrgB proteins include variants of SEQ ID NO: 238. Preferred fragments of (b) comprise an epitope from SEQ ID NO: 238. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 238 while retaining at least one epitope of SEQ ID NO: 238. Other fragments omit one or more protein domains.

RrgC is one of the surface subunits of the pneumococcal pilus [70]. For reference purposes, the amino acid sequence of RrgC is SEQ ID NO: 176 herein.

Preferred RrgC polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 176; and/or (b) comprising a fragment of at least ‘n’ consecutive amino acids of SEQ ID NO: 176, wherein ‘n’ is 7 or more (e.g. 8, 10, 12, 14, 16, 18, 20, 25, 30, 35, 40, 50, 60, 70, 80, 90, 100, 150, 200, 250 or more). These RrgC proteins include variants of SEQ ID NO: 176. Preferred fragments of (b) comprise an epitope from SEQ ID NO: 176. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 176 while retaining at least one epitope of SEQ ID NO: 176. Other fragments omit one or more protein domains.

As discussed in reference 73, and below the RrgB protein has four domains, D1, D2, D3 and D4. Immunisation with full length RrgB or with separate domains of RrgB was shown in reference 236 to provide protection in active immunisation experiments. The D1 and D4 domains were shown to provide the most significant protective efficacy and the epitopes identified in these domains are expected to be involved in the protective mechanism.

The RrgB pilus subunit has at least three clades. Reference amino acid sequences for the three full length clades are SEQ ID NOs: 236, 237 and 238 herein. The clades are well conserved at their N- and C-termini but deviate in between. SEQ ID NOs: 236 and 237 are 46% identical; SEQ ID NOs: 236 and 238 are 51% identical; SEQ ID NOs: 237 and 238 are 65% identical. Epitopes have been identified at residue numbers 40 to 59 of the D1 domain and at residue numbers 494 to 508 of the D4 domain. The epitopes in each of the three clades are identified in the following table:

| Full Length | Epitope in | Epitope |
|---|---|---|
| Sequence (Clade) | D1 Domain | in D4 Domain |
| SEQ ID | HKLLATDGDMDKIANELETG | VTTKDALDRAVAAYN |
| NO.236 (I) | (SEQ ID NO: 335) | (SEQ ID NO: 336) |
| SEQ ID | VTKTLTIHKLLLSEDDLKTW | YTNAADKQAAQALVD |
| NO.237 (II) | (SEQ ID NO: 337) | (SEQ ID NO: 338) |
| SEQ ID | HKLLMTDQELDAWNSDAITT | KQALDAAIAAYTNAA |
| NO.238 (III) | (SEQ ID NO: 339) | (SEQ ID NO: 340) |

The identification of these epitopes allowed immunogenic compositions to be provided that do not contain the full length RrgB sequence, and instead contain fragments comprising the identified epitopes. These smaller fragments may be easier to produce and administer for therapeutic benefit, but retain the ability to generate an immune response against the full length RrgB protein.

Thus, a first aspect the invention provides an immunogenic composition comprising:
(a) a first amino acid sequence, where the first amino acid sequence comprises or consists of: SEQ ID NO.335, or an amino acid sequence having at least a⁶X9 sequence identity to SEQ ID NO: 335, or an amino acid sequence that competes with SEQ ID NO.335 for binding to an antibody raised against SEQ ID NO.335, or a fragment of at least u contiguous amino acids from SEQ ID NO.335; and/or
(b) a second amino acid sequence, where the second amino acid sequence comprises or consists of: SEQ ID NO.336, or an amino acid sequence having at least b⁶X9 sequence identity to SEQ ID NO: 336, or an amino acid sequence that competes with SEQ ID NO.336 for binding to an antibody raised against SEQ ID NO.336, or an a fragment of at least v contiguous amino acids from SEQ ID NO.336; and/or
(c) a third amino acid sequence, where the third amino acid sequence comprises or consists of: SEQ ID NO.337, or an amino acid sequence having at least c%^{,} sequence identity to SEQ ID NO: 337, or an amino acid sequence that competes with SEQ ID NO.337 for binding to an antibody raised against SEQ ID NO.337, or a fragment of at least w contiguous amino acids from SEQ ID NO.337; and/or
(d) a fourth amino acid sequence, where the fourth amino acid sequence comprises or consists of: SEQ ID NO.338, or an amino acid sequence having at least d%, sequence identity to SEQ ID NO: 338, or an amino acid sequence that competes with SEQ ID NO.338 for binding to an antibody raised against SEQ ID NO.338, or a fragment of at least x contiguous amino acids from SEQ ID NO.338; and/or
(e) a fifth amino acid sequence, where the fifth amino acid sequence comprises or consists of: SEQ ID NO.339, or an amino acid sequence having at least e%, sequence identity to SEQ ID NO: 339, or an amino acid sequence that competes with SEQ ID NO.339 for binding to an antibody raised against SEQ ID NO.339, or a fragment of at least y contiguous amino acids from SEQ ID NO.339; and/or
(f) a sixth amino acid sequence, where the sixth amino acid sequence comprises or consists of: SEQ ID NO.340, or an amino acid sequence having at least f% sequence identity to SEQ ID NO: 340, or an amino acid sequence that competes with SEQ ID NO.340 for binding to an antibody raised against SEQ ID NO.340, or a fragment of at least z contiguous amino acids from SEQ ID NO.340.

Serum raised against a given RrgB Glade is active against pneumococci which express that Glade, but is not active against strains which express one of the other two clades i.e. there is intra-clade cross-protection, but not inter-Glade cross-protection. According to one embodiment of the invention, therefore, an immunogenic composition includes epitopes from at least two different clades of RrgB. As detailed in the Table above, SEQ ID NOs. 335 and 336 are from a first Glade, SEQ ID NOs. 337 and 338 are from a second Glade and SEQ ID NOs. 339 and 340 are from a third Glade. An epitope identified above may be combined with an epitope, or a longer sequence containing multiple epitopes, from a different Glade. The different clades may be present in the immunogenic composition as separate polypeptides or may be fused as a single polypeptide chain. The inclusion of multiple RrgB clades as vaccine components improves the strain coverage of the immunogenic composition against pilus-containing pneumococci. Furthermore, it has been observed that there is a significant association between pilus-1 presence and antibiotic resistance; this observation suggests that immunising against pilus-1 using an immunogenic composition including multiple RrgB clades will have the additional advantage of protecting against pneumococci that are resistant to antibiotic treatment.

Thus the invention provides a polypeptide comprising a first, second, third, fourth, fifth and/or sixth amino acid sequence as defined above in the first aspect.

The invention also provides a polypeptide comprising amino acid sequence:

-A-{-X-L}ₙ-B-

wherein: X is an amino acid sequence of first amino acid sequence, second amino acid sequence, third amino acid sequence, fourth amino acid sequence, fifth amino acid sequence or sixth amino acid sequence as defined above; L is an optional linker amino acid sequence; A is an optional N-terminal amino acid sequence; B is an optional C-terminal amino acid sequence; n is an integer of 2 or more (e.g. 2, 3, 4, 5, 6, etc.). Optionally, the polypeptide comprises at least two of a first, second third, fourth, fifth and sixth amino acid sequence as defined above. Usually n is 2 or 3, and X moieties are selected from the following:

| N | X₁ | X₂ | X₃ |
|---|---|---|---|
| 2 | First or Second amino | Third or Fourth amino | — |
| | acid sequence | acid sequence | |
| 2 | Third or Fourth amino | First or Second amino | — |
| | acid sequence | acid sequence | |
| 3 | First or Second amino | Third or Fourth amino | Fifth or Sixth amino |
| | acid sequence | acid sequence | acid sequence |
| 3 | First or Second amino | Fifth or Sixth amino | Third or Fourth amino |
| | acid sequence | acid sequence | acid sequence |
| 3 | Third or Fourth amino | Fifth or Sixth amino | First or Second amino |
| | acid sequence | acid sequence | acid sequence |
| 3 | Third or Fourth amino | First or Second amino | Fifth or Sixth amino |
| | acid sequence | acid sequence | acid sequence |
| 3 | Fifth or Sixth amino | Third or Fourth amino | First or Second amino |
| | acid sequence | acid sequence | acid sequence |
| 3 | Fifth or Sixth amino | First or Second amino | Third or Fourth amino |
| | acid sequence | acid sequence | acid sequence |

In each of the combinations exemplified in the table above, the two alternatives for each instance of X_{1,} X₂ and X₃ can optionally be combined, so that both of the recited alternative amino acid sequences are administered. For example, in the first line of the Table, X₁ could contain the first and second amino acid sequence, and/or X₂ could contain the third and fourth amino acid sequence.

The invention also provides a cell (typically a bacterium, such as a pneumococcus) which expresses a first, second, third, fourth, fifth and/or sixth amino acid sequence as defined above in the first aspect.

The First, Second, Third, Fourth, Fifth and Sixth Amino Acid Sequences

The value of a is at least 75 e.g. 80, 85, 90, 92, 94, 95, 96, 97, 98, 99 or more. The value of b is at least 75 e.g. 80, 85, 90, 92, 94, 95, 96, 97, 98, 99 or more. The value of c is at least 75 e.g. 80, 85, 90, 92, 94, 95, 96, 97, 98, 99 or more. The value of d is at least 75 e.g. 80, 85, 90, 92, 94, 95, 96, 97, 98, 99 or more. The value of e is at least 75 e.g. 80, 85, 90, 92, 94, 95, 96, 97, 98, 99 or more. The value off is at least 75 e.g. 80, 85, 90, 92, 94, 95, 96, 97, 98, 99 or more. The values of a, b, c, d, e and f may be the same or different. In some embodiments, a, b, c, d, e andfare identical. Typically, a, b, c, d, e andfare at least 90 e.g. at least 95.

The value of tt is at least 7 e.g. 8, 9, 10, 11, 12, 13, 14, 15, 16, 17, 18, or 19. The value of v is at least 7 e.g. 8, 9, 10, 11, 12, 13 or 14. The value of w is at least 7 e.g. 8, 9, 10, 11, 12, 13, 14, 15, 16, 17, 18, or 19.The value of x is at least 7 e.g. 8, 9, 10, 11, 12, 13, or 14. The value of y is at least 7 e.g. 8, 9, 10, 11, 12, 13, 14, 15, 16, 17, 18, or 19. The value of z is at least 7 e.g. 8, 9, 10, 11, 12, 13, or 14. The values of u,v,w,x, y and z may be the same or different. In some embodiments, u,v,w,x, y and z are identical.

Fragments preferably comprise an epitope from the respective SEQ ID NO: sequence. Other useful fragments lack one or more amino acids (e.g. 1, 2, 3, 4, 5, 6, 7, 8, 9, 10, 15, or more) from the C-terminus and/or one or more amino acids (e.g. 1, 2, 3, 4, 5, 6, 7, 8, 9, 10, 15 or more) from the N-terminus of the respective SEQ ID NO: while retaining at least one epitope thereof. Truncation by 1-5 amino acids at the N-terminus is convenient e.g. removal of aa 1-5 of any of SEQ ID NOs: 335 to 340.

First, second, third, fourth, fifth and sixth polypeptides comprises or consists of the first, second, third, fourth, fifth and/or sixth amino acid sequences, respectively. These polypeptides can consist of, i.e. contain only, the respective amino acid sequence or can contain additional amino acid residues or sequences. Typically, each of the first, second, third, fourth, fifth and sixth polypeptides consists of 50 or fewer, 45 or fewer, 40 or fewer, 35 or fewer, 34 or fewer, 33 or fewer, 30 or fewer, or 25 or fewer amino acid residues.

The RrgB protein can be split into four domains (D1 to D4) between its leader peptide and its LPXTG anchor. These four domains are as follows in SEQ ID NOs: 236 to 238, and the positions in further RrgB sequences which correspond to these residues can readily be identified by alignment:

| | D1 | D2 | D3 | D4 |
|---|---|---|---|---|
| 1 | 31-184 | 185-326 | 327-446 | 447-627 |
| 2 | 31-185 | 186-318 | 319-434 | 435-606 |
| 3 | 31-184 | 185-319 | 320-445 | 446-616 |

Based on passive protection studies, useful fragments of RrgB may retain epitopes from at least domains D1 and/or D4. As shown in reference 236, antibodies have been raised that bind to domain D1, domain D4 and a fragment containing domains D2 to D4.

A polypeptide comprising the first or second amino acid sequence will, when administered to a subject, elicit an antibody response comprising antibodies that bind to the wild-type pneumococcus protein having amino acid sequence SEQ ID NO: 236 (strain TIGR4). In some embodiments these antibodies do not bind to the wild-type pneumococcus protein having amino acid sequence SEQ ID NO: 237 or to the wild-type pneumococcus protein having amino acid sequence SEQ ID NO: 238.

A polypeptide comprising the third or fourth amino acid sequence will, when administered to a subject, elicit an antibody response comprising antibodies that bind to the wild-type pneumococcus protein having amino acid sequence SEQ ID NO: 237 (strain Finland^{6B}-12). In some embodiments these antibodies do not bind to the wild-type pneumococcus protein having amino acid sequence SEQ ID NO: 236 or to the wild-type pneumococcus protein having amino acid sequence SEQ ID NO: 238.

A polypeptide comprising the fifth or sixth amino acid sequence will, when administered to a subject, elicit an antibody response comprising antibodies that bind to the wild-type pneumococcus protein having amino acid sequence SEQ ID NO: 238 (strain Taiwan^{23F}-15). In some embodiments these antibodies do not bind to the wild-type pneumococcus protein having amino acid sequence SEQ ID NO: 236 or to the wild-type pneumococcus protein having amino acid sequence SEQ ID NO: 237.

Although the first, third and fifth amino acid sequences may share some sequences in common, overall they have different amino acid sequences. Similarly, although the second, fourth and sixth amino acid sequences may share some sequences in common, overall they have different amino acid sequences.

Where the invention uses epitopes from only two RrgB clades a composition or polypeptide can include both: (a) a first or second amino acid sequence as defined above; and (b) a third or fourth amino acid sequence as defined above. In an alternative embodiment the composition includes both: (a) a first or second amino acid sequence as defined above; and (b) a fifth or sixth amino acid sequence as defined above. In an alternative embodiment the composition includes both: (a) a third or fourth amino acid sequence as defined above; and (b) a fifth or sixth amino acid sequence as defined above.

Amino acid sequences used with the invention, may, compared to SEQ ID NOs: 335, 336, 337, 338, 339 or 340 include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may have one or more (e.g. 1, 2, 3, 4, 5, 6, 7, 8, 9, 10, etc.) single amino acid deletions relative to a reference sequence. The polypeptides may also include one or more (e.g. 1, 2, 3, 4, 5, 6, 7, 8, 9, 10, etc.) insertions (e.g. each of 1, 2, 3, 4 or 5 amino acids) relative to a reference sequence.

A polypeptide used with the invention may comprise an amino acid sequence that:
(a) is identical (i.e. 100% identical) to SEQ ID NO: 335, 336, 337, 338, 339 or 340;
(b) shares sequence identity SEQ ID NO: 335, 336, 337, 338, 339 or 340;
(c) has 1, 2, 3, 4, 5, 6, 7, 8, 9 or 10 (or more) single amino acid alterations (deletions, insertions, substitutions), which may be at separate locations or may be contiguous, as compared to the sequences of (a) or (b); and
(d) when aligned SEQ ID 335, 336, 337, 338, 339 or 340 using a pairwise alignment algorithm, each moving window of x amino acids from N-terminus to C-terminus (such that for an alignment that extends to p amino acids, where p>x, there are p-x+1 such windows) has at least xy identical aligned amino acids, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if xy is not an integer then it is rounded up to the nearest integer. The preferred pairwise alignment algorithm is the Needleman-Wunsch global alignment algorithm [74], using default parameters (e.g. with Gap opening penalty=10.0, and with Gap extension penalty=0.5, using the EBLOSUM62 scoring matrix). This algorithm is conveniently implemented in the needle tool in the EMBOSS package [75].

Within group (c), deletions or substitutions may be at the N-terminus and/or C-terminus, or may be between the two termini. Thus a truncation is an example of a deletion. Truncations may involve deletion of up to 5 (or more) amino acids at the N-terminus and/or C-terminus.

In general, when a polypeptide of the invention comprises a sequence that is not identical to a complete pneumococcal epitope sequence from SEQ ID NOs: 335, 336, 337, 338, 339 or 340 (e.g.

when it comprises a sequence listing with <100% sequence identity thereto, or when it comprises a fragment thereof), it is preferred in each individual instance that the polypeptide can elicit an antibody that recognises the complete pneumococcal sequence.

For reference, SEQ ID NOs: 236 to 238 and 320 to 331 are 15 unique RrgB sequences which have been identified in 45 different strains. Any of these sequences can be used for implementing the invention.

Other Pneumococcal Protein Antigens

Other S. pneumoniae antigens may be used with the invention, either as individual antigens, or in combinations e.g. in combination with RrgB antigen(s) described herein.

Preferred combinations of pneumococcal polypeptides have been identified. For example, a preferred combination of protein antigens is the following 7 pneumococcal polypeptides: spr0057; spr0286; spr0565; spr1098; spr1345; spr1416; spr1418. This set of antigens is referred to herein as ‘the first antigen group’. Thus the invention provides an immunogenic composition comprising a combination of S. pneumoniae antigens, said combination comprising two or more (i.e. 2, 3, 4, 5, 6 or all 7) antigens selected from the group consisting of: (1) a spr0057 antigen; (2) a spr0286 antigen; (3) a spr0565 antigen; (4) a spr1098 antigen; (5) a spr1345 antigen; (6) a spr1416 antigen; and/or (7) a spr1418 antigen.

Another preferred combination of protein antigens is the following 4 pneumococcal polypeptides: spr0867; spr1431; spr1739; spr2021. This set of antigens is referred to herein as ‘the second antigen group’. Thus the invention provides an immunogenic composition comprising a combination of S. pneumoniae antigens, said combination comprising two or more (i.e. 2, 3 or all 4) antigens selected from the group consisting of: (1) a spr0867 antigen; (2) a spr1431 antigen; (3) a spr1739 antigen; and/or (4) a spr2021 antigen.

Another preferred combination of protein antigens is the following 3 pneumococcal polypeptides: spr0096; spr1433; spr1707. This set of antigens is referred to herein as ‘the third antigen group’. Thus the invention provides an immunogenic composition comprising a combination of S. pneumoniae antigens, said combination comprising two or three antigens selected from the group consisting of: (1) a spr0096 antigen; (2) a spr1433 antigen; and/or (3) a spr1707 antigen.

The combination of 11 pneumococcal polypeptides in the first and second antigen groups is referred to herein as ‘the fourth antigen group’. Thus the invention provides an immunogenic composition comprising a combination of S. pneumoniae antigens, said combination comprising two or more (i.e. 2, 3, 4, 5, 6, 7, 8, 9, 10 or all 11) antigens selected from the group consisting of: (1) a spr0057 antigen; (2) a spr0286 antigen; (3) a spr0565 antigen; (4) a spr1098 antigen; (5) a spr1345 antigen;

(6) a spr1416 antigen; (7) a spr1418 antigen; (8) a spr0867 antigen; (9) a spr1431 antigen; (10) a spr1739 antigen; and/or (11) a spr2021 antigen.

The combination of 10 pneumococcal polypeptides in the first and third antigen groups is referred to herein as ‘the fifth antigen group’. Thus the invention provides an immunogenic composition comprising a combination of S. pneumoniae antigens, said combination comprising two or more (i.e. 2, 3, 4, 5, 6, 7, 8, 9, or all 10) antigens selected from the group consisting of: (1) a spr0057 antigen; (2) a spr0286 antigen; (3) a spr0565 antigen; (4) a spr1098 antigen; (5) a spr1345 antigen; (6) a spr1416 antigen; (7) a spr1418 antigen; (8) a spr0096 antigen; (9) a spr1433 antigen; and/or (10) a spr1707 antigen.

The combination of 7 pneumococcal polypeptides in the second and third antigen groups is referred to herein as ‘the sixth antigen group’. Thus the invention provides an immunogenic composition comprising a combination of S. pneumoniae antigens, said combination comprising two or more (i.e. 2, 3, 4, 5, 6, or all 7) antigens selected from the group consisting of: (1) a spr0867 antigen; (2) a spr1431 antigen; (3) a spr1739 antigen; (4) a spr2021 antigen; (5) a spr0096 antigen; (6) a spr1433 antigen; and/or (7) a spr1707 antigen.

The combination of 14 pneumococcal polypeptides in the first, second and third antigen groups is referred to herein as ‘the seventh antigen group’. Thus the invention provides an immunogenic composition comprising a combination of S. pneumoniae antigens, said combination comprising two or more (i.e. 2, 3, 4, 5, 6, 7, 8, 9, 10, 11, 12, 13, or all 14) antigens selected from the group consisting of: (1) a spr0057 antigen; (2) a spr0286 antigen; (3) a spr0565 antigen; (4) a spr1098 antigen; (5) a spr1345 antigen; (6) a spr1416 antigen; (7) a spr1418 antigen; (8) a spr0867 antigen; (9) a spr1431 antigen; (10) a spr1739 antigen; (11) a spr2021 antigen; (12) a spr0096 antigen; (13) a spr1433 antigen; and/or (14) a spr1707 antigen.

Within the seventh antigen group, a preferred subset of four antigens is the ‘eighth antigen group’, which includes an antigen from each of the first, second and third groups, namely spr0057, spr0096, spr0565 and spr2021. Thus the invention provides an immunogenic composition comprising a combination of S. pneumoniae antigens, said combination comprising two or more (i.e. 2, 3 or all 4) antigens selected from the group consisting of: (1) a spr0057 antigen; (2) a spr0096 antigen; (3) a spr0565 antigen; and/or (4) a spr2021 antigen. Within this eighth group, the composition may comprise: (1), (2) and (3); (1), (2) and (4); (1), (3) and (4); (2), (3) and (4); or (1), (2), (3) and (4). Expression of these four antigens has been immunologically confirmed across a panel of 32 pneumococcal strains with various serotypes.

The ‘ninth antigen group’ is the eighth antigen group plus a RrgB pilus antigen. Thus the invention also provides an immunogenic composition comprising a combination of S. pneumoniae antigens, said combination comprising one or more RrgB pilus antigen(s) and two or more (i.e. 2, 3 or all 4) antigens selected from the group consisting of: (1) a spr0057 antigen; (2) a spr0096 antigen; (3) a spr0565 antigen; and/or (4) a spr2021 antigen.

The ‘tenth antigen group’ is the eighth antigen group plus a Pmp polypeptide. Thus the invention also provides an immunogenic composition comprising a combination of S. pneumoniae antigens, said combination comprising two or more (i.e. 2, 3, 4 or all 5) antigens selected from the group consisting of: (1) a spr0057 antigen; (2) a spr0096 antigen; (3) a spr0565 antigen; (4) a spr2021 antigen; and/or (5) a Pmp polypeptide.

Specific combinations of interest comprise: (i) a spr0057 antigen and a spr0096 antigen; (ii) a spr0057 antigen and a spr2021 antigen; (iii) a spr0057 antigen, a spr0096 antigen and a spr2021 antigen; (iv) a spr0057 antigen and a spr0565 antigen; (v) a spr0565 antigen and a spr2021 antigen; (vi) a spr0057 antigen, a spr0565 antigen and a spr2021 antigen; (vii) a spr0565 antigen, a spr2021 antigen and a spr1739 antigen e.g. detoxified; and (viii) a spr0565 antigen, a spr2021 antigen and a Pmp polypeptide.

Advantageous combinations are those in which two or more antigens act synergistically. Thus the protection against pneumococcal disease achieved by their combined administration exceeds that expected by mere addition of their individual protective efficacy.

In additions to antigens from the various antigen groups, immunogenic compositions may include one or more of the following polypeptides to enhance the anti-pneumococcal immune response elicited by the composition:
One or more subunits of a pneumococcal pilus, such as RrgA, RrgB and/or RrgC.
A ClpP polypeptide.
A LytA polypeptide.
A CPL1 polypeptide.
A PhtA polypeptide.
A PhtB polypeptide.
A PhtD polypeptide.
A PhtE polypeptide.
A CbpD polypeptide
A CbpG polypeptide
A PvaA polypeptide.
A Hic polypeptide.
A Pmp polypeptide.
A ZmpB polypeptide.
A PspA polypeptide
A PsaA polypeptide
A PspC polypeptide.
A PrtA polypeptide.
A Sp91 polypeptide.
A Sp133 polypeptide.
A PiuA polypeptide and/or a PiaA polypeptide.
A spr0222 polypeptide.
An antigen selected from the group consisting of: IC1; IC2; IC3; IC4; IC5; IC6; IC7; IC8; IC9; IC10; IC11; IC12; IC13; IC14; IC15; IC16; IC17; IC18; IC19; IC20; IC21; IC22; IC23; IC24; IC25; IC26; IC27; IC28; IC29; IC30; IC31; IC32; IC33; IC34; IC35; IC36; IC37; IC38; IC39; IC40; IC41; IC42; IC43; IC44; IC45; IC46; IC47; IC48; IC49; IC50; IC51; IC52; IC53; IC54; IC55; IC56; IC57; IC58; IC59; IC60; IC61; IC62; IC63; IC64; IC65; IC66; IC67; IC68; IC69; IC70; IC71; IC72; IC73; IC74; IC75; IC76; IC77; IC78; IC79; IC80; IC81; IC82; IC83; IC84; IC85; IC86; IC88; IC89; IC90; IC91; IC92; IC93; IC94; IC95; IC96; IC97; IC98; IC99; IC100; IC 101; IC102; IC103; IC104; IC105; IC106; IC107; IC108; IC109; IC 110; IC111; IC112; IC113; IC114; IC115; IC116; IC117; IC118; IC119; IC120; IC121; IC122; IC123; IC124; IC125; IC126; IC127; IC128; IC129; IC130; and IC131.
An antigen selected from the group consisting of: ID-204, ID-212, ID-213, ID-214, ID-215, ID-216, ID-217, ID-219, ID-220, ID-225, ID-301, ID-302, ID-303, ID-304, ID-305, ID-306, as disclosed in reference 76.
An antigen selected from the group consisting of: SitlA, Sit1B, Sit1C, Sit2B, Sit2C, Sit2D, Sit3A, Sit3B, Sit3C, Sit3D, ORF1, ORF2, ORF3, ORF4, ORF5, ORF6, ORF6, ORF7, ORF8, ORF9, ORF10, ORF11, ORF12, ORF13, ORF14, MS1, MS2, MS3, MS4, MS5, MS6, MS7, MS8, MS9, MS10 or MS11, as disclosed in reference 77.
An antigen disclosed in reference 78.
An antigen disclosed in Tables 1-3 of reference 79, such as CbiO.
An antigen disclosed in reference 80, such as the 30S ribosomal protein S8.
An antigen selected from the group consisting of: a phosphoenolpyruvate protein phosphotransferase; a phosphomannomutase; a trigger factor; an elongation factor G; a tetracycline resistance protein (tet0); a DNA-directed RNA polymerase alpha-chain; a NADH oxidase; a glutamyl-tRNA amidotransferase subunit A; a N utilization substance protein A homolog; a Xaa-His dipeptidase; a cell division protein ftsz; a zinc metalloproteinase; a L-lactate dehydrogenase; a glyceraldehyde 3-phosphate dehydrogenase (GAPDH); a fructose-biphosphate aldolase; a UDP-glucose 4-epimerase; a GTP binding protein typA/BipA a GMP synthase; a glutamyl-tRNA synthetase; a NADP-specific glutamate dehydrogenase; an elongation factor TS; a phosphoglycerate kinase; a pyridine nucleotide-disulfide oxido-reductase; a 40S ribosomal protein Si; a 6-phosphogluconate dehydrogenase; an aminopeptidase C; a carbomyl-phosphate synthase (large subunit); a PTS system mannose-specific IIAB component; a ribosomal protein S2; a dihydroorotate dehydrogenase; an aspartate carbamoyltransferase; an elongation factor Tu; a pneumococcal surface immunogenic protein A (PsipA); a phosphogycerate kinase; an ABC transporter substrate-binding protein endopeptidase O; a pneumococcal surface immunogenic protein B (PsipB); or a pneumococcal surface immunogenic protein C (PsipC) [81].

IC1 to IC131

In some embodiments a composition will include one or more antigens selected from the group consisting of IC1 to IC131, e.g. in addition to an antigen from one of the antigen groups above. These 131 polypeptides (see below) are disclosed in reference 82, being the 144 polypeptides of Table 3 therein except for those listed as SP0117, SP0641, SP0664, SP1003, SP1004, SP1174, SP1175, SP1573, SP1687, SP1693, SP1937 and SP2190. Within the 132 polypeptides IC1 to IC131, a preferred subset from which the one or more polypeptide(s) may be selected is: IC1; IC8; IC16; IC23; IC31; IC34; IC40; IC45; IC47; IC57; IC58; IC60; and IC69.

spr0057

The original ‘spr0057’ sequence was annotated in reference 205 as ‘Beta-N-acetyl-hexosaminidase precursor’ (see GI:15902101). For reference purposes, the amino acid sequence of full length spr0057 as found in the R6 strain is given as SEQ ID NO: 1 herein.

Preferred spr0057 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 1; and/or (b) comprising a fragment of at least ‘n’ consecutive amino acids of SEQ ID NO: 1, wherein ‘n’ is 7 or more (e.g. 8, 10, 12, 14, 16, 18, 20, 25, 30, 35, 40, 50, 60, 70, 80, 90, 100, 150, 200, 250 or more). These spr0057 proteins include variants of SEQ ID NO: 1. Preferred fragments of (b) comprise an epitope from SEQ ID NO: 1. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 1 while retaining at least one epitope of SEQ ID NO: 1. Other fragments omit one or more protein domains. One suitable fragment is SEQ ID NO: 180, which omits the natural leader peptide and sortase recognition sequences.

Combinations of spr0057 with other pneumococcal antigens have shown good synergistic effects.

spr0286

The original ‘spr0286’ sequence was annotated in reference 205 as ‘Hyaluronate lyase precursor’ (see GI:15902330). For reference purposes, the amino acid sequence of full length spr0286 as found in the R6 strain is given as SEQ ID NO: 2 herein.

Preferred spr0286 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 2; and/or (b) comprising a fragment of at least ‘n’ consecutive amino acids of SEQ ID NO: 2, wherein ‘n’ is 7 or more (e.g. 8, 10, 12, 14, 16, 18, 20, 25, 30, 35, 40, 50, 60, 70, 80, 90, 100, 150, 200, 250 or more). These spr0286 proteins include variants of SEQ ID NO: 2. Preferred fragments of (b) comprise an epitope from SEQ ID NO:

2. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 2 while retaining at least one epitope of SEQ ID NO: 2. Other fragments omit one or more protein domains. One suitable fragment is SEQ ID NO: 181, which omits the natural leader peptide and sortase recognition sequences. Other suitable fragments are SEQ ID NOs: 182 and 183.

spr0565

The original ‘spr0565’ sequence was annotated in reference 205 as ‘beta-galactosidase precursor’ (see GI:15902609). For reference purposes, the amino acid sequence of full length spr0565 as found in the R6 strain is given as SEQ ID NO: 3 herein.

Preferred spr0565 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 3; and/or (b) comprising a fragment of at least ‘n’ consecutive amino acids of SEQ ID NO: 3, wherein ‘n’ is 7 or more (e.g. 8, 10, 12, 14, 16, 18, 20, 25, 30, 35, 40, 50, 60, 70, 80, 90, 100, 150, 200, 250 or more). These spr0565 proteins include variants of SEQ ID NO: 3 (e.g. SEQ ID NO: 66; see below). Preferred fragments of (b) comprise an epitope from SEQ ID NO: 3. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 3 while retaining at least one epitope of SEQ ID NO: 3. Other fragments omit one or more protein domains. One suitable fragment is SEQ ID NO: 184, which omits the natural leader peptide and sortase recognition sequences. Other suitable fragments are SEQ ID NOs: 177 and 178.

A variant form of spr0565 is SEQ ID NO: 66 herein. The use of this variant form for immunisation is reported in reference 82 (SEQ ID NO: 178 therein). Useful spr0565 polypeptides may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 66; and/or (b) comprising a fragment of at least ‘n’ consecutive amino acids of SEQ ID NO: 66, wherein ‘n’ is 7 or more (e.g. 8, 10, 12, 14, 16, 18, 20, 25, 30, 35, 40, 50, 60, 70, 80, 90, 100, 150, 200, 250 or more).

These polypeptides include variants of SEQ ID NO: 66. Preferred fragments of (b) comprise an epitope from SEQ ID NO: 66. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 66 while retaining at least one epitope of SEQ ID NO: 66. Other fragments omit one or more protein domains.

Immunogenic fragments of SEQ ID NO: 66 are identified in table 1 of reference 82.

Because spr0565 is naturally a long polypeptide (>2000 aa) it can be more convenient to express fragments. Thus a suitable form of spr0565 for use with the invention may be less than 1500 amino acids long (e.g. <1400, <1300, <1200, <1100, etc.). Such short forms of spr0565 include ‘spr0565A’ (SEQ ID NO: 177) and ‘spr0565B’ (SEQ ID NO: 178).

Combinations of spr0565 with other pneumococcal antigens have shown good synergistic effects.

spr1 098

The original ‘spr1098’ sequence was annotated in reference 205 as ‘Sortase’ (see GI:15903141). For reference purposes, the amino acid sequence of full length spr1098 as found in the R6 strain is given as SEQ ID NO: 4 herein.

Preferred spr1098 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 4; and/or (b) comprising a fragment of at least ‘n’ consecutive amino acids of SEQ ID NO: 4, wherein ‘n’ is 7 or more (e.g. 8, 10, 12, 14, 16, 18, 20, 25, 30, 35, 40, 50, 60, 70, 80, 90, 100, 150, 200, 250 or more). These spr1098 proteins include variants of SEQ ID NO: 4. Preferred fragments of (b) comprise an epitope from SEQ ID NO: 4. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 4 while retaining at least one epitope of SEQ ID NO: 4. Other fragments omit one or more protein domains. One suitable fragment is SEQ ID NO: 187, which omits the natural leader peptide sequence.

spr 1 345

The original ‘spr1345’ sequence was annotated in reference 205 as ‘hypothetical protein’ (see GI:15903388). For reference purposes, the amino acid sequence of full length spr1345 as found in the R6 strain is given as SEQ ID NO: 5 herein.

Preferred spr1345 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 5; and/or (b) comprising a fragment of at least ‘n’ consecutive amino acids of SEQ ID NO: 5, wherein ‘n’ is 7 or more (e.g. 8, 10, 12, 14, 16, 18, 20, 25, 30, 35, 40, 50, 60, 70, 80, 90, 100, 150, 200, 250 or more). These spr1345 proteins include variants of SEQ ID NO: 5. Preferred fragments of (b) comprise an epitope from SEQ ID NO: 5. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 5 while retaining at least one epitope of SEQ ID NO: 5. Other fragments omit one or more protein domains. One suitable fragment is SEQ ID NO: 188, which omits the natural leader peptide and sortase recognition sequences.

spr1416

The original ‘spr1416’ sequence was annotated in reference 205 as ‘hypothetical protein’ (see GI:15903459). For reference purposes, the amino acid sequence of full length spr1416 as found in the R6 strain is given as SEQ ID NO: 6 herein.

Preferred spr1416 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 6; and/or (b) comprising a fragment of at least ‘n’ consecutive amino acids of SEQ ID NO: 6, wherein ‘n’ is 7 or more (e.g. 8, 10, 12, 14, 16, 18, 20, 25, 30, 35, 40, 50, 60, 70, 80, 90, 100, 150, 200, 250 or more). These spr1416 proteins include variants of SEQ ID NO: 6. Preferred fragments of (b) comprise an epitope from SEQ ID NO: 6. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 6 while retaining at least one epitope of SEQ ID NO: 6. Other fragments omit one or more protein domains.

spr1418

The original ‘spr1418’ sequence was annotated in reference 205 as ‘hypothetical protein’ (see GI:15903461). For reference purposes, the amino acid sequence of full length spr1418 as found in the R6 strain is given as SEQ ID NO: 7 herein.

Preferred spr1418 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 7; and/or (b) comprising a fragment of at least ‘n’ consecutive amino acids of SEQ ID NO: 7, wherein ‘n’ is 7 or more (e.g. 8, 10, 12, 14, 16, 18, 20, 25, 30, 35, 40, 50, 60, 70, 80, 90, 100, 150, 200, 250 or more). These spr1418 proteins include variants of SEQ ID NO: 7. Preferred fragments of (b) comprise an epitope from SEQ ID NO: 7. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 7 while retaining at least one epitope of SEQ ID NO: 7. Other fragments omit one or more protein domains.

spr0867

The original ‘spr0867’ sequence was annotated in reference 205 as ‘Endo-beta-N-acetylglucosaminidase’ (see GI:15902911). For reference purposes, the amino acid sequence of full length spr0867 as found in the R6 strain is given as SEQ ID NO: 8 herein.

Preferred spr0867 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 8; and/or (b) comprising a fragment of at least ‘n’ consecutive amino acids of SEQ ID NO: 8, wherein ‘n’ is 7 or more (e.g. 8, 10, 12, 14, 16, 18, 20, 25, 30, 35, 40, 50, 60, 70, 80, 90, 100, 150, 200, 250 or more). These spr0867 proteins include variants of SEQ ID NO: 8. Preferred fragments of (b) comprise an epitope from SEQ ID NO: 8. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 8 while retaining at least one epitope of SEQ ID NO: 8. Other fragments omit one or more protein domains. One suitable fragment is SEQ ID NO: 185, which omits the natural leader peptide sequence.

spr1431

The original ‘spr1431’ sequence was annotated in reference 205 as ‘1,4-beta-N-acetylmuramidase’ (see GI:15903474). It is also known as ‘LytC’, and its use for immunisation is reported in reference 103. For reference purposes, the amino acid sequence of full length spr1431 as found in the R6 strain is given as SEQ ID NO: 9 herein.

Preferred spr1431 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 9; and/or (b) comprising a fragment of at least ‘n’ consecutive amino acids of SEQ ID NO: 9, wherein ‘n’ is 7 or more (e.g. 8, 10, 12, 14, 16, 18, 20, 25, 30, 35, 40, 50, 60, 70, 80, 90, 100, 150, 200, 250 or more). These spr1431 proteins include variants of SEQ ID NO: 9. Preferred fragments of (b) comprise an epitope from SEQ ID NO: 9. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 9 while retaining at least one epitope of SEQ ID NO: 9. Other fragments omit one or more protein domains. One suitable fragment is SEQ ID NO: 189, which omits the natural leader peptide sequence.

spr 1 739

The ‘spr1739’ polypeptide is pneumolysin (e.g. see GI:15903781). For reference purposes, the amino acid sequence of full length spr1739 as found in the R6 strain is given as SEQ ID NO: 10 herein.

Preferred spr1739 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 10; and/or (b) comprising a fragment of at least ‘n’ consecutive amino acids of SEQ ID NO: 10, wherein ‘n’ is 7 or more (e.g. 8, 10, 12, 14, 16, 18, 20, 25, 30, 35, 40, 50, 60, 70, 80, 90, 100, 150, 200, 250 or more). These spr1739 proteins include variants of SEQ ID NO: 10. Preferred fragments of (b) comprise an epitope from SEQ ID NO: 10. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 10 while retaining at least one epitope of SEQ ID NO: 10. Other fragments omit one or more protein domains.

Mutant forms of pneumolysin for vaccination use are known in the art [123, 83-88], and these mutant forms may be used with the invention. Detoxification can be achieved by C-terminal truncation (e.g. see ref. 89) e.g. deleting 34 amino acids, 45 amino acids, 7 amino acids [90], etc. Further mutations, numbered according to SEQ ID NO: 20, include Pro325-*Leu (e.g. SEQ ID NO: 169) and/or Trp433-*Phe (e.g. SEQ ID NO: 171). These mutations may be combined with C-terminal truncations e.g. to combine a Pro325-*Leu mutation with a 7-mer truncation (e.g. SEQ ID NO: 170).

spr2021

The original ‘spr2021’ sequence was annotated in reference 205 as ‘General stress protein GSP-781’ (see GI:15904062). For reference purposes, the amino acid sequence of full length spr2021 as found in the R6 strain is given as SEQ ID NO: 11 herein.

Preferred spr2021 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 11; and/or (b) comprising a fragment of at least ‘n’ consecutive amino acids of SEQ ID NO: 11, wherein ‘n’ is 7 or more (e.g. 8, 10, 12, 14, 16, 18, 20, 25, 30, 35, 40, 50, 60, 70, 80, 90, 100, 150, 200, 250 or more). These spr2021 proteins include variants of SEQ ID NO: 11. Preferred fragments of (b) comprise an epitope from SEQ ID NO: 11. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 11 while retaining at least one epitope of SEQ ID NO: 11. Other fragments omit one or more protein domains. One suitable fragment is SEQ ID NO: 190, which omits the natural leader peptide sequence.

Combinations of spr2021 with other pneumococcal antigens have shown good synergistic effects.

Reference 82 annotates spr2021 as a secreted 45 kDa protein with homology to GbpB and discloses its use as an immunogen (SEQ ID NO: 243 therein; SP2216). Immunogenic fragments of spr2021 are identified in table 1 of reference 82 (page 73). Another useful fragment of spr2021 is disclosed as SEQ ID NO: 1 of reference 91 (amino acids 28-278 of SEQ ID NO: 11 herein).

spr0096

The original ‘spr0096’ sequence was annotated in reference 205 as ‘hypothetical protein’ (see GI:15902140). For reference purposes, the amino acid sequence of full length spr0096 as found in the R6 strain is given as SEQ ID NO: 12 herein.

Preferred spr0096 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 12; and/or (b) comprising a fragment of at least ‘n’ consecutive amino acids of SEQ ID NO: 12, wherein ‘n’ is 7 or more (e.g. 8, 10, 12, 14, 16, 18, 20, 25, 30, 35, 40, 50, 60, 70, 80, 90, 100, 150, 200, 250 or more). These spr0096 proteins include variants of SEQ ID NO: 12 (e.g. SEQ ID NO: 40; see below). Preferred fragments of (b) comprise an epitope from SEQ ID NO: 12. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 12 while retaining at least one epitope of SEQ ID NO: 12. Other fragments omit one or more protein domains.

Combinations of spr0096 with other pneumococcal antigens have shown good synergistic effects.

A variant form of spr0096, with an insert near its C-terminus relative to SEQ ID NO: 12, is SEQ ID NO: 40 herein. The use of this variant for immunisation is reported in reference 82 (SEQ ID NO: 150 therein), where it is annotated as a LysM domain protein. Thus a spr0096 for use with the invention may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 40; and/or (b) comprising a fragment of at least ‘n’ consecutive amino acids of SEQ ID NO: 40, wherein ‘n’ is 7 or more (e.g. 8, 10, 12, 14, 16, 18, 20, 25, 30, 35, 40, 50, 60, 70, 80, 90, 100, 150, 200, 250 or more). These polypeptides include variants of SEQ ID NO: 40. Preferred fragments of (b) comprise an epitope from SEQ ID NO: 40. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 40 while retaining at least one epitope of SEQ ID NO: 40. Other fragments omit one or more protein domains. Immunogenic fragments of SEQID NO: 40 are identified in table 1 of reference 82.

A spr0096 polypeptide may be used in the form of a dimer e.g. a homodimer.

spr 1433

The original ‘spr1433’ sequence was annotated in reference 205 as ‘hypothetical protein’ (see GI:15903476). For reference purposes, the amino acid sequence of full length spr1433 as found in the R6 strain is given as SEQ ID NO: 13 herein.

Preferred spr1433 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 13; and/or (b) comprising a fragment of at least ‘n’ consecutive amino acids of SEQ ID NO: 13, wherein ‘n’ is 7 or more (e.g. 8, 10, 12, 14, 16, 18, 20, 25, 30, 35, 40, 50, 60, 70, 80, 90, 100, 150, 200, 250 or more). These spr1433 proteins include variants of SEQ ID NO: 13. Preferred fragments of (b) comprise an epitope from SEQ ID NO: 13. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 13 while retaining at least one epitope of SEQ ID NO: 13. Other fragments omit one or more protein domains.

spr 1 707

The original ‘spr1707’ sequence was annotated in reference 205 as ‘ABC transporter substrate-binding protein—oligopeptide transport’ (see GI:15903749). For reference purposes, the amino acid sequence of full length spr1707 as found in the R6 strain is given as SEQ ID NO: 14 herein.

Preferred spr1707 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 14; and/or (b) comprising a fragment of at least ‘n’ consecutive amino acids of SEQ ID NO: 14, wherein ‘n’ is 7 or more (e.g. 8, 10, 12, 14, 16, 18, 20, 25, 30, 35, 40, 50, 60, 70, 80, 90, 100, 150, 200, 250 or more). These spr1707 proteins include variants of SEQ ID NO: 14 (e.g. SEQ ID NO: 100; see below). Preferred fragments of (b) comprise an epitope from SEQ ID NO: 14. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 14 while retaining at least one epitope of SEQ ID NO: 14. Other fragments omit one or more protein domains.

A variant form of spr1707, differing from SEQ ID NO: 14 by 4 amino acids, is SEQ ID NO: 100 herein. The use of SEQ ID NO: 100 for immunisation is reported in reference 82 (SEQ ID NO: 220 therein). Thus a spr1707 polypeptide for use with the invention may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 100; and/or (b) comprising a fragment of at least ‘n’ consecutive amino acids of SEQ ID NO: 100, wherein ‘n’ is 7 or more (e.g. 8, 10, 12, 14, 16, 18, 20, 25, 30, 35, 40, 50, 60, 70, 80, 90, 100, 150, 200, 250 or more). These polypeptides include variants of SEQ ID NO: 100. Preferred fragments of (b) comprise an epitope from SEQ ID NO: 100. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 100 while retaining at least one epitope of SEQ ID NO: 100. Other fragments omit one or more protein domains.

Immunogenic fragments of SEQ ID NO: 100 are identified in table 1 of reference 82.

ClpP

ClpP is the ATP-dependent Clp protease proteolytic subunit. For reference purposes, the amino acid sequence of full length ClpP is SEQ ID NO: 16 herein. In the R6 genome ClpP is spr0656 [205].

Preferred ClpP polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 16; and/or (b) comprising a fragment of at least ‘n’ consecutive amino acids of SEQ ID NO: 16, wherein ‘n’ is 7 or more (e.g. 8, 10, 12, 14, 16, 18, 20, 25, 30, 35, 40, 50, 60, 70, 80, 90, 100, 150, 200, 250 or more). These ClpP proteins include variants of SEQ ID NO: 16. Preferred fragments of (b) comprise an epitope from SEQ ID NO: 16. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 16 while retaining at least one epitope of SEQ ID NO: 16. Other fragments omit one or more protein domains.

The use of ClpP for immunisation is reported in references 92 and 93. It may advantageously be used in combination with PspA and PsaA and/or PspC [92].

LytA

LytA is the N-acetylmuramoyl-L-alanine amidase (autolysin). For reference purposes, the amino acid sequence of full length LytA is SEQ ID NO: 17 herein. In the R6 genome LytA is spr1754 [205].

Preferred LytA polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 17; and/or (b) comprising a fragment of at least ‘n’ consecutive amino acids of SEQ ID NO: 17, wherein ‘n’ is 7 or more (e.g. 8, 10, 12, 14, 16, 18, 20, 25, 30, 35, 40, 50, 60, 70, 80, 90, 100, 150, 200, 250 or more). These LytA proteins include variants of SEQ ID NO: 17 (e.g. GI:18568354). Preferred fragments of (b) comprise an epitope from SEQ ID NO: 17. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 17 while retaining at least one epitope of SEQ ID NO: 17. Other fragments omit one or more protein domains.

The use of LytA for immunisation is reported in reference 94, particularly in a form comprising the LytA choline binding domain fused to a heterologous promiscuous T helper epitope.

PhtA

PhtA is the Pneumococcal histidine triad protein A. For reference purposes, the amino acid sequence of full length PhtA precursor is SEQ ID NO: 18 herein. In the R6 genome PhtA is spr1061 [205].

Preferred PhtA polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 18; and/or (b) comprising a fragment of at least ‘n’ consecutive amino acids of SEQ ID NO: 18, wherein ‘n’ is 7 or more (e.g. 8, 10, 12, 14, 16, 18, 20, 25, 30, 35, 40, 50, 60, 70, 80, 90, 100, 150, 200, 250 or more). These PhtA proteins include variants of SEQ ID NO: 18. Preferred fragments of (b) comprise an epitope from SEQ ID NO: 18. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 18 while retaining at least one epitope of SEQ ID NO: 18. Other fragments omit one or more protein domains.

The use of PhtA for immunisation is reported in references 95 and 96.

PhtB

PhtB is the pneumococcal histidine triad protein B. For reference purposes, the amino acid sequence of full length PhtB precursor is SEQ ID NO: 19 herein. Xaa at residue 578 can be Lysine.

Preferred PhtB polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 19; and/or (b) comprising a fragment of at least ‘n’ consecutive amino acids of SEQ ID NO: 19, wherein ‘n’ is 7 or more (e.g. 8, 10, 12, 14, 16, 18, 20, 25, 30, 35, 40, 50, 60, 70, 80, 90, 100, 150, 200, 250 or more). These PhtB proteins include variants of SEQ ID NO: 19. Preferred fragments of (b) comprise an epitope from SEQ ID NO: 19. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 19 while retaining at least one epitope of SEQ ID NO: 19. Other fragments omit one or more protein domains.

The use of PhtB for immunisation is reported in references 97 and 98.

PhtD

PhtD is the Pneumococcal histidine triad protein D. For reference purposes, the amino acid sequence of full length PhtD precursor is SEQ ID NO: 20 herein. In the R6 genome PhtD is spr0907 [205].

Preferred PhtD polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 20; and/or (b) comprising a fragment of at least ‘n’ consecutive amino acids of SEQ ID NO: 20, wherein ‘n’ is 7 or more (e.g. 8, 10, 12, 14, 16, 18, 20, 25, 30, 35, 40, 50, 60, 70, 80, 90, 100, 150, 200, 250 or more). These PhtD proteins include variants of SEQ ID NO: 20. Preferred fragments of (b) comprise an epitope from SEQ ID NO: 20. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 20 while retaining at least one epitope of SEQ ID NO: 20. Other fragments omit one or more protein domains.

The use of PhtD for immunisation is reported in references 95, 96 and 99.

PhtE

PhtE is the Pneumococcal histidine triad protein E. For reference purposes, the amino acid sequence of full length PhtE precursor is SEQ ID NO: 21 herein. In the R6 genome PhtE is spr0908 [205].

Preferred PhtE polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 21; and/or (b) comprising a fragment of at least ‘n’ consecutive amino acids of SEQ ID NO: 21, wherein ‘n’ is 7 or more (e.g. 8, 10, 12, 14, 16, 18, 20, 25, 30, 35, 40, 50, 60, 70, 80, 90, 100, 150, 200, 250 or more). These PhtE proteins include variants of SEQ ID NO: 21. Preferred fragments of (b) comprise an epitope from SEQ ID NO: 21. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 21 while retaining at least one epitope of SEQ ID NO: 21. Other fragments omit one or more protein domains.

The use of PhtE for immunisation is reported in references 95 and 96.

ZmpB

ZmpB is the zinc metalloprotease. For reference purposes, the amino acid sequence of full length ZmpB is SEQ ID NO: 22 herein. In the R6 genome ZmpB is spr0581 [205].

Preferred ZmpB polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 22; and/or (b) comprising a fragment of at least ‘n’ consecutive amino acids of SEQ ID NO: 22, wherein ‘n’ is 7 or more (e.g. 8, 10, 12, 14, 16, 18, 20, 25, 30, 35, 40, 50, 60, 70, 80, 90, 100, 150, 200, 250 or more). These ZmpB proteins include variants of SEQ ID NO: 22. Preferred fragments of (b) comprise an epitope from SEQ ID NO: 22. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 22 while retaining at least one epitope of SEQ ID NO: 22. Other fragments omit one or more protein domains.

CbpD

CbpD is the Choline binding protein D. For reference purposes, the amino acid sequence of full length CbpD is SEQ ID NO: 23 herein. In the R6 genome CbpD is spr2006 [205].

Preferred CbpD polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 23; and/or (b) comprising a fragment of at least ‘n’ consecutive amino acids of SEQ ID NO: 23, wherein ‘n’ is 7 or more (e.g. 8, 10, 12, 14, 16, 18, 20, 25, 30, 35, 40, 50, 60, 70, 80, 90, 100, 150, 200, 250 or more). These CbpD proteins include variants of SEQ ID NO: 23 (e.g. SEQ ID NO: 119; see below). Preferred fragments of (b) comprise an epitope from SEQ ID NO: 23. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 23 while retaining at least one epitope of SEQ ID NO: 23. Other fragments omit one or more protein domains.

The use of CbpD for immunisation is reported in reference 103.

A variant of SEQ ID NO: 23 is SEQ ID NO: 119 herein. The use of SEQ ID NO: 119 for immunisation is reported in reference 82 (SEQ ID NO: 241 therein). Thus a CbpD polypeptide for use with the invention may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 119; and/or (b) comprising a fragment of at least ‘n’ consecutive amino acids of SEQ ID NO: 119, wherein ‘n’ is 7 or more (e.g. 8, 10, 12, 14, 16, 18, 20, 25, 30, 35, 40, 50, 60, 70, 80, 90, 100, 150, 200, 250 or more). These CbpD proteins include variants of SEQ ID NO: 119. Preferred fragments of (b) comprise an epitope from SEQ ID NO: 119. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 119 while retaining at least one epitope of SEQ ID NO: 119. Other fragments omit one or more protein domains.

Immunogenic fragments of SEQ ID NO: 119 are identified in table 1 of reference 82.

CbpG

CbpG is the Choline binding protein G. For reference purposes, the amino acid sequence of full length CbpG is SEQ ID NO: 24 herein. In the R6 genome CbpG is spr0350 [205].

Preferred CbpG polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 24; and/or (b) comprising a fragment of at least ‘n’ consecutive amino acids of SEQ ID NO: 24, wherein ‘n’ is 7 or more (e.g. 8, 10, 12, 14, 16, 18, 20, 25, 30, 35, 40, 50, 60, 70, 80, 90, 100, 150, 200, 250 or more). These CbpG proteins include variants of SEQ ID NO: 24. Preferred fragments of (b) comprise an epitope from SEQ ID NO: 24. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 24 while retaining at least one epitope of SEQ ID NO: 24. Other fragments omit one or more protein domains.

The use of CbpG for immunisation is reported in reference 103.

PvaA

PvaA (Streptococcus pneumoniae pneumococcal vaccine antigen A) is also known as sp101. For reference purposes, the amino acid sequence of full length PvaA is SEQ ID NO: 25 herein. In the R6 genome PvaA is spr0930 [205].

Preferred PvaA polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 25; and/or (b) comprising a fragment of at least ‘n’ consecutive amino acids of SEQ ID NO: 25, wherein ‘n’ is 7 or more (e.g. 8, 10, 12, 14, 16, 18, 20, 25, 30, 35, 40, 50, 60, 70, 80, 90, 100, 150, 200, 250 or more). These PvaA proteins include variants of SEQ ID NO: 25. Preferred fragments of (b) comprise an epitope from SEQ ID NO: 25. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 25 while retaining at least one epitope of SEQ ID NO: 25. Other fragments omit one or more protein domains.

The use of PvaA for immunisation is reported in references 100 and 101.

CPL1

CPL1 is the pneumococcal phage CP1 lysozyme. For reference purposes, the amino acid sequence of full length CPL1 is SEQ ID NO: 26 herein.

Preferred CPL1 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 26; and/or (b) comprising a fragment of at least ‘n’ consecutive amino acids of SEQ ID NO: 26, wherein ‘n’ is 7 or more (e.g. 8, 10, 12, 14, 16, 18, 20, 25, 30, 35, 40, 50, 60, 70, 80, 90, 100, 150, 200, 250 or more). These CPL1 proteins include variants of SEQ ID NO: 26. Preferred fragments of (b) comprise an epitope from SEQ ID NO: 26. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 26 while retaining at least one epitope of SEQ ID NO: 26. Other fragments omit one or more protein domains.

The use of CPL1 for immunisation is reported in reference 94, particularly in a form comprising the CPL1 choline binding domain fused to a heterologous promiscuous T helper epitope.

PspC

PspC is the pneumococcal surface protein C [102] and is also known as choline-binding protein A (CbpA). Its use for immunisation is reported in references 100 and 103. In the R6 strain it is spr1995 and, for reference, the amino acid sequence of full length spr1995 is SEQ ID NO: 15 herein.

Preferred PspC polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 15; and/or (b) comprising a fragment of at least ‘n’ consecutive amino acids of SEQ ID NO: 15, wherein ‘n’ is 7 or more (e.g. 8, 10, 12, 14, 16, 18, 20, 25, 30, 35, 40, 50, 60, 70, 80, 90, 100, 150, 200, 250 or more). These spr1995 proteins include variants of SEQ ID NO: 15 (e.g. SEQ ID NO: 27; see below). Preferred fragments of (b) comprise an epitope from SEQ ID NO: 15. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 15 while retaining at least one epitope of SEQ ID NO: 15. Other fragments omit one or more protein domains.

A variant of PspC is known as ‘Hic’. It is similar to PspC, as shown in FIG. 1 of reference 104, where it is reported to bind to factor H (fH). For reference purposes, the amino acid sequence of full length Hic is SEQ ID NO: 27 herein. A Hic protein may be used with the invention in addition to or in place of a PspC polypeptide.

Preferred Hic polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 27; and/or (b) comprising a fragment of at least ‘n’ consecutive amino acids of SEQ ID NO: 27, wherein ‘n’ is 7 or more (e.g. 8, 10, 12, 14, 16, 18, 20, 25, 30, 35, 40, 50, 60, 70, 80, 90, 100, 150, 200, 250 or more). These Hic proteins include variants of SEQ ID NO: 27. Preferred fragments of (b) comprise an epitope from SEQ ID NO: 27. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 27 while retaining at least one epitope of SEQ ID NO: 27. Other fragments omit one or more protein domains.

PspC and/or Hic can advantageously be used in combination with PspA and/or PsaA.

Pmp

Pmp is a peptidylprolyl isomerase, also known as protease maturation protein. For reference purposes, the amino acid sequence of full length Pmp is SEQ ID NO: 28 herein. In the R6 genome Pmp is spr0884 [205].

Preferred Pmp polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 28; and/or (b) comprising a fragment of at least ‘n’ consecutive amino acids of SEQ ID NO: 28, wherein ‘n’ is 7 or more (e.g. 8, 10, 12, 14, 16, 18, 20, 25, 30, 35, 40, 50, 60, 70, 80, 90, 100, 150, 200, 250 or more). These Pmp proteins include variants of SEQ ID NO: 28. Preferred fragments of (b) comprise an epitope from SEQ ID NO: 28. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 28 while retaining at least one epitope of SEQ ID NO: 28. Other fragments omit one or more protein domains. One suitable fragment is SEQ ID NO: 186, which omits the natural leader peptide sequence.

The use of Pmp for immunisation is reported in reference 105.

PspA

PspA is the Pneumococcal surface protein A. For reference purposes, the amino acid sequence of full length PspA is SEQ ID NO: 29 herein. In the R6 genome PspA is spr0121 [205].

Preferred PspA polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 29; and/or (b) comprising a fragment of at least ‘n’ consecutive amino acids of SEQ ID NO: 29, wherein ‘n’ is 7 or more (e.g. 8, 10, 12, 14, 16, 18, 20, 25, 30, 35, 40, 50, 60, 70, 80, 90, 100, 150, 200, 250 or more). These PspA proteins include variants of SEQ ID NO: 29. Preferred fragments of (b) comprise an epitope from SEQ ID NO: 29. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 29 while retaining at least one epitope of SEQ ID NO: 29. Other fragments omit one or more protein domains.

The use of PspA for immunisation is reported inter alia in reference 106. It can advantageously be administered in combination with PspC.

PsaA

PsaA is the Pneumococcal surface adhesin. For reference purposes, the amino acid sequence of full length PsaA is SEQ ID NO: 30 herein.

Preferred PsaA polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 30; and/or (b) comprising a fragment of at least ‘n’ consecutive amino acids of SEQ ID NO: 30, wherein ‘n’ is 7 or more (e.g. 8, 10, 12, 14, 16, 18, 20, 25, 30, 35, 40, 50, 60, 70, 80, 90, 100, 150, 200, 250 or more). These PsaA proteins include variants of SEQ ID NO: 30. Preferred fragments of (b) comprise an epitope from SEQ ID NO: 30. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 30 while retaining at least one epitope of SEQ ID NO: 30. Other fragments omit one or more protein domains. A useful fragment of PsaA is disclosed as SEQ ID NO: 3 in reference 91 (corresponding to amino acids 21-309 of SEQ ID NO: 30 herein).

The use of PsaA for immunisation is reported in reference 107. It can be used in combination with PspA and/or PspC.

PrtA

PrtA is the cell wall-associated serine proteinase. It has also been known as sp128 and sp130, and is in a subtilisin-like serine protease. For reference purposes, the amino acid sequence of full length PrtA precursor is SEQ ID NO: 31 herein. In the R6 genome PrtA is spr0561 [205].

Preferred PrtA polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 31; and/or (b) comprising a fragment of at least ‘n’ consecutive amino acids of SEQ ID NO: 31, wherein ‘n’ is 7 or more (e.g. 8, 10, 12, 14, 16, 18, 20, 25, 30, 35, 40, 50, 60, 70, 80, 90, 100, 150, 200, 250 or more). These PrtA proteins include variants of SEQ ID NO: 31. Preferred fragments of (b) comprise an epitope from SEQ ID NO: 31. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 31 while retaining at least one epitope of SEQ ID NO: 31. Other fragments omit one or more protein domains.

The use of PrtA for immunisation is reported in references 108 & 109, and also in reference 100.

Sp133

Sp133 is a conserved pneumococcal antigen. For reference purposes, the amino acid sequence of full length Sp133 is SEQ ID NO: 32 herein. In the R6 genome Sp133 is spr0931 [205].

Preferred Sp133 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 32; and/or (b) comprising a fragment of at least ‘n’ consecutive amino acids of SEQ ID NO: 32, wherein ‘n’ is 7 or more (e.g. 8, 10, 12, 14, 16, 18, 20, 25, 30, 35, 40, 50, 60, 70, 80, 90, 100, 150, 200, 250 or more). These Sp133 proteins include variants of SEQ ID NO: 32. Preferred fragments of (b) comprise an epitope from SEQ ID NO: 32. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 32 while retaining at least one epitope of SEQ ID NO: 32. Other fragments omit one or more protein domains.

The use of Sp133 for immunisation is reported in reference 110.

PiaA

PiaA is the membrane permease involved in iron acquisition by pneumococcus. For reference purposes, the amino acid sequence of full length PiaA is SEQ ID NO: 33 herein. In the R6 genome PiaA is spr0935 [205].

Preferred PiaA polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 33; and/or (b) comprising a fragment of at least ‘n’ consecutive amino acids of SEQ ID NO: 33, wherein ‘n’ is 7 or more (e.g. 8, 10, 12, 14, 16, 18, 20, 25, 30, 35, 40, 50, 60, 70, 80, 90, 100, 150, 200, 250 or more). These PiaA proteins include variants of SEQ ID NO: 33. Preferred fragments of (b) comprise an epitope from SEQ ID NO: 33. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 33 while retaining at least one epitope of SEQ ID NO: 33. Other fragments omit one or more protein domains.

The use of PiaA for immunisation is reported in references 111, 112 and 113, particularly in combination with PiuA.

PiuA

PiuA is the ABC transporter substrate-binding protein for ferric iron transport. It is also known as FatB. For reference purposes, the amino acid sequence of full length PiuA is SEQ ID NO: 34 herein. In the R6 genome PiuA is spr1687 [205].

Preferred PiuA polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 34; and/or (b) comprising a fragment of at least ‘n’ consecutive amino acids of SEQ ID NO: 34, wherein ‘n’ is 7 or more (e.g. 8, 10, 12, 14, 16, 18, 20, 25, 30, 35, 40, 50, 60, 70, 80, 90, 100, 150, 200, 250 or more). These PiuA proteins include variants of SEQ ID NO: 34. Preferred fragments of (b) comprise an epitope from SEQ ID NO: 34. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 34 while retaining at least one epitope of SEQ ID NO: 34. Other fragments omit one or more protein domains.

The use of PiuA for immunisation is reported in refs 111 to 113, particularly in combination with PiaA.

IC1

IC1 is annotated in reference 82 as a hypothetical protein. For reference purposes, the amino acid sequence of full length IC1 is SEQ ID NO: 35 herein. In the R6 genome IC1 is spr0008 [205]. The use of IC1 for immunisation is reported in reference 82 (SEQ ID NO: 145 therein).

Preferred IC1 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 35; and/or (b) comprising a fragment of at least ‘n’ consecutive amino acids of SEQ ID NO: 35, wherein ‘n’ is 7 or more (e.g. 8, 10, 12, 14, 16, 18, 20, 25, 30, 35, 40, 50, 60, 70, 80, 90, 100, 150, 200, 250 or more). These IC1 proteins include variants of SEQ ID NO: 35. Preferred fragments of (b) comprise an epitope from SEQ ID NO: 35. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 35 while retaining at least one epitope of SEQ ID NO: 35. Other fragments omit one or more protein domains. Immunogenic fragments of IC1 are identified in table 1 of reference 82.

IC2

IC2 is the polA DNA polymerase I. For reference purposes, the amino acid sequence of full length IC2 is SEQ ID NO: 36 herein. In the R6 genome IC2 is spr0032 [205]. The use of IC2 for immunisation is reported in reference 82 (SEQ ID NO: 146 therein).

Preferred IC2 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 36; and/or (b) comprising a fragment of at least ‘n’ consecutive amino acids of SEQ ID NO: 36, wherein ‘n’ is 7 or more (e.g. 8, 10, 12, 14, 16, 18, 20, 25, 30, 35, 40, 50, 60, 70, 80, 90, 100, 150, 200, 250 or more). These IC2 proteins include variants of SEQ ID NO: 36. Preferred fragments of (b) comprise an epitope from SEQ ID NO: 36. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 36 while retaining at least one epitope of SEQ ID NO: 36. Other fragments omit one or more protein domains. Immunogenic fragments of IC2 are identified in table 1 of reference 82.

IC3

IC3 is a choline-binding protein. For reference purposes, the amino acid sequence of full length IC3 is SEQ ID NO: 37 herein. In the R6 genome IC3 is spr1945 [205]. The use of IC3 for immunisation is reported in reference 82 (SEQ ID NO: 147 therein).

Preferred IC3 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 37; and/or (b) comprising a fragment of at least ‘n’ consecutive amino acids of SEQ ID NO: 37, wherein ‘n’ is 7 or more (e.g. 8, 10, 12, 14, 16, 18, 20, 25, 30, 35, 40, 50, 60, 70, 80, 90, 100, 150, 200, 250 or more). These IC3 proteins include variants of SEQ ID NO: 37. Preferred fragments of (b) comprise an epitope from SEQ ID NO: 37. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 37 while retaining at least one epitope of SEQ ID NO: 37. Other fragments omit one or more protein domains. Immunogenic fragments of IC3 are identified in table 1 of reference 82.

IC4

IC4 is an IgA1 protease. For reference purposes, the amino acid sequence of full length IC4 is SEQ ID NO: 38 herein. In the R6 genome IC4 is spr1042 [205]. The use of IC4 for immunisation is reported in reference 82 (SEQ ID NO: 148 therein).

Preferred IC4 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 38; and/or (b) comprising a fragment of at least ‘n’ consecutive amino acids of SEQ ID NO: 38, wherein ‘n’ is 7 or more (e.g. 8, 10, 12, 14, 16, 18, 20, 25, 30, 35, 40, 50, 60, 70, 80, 90, 100, 150, 200, 250 or more). These IC4 proteins include variants of SEQ ID NO: 38. Preferred fragments of (b) comprise an epitope from SEQ ID NO: 38. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 38 while retaining at least one epitope of SEQ ID NO: 38. Other fragments omit one or more protein domains. Immunogenic fragments of IC4 are identified in table 1 of reference 82.

IC5

IC5 is annotated as a hypothetical protein, but is maybe a cell wall surface anchor. For reference purposes, the amino acid sequence of full length IC5 is SEQ ID NO: 39 herein. In the R6 genome IC5 is spr0075 [205]. The use of IC5 for immunisation is reported in reference 82 (SEQ ID NO: 149 therein).

Preferred IC5 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 39; and/or (b) comprising a fragment of at least ‘n’ consecutive amino acids of SEQ ID NO: 39, wherein ‘n’ is 7 or more (e.g. 8, 10, 12, 14, 16, 18, 20, 25, 30, 35, 40, 50, 60, 70, 80, 90, 100, 150, 200, 250 or more). These IC5 proteins include variants of SEQ ID NO: 39. Preferred fragments of (b) comprise an epitope from SEQ ID NO: 39. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 39 while retaining at least one epitope of SEQ ID NO: 39. Other fragments omit one or more protein domains. Immunogenic fragments of IC5 are identified in table 1 of reference 82.

IC6

IC6 is a variant form of spr0096, as reported above (SEQ ID NO: 40 herein). Useful IC6 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 40; and/or (b) comprising a fragment of at least ‘n’ consecutive amino acids of SEQ ID NO: 40, wherein ‘n’ is 7 or more (e.g. 8, 10, 12, 14, 16, 18, 20, 25, 30, 35, 40, 50, 60, 70, 80, 90, 100, 150, 200, 250 or more). These IC6 proteins include variants of SEQ ID NO: 40. Preferred fragments of (b) comprise an epitope from SEQ ID NO: 40. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 40 while retaining at least one epitope of SEQ ID NO: 40. Other fragments omit one or more protein domains.

IC7

IC7 is annotated in reference 82 as a hypothetical protein. For reference purposes, the amino acid sequence of full length IC7 is SEQ ID NO: 41 herein. In the R6 genome IC7 is spr0174 [205]. The use of IC7 for immunisation is reported in reference 82 (SEQ ID NO: 152 therein).

Preferred IC7 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 41; and/or (b) comprising a fragment of at least ‘n’ consecutive amino acids of SEQ ID NO: 41, wherein ‘n’ is 7 or more (e.g. 8, 10, 12, 14, 16, 18, 20, 25, 30, 35, 40, 50, 60, 70, 80, 90, 100, 150, 200, 250 or more). These IC7 proteins include variants of SEQ ID NO: 41. Preferred fragments of (b) comprise an epitope from SEQ ID NO: 41. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 41 while retaining at least one epitope of SEQ ID NO: 41. Other fragments omit one or more protein domains. Immunogenic fragments of IC7 are identified in table 1 of reference 82.

IC8

IC8 is a Dihydrofolate:folylpolyglutamate synthetase. For reference purposes, the amino acid sequence of full length IC8 is SEQ ID NO: 42 herein. In the R6 genome IC8 is spr0178 [205]. The use of IC8 for immunisation is reported in reference 82 (SEQ ID NO: 153 therein). Preferred IC8 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 42; and/or (b) comprising a fragment of at least ‘n’ consecutive amino acids of SEQ ID NO: 42, wherein ‘n’ is 7 or more (e.g. 8, 10, 12, 14, 16, 18, 20, 25, 30, 35, 40, 50, 60, 70, 80, 90, 100, 150, 200, 250 or more). These IC8 proteins include variants of SEQ ID NO: 42. Preferred fragments of (b) comprise an epitope from SEQ ID NO: 42. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 42 while retaining at least one epitope of SEQ ID NO: 42. Other fragments omit one or more protein domains. Immunogenic fragments of IC8 are identified in table 1 of reference 82.

IC9

IC9 is a 50S ribosomal protein L2. For reference purposes, the amino acid sequence of full length IC9 is SEQ ID NO: 43 herein. In the R6 genome IC9 is spr0191 [205]. The use of IC9 for immunisation is reported in reference 82 (SEQ ID NO: 154 therein).

Preferred IC9 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 43; and/or (b) comprising a fragment of at least ‘n’ consecutive amino acids of SEQ ID NO: 43, wherein ‘n’ is 7 or more (e.g. 8, 10, 12, 14, 16, 18, 20, 25, 30, 35, 40, 50, 60, 70, 80, 90, 100, 150, 200, 250 or more). These IC9 proteins include variants of SEQ ID NO: 43. Preferred fragments of (b) comprise an epitope from SEQ ID NO: 43. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 43 while retaining at least one epitope of SEQ ID NO: 43. Other fragments omit one or more protein domains. Immunogenic fragments of IC9 are identified in table 1 of reference 82.

IC10

IC10 is a 30S Ribosomal protein S14. For reference purposes, the amino acid sequence of full length IC10 is SEQ ID NO: 44 herein. In the R6 genome IC10 is spr0202 [205]. The use of IC10 for immunisation is reported in reference 82 (SEQ ID NO: 155 therein).

Preferred IC10 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 44; and/or (b) comprising a fragment of at least ‘n’ consecutive amino acids of SEQ ID NO: 44, wherein ‘n’ is 7 or more (e.g. 8, 10, 12, 14, 16, 18, 20, 25, 30, 35, 40, 50, 60, 70, 80, 90, 100, 150, 200, 250 or more). These IC10 proteins include variants of SEQ ID NO: 44. Preferred fragments of (b) comprise an epitope from SEQ ID NO: 44. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 44 while retaining at least one epitope of SEQ ID NO: 44. Other fragments omit one or more protein domains. Immunogenic fragments of IC10 are identified in table 1 of reference 82.

IC11

IC11 is annotated in reference 82 as a hypothetical protein. For reference purposes, the amino acid sequence of full length IC11 is SEQ ID NO: 45 herein. In the R6 genome IC11 is spr0218 [205]. The use of IC11 for immunisation is reported in reference 82 (SEQ ID NO: 156 therein).

Preferred IC11 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 45; and/or (b) comprising a fragment of at least ‘n’ consecutive amino acids of SEQ ID NO: 45, wherein ‘n’ is 7 or more (e.g. 8, 10, 12, 14, 16, 18, 20, 25, 30, 35, 40, 50, 60, 70, 80, 90, 100, 150, 200, 250 or more). These IC11 proteins include variants of SEQ ID NO: 45. Preferred fragments of (b) comprise an epitope from SEQ ID NO: 45. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 45 while retaining at least one epitope of SEQ ID NO: 45. Other fragments omit one or more protein domains. Immunogenic fragments of IC11 are identified in table 1 of reference 82.

IC12

IC12 is a Formate acetyltransferase 3. For reference purposes, the amino acid sequence of full length IC12 is SEQ ID NO: 46 herein. In the R6 genome IC12 is spr0232 [205]. The use of IC12 for immunisation is reported in reference 82 (SEQ ID NO: 157 therein).

Preferred IC12 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 46; and/or (b) comprising a fragment of at least ‘n’ consecutive amino acids of SEQ ID NO: 46, wherein ‘n’ is 7 or more (e.g. 8, 10, 12, 14, 16, 18, 20, 25, 30, 35, 40, 50, 60, 70, 80, 90, 100, 150, 200, 250 or more). These IC12 proteins include variants of SEQ ID NO: 46. Preferred fragments of (b) comprise an epitope from SEQ ID NO: 46. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 46 while retaining at least one epitope of SEQ ID NO: 46. Other fragments omit one or more protein domains. Immunogenic fragments of IC12 are identified in table 1 of reference 82.

IC13

IC13 is a 30S ribosomal protein S9. For reference purposes, the amino acid sequence of full length IC13 is SEQ ID NO: 47 herein. In the R6 genome IC13 is spr0272 [205]. The use of IC13 for immunisation is reported in reference 82 (SEQ ID NO: 158 therein).

Preferred IC13 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 47; and/or (b) comprising a fragment of at least ‘n’ consecutive amino acids of SEQ ID NO: 47, wherein ‘n’ is 7 or more (e.g. 8, 10, 12, 14, 16, 18, 20, 25, 30, 35, 40, 50, 60, 70, 80, 90, 100, 150, 200, 250 or more). These IC13 proteins include variants of SEQ ID NO: 47. Preferred fragments of (b) comprise an epitope from SEQ ID NO: 47. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 47 while retaining at least one epitope of SEQ ID NO: 47. Other fragments omit one or more protein domains. Immunogenic fragments of IC13 are identified in table 1 of reference 82.

IC14

IC14 is a Transcription regulator. For reference purposes, the amino acid sequence of full length IC14 is SEQ ID NO: 48 herein. In the R6 genome IC14 is spr0298 [205]. The use of IC14 for immunisation is reported in reference 82 (SEQ ID NO: 159 therein).

Preferred IC14 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 48; and/or (b) comprising a fragment of at least ‘n’ consecutive amino acids of SEQ ID NO: 48, wherein ‘n’ is 7 or more (e.g. 8, 10, 12, 14, 16, 18, 20, 25, 30, 35, 40, 50, 60, 70, 80, 90, 100, 150, 200, 250 or more). These IC14 proteins include variants of SEQ ID NO: 48. Preferred fragments of (b) comprise an epitope from SEQ ID NO: 48. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 48 while retaining at least one epitope of SEQ ID NO: 48. Other fragments omit one or more protein domains. Immunogenic fragments of IC14 are identified in table 1 of reference 82.

IC15

IC15 is annotated in reference 82 as a cell wall surface anchor family protein. For reference purposes, the amino acid sequence of full length IC15 is SEQ ID NO: 49 herein. In the R6 genome IC15 is spr0328 [205]. The use of IC15 for immunisation is reported in reference 82 (SEQ ID NO: 160 therein), and it is shown to be protective in reference 114 (antigen SP0368).

Preferred IC15 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 49; and/or (b) comprising a fragment of at least ‘n’ consecutive amino acids of SEQ ID NO: 49, wherein ‘n’ is 7 or more (e.g. 8, 10, 12, 14, 16, 18, 20, 25, 30, 35, 40, 50, 60, 70, 80, 90, 100, 150, 200, 250 or more). These IC15 proteins include variants of SEQ ID NO: 49. Preferred fragments of (b) comprise an epitope from SEQ ID NO: 49. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 49 while retaining at least one epitope of SEQ ID NO: 49. Other fragments omit one or more protein domains. Immunogenic fragments of IC15 are identified in table 1 of reference 82.

IC16

IC16 is a Penicillin-binding protein 1A. For reference purposes, the amino acid sequence of full length IC16 is SEQ ID NO: 50 herein. In the R6 genome IC16 is spr0329 [205]. The use of IC16 for immunisation is reported in reference 82 (SEQ ID NO: 161 therein).

Preferred IC16 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 50; and/or (b) comprising a fragment of at least ‘n’ consecutive amino acids of SEQ ID NO: 50, wherein ‘n’ is 7 or more (e.g. 8, 10, 12, 14, 16, 18, 20, 25, 30, 35, 40, 50, 60, 70, 80, 90, 100, 150, 200, 250 or more). These IC16 proteins include variants of SEQ ID NO: 50. Preferred fragments of (b) comprise an epitope from SEQ ID NO: 50. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 50 while retaining at least one epitope of SEQ ID NO: 50. Other fragments omit one or more protein domains. Immunogenic fragments of IC16 are identified in table 1 of reference 82.

IC17

IC17 is annotated in reference 82 as a hypothetical protein. For reference purposes, the amino acid sequence of full length IC17 is SEQ ID NO: 51 herein. In the R6 genome IC17 is spr0334 [205]. The use of IC17 for immunisation is reported in reference 82 (SEQ ID NO: 162 therein).

Preferred IC17 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 51; and/or (b) comprising a fragment of at least ‘n’ consecutive amino acids of SEQ ID NO: 51, wherein ‘n’ is 7 or more (e.g. 8, 10, 12, 14, 16, 18, 20, 25, 30, 35, 40, 50, 60, 70, 80, 90, 100, 150, 200, 250 or more). These IC17 proteins include variants of SEQ ID NO: 51. Preferred fragments of (b) comprise an epitope from SEQ ID NO: 51. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 51 while retaining at least one epitope of SEQ ID NO: 51. Other fragments omit one or more protein domains. Immunogenic fragments of IC17 are identified in table 1 of reference 82.

IC18

IC18 is annotated in reference 82 as choline-binding protein F. For reference purposes, the amino acid sequence of full length IC18 is SEQ ID NO: 52 herein. In the R6 genome IC18 is spr0337 [205]. The use of IC18 for immunisation is reported in reference 82 (SEQ ID NO: 163 therein).

Preferred IC18 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 52; and/or (b) comprising a fragment of at least ‘n’ consecutive amino acids of SEQ ID NO: 52, wherein ‘n’ is 7 or more (e.g. 8, 10, 12, 14, 16, 18, 20, 25, 30, 35, 40, 50, 60, 70, 80, 90, 100, 150, 200, 250 or more). These IC18 proteins include variants of SEQ ID NO: 52. Preferred fragments of (b) comprise an epitope from SEQ ID NO: 52. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 52 while retaining at least one epitope of SEQ ID NO: 52. Other fragments omit one or more protein domains. Immunogenic fragments of IC18 are identified in table 1 of reference 82.

IC19

IC19 is annotated in reference 82 as a choline-binding protein J (cbpJ). For reference purposes, the amino acid sequence of full length IC19 is SEQ ID NO: 53 herein. The use of IC19 for immunisation is reported in reference 82 (SEQ ID NO: 164 therein).

Preferred IC19 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 53; and/or (b) comprising a fragment of at least ‘n’ consecutive amino acids of SEQ ID NO: 53, wherein ‘n’ is 7 or more (e.g. 8, 10, 12, 14, 16, 18, 20, 25, 30, 35, 40, 50, 60, 70, 80, 90, 100, 150, 200, 250 or more). These IC19 proteins include variants of SEQ ID NO: 53. Preferred fragments of (b) comprise an epitope from SEQ ID NO: 53. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 53 while retaining at least one epitope of SEQ ID NO: 53. Other fragments omit one or more protein domains. Immunogenic fragments of IC19 are identified in table 1 of reference 82.

IC20

IC20 is a choline binding protein G. For reference purposes, the amino acid sequence of full length IC20 is SEQ ID NO: 54 herein. In the R6 genome IC20 is spr0349 [205]. The use of IC20 for immunisation is reported in reference 82 (SEQ ID NO: 165 therein).

Preferred IC20 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 54; and/or (b) comprising a fragment of at least ‘n’ consecutive amino acids of SEQ ID NO: 54, wherein ‘n’ is 7 or more (e.g. 8, 10, 12, 14, 16, 18, 20, 25, 30, 35, 40, 50, 60, 70, 80, 90, 100, 150, 200, 250 or more). These IC20 proteins include variants of SEQ ID NO: 54. Preferred fragments of (b) comprise an epitope from SEQ ID NO: 54. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 54 while retaining at least one epitope of SEQ ID NO: 54. Other fragments omit one or more protein domains. Immunogenic fragments of IC20 are identified in table 1 of reference 82.

IC21

IC21 is annotated in reference 82 as a hypothetical protein. For reference purposes, the amino acid sequence of full length IC21 is SEQ ID NO: 55 herein. In the R6 genome IC21 is spr0410 [205]. The use of IC21 for immunisation is reported in reference 82 (SEQ ID NO: 166 therein).

Preferred IC21 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 55; and/or (b) comprising a fragment of at least ‘n’ consecutive amino acids of SEQ ID NO: 55, wherein ‘n’ is 7 or more (e.g. 8, 10, 12, 14, 16, 18, 20, 25, 30, 35, 40, 50, 60, 70, 80, 90, 100, 150, 200, 250 or more). These IC21 proteins include variants of SEQ ID NO: 55. Preferred fragments of (b) comprise an epitope from SEQ ID NO: 55. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 55 while retaining at least one epitope of SEQ ID NO: 55. Other fragments omit one or more protein domains. Immunogenic fragments of IC21 are identified in table 1 of reference 82.

IC22

IC22 is annotated in reference 82 as cell wall surface anchor family protein. For reference purposes, the amino acid sequence of full length IC22 is SEQ ID NO: 56 herein. In the R6 genome IC22 is spr0051 [205]. The use of IC22 for immunisation is reported in reference 82 (SEQ ID NO: 167 therein).

Preferred IC22 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 56; and/or (b) comprising a fragment of at least ‘n’ consecutive amino acids of SEQ ID NO: 56, wherein ‘n’ is 7 or more (e.g. 8, 10, 12, 14, 16, 18, 20, 25, 30, 35, 40, 50, 60, 70, 80, 90, 100, 150, 200, 250 or more). These IC22 proteins include variants of SEQ ID NO: 56. Preferred fragments of (b) comprise an epitope from SEQ ID NO: 56. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 56 while retaining at least one epitope of SEQ ID NO: 56. Other fragments omit one or more protein domains. Immunogenic fragments of IC22 are identified in table 1 of reference 82.

IC23

IC23 is a Sortase (cf. spr1098). For reference purposes, the amino acid sequence of full length IC23 is SEQ ID NO: 57 herein. The use of IC23 for immunisation is reported in reference 82 (SEQ ID NO: 168 therein).

Preferred IC23 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 57; and/or (b) comprising a fragment of at least ‘n’ consecutive amino acids of SEQ ID NO: 57, wherein ‘n’ is 7 or more (e.g. 8, 10, 12, 14, 16, 18, 20, 25, 30, 35, 40, 50, 60, 70, 80, 90, 100, 150, 200, 250 or more). These IC23 proteins include variants of SEQ ID NO: 57. Preferred fragments of (b) comprise an epitope from SEQ ID NO: 57. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 57 while retaining at least one epitope of SEQ ID NO: 57. Other fragments omit one or more protein domains. Immunogenic fragments of IC23 are identified in table 1 of reference 82.

IC24

IC24 is a Sortase (cf. spr1098). For reference purposes, the amino acid sequence of full length IC24 is SEQ ID NO: 58 herein. The use of IC24 for immunisation is reported in reference 82 (SEQ ID NO: 169 therein).

Preferred IC24 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 58; and/or (b) comprising a fragment of at least ‘n’ consecutive amino acids of SEQ ID NO: 58, wherein ‘n’ is 7 or more (e.g. 8, 10, 12, 14, 16, 18, 20, 25, 30, 35, 40, 50, 60, 70, 80, 90, 100, 150, 200, 250 or more). These IC24 proteins include variants of SEQ ID NO: 58. Preferred fragments of (b) comprise an epitope from SEQ ID NO: 58. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 58 while retaining at least one epitope of SEQ ID NO: 58. Other fragments omit one or more protein domains. Immunogenic fragments of IC24 are identified in table 1 of reference 82.

IC25

IC25 is annotated in reference 82 as a putative endo-P-N-acetylglucosaminidase. For reference purposes, the amino acid sequence of full length IC25 is SEQ ID NO: 59 herein. In the R6 genome IC25 is spr0440 [205]. The use of IC25 for immunisation is reported in reference 82 (SEQ ID NO: 170 therein).

Preferred IC25 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 59; and/or (b) comprising a fragment of at least ‘n’ consecutive amino acids of SEQ ID NO: 59, wherein ‘n’ is 7 or more (e.g. 8, 10, 12, 14, 16, 18, 20, 25, 30, 35, 40, 50, 60, 70, 80, 90, 100, 150, 200, 250 or more). These IC25 proteins include variants of SEQ ID NO: 59. Preferred fragments of (b) comprise an epitope from SEQ ID NO: 59. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 59 while retaining at least one epitope of SEQ ID NO: 59. Other fragments omit one or more protein domains. Immunogenic fragments of IC25 are identified in table 1 of reference 82.

IC26

IC26 is a EcoE type I restriction modification enzyme. For reference purposes, the amino acid sequence of full length IC26 is SEQ ID NO: 60 herein. In the R6 genome IC26 is spr0449 [205]. The use of IC26 for immunisation is reported in reference 82 (SEQ ID NO: 171 therein).

Preferred IC26 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 60; and/or (b) comprising a fragment of at least ‘n’ consecutive amino acids of SEQ ID NO: 60, wherein ‘n’ is 7 or more (e.g. 8, 10, 12, 14, 16, 18, 20, 25, 30, 35, 40, 50, 60, 70, 80, 90, 100, 150, 200, 250 or more). These IC26 proteins include variants of SEQ ID NO: 60. Preferred fragments of (b) comprise an epitope from SEQ ID NO: 60. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 60 while retaining at least one epitope of SEQ ID NO: 60. Other fragments omit one or more protein domains. Immunogenic fragments of IC26 are identified in table 1 of reference 82.

IC27

IC27 is annotated in reference 82 as dnaJ protein. For reference purposes, the amino acid sequence of full length IC27 is SEQ ID NO: 61 herein. In the R6 genome IC27 is spr0456 [205]. The use of IC27 for immunisation is reported in reference 82 (SEQ ID NO: 172 therein).

Preferred IC27 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 61; and/or (b) comprising a fragment of at least ‘n’ consecutive amino acids of SEQ ID NO: 61, wherein ‘n’ is 7 or more (e.g. 8, 10, 12, 14, 16, 18, 20, 25, 30, 35, 40, 50, 60, 70, 80, 90, 100, 150, 200, 250 or more). These IC27 proteins include variants of SEQ ID NO: 61. Preferred fragments of (b) comprise an epitope from SEQ ID NO: 61. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 61 while retaining at least one epitope of SEQ ID NO: 61. Other fragments omit one or more protein domains. Immunogenic fragments of IC27 are identified in table 1 of reference 82.

IC28

IC28 is annotated in reference 82 as a BlpC ABC transporter (blpB). For reference purposes, the amino acid sequence of full length IC28 is SEQ ID NO: 62 herein. In the R6 genome IC28 is spr0466 [205]. The use of IC28 for immunisation is reported in reference 82 (SEQ ID NO: 173 therein).

Preferred IC28 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 62; and/or (b) comprising a fragment of at least ‘n’ consecutive amino acids of SEQ ID NO: 62, wherein ‘n’ is 7 or more (e.g. 8, 10, 12, 14, 16, 18, 20, 25, 30, 35, 40, 50, 60, 70, 80, 90, 100, 150, 200, 250 or more). These IC28 proteins include variants of SEQ ID NO: 62. Preferred fragments of (b) comprise an epitope from SEQ ID NO: 62. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 62 while retaining at least one epitope of SEQ ID NO: 62. Other fragments omit one or more protein domains. Immunogenic fragments of IC28 are identified in table 1 of reference 82.

IC29

IC29 is annotated in reference 82 as a hypothetical protein. For reference purposes, the amino acid sequence of full length IC29 is SEQ ID NO: 63 herein. In the R6 genome IC29 is spr0488 [205]. The use of IC29 for immunisation is reported in reference 82 (SEQ ID NO: 174 therein).

Preferred IC29 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 63; and/or (b) comprising a fragment of at least ‘n’ consecutive amino acids of SEQ ID NO: 63, wherein ‘n’ is 7 or more (e.g. 8, 10, 12, 14, 16, 18, 20, 25, 30, 35, 40, 50, 60, 70, 80, 90, 100, 150, 200, 250 or more). These IC29 proteins include variants of SEQ ID NO: 63. Preferred fragments of (b) comprise an epitope from SEQ ID NO: 63. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 63 while retaining at least one epitope of SEQ ID NO: 63. Other fragments omit one or more protein domains. Immunogenic fragments of IC29 are identified in table 1 of reference 82.

IC30

IC30 is a ABC transporter substrate-binding protein. For reference purposes, the amino acid sequence of full length IC30 is SEQ ID NO: 64 herein. In the R6 genome IC30 is spr0534 [205]. The use of IC30 for immunisation is reported in reference 82 (SEQ ID NO: 175 therein).

Preferred IC30 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 64; and/or (b) comprising a fragment of at least ‘n’ consecutive amino acids of SEQ ID NO: 64, wherein ‘n’ is 7 or more (e.g. 8, 10, 12, 14, 16, 18, 20, 25, 30, 35, 40, 50, 60, 70, 80, 90, 100, 150, 200, 250 or more). These IC30 proteins include variants of SEQ ID NO: 64. Preferred fragments of (b) comprise an epitope from SEQ ID NO: 64. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 64 while retaining at least one epitope of SEQ ID NO: 64. Other fragments omit one or more protein domains. Immunogenic fragments of IC30 are identified in table 1 of reference 82.

IC31

IC31 is annotated in reference 82 as a metallo-P-lactamase superfamily protein. For reference purposes, the amino acid sequence of full length IC31 is SEQ ID NO: 65 herein. In the R6 genome IC31 is spr0538 [205]. The use of IC31 for immunisation is reported in reference 82 (SEQ ID NO: 176 therein).

Preferred IC31 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 65; and/or (b) comprising a fragment of at least ‘n’ consecutive amino acids of SEQ ID NO: 65, wherein ‘n’ is 7 or more (e.g. 8, 10, 12, 14, 16, 18, 20, 25, 30, 35, 40, 50, 60, 70, 80, 90, 100, 150, 200, 250 or more). These IC31 proteins include variants of SEQ ID NO: 65. Preferred fragments of (b) comprise an epitope from SEQ ID NO: 65. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 65 while retaining at least one epitope of SEQ ID NO: 65. Other fragments omit one or more protein domains. Immunogenic fragments of IC31 are identified in table 1 of reference 82.

IC32

IC32 is a variant form of spr0565, as mentioned above (SEQ ID NO: 66 herein). Useful IC32 polypeptides may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 66; and/or (b) comprising a fragment of at least ‘n’ consecutive amino acids of SEQ ID NO: 66, wherein ‘n’ is 7 or more (e.g. 8, 10, 12, 14, 16, 18, 20, 25, 30, 35, 40, 50, 60, 70, 80, 90, 100, 150, 200, 250 or more). These IC32 polypeptides include variants of SEQ ID NO: 66. Preferred fragments of (b) comprise an epitope from SEQ ID NO: 66. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 66 while retaining at least one epitope of SEQ ID NO: 66. Other fragments omit one or more protein domains. Immunogenic fragments of SEQ ID NO: 66 are identified in table 1 of reference 82.

IC33

IC33 is annotated in reference 82 as a putative pneumococcal surface protein. For reference purposes, the amino acid sequence of full length IC33 is SEQ ID NO: 67 herein. In the R6 genome IC33 is spr0583 [205]. The use of IC33 for immunisation is reported in reference 82 (SEQ ID NO: 180 therein) and it is shown to be protective in reference 114 (antigen SP0667).

Preferred IC33 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 67; and/or (b) comprising a fragment of at least ‘n’ consecutive amino acids of SEQ ID NO: 67, wherein ‘n’ is 7 or more (e.g. 8, 10, 12, 14, 16, 18, 20, 25, 30, 35, 40, 50, 60, 70, 80, 90, 100, 150, 200, 250 or more). These IC33 proteins include variants of SEQ ID NO: 67. Preferred fragments of (b) comprise an epitope from SEQ ID NO: 67. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 67 while retaining at least one epitope of SEQ ID NO: 67. Other fragments omit one or more protein domains. Immunogenic fragments of IC33 are identified in table 1 of reference 82.

IC34

IC34 is a UDP-N-acetylmuramoyl-L-alanyl-D-glutamate synthetase. For reference purposes, the amino acid sequence of full length IC34 is SEQ ID NO: 68 herein. In the R6 genome IC34 is spr0603 [205]. The use of IC34 for immunisation is reported in reference 82 (SEQ ID NO: 181 therein).

Preferred IC34 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 68; and/or (b) comprising a fragment of at least ‘n’ consecutive amino acids of SEQ ID NO: 68, wherein ‘n’ is 7 or more (e.g. 8, 10, 12, 14, 16, 18, 20, 25, 30, 35, 40, 50, 60, 70, 80, 90, 100, 150, 200, 250 or more). These IC34 proteins include variants of SEQ ID NO: 68. Preferred fragments of (b) comprise an epitope from SEQ ID NO: 68. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 68 while retaining at least one epitope of SEQ ID NO: 68. Other fragments omit one or more protein domains. Immunogenic fragments of IC34 are identified in table 1 of reference 82.

IC35

IC35 is a ABC transporter substrate-binding protein. For reference purposes, the amino acid sequence of full length IC35 is SEQ ID NO: 69 herein. In the R6 genome IC35 is spr0659 [205]. The use of IC35 for immunisation is reported in reference 82 (SEQ ID NO: 182 therein).

Preferred IC35 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 69; and/or (b) comprising a fragment of at least ‘n’ consecutive amino acids of SEQ ID NO: 69, wherein ‘n’ is 7 or more (e.g. 8, 10, 12, 14, 16, 18, 20, 25, 30, 35, 40, 50, 60, 70, 80, 90, 100, 150, 200, 250 or more). These IC35 proteins include variants of SEQ ID NO: 69. Preferred fragments of (b) comprise an epitope from SEQ ID NO: 69. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 69 while retaining at least one epitope of SEQ ID NO: 69. Other fragments omit one or more protein domains. Immunogenic fragments of IC35 are identified in table 1 of reference 82.

IC36

IC36 is a ABC transporter ATP-binding protein. For reference purposes, the amino acid sequence of full length IC36 is SEQ ID NO: 70 herein. In the R6 genome IC36 is spr0678 [205]. The use of IC36 for immunisation is reported in reference 82 (SEQ ID NO: 183 therein).

Preferred IC36 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 70; and/or (b) comprising a fragment of at least ‘n’ consecutive amino acids of SEQ ID NO: 70, wherein ‘n’ is 7 or more (e.g. 8, 10, 12, 14, 16, 18, 20, 25, 30, 35, 40, 50, 60, 70, 80, 90, 100, 150, 200, 250 or more). These IC36 proteins include variants of SEQ ID NO: 70. Preferred fragments of (b) comprise an epitope from SEQ ID NO: 70. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 70 while retaining at least one epitope of SEQ ID NO: 70. Other fragments omit one or more protein domains. Immunogenic fragments of IC36 are identified in table 1 of reference 82.

IC37

IC37 is annotated in reference 82 as a hypothetical protein. For reference purposes, the amino acid sequence of full length IC37 is SEQ ID NO: 71 herein. In the R6 genome IC37 is spr0693 [205]. The use of IC37 for immunisation is reported in reference 82 (SEQ ID NO: 184 therein).

Preferred IC37 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 71; and/or (b) comprising a fragment of at least ‘n’ consecutive amino acids of SEQ ID NO: 71, wherein ‘n’ is 7 or more (e.g. 8, 10, 12, 14, 16, 18, 20, 25, 30, 35, 40, 50, 60, 70, 80, 90, 100, 150, 200, 250 or more). These IC37 proteins include variants of SEQ ID NO: 71. Preferred fragments of (b) comprise an epitope from SEQ ID NO: 71. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 71 while retaining at least one epitope of SEQ ID NO: 71. Other fragments omit one or more protein domains. Immunogenic fragments of IC37 are identified in table 1 of reference 82.

IC38

IC38 is annotated in reference 82 as a nodulin-related protein with truncation. For reference purposes, the amino acid sequence of full length IC38 is SEQ ID NO: 72 herein. In the R6 genome IC38 is spr0814 [205]. The use of IC38 for immunisation is reported in reference 82 (SEQ ID NO: 185 therein).

Preferred IC38 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 72; and/or (b) comprising a fragment of at least ‘n’ consecutive amino acids of SEQ ID NO: 72, wherein ‘n’ is 7 or more (e.g. 8, 10, 12, 14, 16, 18, 20, 25, 30, 35, 40, 50, 60, 70, 80, 90, 100, 150, 200, 250 or more). These IC38 proteins include variants of SEQ ID NO: 72. Preferred fragments of (b) comprise an epitope from SEQ ID NO: 72. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 72 while retaining at least one epitope of SEQ ID NO: 72. Other fragments omit one or more protein domains. Immunogenic fragments of IC38 are identified in table 1 of reference 82.

IC39

IC39 is a Teichoic acid phosphorylcholine esterase/choline binding protein E (cbpE). It may also be known as ‘LytD’. For reference purposes, the amino acid sequence of full length IC39 is SEQ ID NO: 73 herein. In the R6 genome IC39 is spr0831 [205]. The use of IC39 for immunisation is reported in reference 82 (SEQ ID NO: 186 therein).

Preferred IC39 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 73; and/or (b) comprising a fragment of at least ‘n’ consecutive amino acids of SEQ ID NO: 73, wherein ‘n’ is 7 or more (e.g. 8, 10, 12, 14, 16, 18, 20, 25, 30, 35, 40, 50, 60, 70, 80, 90, 100, 150, 200, 250 or more). These IC39 proteins include variants of SEQ ID NO: 73. Preferred fragments of (b) comprise an epitope from SEQ ID NO: 73. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 73 while retaining at least one epitope of SEQ ID NO: 73. Other fragments omit one or more protein domains. Immunogenic fragments of IC39 are identified in table 1 of reference 82.

IC40

IC40 is a glucose-inhibited division protein A. For reference purposes, the amino acid sequence of full length IC40 is SEQ ID NO: 74 herein. In the R6 genome IC40 is spr0844 [205]. The use of IC40 for immunisation is reported in reference 82 (SEQ ID NO: 187 therein).

Preferred IC40 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 74; and/or (b) comprising a fragment of at least ‘n’ consecutive amino acids of SEQ ID NO: 74, wherein ‘n’ is 7 or more (e.g. 8, 10, 12, 14, 16, 18, 20, 25, 30, 35, 40, 50, 60, 70, 80, 90, 100, 150, 200, 250 or more). These IC40 proteins include variants of SEQ ID NO: 74. Preferred fragments of (b) comprise an epitope from SEQ ID NO: 74. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 74 while retaining at least one epitope of SEQ ID NO: 74. Other fragments omit one or more protein domains. Immunogenic fragments of IC40 are identified in table 1 of reference 82.

IC41

IC41 is a Alanine dehydrogenase, truncation. For reference purposes, the amino acid sequence of full length IC41 is SEQ ID NO: 75 herein. In the R6 genome IC41 is spr0854 [205]. The use of IC41 for immunisation is reported in reference 82 (SEQ ID NO: 188 therein).

Preferred IC41 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 75; and/or (b) comprising a fragment of at least ‘n’ consecutive amino acids of SEQ ID NO: 75, wherein ‘n’ is 7 or more (e.g. 8, 10, 12, 14, 16, 18, 20, 25, 30, 35, 40, 50, 60, 70, 80, 90, 100, 150, 200, 250 or more). These IC41 proteins include variants of SEQ ID NO: 75. Preferred fragments of (b) comprise an epitope from SEQ ID NO: 75. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 75 while retaining at least one epitope of SEQ ID NO: 75. Other fragments omit one or more protein domains. Immunogenic fragments of IC41 are identified in table 1 of reference 82.

IC42

IC42 is a glycogen syntase. For reference purposes, the amino acid sequence of full length IC42 is SEQ ID NO: 76 herein. In the R6 genome IC42 is spr1032 [205]. The use of IC42 for immunisation is reported in reference 82 (SEQ ID NO: 191 therein).

Preferred IC42 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 76; and/or (b) comprising a fragment of at least ‘n’ consecutive amino acids of SEQ ID NO: 76, wherein ‘n’ is 7 or more (e.g. 8, 10, 12, 14, 16, 18, 20, 25, 30, 35, 40, 50, 60, 70, 80, 90, 100, 150, 200, 250 or more). These IC42 proteins include variants of SEQ ID NO: 76. Preferred fragments of (b) comprise an epitope from SEQ ID NO: 76. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 76 while retaining at least one epitope of SEQ ID NO: 76. Other fragments omit one or more protein domains. Immunogenic fragments of IC42 are identified in table 1 of reference 82.

IC43

IC43 is a Immunoglobulin Al protease. For reference purposes, the amino acid sequence of full length IC43 is SEQ ID NO: 77 herein. The use of IC43 for immunisation is reported in reference 82 (SEQ ID NO: 192 therein).

Preferred IC43 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 77; and/or (b) comprising a fragment of at least ‘n’ consecutive amino acids of SEQ ID NO: 77, wherein ‘n’ is 7 or more (e.g. 8, 10, 12, 14, 16, 18, 20, 25, 30, 35, 40, 50, 60, 70, 80, 90, 100, 150, 200, 250 or more). These IC43 proteins include variants of SEQ ID NO: 77. Preferred fragments of (b) comprise an epitope from SEQ ID NO: 77. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 77 while retaining at least one epitope of SEQ ID NO: 77. Other fragments omit one or more protein domains. Immunogenic fragments of IC43 are identified in table 1 of reference 82.

IC44

IC44 is a Uncharacterized restriction enzyme. For reference purposes, the amino acid sequence of full length IC44 is SEQ ID NO: 78 herein. In the R6 genome IC44 is spr1101 [205]. The use of IC44 for immunisation is reported in reference 82 (SEQ ID NO: 195 therein).

Preferred IC44 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 78; and/or (b) comprising a fragment of at least ‘n’ consecutive amino acids of SEQ ID NO: 78, wherein ‘n’ is 7 or more (e.g. 8, 10, 12, 14, 16, 18, 20, 25, 30, 35, 40, 50, 60, 70, 80, 90, 100, 150, 200, 250 or more). These IC44 proteins include variants of SEQ ID NO: 78. Preferred fragments of (b) comprise an epitope from SEQ ID NO: 78. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 78 while retaining at least one epitope of SEQ ID NO: 78. Other fragments omit one or more protein domains. Immunogenic fragments of IC44 are identified in table 1 of reference 82.

IC45

IC45 is a Response regulator. For reference purposes, the amino acid sequence of full length IC45 is SEQ ID NO: 79 herein. In the R6 genome IC45 is spr1107 [205]. The use of IC45 for immunisation is reported in reference 82 (SEQ ID NO: 196 therein).

Preferred IC45 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 79; and/or (b) comprising a fragment of at least ‘n’ consecutive amino acids of SEQ ID NO: 79, wherein ‘n’ is 7 or more (e.g. 8, 10, 12, 14, 16, 18, 20, 25, 30, 35, 40, 50, 60, 70, 80, 90, 100, 150, 200, 250 or more). These IC45 proteins include variants of SEQ ID NO: 79. Preferred fragments of (b) comprise an epitope from SEQ ID NO: 79. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 79 while retaining at least one epitope of SEQ ID NO: 79. Other fragments omit one or more protein domains. Immunogenic fragments of IC45 are identified in table 1 of reference 82.

IC46

IC46 is a ABC transporter membrane spanning permease. For reference purposes, the amino acid sequence of full length IC46 is SEQ ID NO: 80 herein. In the R6 genome IC46 is spr1120 [205]. The use of IC46 for immunisation is reported in reference 82 (SEQ ID NO: 197 therein).

Preferred IC46 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 80; and/or (b) comprising a fragment of at least ‘n’ consecutive amino acids of SEQ ID NO: 80, wherein ‘n’ is 7 or more (e.g. 8, 10, 12, 14, 16, 18, 20, 25, 30, 35, 40, 50, 60, 70, 80, 90, 100, 150, 200, 250 or more). These IC46 proteins include variants of SEQ ID NO: 80. Preferred fragments of (b) comprise an epitope from SEQ ID NO: 80. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 80 while retaining at least one epitope of SEQ ID NO: 80. Other fragments omit one or more protein domains. Immunogenic fragments of IC46 are identified in table 1 of reference 82.

IC47

IC47 is a Signal recognition particle. For reference purposes, the amino acid sequence of full length IC47 is SEQ ID NO: 81 herein. In the R6 genome IC47 is spr1166 [205]. The use of IC47 for immunisation is reported in reference 82 (SEQ ID NO: 198 therein).

Preferred IC47 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 81; and/or (b) comprising a fragment of at least ‘n’ consecutive amino acids of SEQ ID NO: 81, wherein ‘n’ is 7 or more (e.g. 8, 10, 12, 14, 16, 18, 20, 25, 30, 35, 40, 50, 60, 70, 80, 90, 100, 150, 200, 250 or more). These IC47 proteins include variants of SEQ ID NO: 81. Preferred fragments of (b) comprise an epitope from SEQ ID NO: 81. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 81 while retaining at least one epitope of SEQ ID NO:

81. Other fragments omit one or more protein domains. Immunogenic fragments of IC47 are identified in table 1 of reference 82.

IC48

IC48 is a N-acetylmannosamine-6-phosphate 2-epimerase. For reference purposes, the amino acid sequence of full length IC48 is SEQ ID NO: 82 herein. In the R6 genome IC48 is spr1529 [205]. The use of IC48 for immunisation is reported in reference 82 (SEQ ID NO: 199 therein).

Preferred IC48 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 82; and/or (b) comprising a fragment of at least ‘n’ consecutive amino acids of SEQ ID NO: 82, wherein ‘n’ is 7 or more (e.g. 8, 10, 12, 14, 16, 18, 20, 25, 30, 35, 40, 50, 60, 70, 80, 90, 100, 150, 200, 250 or more). These IC48 proteins include variants of SEQ ID NO: 82. Preferred fragments of (b) comprise an epitope from SEQ ID NO: 82. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 82 while retaining at least one epitope of SEQ ID NO: 82. Other fragments omit one or more protein domains. Immunogenic fragments of IC48 are identified in table 1 of reference 82.

IC49

IC49 is a chorismate synthase. For reference purposes, the amino acid sequence of full length IC49 is SEQ ID NO: 83 herein. In the R6 genome IC49 is spr1232 [205]. The use of IC49 for immunisation is reported in reference 82 (SEQ ID NO: 200 therein).

Preferred IC49 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 83; and/or (b) comprising a fragment of at least ‘n’ consecutive amino acids of SEQ ID NO: 83, wherein ‘n’ is 7 or more (e.g. 8, 10, 12, 14, 16, 18, 20, 25, 30, 35, 40, 50, 60, 70, 80, 90, 100, 150, 200, 250 or more). These IC49 proteins include variants of SEQ ID NO: 83. Preferred fragments of (b) comprise an epitope from SEQ ID NO: 83. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 83 while retaining at least one epitope of SEQ ID NO: 83. Other fragments omit one or more protein domains. Immunogenic fragments of IC49 are identified in table 1 of reference 82.

IC50

IC50 is annotated in reference 82 as a hypothetical protein. For reference purposes, the amino acid sequence of full length IC50 is SEQ ID NO: 84 herein. In the R6 genome IC50 is spr1236 [205]. The use of IC50 for immunisation is reported in reference 82 (SEQ ID NO: 201 therein).

Preferred IC50 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 84; and/or (b) comprising a fragment of at least ‘n’ consecutive amino acids of SEQ ID NO: 84, wherein ‘n’ is 7 or more (e.g. 8, 10, 12, 14, 16, 18, 20, 25, 30, 35, 40, 50, 60, 70, 80, 90, 100, 150, 200, 250 or more). These IC50 proteins include variants of SEQ ID NO: 84. Preferred fragments of (b) comprise an epitope from SEQ ID NO: 84. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 84 while retaining at least one epitope of SEQ ID NO: 84. Other fragments omit one or more protein domains. Immunogenic fragments of IC50 are identified in table 1 of reference 82.

IC51

IC51 is a Protease. For reference purposes, the amino acid sequence of full length IC51 is SEQ ID NO: 85 herein. In the R6 genome IC51 is spr1284 [205]. The use of IC51 for immunisation is reported in reference 82 (SEQ ID NO: 202 therein).

Preferred IC51 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 85; and/or (b) comprising a fragment of at least ‘n’ consecutive amino acids of SEQ ID NO: 85, wherein ‘n’ is 7 or more (e.g. 8, 10, 12, 14, 16, 18, 20, 25, 30, 35, 40, 50, 60, 70, 80, 90, 100, 150, 200, 250 or more). These IC51 proteins include variants of SEQ ID NO: 85. Preferred fragments of (b) comprise an epitope from SEQ ID NO: 85. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 85 while retaining at least one epitope of SEQ ID NO: 85. Other fragments omit one or more protein domains. Immunogenic fragments of IC51 are identified in table 1 of reference 82.

IC52

IC52 is a annotated in reference 82 as an oxidoreductase or aldo/keto reductase. For reference purposes, the amino acid sequence of full length IC52 is SEQ ID NO: 86 herein. In the R6 genome IC52 is spr1332 [205]. The use of IC52 for immunisation is reported in reference 82 (SEQ ID NO: 203 therein).

Preferred IC52 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 86; and/or (b) comprising a fragment of at least ‘n’ consecutive amino acids of SEQ ID NO: 86, wherein ‘n’ is 7 or more (e.g. 8, 10, 12, 14, 16, 18, 20, 25, 30, 35, 40, 50, 60, 70, 80, 90, 100, 150, 200, 250 or more). These IC52 proteins include variants of SEQ ID NO: 86. Preferred fragments of (b) comprise an epitope from SEQ ID NO: 86. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 86 while retaining at least one epitope of SEQ ID NO: 86. Other fragments omit one or more protein domains. Immunogenic fragments of IC52 are identified in table 1 of reference 82.

IC53

IC53 is annotated in reference 82 as a hypothetical protein. For reference purposes, the amino acid sequence of full length IC53 is SEQ ID NO: 87 herein. In the R6 genome IC53 is spr1370 [205]. The use of IC53 for immunisation is reported in reference 82 (SEQ ID NO: 204 therein).

Preferred IC53 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 87; and/or (b) comprising a fragment of at least ‘n’ consecutive amino acids of SEQ ID NO: 87, wherein ‘n’ is 7 or more (e.g. 8, 10, 12, 14, 16, 18, 20, 25, 30, 35, 40, 50, 60, 70, 80, 90, 100, 150, 200, 250 or more). These IC53 proteins include variants of SEQ ID NO: 87. Preferred fragments of (b) comprise an epitope from SEQ ID NO: 87. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 87 while retaining at least one epitope of SEQ ID NO: 87. Other fragments omit one or more protein domains. Immunogenic fragments of IC53 are identified in table 1 of reference 82.

IC54

IC54 is annotated as a conserved domain protein. For reference purposes, the amino acid sequence of full length IC54 is SEQ ID NO: 88 herein. In the R6 genome IC54 is spr1374 [205]. The use of IC54 for immunisation is reported in reference 82 (SEQ ID NO: 205 therein).

Preferred IC54 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 88; and/or (b) comprising a fragment of at least ‘n’ consecutive amino acids of SEQ ID NO: 88, wherein ‘n’ is 7 or more (e.g. 8, 10, 12, 14, 16, 18, 20, 25, 30, 35, 40, 50, 60, 70, 80, 90, 100, 150, 200, 250 or more). These IC54 proteins include variants of SEQ ID NO: 88. Preferred fragments of (b) comprise an epitope from SEQ ID NO: 88. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 88 while retaining at least one epitope of SEQ ID NO: 88. Other fragments omit one or more protein domains. Immunogenic fragments of IC54 are identified in table 1 of reference 82.

IC55

IC55 is a ABC transporter substrate-binding protein. For reference purposes, the amino acid sequence of full length IC55 is SEQ ID NO: 89 herein. In the R6 genome IC55 is spr1382 [205]. The use of IC55 for immunisation is reported in reference 82 (SEQ ID NO: 206 therein).

Preferred IC55 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 89; and/or (b) comprising a fragment of at least ‘n’ consecutive amino acids of SEQ ID NO: 89, wherein ‘n’ is 7 or more (e.g. 8, 10, 12, 14, 16, 18, 20, 25, 30, 35, 40, 50, 60, 70, 80, 90, 100, 150, 200, 250 or more). These IC55 proteins include variants of SEQ ID NO: 89. Preferred fragments of (b) comprise an epitope from SEQ ID NO: 89. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 89 while retaining at least one epitope of SEQ ID NO: 89. Other fragments omit one or more protein domains. Immunogenic fragments of IC55 are identified in table 1 of reference 82.

IC56

IC56 is annotated in reference 82 as a hypothetical protein. For reference purposes, the amino acid sequence of full length IC56 is SEQ ID NO: 90 herein. In the R6 genome IC56 is spr1457 [205]. The use of IC56 for immunisation is reported in reference 82 (SEQ ID NO: 208 therein).

Preferred IC56 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 90; and/or (b) comprising a fragment of at least ‘n’ consecutive amino acids of SEQ ID NO: 90, wherein ‘n’ is 7 or more (e.g. 8, 10, 12, 14, 16, 18, 20, 25, 30, 35, 40, 50, 60, 70, 80, 90, 100, 150, 200, 250 or more). These IC56 proteins include variants of SEQ ID NO: 90. Preferred fragments of (b) comprise an epitope from SEQ ID NO: 90. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 90 while retaining at least one epitope of SEQ ID NO: 90. Other fragments omit one or more protein domains. Immunogenic fragments of IC56 are identified in table 1 of reference 82.

IC57

IC57 is a Cell-division initiation protein. For reference purposes, the amino acid sequence of full length IC57 is SEQ ID NO: 91 herein. In the R6 genome IC57 is spr1505 [205]. The use of IC57 for immunisation is reported in reference 82 (SEQ ID NO: 209 therein).

Preferred IC57 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 91; and/or (b) comprising a fragment of at least ‘n’ consecutive amino acids of SEQ ID NO: 91, wherein ‘n’ is 7 or more (e.g. 8, 10, 12, 14, 16, 18, 20, 25, 30, 35, 40, 50, 60, 70, 80, 90, 100, 150, 200, 250 or more). These IC57 proteins include variants of SEQ ID NO: 91. Preferred fragments of (b) comprise an epitope from SEQ ID NO: 91. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 91 while retaining at least one epitope of SEQ ID NO: 91. Other fragments omit one or more protein domains. Immunogenic fragments of IC57 are identified in table 1 of reference 82.

IC58

IC58 is annotated in reference 82 as ylmF protein. For reference purposes, the amino acid sequence of full length IC58 is SEQ ID NO: 92 herein. In the R6 genome IC58 is spr1508 [205]. The use of IC58 for immunisation is reported in reference 82 (SEQ ID NO: 210 therein).

Preferred IC58 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 92; and/or (b) comprising a fragment of at least ‘n’ consecutive amino acids of SEQ ID NO: 92, wherein ‘n’ is 7 or more (e.g. 8, 10, 12, 14, 16, 18, 20, 25, 30, 35, 40, 50, 60, 70, 80, 90, 100, 150, 200, 250 or more). These IC58 proteins include variants of SEQ ID NO: 92. Preferred fragments of (b) comprise an epitope from SEQ ID NO: 92. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 92 while retaining at least one epitope of SEQ ID NO: 92. Other fragments omit one or more protein domains. Immunogenic fragments of IC58 are identified in table 1 of reference 82.

IC59

IC59 is a N-acetylneuraminate lyase subunit. For reference purposes, the amino acid sequence of full length IC59 is SEQ ID NO: 93 herein. In the R6 genome IC59 is spr1186 [205]. The use of IC59 for immunisation is reported in reference 82 (SEQ ID NO: 211 therein).

Preferred IC59 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 93; and/or (b) comprising a fragment of at least ‘n’ consecutive amino acids of SEQ ID NO: 93, wherein ‘n’ is 7 or more (e.g. 8, 10, 12, 14, 16, 18, 20, 25, 30, 35, 40, 50, 60, 70, 80, 90, 100, 150, 200, 250 or more). These IC59 proteins include variants of SEQ ID NO: 93. Preferred fragments of (b) comprise an epitope from SEQ ID NO: 93. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 93 while retaining at least one epitope of SEQ ID NO: 93. Other fragments omit one or more protein domains. Immunogenic fragments of IC59 are identified in table 1 of reference 82.

IC60

IC60 is a Eukaryotic-type serine/threonine kinase (StkP). For reference purposes, the amino acid sequence of full length IC60 is SEQ ID NO: 94 herein. In the R6 genome IC60 is spr1577 [205]. The use of IC60 for immunisation is reported in reference 82 (SEQ ID NO: 214 therein), and it is reported to be a lead vaccine candidate in reference 114.

Preferred IC60 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 94; and/or (b) comprising a fragment of at least ‘n’ consecutive amino acids of SEQ ID NO: 94, wherein ‘n’ is 7 or more (e.g. 8, 10, 12, 14, 16, 18, 20, 25, 30, 35, 40, 50, 60, 70, 80, 90, 100, 150, 200, 250 or more). These IC60 proteins include variants of SEQ ID NO: 94. Preferred fragments of (b) comprise an epitope from SEQ ID NO: 94. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 94 while retaining at least one epitope of SEQ ID NO: 94. Other fragments omit one or more protein domains. Immunogenic fragments of IC60 are identified in table 1 of reference 82. A further useful fragment is disclosed as SEQ ID NO: 2 in reference 91 (corresponding to amino acids 345-659 of SEQ ID NO: 94 herein).

IC61

IC61 is a methionyl-tRNA formyltransferase. For reference purposes, the amino acid sequence of full length IC61 is SEQ ID NO: 95 herein. In the R6 genome IC61 is spr1580 [205]. The use of IC61 for immunisation is reported in reference 82 (SEQ ID NO: 215 therein).

Preferred IC61 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 95; and/or (b) comprising a fragment of at least ‘n’ consecutive amino acids of SEQ ID NO: 95, wherein ‘n’ is 7 or more (e.g. 8, 10, 12, 14, 16, 18, 20, 25, 30, 35, 40, 50, 60, 70, 80, 90, 100, 150, 200, 250 or more). These IC61 proteins include variants of SEQ ID NO: 95. Preferred fragments of (b) comprise an epitope from SEQ ID NO: 95.

Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 95 while retaining at least one epitope of SEQ ID NO: 95. Other fragments omit one or more protein domains. Immunogenic fragments of IC61 are identified in table 1 of reference 82.

IC62

IC62 is a translocase. For reference purposes, the amino acid sequence of full length IC62 is SEQ ID NO: 96 herein. In the R6 genome IC62 is spr1544 [205]. The use of IC62 for immunisation is reported in reference 82 (SEQ ID NO: 216 therein).

Preferred IC62 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 96; and/or (b) comprising a fragment of at least ‘n’ consecutive amino acids of SEQ ID NO: 96, wherein ‘n’ is 7 or more (e.g. 8, 10, 12, 14, 16, 18, 20, 25, 30, 35, 40, 50, 60, 70, 80, 90, 100, 150, 200, 250 or more). These IC62 proteins include variants of SEQ ID NO: 96. Preferred fragments of (b) comprise an epitope from SEQ ID NO: 96. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 96 while retaining at least one epitope of SEQ ID NO: 96. Other fragments omit one or more protein domains. Immunogenic fragments of IC62 are identified in table 1 of reference 82.

IC63

IC63 is annotated in reference 82 as a cell wall surface anchor family protein. For reference purposes, the amino acid sequence of full length IC63 is SEQ ID NO: 97 herein. In the R6 genome IC63 is spr1403 [205]. The use of IC63 for immunisation is reported in reference 82 (SEQ ID NO: 217 therein).

Preferred IC63 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 97; and/or (b) comprising a fragment of at least ‘n’ consecutive amino acids of SEQ ID NO: 97, wherein ‘n’ is 7 or more (e.g. 8, 10, 12, 14, 16, 18, 20, 25, 30, 35, 40, 50, 60, 70, 80, 90, 100, 150, 200, 250 or more). These IC63 proteins include variants of SEQ ID NO: 97. Preferred fragments of (b) comprise an epitope from SEQ ID NO: 97. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 97 while retaining at least one epitope of SEQ ID NO: 97. Other fragments omit one or more protein domains. Immunogenic fragments of IC63 are identified in table 1 of reference 82.

IC64

IC64 is annotated in reference 82 as a putative general stress protein 24. For reference purposes, the amino acid sequence of full length IC64 is SEQ ID NO: 98 herein. In the R6 genome IC64 is spr1625 [205]. The use of IC64 for immunisation is reported in reference 82 (SEQ ID NO: 218 therein).

Preferred IC64 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 98; and/or (b) comprising a fragment of at least ‘n’ consecutive amino acids of SEQ ID NO: 98, wherein ‘n’ is 7 or more (e.g. 8, 10, 12, 14, 16, 18, 20, 25, 30, 35, 40, 50, 60, 70, 80, 90, 100, 150, 200, 250 or more). These IC64 proteins include variants of SEQ ID NO: 98. Preferred fragments of (b) comprise an epitope from SEQ ID NO: 98. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 98 while retaining at least one epitope of SEQ ID NO: 98. Other fragments omit one or more protein domains. Immunogenic fragments of IC64 are identified in table 1 of reference 82.

IC65

IC65 is a ABC transporter ATP-binding protein. For reference purposes, the amino acid sequence of full length IC65 is SEQ ID NO: 99 herein. In the R6 genome IC65 is spr1704 [205]. The use of IC65 for immunisation is reported in reference 82 (SEQ ID NO: 219 therein).

Preferred IC65 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 99; and/or (b) comprising a fragment of at least ‘n’ consecutive amino acids of SEQ ID NO: 99, wherein ‘n’ is 7 or more (e.g. 8, 10, 12, 14, 16, 18, 20, 25, 30, 35, 40, 50, 60, 70, 80, 90, 100, 150, 200, 250 or more). These IC65 proteins include variants of SEQ ID NO: 99. Preferred fragments of (b) comprise an epitope from SEQ ID NO: 99.

Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 99 while retaining at least one epitope of SEQ ID NO: 99. Other fragments omit one or more protein domains. Immunogenic fragments of IC65 are identified in table 1 of reference 82.

IC66

IC66 is, as mentioned above, a variant form of spr1707. Useful IC66 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 100; and/or (b) comprising a fragment of at least ‘n’ consecutive amino acids of SEQ ID NO: 100, wherein ‘n’ is 7 or more (e.g. 8, 10, 12, 14, 16, 18, 20, 25, 30, 35, 40, 50, 60, 70, 80, 90, 100, 150, 200, 250 or more). These IC66 proteins include variants of SEQ ID NO: 100. Preferred fragments of (b) comprise an epitope from SEQ ID NO: 100. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 100 while retaining at least one epitope of SEQ ID NO: 100. Other fragments omit one or more protein domains.

IC67

IC67 is a Subtilisin-like serine protease. For reference purposes, the amino acid sequence of full length IC67 is SEQ ID NO: 101 herein. In the R6 genome IC67 is spr1771 [205]. The use of IC67 for immunisation is reported in reference 82 (SEQ ID NO: 222 therein).

Preferred IC67 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 101; and/or (b) comprising a fragment of at least ‘n’ consecutive amino acids of SEQ ID NO: 101, wherein ‘n’ is 7 or more (e.g. 8, 10, 12, 14, 16, 18, 20, 25, 30, 35, 40, 50, 60, 70, 80, 90, 100, 150, 200, 250 or more). These IC67 proteins include variants of SEQ ID NO: 101. Preferred fragments of (b) comprise an epitope from SEQ ID NO: 101. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 101 while retaining at least one epitope of SEQ ID NO: 101. Other fragments omit one or more protein domains. Immunogenic fragments of IC67 are identified in table 1 of reference 82.

IC68

IC68 is a Cmp-binding-factor 1. For reference purposes, the amino acid sequence of full length IC68 is SEQ ID NO: 102 herein. In the R6 genome IC68 is spr1794 [205]. The use of IC68 for immunisation is reported in reference 82 (SEQ ID NO: 223 therein).

Preferred IC68 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 102; and/or (b) comprising a fragment of at least ‘n’ consecutive amino acids of SEQ ID NO: 102, wherein ‘n’ is 7 or more (e.g. 8, 10, 12, 14, 16, 18, 20, 25, 30, 35, 40, 50, 60, 70, 80, 90, 100, 150, 200, 250 or more). These IC68 proteins include variants of SEQ ID NO: 102. Preferred fragments of (b) comprise an epitope from SEQ ID NO: 102. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 102 while retaining at least one epitope of SEQ ID NO: 102. Other fragments omit one or more protein domains. Immunogenic fragments of IC68 are identified in table 1 of reference 82.

IC69

IC69 is annotated in reference 82 as cell wall surface anchor family protein. For reference purposes, the amino acid sequence of full length IC69 is SEQ ID NO: 103 herein. In the R6 genome IC69 is spr1806 [205]. The use of IC69 for immunisation is reported in reference 82 (SEQ ID NO: 224 therein).

Preferred IC69 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 103; and/or (b) comprising a fragment of at least ‘n’ consecutive amino acids of SEQ ID NO: 103, wherein ‘n’ is 7 or more (e.g. 8, 10, 12, 14, 16, 18, 20, 25, 30, 35, 40, 50, 60, 70, 80, 90, 100, 150, 200, 250 or more). These IC69 proteins include variants of SEQ ID NO: 103. Preferred fragments of (b) comprise an epitope from SEQ ID NO: 103. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 103 while retaining at least one epitope of SEQ ID NO: 103. Other fragments omit one or more protein domains. Immunogenic fragments of IC69 are identified in table 1 of reference 82.

IC70

IC70 is a Catabolite control protein A. For reference purposes, the amino acid sequence of full length IC70 is SEQ ID NO: 104 herein. In the R6 genome IC70 is spr1813 [205]. The use of IC70 for immunisation is reported in reference 82 (SEQ ID NO: 225 therein).

Preferred IC70 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 104; and/or (b) comprising a fragment of at least ‘n’ consecutive amino acids of SEQ ID NO: 104, wherein ‘n’ is 7 or more (e.g. 8, 10, 12, 14, 16, 18, 20, 25, 30, 35, 40, 50, 60, 70, 80, 90, 100, 150, 200, 250 or more). These IC70 proteins include variants of SEQ ID NO: 104. Preferred fragments of (b) comprise an epitope from SEQ ID NO: 104. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 104 while retaining at least one epitope of SEQ ID NO: 104. Other fragments omit one or more protein domains. Immunogenic fragments of IC70 are identified in table 1 of reference 82.

IC71

IC71 is a Beta-glucosidase. For reference purposes, the amino acid sequence of full length IC71 is SEQ ID NO: 105 herein. In the R6 genome IC71 is spr1833 [205]. The use of IC71 for immunisation is reported in reference 82 (SEQ ID NO: 226 therein).

Preferred IC71 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 105; and/or (b) comprising a fragment of at least ‘n’ consecutive amino acids of SEQ ID NO: 105, wherein ‘n’ is 7 or more (e.g. 8, 10, 12, 14, 16, 18, 20, 25, 30, 35, 40, 50, 60, 70, 80, 90, 100, 150, 200, 250 or more). These IC71 proteins include variants of SEQ ID NO: 105. Preferred fragments of (b) comprise an epitope from SEQ ID NO: 105. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 105 while retaining at least one epitope of SEQ ID NO: 105. Other fragments omit one or more protein domains. Immunogenic fragments of IC71 are identified in table 1 of reference 82.

IC72

IC72 is annotated in reference 82 as a hypothetical protein. For reference purposes, the amino acid sequence of full length IC72 is SEQ ID NO: 106 herein. In the R6 genome IC72 is spr1838 [205]. The use of IC72 for immunisation is reported in reference 82 (SEQ ID NO: 227 therein).

Preferred IC72 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 106; and/or (b) comprising a fragment of at least ‘n’ consecutive amino acids of SEQ ID NO: 106, wherein ‘n’ is 7 or more (e.g. 8, 10, 12, 14, 16, 18, 20, 25, 30, 35, 40, 50, 60, 70, 80, 90, 100, 150, 200, 250 or more). These IC72 proteins include variants of SEQ ID NO: 106. Preferred fragments of (b) comprise an epitope from SEQ ID NO: 106. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 106 while retaining at least one epitope of SEQ ID NO: 106. Other fragments omit one or more protein domains. Immunogenic fragments of IC72 are identified in table 1 of reference 82.

IC73

IC73 is annotated in reference 82 as a hypothetical protein. For reference purposes, the amino acid sequence of full length IC73 is SEQ ID NO: 107 herein. In the R6 genome IC73 is spr1850 [205]. The use of IC73 for immunisation is reported in reference 82 (SEQ ID NO: 228 therein).

Preferred IC73 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 107; and/or (b) comprising a fragment of at least ‘n’ consecutive amino acids of SEQ ID NO: 107, wherein ‘n’ is 7 or more (e.g. 8, 10, 12, 14, 16, 18, 20, 25, 30, 35, 40, 50, 60, 70, 80, 90, 100, 150, 200, 250 or more). These IC73 proteins include variants of SEQ ID NO: 107. Preferred fragments of (b) comprise an epitope from SEQ ID NO: 107. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 107 while retaining at least one epitope of SEQ ID NO: 107. Other fragments omit one or more protein domains. Immunogenic fragments of IC73 are identified in table 1 of reference 82.

IC74

IC74 is annotated in reference 82 as a hypothetical protein. For reference purposes, the amino acid sequence of full length IC74 is SEQ ID NO: 108 herein. In the R6 genome IC74 is spr1859 [205]. The use of IC74 for immunisation is reported in reference 82 (SEQ ID NO: 229 therein).

Preferred IC74 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 108; and/or (b) comprising a fragment of at least ‘n’ consecutive amino acids of SEQ ID NO: 108, wherein ‘n’ is 7 or more (e.g. 8, 10, 12, 14, 16, 18, 20, 25, 30, 35, 40, 50, 60, 70, 80, 90, 100, 150, 200, 250 or more). These IC74 proteins include variants of SEQ ID NO: 108. Preferred fragments of (b) comprise an epitope from SEQ ID NO: 108. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 108 while retaining at least one epitope of SEQ ID NO: 108. Other fragments omit one or more protein domains. Immunogenic fragments of IC74 are identified in table 1 of reference 82.

IC75

IC75 is a Competence protein. For reference purposes, the amino acid sequence of full length IC75 is SEQ ID NO: 109 herein. In the R6 genome IC75 is spr1862 [205]. The use of IC75 for immunisation is reported in reference 82 (SEQ ID NO: 230 therein).

Preferred IC75 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 109; and/or (b) comprising a fragment of at least ‘n’ consecutive amino acids of SEQ ID NO: 109, wherein ‘n’ is 7 or more (e.g. 8, 10, 12, 14, 16, 18, 20, 25, 30, 35, 40, 50, 60, 70, 80, 90, 100, 150, 200, 250 or more). These IC75 proteins include variants of SEQ ID NO: 109. Preferred fragments of (b) comprise an epitope from SEQ ID NO: 109. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 109 while retaining at least one epitope of SEQ ID NO: 109. Other fragments omit one or more protein domains. Immunogenic fragments of IC75 are identified in table 1 of reference 82.

IC76

IC76 is a UTP-glucose-1-phosphate uridylyltransferase. For reference purposes, the amino acid sequence of full length IC76 is SEQ ID NO: 110 herein. In the R6 genome IC76 is spr1903 [205]. The use of IC76 for immunisation is reported in reference 82 (SEQ ID NO: 231 therein).

Preferred IC76 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 110; and/or (b) comprising a fragment of at least ‘n’ consecutive amino acids of SEQ ID NO: 110, wherein ‘n’ is 7 or more (e.g. 8, 10, 12, 14, 16, 18, 20, 25, 30, 35, 40, 50, 60, 70, 80, 90, 100, 150, 200, 250 or more). These IC76 proteins include variants of SEQ ID NO: 110. Preferred fragments of (b) comprise an epitope from SEQ ID NO: 110. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 110 while retaining at least one epitope of SEQ ID NO: 110. Other fragments omit one or more protein domains. Immunogenic fragments of IC76 are identified in table 1 of reference 82.

IC77

IC77 is a Penicillin-binding protein 1b. For reference purposes, the amino acid sequence of full length IC77 is SEQ ID NO: 111 herein. In the R6 genome IC77 is spr1909 [205]. The use of IC77 for immunisation is reported in reference 82 (SEQ ID NO: 232 therein).

Preferred IC77 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 111; and/or (b) comprising a fragment of at least ‘n’ consecutive amino acids of SEQ ID NO: 111, wherein ‘n’ is 7 or more (e.g. 8, 10, 12, 14, 16, 18, 20, 25, 30, 35, 40, 50, 60, 70, 80, 90, 100, 150, 200, 250 or more). These IC77 proteins include variants of SEQ ID NO: 111. Preferred fragments of (b) comprise an epitope from SEQ ID NO: 111. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 111 while retaining at least one epitope of SEQ ID NO: 111. Other fragments omit one or more protein domains. Immunogenic fragments of IC77 are identified in table 1 of reference 82.

IC78

IC78 is a ABC transporter substrate-binding protein- maltose/maltodextrin. For reference purposes, the amino acid sequence of full length IC78 is SEQ ID NO: 112 herein. In the R6 genome IC78 is spr1918 [205]. The use of IC78 for immunisation is reported in reference 82 (SEQ ID NO: 233 therein).

Preferred IC78 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 112; and/or (b) comprising a fragment of at least ‘n’ consecutive amino acids of SEQ ID NO: 112, wherein ‘n’ is 7 or more (e.g. 8, 10, 12, 14, 16, 18, 20, 25, 30, 35, 40, 50, 60, 70, 80, 90, 100, 150, 200, 250 or more). These IC78 proteins include variants of SEQ ID NO: 112. Preferred fragments of (b) comprise an epitope from SEQ ID NO: 112. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 112 while retaining at least one epitope of SEQ ID NO: 112. Other fragments omit one or more protein domains. Immunogenic fragments of IC78 are identified in table 1 of reference 82.

IC79

IC79 is annotated in reference 82 as a hypothetical protein. For reference purposes, the amino acid sequence of full length IC79 is SEQ ID NO: 113 herein. In the R6 genome IC79 is spr2120 [205]. The use of IC79 for immunisation is reported in reference 82 (SEQ ID NO: 234 therein).

Preferred IC79 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 113; and/or (b) comprising a fragment of at least ‘n’ consecutive amino acids of SEQ ID NO: 113, wherein ‘n’ is 7 or more (e.g. 8, 10, 12, 14, 16, 18, 20, 25, 30, 35, 40, 50, 60, 70, 80, 90, 100, 150, 200, 250 or more). These IC79 proteins include variants of SEQ ID NO: 113. Preferred fragments of (b) comprise an epitope from SEQ ID NO: 113. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 113 while retaining at least one epitope of SEQ ID NO: 113. Other fragments omit one or more protein domains. Immunogenic fragments of IC79 are identified in table 1 of reference 82.

IC80

IC80 is a Putative transketolase n-terminal section. For reference purposes, the amino acid sequence of full length IC80 is SEQ ID NO: 114 herein. In the R6 genome IC80 is spr1937 [205]. The use of IC80 for immunisation is reported in reference 82 (SEQ ID NO: 235 therein).

Preferred IC80 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 114; and/or (b) comprising a fragment of at least ‘n’ consecutive amino acids of SEQ ID NO: 114, wherein ‘n’ is 7 or more (e.g. 8, 10, 12, 14, 16, 18, 20, 25, 30, 35, 40, 50, 60, 70, 80, 90, 100, 150, 200, 250 or more). These IC80 proteins include variants of SEQ ID NO: 114. Preferred fragments of (b) comprise an epitope from SEQ ID NO: 114. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 114 while retaining at least one epitope of SEQ ID NO: 114. Other fragments omit one or more protein domains. Immunogenic fragments of IC80 are identified in table 1 of reference 82.

IC81

IC81 is a Choline-binding protein. For reference purposes, the amino acid sequence of full length IC81 is SEQ ID NO: 115 herein. Its C-terminus is related to IC3. The use of IC81 for immunisation is reported in reference 82 (SEQ ID NO: 236 therein).

Preferred IC81 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 115; and/or (b) comprising a fragment of at least ‘n’ consecutive amino acids of SEQ ID NO: 115, wherein ‘n’ is 7 or more (e.g. 8, 10, 12, 14, 16, 18, 20, 25, 30, 35, 40, 50, 60, 70, 80, 90, 100, 150, 200, 250 or more). These IC81 proteins include variants of SEQ ID NO: 115. Preferred fragments of (b) comprise an epitope from SEQ ID NO: 115. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 115 while retaining at least one epitope of SEQ ID NO: 115. Other fragments omit one or more protein domains. Immunogenic fragments of IC81 are identified in table 1 of reference 82.

IC82

IC82 is a glycosyl hydrolase-related protein. For reference purposes, the amino acid sequence of full length IC82 is SEQ ID NO: 116 herein. In the R6 genome IC82 is spr2141 [205]. The use of IC82 for immunisation is reported in reference 82 (SEQ ID NO: 237 therein).

Preferred IC82 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 116; and/or (b) comprising a fragment of at least ‘n’ consecutive amino acids of SEQ ID NO: 116, wherein ‘n’ is 7 or more (e.g. 8, 10, 12, 14, 16, 18, 20, 25, 30, 35, 40, 50, 60, 70, 80, 90, 100, 150, 200, 250 or more). These IC82 proteins include variants of SEQ ID NO: 116. Preferred fragments of (b) comprise an epitope from SEQ ID NO: 116. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 116 while retaining at least one epitope of SEQ ID NO: 116. Other fragments omit one or more protein domains. Immunogenic fragments of IC82 are identified in table 1 of reference 82.

IC83

IC83 is annotated in reference 82 as a hypothetical protein . For reference purposes, the amino acid sequence of full length IC83 is SEQ ID NO: 117 herein. In the R6 genome IC83 is spr1983 [205]. The use of IC83 for immunisation is reported in reference 82 (SEQ ID NO: 238 therein).

Preferred IC83 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 117; and/or (b) comprising a fragment of at least ‘n’ consecutive amino acids of SEQ ID NO: 117, wherein ‘n’ is 7 or more (e.g. 8, 10, 12, 14, 16, 18, 20, 25, 30, 35, 40, 50, 60, 70, 80, 90, 100, 150, 200, 250 or more). These IC83 proteins include variants of SEQ ID NO: 117. Preferred fragments of (b) comprise an epitope from SEQ ID NO: 117. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 117 while retaining at least one epitope of SEQ ID NO: 117. Other fragments omit one or more protein domains. Immunogenic fragments of IC83 are identified in table 1 of reference 82.

IC84

IC84 is a Class III stress response-related ATPase. For reference purposes, the amino acid sequence of full length IC84 is SEQ ID NO: 118 herein. In the R6 genome IC84 is spr2000 [205]. The use of IC84 for immunisation is reported in reference 82 (SEQ ID NO: 240 therein).

Preferred IC84 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 118; and/or (b) comprising a fragment of at least ‘n’ consecutive amino acids of SEQ ID NO: 118, wherein ‘n’ is 7 or more (e.g. 8, 10, 12, 14, 16, 18, 20, 25, 30, 35, 40, 50, 60, 70, 80, 90, 100, 150, 200, 250 or more). These IC84 proteins include variants of SEQ ID NO: 118. Preferred fragments of (b) comprise an epitope from SEQ ID NO: 118. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 118 while retaining at least one epitope of SEQ ID NO: 118. Other fragments omit one or more protein domains. Immunogenic fragments of IC84 are identified in table 1 of reference 82.

IC85

IC85 is a variant of SEQ ID NO: 23, mentioned above (SEQ ID NO: 119). Useful IC85 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 119; and/or (b) comprising a fragment of at least ‘n’ consecutive amino acids of SEQ ID NO: 119, wherein ‘n’ is 7 or more (e.g. 8, 10, 12, 14, 16, 18, 20, 25, 30, 35, 40, 50, 60, 70, 80, 90, 100, 150, 200, 250 or more). These IC85 proteins include variants of SEQ ID NO: 119. Preferred fragments of (b) comprise an epitope from SEQ ID NO: 119. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 119 while retaining at least one epitope of SEQ ID NO: 119. Other fragments omit one or more protein domains.

IC86

IC86 is a 50S ribosomal protein L9. For reference purposes, the amino acid sequence of full length IC86 is SEQ ID NO: 120 herein. In the R6 genome IC86 is spr2009 [205]. The use of IC86 for immunisation is reported in reference 82 (SEQ ID NO: 242 therein).

Preferred IC86 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 120; and/or (b) comprising a fragment of at least ‘n’ consecutive amino acids of SEQ ID NO: 120, wherein ‘n’ is 7 or more (e.g. 8, 10, 12, 14, 16, 18, 20, 25, 30, 35, 40, 50, 60, 70, 80, 90, 100, 150, 200, 250 or more). These IC86 proteins include variants of SEQ ID NO: 120. Preferred fragments of (b) comprise an epitope from SEQ ID NO: 120. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 120 while retaining at least one epitope of SEQ ID NO: 120. Other fragments omit one or more protein domains. Immunogenic fragments of IC86 are identified in table 1 of reference 82.

IC87

IC87 is annotated in reference 82 as a hypothetical protein. For reference purposes, the amino acid sequence of full length IC87 is SEQ ID NO: 166 herein. In the R6 genome IC87 is spr0987 [205]. The use of IC87 for immunisation is reported in reference 82 (SEQ ID NO: 288 therein).

Preferred IC87 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 166; and/or (b) comprising a fragment of at least ‘n’ consecutive amino acids of SEQ ID NO: 166, wherein ‘n’ is 7 or more (e.g. 8, 10, 12, 14, 16, 18, 20, 25, 30, 35, 40, 50, 60, 70, 80, 90, 100, 150, 200, 250 or more). These IC87 proteins include variants of SEQ ID NO: 166. Preferred fragments of (b) comprise an epitope from SEQ ID NO: 166. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 166 while retaining at least one epitope of SEQ ID NO: 166. Other fragments omit one or more protein domains. Immunogenic fragments of IC87 are identified in table 1 of reference 82.

IC88

IC88 is a Choline binding protein. For reference purposes, the amino acid sequence of full length IC88 is SEQ ID NO: 122 herein. In the R6 genome IC88 is spr1274 [205]. The use of IC88 for immunisation is reported in reference 82 (SEQ ID NO: 244 therein).

Preferred IC88 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 122; and/or (b) comprising a fragment of at least ‘n’ consecutive amino acids of SEQ ID NO: 122, wherein ‘n’ is 7 or more (e.g. 8, 10, 12, 14, 16, 18, 20, 25, 30, 35, 40, 50, 60, 70, 80, 90, 100, 150, 200, 250 or more). These IC88 proteins include variants of SEQ ID NO: 122. Preferred fragments of (b) comprise an epitope from SEQ ID NO: 122. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 122 while retaining at least one epitope of SEQ ID NO: 122. Other fragments omit one or more protein domains. Immunogenic fragments of IC88 are identified in table 1 of reference 82.

IC89

IC89 is annotated in reference 82 as a hypothetical protein. For reference purposes, the amino acid sequence of full length IC89 is SEQ ID NO: 123 herein. The use of IC89 for immunisation is reported in reference 82 (SEQ ID NO: 245 therein).

Preferred IC89 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 123; and/or (b) comprising a fragment of at least ‘n’ consecutive amino acids of SEQ ID NO: 123, wherein ‘n’ is 7 or more (e.g. 8, 10, 12, 14, 16, 18, 20, 25, 30, 35, 40, 50, 60, 70, 80, 90, 100, 150, 200, 250 or more). These IC89 proteins include variants of SEQ ID NO: 123. Preferred fragments of (b) comprise an epitope from SEQ ID NO: 123. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 123 while retaining at least one epitope of SEQ ID NO: 123. Other fragments omit one or more protein domains. Immunogenic fragments of IC89 are identified in table 1 of reference 82.

IC90

IC90 is annotated in reference 82 as a hypothetical protein. For reference purposes, the amino acid sequence of full length IC90 is SEQ ID NO: 124 herein. The use of IC90 for immunisation is reported in reference 82 (SEQ ID NO: 246 therein).

Preferred IC90 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 124; and/or (b) comprising a fragment of at least ‘n’ consecutive amino acids of SEQ ID NO: 124, wherein ‘n’ is 7 or more (e.g. 8, 10, 12, 14, 16, 18, 20, 25, 30, 35, 40, 50, 60, 70, 80, 90, 100, 150, 200, 250 or more). These IC90 proteins include variants of SEQ ID NO: 124. Preferred fragments of (b) comprise an epitope from SEQ ID NO: 124. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 124 while retaining at least one epitope of SEQ ID NO: 124. Other fragments omit one or more protein domains. Immunogenic fragments of IC90 are identified in table 1 of reference 82.

IC91

IC91 is annotated in reference 82 as a hypothetical protein. For reference purposes, the amino acid sequence of full length IC91 is SEQ ID NO: 125 herein. In the R6 genome IC91 is spr0415 [205]. The use of IC91 for immunisation is reported in reference 82 (SEQ ID NO: 247 therein).

Preferred IC91 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 125; and/or (b) comprising a fragment of at least ‘n’ consecutive amino acids of SEQ ID NO: 125, wherein ‘n’ is 7 or more (e.g. 8, 10, 12, 14, 16, 18, 20, 25, 30, 35, 40, 50, 60, 70, 80, 90, 100, 150, 200, 250 or more). These IC91 proteins include variants of SEQ ID NO: 125. Preferred fragments of (b) comprise an epitope from SEQ ID NO: 125. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 125 while retaining at least one epitope of SEQ ID NO: 125. Other fragments omit one or more protein domains. Immunogenic fragments of IC91 are identified in table 1 of reference 82.

IC92

IC92 is annotated in reference 82 as a hypothetical protein. For reference purposes, the amino acid sequence of full length IC92 is SEQ ID NO: 126 herein. In the R6 genome IC92 is spr0695 [205]. The use of IC92 for immunisation is reported in reference 82 (SEQ ID NO: 248 therein).

Preferred IC92 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 126; and/or (b) comprising a fragment of at least ‘n’ consecutive amino acids of SEQ ID NO: 126, wherein ‘n’ is 7 or more (e.g. 8, 10, 12, 14, 16, 18, 20, 25, 30, 35, 40, 50, 60, 70, 80, 90, 100, 150, 200, 250 or more). These IC92 proteins include variants of SEQ ID NO: 126. Preferred fragments of (b) comprise an epitope from SEQ ID NO: 126. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 126 while retaining at least one epitope of SEQ ID NO: 126. Other fragments omit one or more protein domains. Immunogenic fragments of IC92 are identified in table 1 of reference 82.

IC93

IC93 is annotated in reference 82 as a hypothetical protein. For reference purposes, the amino acid sequence of full length IC93 is SEQ ID NO: 127 herein. In the R6 genome IC93 is spr1334 [205]. The use of IC93 for immunisation is reported in reference 82 (SEQ ID NO: 249 therein).

Preferred IC93 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 127; and/or (b) comprising a fragment of at least ‘n’ consecutive amino acids of SEQ ID NO: 127, wherein ‘n’ is 7 or more (e.g. 8, 10, 12, 14, 16, 18, 20, 25, 30, 35, 40, 50, 60, 70, 80, 90, 100, 150, 200, 250 or more). These IC93 proteins include variants of SEQ ID NO: 127. Preferred fragments of (b) comprise an epitope from SEQ ID NO: 127. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 127 while retaining at least one epitope of SEQ ID NO: 127. Other fragments omit one or more protein domains. Immunogenic fragments of IC93 are identified in table 1 of reference 82.

IC94

IC94 is annotated in reference 82 as a hypothetical protein. For reference purposes, the amino acid sequence of full length IC94 is SEQ ID NO: 128 herein. In the R6 genome IC94 is spr0242 [205]. The use of IC94 for immunisation is reported in reference 82 (SEQ ID NO: 250 therein).

Preferred IC94 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 128; and/or (b) comprising a fragment of at least ‘n’ consecutive amino acids of SEQ ID NO: 128, wherein ‘n’ is 7 or more (e.g. 8, 10, 12, 14, 16, 18, 20, 25, 30, 35, 40, 50, 60, 70, 80, 90, 100, 150, 200, 250 or more). These IC94 proteins include variants of SEQ ID NO: 128. Preferred fragments of (b) comprise an epitope from SEQ ID NO: 128. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 128 while retaining at least one epitope of SEQ ID NO: 128. Other fragments omit one or more protein domains. Immunogenic fragments of IC94 are identified in table 1 of reference 82.

IC95

IC95 is annotated in reference 82 as a hypothetical protein. For reference purposes, the amino acid sequence of full length IC95 is SEQ ID NO: 129 herein. In the R6 genome IC95 is spr1367 [205]. The use of IC95 for immunisation is reported in reference 82 (SEQ ID NO: 251 therein).

Preferred IC95 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 129; and/or (b) comprising a fragment of at least ‘n’ consecutive amino acids of SEQ ID NO: 129, wherein ‘n’ is 7 or more (e.g. 8, 10, 12, 14, 16, 18, 20, 25, 30, 35, 40, 50, 60, 70, 80, 90, 100, 150, 200, 250 or more). These IC95 proteins include variants of SEQ ID NO: 129. Preferred fragments of (b) comprise an epitope from SEQ ID NO: 129. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 129 while retaining at least one epitope of SEQ ID NO: 129. Other fragments omit one or more protein domains. Immunogenic fragments of IC95 are identified in table 1 of reference 82.

IC96

IC96 is annotated in reference 82 as a hypothetical protein. For reference purposes, the amino acid sequence of full length IC96 is SEQ ID NO: 130 herein. The use of IC96 for immunisation is reported in reference 82 (SEQ ID NO: 252 therein).

Preferred IC96 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 130; and/or (b) comprising a fragment of at least ‘n’ consecutive amino acids of SEQ ID NO: 130, wherein ‘n’ is 7 or more (e.g. 8, 10, 12, 14, 16, 18, 20, 25, 30, 35, 40, 50, 60, 70, 80, 90, 100, 150, 200, 250 or more). These IC96 proteins include variants of SEQ ID NO: 130. Preferred fragments of (b) comprise an epitope from SEQ ID NO: 130. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 130 while retaining at least one epitope of SEQ ID NO: 130. Other fragments omit one or more protein domains. Immunogenic fragments of IC96 are identified in table 1 of reference 82.

IC97

IC97 is annotated in reference 82 as a hypothetical protein. For reference purposes, the amino acid sequence of full length IC97 is SEQ ID NO: 131 herein. In the R6 genome IC97 is spr1502 [205]. The use of IC97 for immunisation is reported in reference 82 (SEQ ID NO: 253 therein).

Preferred IC97 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 131; and/or (b) comprising a fragment of at least ‘n’ consecutive amino acids of SEQ ID NO: 131, wherein ‘n’ is 7 or more (e.g. 8, 10, 12, 14, 16, 18, 20, 25, 30, 35, 40, 50, 60, 70, 80, 90, 100, 150, 200, 250 or more). These IC97 proteins include variants of SEQ ID NO: 131. Preferred fragments of (b) comprise an epitope from SEQ ID NO: 131. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 131 while retaining at least one epitope of SEQ ID NO: 131. Other fragments omit one or more protein domains. Immunogenic fragments of IC97 are identified in table 1 of reference 82.

IC98

IC98 is annotated in reference 82 as a hypothetical protein. For reference purposes, the amino acid sequence of full length IC98 is SEQ ID NO: 132 herein. In the R6 genome IC98 is spr0730 [205]. The use of IC98 for immunisation is reported in reference 82 (SEQ ID NO: 254 therein).

Preferred IC98 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 132; and/or (b) comprising a fragment of at least ‘n’ consecutive amino acids of SEQ ID NO: 132, wherein ‘n’ is 7 or more (e.g. 8, 10, 12, 14, 16, 18, 20, 25, 30, 35, 40, 50, 60, 70, 80, 90, 100, 150, 200, 250 or more). These IC98 proteins include variants of SEQ ID NO: 132. Preferred fragments of (b) comprise an epitope from SEQ ID NO: 132. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 132 while retaining at least one epitope of SEQ ID NO: 132. Other fragments omit one or more protein domains. Immunogenic fragments of IC98 are identified in table 1 of reference 82.

IC99

IC99 is annotated in reference 82 as a hypothetical protein. For reference purposes, the amino acid sequence of full length IC99 is SEQ ID NO: 133 herein. In the R6 genome IC99 is spr1961 [205]. The use of IC99 for immunisation is reported in reference 82 (SEQ ID NO: 255 therein).

Preferred IC99 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 133; and/or (b) comprising a fragment of at least ‘n’ consecutive amino acids of SEQ ID NO: 133, wherein ‘n’ is 7 or more (e.g. 8, 10, 12, 14, 16, 18, 20, 25, 30, 35, 40, 50, 60, 70, 80, 90, 100, 150, 200, 250 or more). These IC99 proteins include variants of SEQ ID NO: 133. Preferred fragments of (b) comprise an epitope from SEQ ID NO: 133. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 133 while retaining at least one epitope of SEQ ID NO: 133. Other fragments omit one or more protein domains. Immunogenic fragments of IC99 are identified in table 1 of reference 82.

IC100

IC100 is annotated in reference 82 as a hypothetical protein. For reference purposes, the amino acid sequence of full length IC100 is SEQ ID NO: 134 herein. The use of IC100 for immunisation is reported in reference 82 (SEQ ID NO: 256 therein).

Preferred IC100 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 134; and/or (b) comprising a fragment of at least ‘n’ consecutive amino acids of SEQ ID NO: 134, wherein ‘n’ is 7 or more (e.g. 8, 10, 12, 14, 16, 18, 20, 25, 30, 35, 40, 50, 60, 70, 80, 90, 100, 150, 200, 250 or more). These IC100 proteins include variants of SEQ ID NO: 134. Preferred fragments of (b) comprise an epitope from SEQ ID NO: 134. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 134 while retaining at least one epitope of SEQ ID NO: 134. Other fragments omit one or more protein domains. Immunogenic fragments of IC 100 are identified in table 1 of reference 82.

IC101

IC101 is annotated in reference 82 as a hypothetical protein. For reference purposes, the amino acid sequence of full length IC101 is SEQ ID NO: 135 herein. In the R6 genome IC101 is spr0516 [205]. The use of IC101 for immunisation is reported in reference 82 (SEQ ID NO: 257 therein).

Preferred IC101 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 135; and/or (b) comprising a fragment of at least ‘n’ consecutive amino acids of SEQ ID NO: 135, wherein ‘n’ is 7 or more (e.g. 8, 10, 12, 14, 16, 18, 20, 25, 30, 35, 40, 50, 60, 70, 80, 90, 100, 150, 200, 250 or more). These IC101 proteins include variants of SEQ ID NO: 135. Preferred fragments of (b) comprise an epitope from SEQ ID NO: 135. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 135 while retaining at least one epitope of SEQ ID NO: 135. Other fragments omit one or more protein domains. Immunogenic fragments of IC101 are identified in table 1 of reference 82.

IC102

IC102 is annotated in reference 82 as a hypothetical protein. For reference purposes, the amino acid sequence of full length IC102 is SEQ ID NO: 136 herein. In the R6 genome IC102 is spr1785 [205]. The use of IC102 for immunisation is reported in reference 82 (SEQ ID NO: 258 therein).

Preferred IC102 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 136; and/or (b) comprising a fragment of at least ‘n’ consecutive amino acids of SEQ ID NO: 136, wherein ‘n’ is 7 or more (e.g. 8, 10, 12, 14, 16, 18, 20, 25, 30, 35, 40, 50, 60, 70, 80, 90, 100, 150, 200, 250 or more). These IC102 proteins include variants of SEQ ID NO: 136. Preferred fragments of (b) comprise an epitope from SEQ ID NO: 136. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 136 while retaining at least one epitope of SEQ ID NO: 136. Other fragments omit one or more protein domains. Immunogenic fragments of IC102 are identified in table 1 of reference 82.

IC103

IC103 is annotated in reference 82 as a hypothetical protein. For reference purposes, the amino acid sequence of full length IC103 is SEQ ID NO: 137 herein. In the R6 genome IC103 is spr0215 [205]. The use of IC103 for immunisation is reported in reference 82 (SEQ ID NO: 259 therein).

Preferred IC103 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 137; and/or (b) comprising a fragment of at least ‘n’ consecutive amino acids of SEQ ID NO: 137, wherein ‘n’ is 7 or more (e.g. 8, 10, 12, 14, 16, 18, 20, 25, 30, 35, 40, 50, 60, 70, 80, 90, 100, 150, 200, 250 or more). These IC103 proteins include variants of SEQ ID NO: 137. Preferred fragments of (b) comprise an epitope from SEQ ID NO: 137. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 137 while retaining at least one epitope of SEQ ID NO: 137. Other fragments omit one or more protein domains. Immunogenic fragments of IC 103 are identified in table 1 of reference 82.

IC104

IC104 is annotated in reference 82 as a hypothetical protein. For reference purposes, the amino acid sequence of full length IC104 is SEQ ID NO: 138 herein. In the R6 genome IC104 is spr1815 [205]. The use of IC104 for immunisation is reported in reference 82 (SEQ ID NO: 260 therein).

Preferred IC104 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 138; and/or (b) comprising a fragment of at least ‘n’ consecutive amino acids of SEQ ID NO: 138, wherein ‘n’ is 7 or more (e.g. 8, 10, 12, 14, 16, 18, 20, 25, 30, 35, 40, 50, 60, 70, 80, 90, 100, 150, 200, 250 or more). These IC104 proteins include variants of SEQ ID NO: 138. Preferred fragments of (b) comprise an epitope from SEQ ID NO: 138. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 138 while retaining at least one epitope of SEQ ID NO: 138. Other fragments omit one or more protein domains. Immunogenic fragments of IC104 are identified in table 1 of reference 82.

IC105

IC105 is annotated in reference 82 as a hypothetical protein. For reference purposes, the amino acid sequence of full length IC105 is SEQ ID NO: 139 herein. In the R6 genome IC105 is spr0102 [205]. The use of IC105 for immunisation is reported in reference 82 (SEQ ID NO: 261 therein).

Preferred IC105 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 139; and/or (b) comprising a fragment of at least ‘n’ consecutive amino acids of SEQ ID NO: 139, wherein ‘n’ is 7 or more (e.g. 8, 10, 12, 14, 16, 18, 20, 25, 30, 35, 40, 50, 60, 70, 80, 90, 100, 150, 200, 250 or more). These IC105 proteins include variants of SEQ ID NO: 139. Preferred fragments of (b) comprise an epitope from SEQ ID NO: 139. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 139 while retaining at least one epitope of SEQ ID NO: 139. Other fragments omit one or more protein domains. Immunogenic fragments of IC105 are identified in table 1 of reference 82.

IC106

IC106 is annotated in reference 82 as a hypothetical protein. For reference purposes, the amino acid sequence of full length IC106 is SEQ ID NO: 140 herein. In the R6 genome IC106 is spr1994 [205]. The use of IC106 for immunisation is reported in reference 82 (SEQ ID NO: 262 therein).

Preferred IC106 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 140; and/or (b) comprising a fragment of at least ‘n’ consecutive amino acids of SEQ ID NO: 140, wherein ‘n’ is 7 or more (e.g. 8, 10, 12, 14, 16, 18, 20, 25, 30, 35, 40, 50, 60, 70, 80, 90, 100, 150, 200, 250 or more). These IC106 proteins include variants of SEQ ID NO: 140. Preferred fragments of (b) comprise an epitope from SEQ ID NO: 140. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 140 while retaining at least one epitope of SEQ ID NO: 140. Other fragments omit one or more protein domains. Immunogenic fragments of IC 106 are identified in table 1 of reference 82.

IC107

IC107 is annotated in reference 82 as a hypothetical protein. For reference purposes, the amino acid sequence of full length IC107 is SEQ ID NO: 141 herein. The use of IC107 for immunisation is reported in reference 82 (SEQ ID NO: 263 therein).

Preferred IC107 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 141; and/or (b) comprising a fragment of at least ‘n’ consecutive amino acids of SEQ ID NO: 141, wherein ‘n’ is 7 or more (e.g. 8, 10, 12, 14, 16, 18, 20, 25, 30, 35, 40, 50, 60, 70, 80, 90, 100, 150, 200, 250 or more). These IC107 proteins include variants of SEQ ID NO: 141. Preferred fragments of (b) comprise an epitope from SEQ ID NO: 141. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 141 while retaining at least one epitope of SEQ ID NO: 141. Other fragments omit one or more protein domains. Immunogenic fragments of IC 107 are identified in table 1 of reference 82.

IC108

IC108 is annotated in reference 82 as a hypothetical protein. For reference purposes, the amino acid sequence of full length IC108 is SEQ ID NO: 142 herein. The use of IC108 for immunisation is reported in reference 82 (SEQ ID NO: 264 therein).

Preferred IC108 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 142; and/or (b) comprising a fragment of at least ‘n’ consecutive amino acids of SEQ ID NO: 142, wherein ‘n’ is 7 or more (e.g. 8, 10, 12, 14, 16, 18, 20, 25, 30, 35, 40, 50, 60, 70, 80, 90, 100, 150, 200, 250 or more). These IC108 proteins include variants of SEQ ID NO: 142. Preferred fragments of (b) comprise an epitope from SEQ ID NO: 142. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 142 while retaining at least one epitope of SEQ ID NO: 142. Other fragments omit one or more protein domains. Immunogenic fragments of IC 108 are identified in table 1 of reference 82.

IC109

IC109 is annotated in reference 82 as a hypothetical protein. For reference purposes, the amino acid sequence of full length IC109 is SEQ ID NO: 143 herein. In the R6 genome IC109 is spr0309 [205]. The use of IC109 for immunisation is reported in reference 82 (SEQ ID NO: 265 therein).

Preferred IC109 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 143; and/or (b) comprising a fragment of at least ‘n’ consecutive amino acids of SEQ ID NO: 143, wherein ‘n’ is 7 or more (e.g. 8, 10, 12, 14, 16, 18, 20, 25, 30, 35, 40, 50, 60, 70, 80, 90, 100, 150, 200, 250 or more). These IC109 proteins include variants of SEQ ID NO: 143. Preferred fragments of (b) comprise an epitope from SEQ ID NO: 143. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 143 while retaining at least one epitope of SEQ ID NO: 143. Other fragments omit one or more protein domains. Immunogenic fragments of IC 109 are identified in table 1 of reference 82.

IC110

IC110 is annotated in reference 82 as a hypothetical protein. For reference purposes, the amino acid sequence of full length IC110 is SEQ ID NO: 144 herein. In the R6 genome IC110 is spr1070 [205]. The use of IC110 for immunisation is reported in reference 82 (SEQ ID NO: 266 therein).

Preferred IC110 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 144; and/or (b) comprising a fragment of at least ‘n’ consecutive amino acids of SEQ ID NO: 144, wherein ‘n’ is 7 or more (e.g. 8, 10, 12, 14, 16, 18, 20, 25, 30, 35, 40, 50, 60, 70, 80, 90, 100, 150, 200, 250 or more). These IC110 proteins include variants of SEQ ID NO: 144. Preferred fragments of (b) comprise an epitope from SEQ ID NO: 144. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 144 while retaining at least one epitope of SEQ ID NO: 144. Other fragments omit one or more protein domains. Immunogenic fragments of IC 110 are identified in table 1 of reference 82.

IC111

IC111 is annotated in reference 82 as a hypothetical protein. For reference purposes, the amino acid sequence of full length IC111 is SEQ ID NO: 145 herein. In the R6 genome IC111 is spr0258 [205]. The use of IC111 for immunisation is reported in reference 82 (SEQ ID NO: 267 therein).

Preferred IC111 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 145; and/or (b) comprising a fragment of at least ‘n’ consecutive amino acids of SEQ ID NO: 145, wherein ‘n’ is 7 or more (e.g. 8, 10, 12, 14, 16, 18, 20, 25, 30, 35, 40, 50, 60, 70, 80, 90, 100, 150, 200, 250 or more). These IC111 proteins include variants of SEQ ID NO: 145. Preferred fragments of (b) comprise an epitope from SEQ ID NO: 145. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 145 while retaining at least one epitope of SEQ ID NO: 145. Other fragments omit one or more protein domains. Immunogenic fragments of IC 111 are identified in table 1 of reference 82.

IC112

IC112 is annotated in reference 82 as a hypothetical protein. For reference purposes, the amino acid sequence of full length IC112 is SEQ ID NO: 146 herein. In the R6 genome IC112 is spr0254 [205]. The use of IC112 for immunisation is reported in reference 82 (SEQ ID NO: 268 therein).

Preferred IC112 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 146; and/or (b) comprising a fragment of at least ‘n’ consecutive amino acids of SEQ ID NO: 146, wherein ‘n’ is 7 or more (e.g. 8, 10, 12, 14, 16, 18, 20, 25, 30, 35, 40, 50, 60, 70, 80, 90, 100, 150, 200, 250 or more). These IC112 proteins include variants of SEQ ID NO: 146. Preferred fragments of (b) comprise an epitope from SEQ ID NO: 146. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 146 while retaining at least one epitope of SEQ ID NO: 146. Other fragments omit one or more protein domains. Immunogenic fragments of IC112 are identified in table 1 of reference 82.

IC113

IC113 is annotated in reference 82 as a hypothetical protein. For reference purposes, the amino acid sequence of full length IC113 is SEQ ID NO: 147 herein. In the R6 genome IC113 is spr0171 [205]. The use of IC113 for immunisation is reported in reference 82 (SEQ ID NO: 269 therein).

Preferred IC113 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 147; and/or (b) comprising a fragment of at least ‘n’ consecutive amino acids of SEQ ID NO: 147, wherein ‘n’ is 7 or more (e.g. 8, 10, 12, 14, 16, 18, 20, 25, 30, 35, 40, 50, 60, 70, 80, 90, 100, 150, 200, 250 or more). These IC113 proteins include variants of SEQ ID NO: 147. Preferred fragments of (b) comprise an epitope from SEQ ID NO: 147. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 147 while retaining at least one epitope of SEQ ID NO: 147. Other fragments omit one or more protein domains. Immunogenic fragments of IC 113 are identified in table 1 of reference 82.

IC114

IC114 is annotated in reference 82 as a hypothetical protein. For reference purposes, the amino acid sequence of full length IC114 is SEQ ID NO: 148 herein. The use of IC114 for immunisation is reported in reference 82 (SEQ ID NO: 270 therein).

Preferred IC114 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 148; and/or (b) comprising a fragment of at least ‘n’ consecutive amino acids of SEQ ID NO: 148, wherein ‘n’ is 7 or more (e.g. 8, 10, 12, 14, 16, 18, 20, 25, 30, 35, 40, 50, 60, 70, 80, 90, 100, 150, 200, 250 or more). These IC114 proteins include variants of SEQ ID NO: 148. Preferred fragments of (b) comprise an epitope from SEQ ID NO: 148. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 148 while retaining at least one epitope of SEQ ID NO: 148. Other fragments omit one or more protein domains. Immunogenic fragments of IC 114 are identified in table 1 of reference 82.

IC115

IC115 is annotated in reference 82 as a hypothetical protein. For reference purposes, the amino acid sequence of full length IC115 is SEQ ID NO: 149 herein. In the R6 genome IC115 is spr0464 [205]. The use of IC115 for immunisation is reported in reference 82 (SEQ ID NO: 271 therein).

Preferred IC115 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 149; and/or (b) comprising a fragment of at least ‘n’ consecutive amino acids of SEQ ID NO: 149, wherein ‘n’ is 7 or more (e.g. 8, 10, 12, 14, 16, 18, 20, 25, 30, 35, 40, 50, 60, 70, 80, 90, 100, 150, 200, 250 or more). These IC115 proteins include variants of SEQ ID NO: 149. Preferred fragments of (b) comprise an epitope from SEQ ID NO: 149. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 149 while retaining at least one epitope of SEQ ID NO: 149. Other fragments omit one or more protein domains. Immunogenic fragments of IC 115 are identified in table 1 of reference 82.

IC116

IC116 is annotated in reference 82 as a hypothetical protein. For reference purposes, the amino acid sequence of full length IC116 is SEQ ID NO: 150 herein. In the R6 genome IC116 is spr0026 [205]. The use of IC116 for immunisation is reported in reference 82 (SEQ ID NO: 272 therein).

Preferred IC116 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 150; and/or (b) comprising a fragment of at least ‘n’ consecutive amino acids of SEQ ID NO: 150, wherein ‘n’ is 7 or more (e.g. 8, 10, 12, 14, 16, 18, 20, 25, 30, 35, 40, 50, 60, 70, 80, 90, 100, 150, 200, 250 or more). These IC116 proteins include variants of SEQ ID NO: 150. Preferred fragments of (b) comprise an epitope from SEQ ID NO: 150. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 150 while retaining at least one epitope of SEQ ID NO: 150. Other fragments omit one or more protein domains. Immunogenic fragments of IC 116 are identified in table 1 of reference 82.

IC117

IC117 is annotated in reference 82 as a hypothetical protein. For reference purposes, the amino acid sequence of full length IC117 is SEQ ID NO: 151 herein. In the R6 genome IC117 is spr1652 [205]. The use of IC117 for immunisation is reported in reference 82 (SEQ ID NO: 273 therein).

Preferred IC117 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 151; and/or (b) comprising a fragment of at least ‘n’ consecutive amino acids of SEQ ID NO: 151, wherein ‘n’ is 7 or more (e.g. 8, 10, 12, 14, 16, 18, 20, 25, 30, 35, 40, 50, 60, 70, 80, 90, 100, 150, 200, 250 or more). These IC117 proteins include variants of SEQ ID NO: 151. Preferred fragments of (b) comprise an epitope from SEQ ID NO: 151. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 151 while retaining at least one epitope of SEQ ID NO: 151. Other fragments omit one or more protein domains. Immunogenic fragments of IC 117 are identified in table 1 of reference 82.

IC118

IC118 is annotated in reference 82 as a hypothetical protein. For reference purposes, the amino acid sequence of full length IC118 is SEQ ID NO: 152 herein. In the R6 genome IC118 is spr1783 [205]. The use of IC118 for immunisation is reported in reference 82 (SEQ ID NO: 274 therein).

Preferred IC118 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 152; and/or (b) comprising a fragment of at least ‘n’ consecutive amino acids of SEQ ID NO: 152, wherein ‘n’ is 7 or more (e.g. 8, 10, 12, 14, 16, 18, 20, 25, 30, 35, 40, 50, 60, 70, 80, 90, 100, 150, 200, 250 or more). These IC118 proteins include variants of SEQ ID NO: 152. Preferred fragments of (b) comprise an epitope from SEQ ID NO: 152. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 152 while retaining at least one epitope of SEQ ID NO: 152. Other fragments omit one or more protein domains. Immunogenic fragments of IC 118 are identified in table 1 of reference 82.

IC119

IC119 is annotated in reference 82 as a hypothetical protein. For reference purposes, the amino acid sequence of full length IC119 is SEQ ID NO: 153 herein. The use of IC119 for immunisation is reported in reference 82 (SEQ ID NO: 275 therein).

Preferred IC119 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 153; and/or (b) comprising a fragment of at least ‘n’ consecutive amino acids of SEQ ID NO: 153, wherein ‘n’ is 7 or more (e.g. 8, 10, 12, 14, 16, 18, 20, 25, 30, 35, 40, 50, 60, 70, 80, 90, 100, 150, 200, 250 or more). These IC119 proteins include variants of SEQ ID NO: 153. Preferred fragments of (b) comprise an epitope from SEQ ID NO: 153. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 153 while retaining at least one epitope of SEQ ID NO: 153. Other fragments omit one or more protein domains. Immunogenic fragments of IC 119 are identified in table 1 of reference 82.

IC120

IC120 is annotated in reference 82 as a hypothetical protein. For reference purposes, the amino acid sequence of full length IC120 is SEQ ID NO: 154 herein. In the R6 genome IC120 is spr1153 [205]. The use of IC120 for immunisation is reported in reference 82 (SEQ ID NO: 276 therein).

Preferred IC120 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 154; and/or (b) comprising a fragment of at least ‘n’ consecutive amino acids of SEQ ID NO: 154, wherein ‘n’ is 7 or more (e.g. 8, 10, 12, 14, 16, 18, 20, 25, 30, 35, 40, 50, 60, 70, 80, 90, 100, 150, 200, 250 or more). These IC120 proteins include variants of SEQ ID NO: 154. Preferred fragments of (b) comprise an epitope from SEQ ID NO: 154. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 154 while retaining at least one epitope of SEQ ID NO: 154. Other fragments omit one or more protein domains. Immunogenic fragments of IC 120 are identified in table 1 of reference 82.

IC121

IC121 is annotated in reference 82 as a hypothetical protein. For reference purposes, the amino acid sequence of full length IC121 is SEQ ID NO: 155 herein. In the R6 genome IC121 is spr1977 [205]. The use of IC121 for immunisation is reported in reference 82 (SEQ ID NO: 277 therein).

Preferred IC121 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 155; and/or (b) comprising a fragment of at least ‘n’ consecutive amino acids of SEQ ID NO: 155, wherein ‘n’ is 7 or more (e.g. 8, 10, 12, 14, 16, 18, 20, 25, 30, 35, 40, 50, 60, 70, 80, 90, 100, 150, 200, 250 or more). These IC121 proteins include variants of SEQ ID NO: 155. Preferred fragments of (b) comprise an epitope from SEQ ID NO: 155. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 155 while retaining at least one epitope of SEQ ID NO: 155. Other fragments omit one or more protein domains. Immunogenic fragments of IC 121 are identified in table 1 of reference 82.

IC122

IC122 is annotated in reference 82 as a hypothetical protein. For reference purposes, the amino acid sequence of full length IC122 is SEQ ID NO: 156 herein. The use of IC122 for immunisation is reported in reference 82 (SEQ ID NO: 278 therein).

Preferred IC122 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 156; and/or (b) comprising a fragment of at least ‘n’ consecutive amino acids of SEQ ID NO: 156, wherein ‘n’ is 7 or more (e.g. 8, 10, 12, 14, 16, 18, 20, 25, 30, 35, 40, 50, 60, 70, 80, 90, 100, 150, 200, 250 or more). These IC122 proteins include variants of SEQ ID NO: 156. Preferred fragments of (b) comprise an epitope from SEQ ID NO: 156. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 156 while retaining at least one epitope of SEQ ID NO: 156. Other fragments omit one or more protein domains. Immunogenic fragments of IC 122 are identified in table 1 of reference 82.

IC123

IC123 is annotated in reference 82 as a hypothetical protein. For reference purposes, the amino acid sequence of full length IC123 is SEQ ID NO: 157 herein. In the R6 genome IC123 is spr1049 [205]. The use of IC123 for immunisation is reported in reference 82 (SEQ ID NO: 279 therein).

Preferred IC123 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 157; and/or (b) comprising a fragment of at least ‘n’ consecutive amino acids of SEQ ID NO: 157, wherein ‘n’ is 7 or more (e.g. 8, 10, 12, 14, 16, 18, 20, 25, 30, 35, 40, 50, 60, 70, 80, 90, 100, 150, 200, 250 or more). These IC123 proteins include variants of SEQ ID NO: 157. Preferred fragments of (b) comprise an epitope from SEQ ID NO: 157. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 157 while retaining at least one epitope of SEQ ID NO: 157. Other fragments omit one or more protein domains. Immunogenic fragments of IC 123 are identified in table 1 of reference 82.

IC124

IC124 is annotated in reference 82 as a hypothetical protein. For reference purposes, the amino acid sequence of full length IC124 is SEQ ID NO: 158 herein. In the R6 genome IC124 is spr1811 [205]. The use of IC124 for immunisation is reported in reference 82 (SEQ ID NO: 280 therein).

Preferred IC124 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 158; and/or (b) comprising a fragment of at least ‘n’ consecutive amino acids of SEQ ID NO: 158, wherein ‘n’ is 7 or more (e.g. 8, 10, 12, 14, 16, 18, 20, 25, 30, 35, 40, 50, 60, 70, 80, 90, 100, 150, 200, 250 or more). These IC124 proteins include variants of SEQ ID NO: 158. Preferred fragments of (b) comprise an epitope from SEQ ID NO: 158. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 158 while retaining at least one epitope of SEQ ID NO: 158. Other fragments omit one or more protein domains. Immunogenic fragments of IC124 are identified in table 1 of reference 82.

IC125

IC125 is annotated in reference 82 as a hypothetical protein. For reference purposes, the amino acid sequence of full length IC125 is SEQ ID NO: 159 herein. In the R6 genome IC125 is spr0381 [205]. The use of IC125 for immunisation is reported in reference 82 (SEQ ID NO: 281 therein).

Preferred IC125 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 159; and/or (b) comprising a fragment of at least ‘n’ consecutive amino acids of SEQ ID NO: 159, wherein ‘n’ is 7 or more (e.g. 8, 10, 12, 14, 16, 18, 20, 25, 30, 35, 40, 50, 60, 70, 80, 90, 100, 150, 200, 250 or more). These IC125 proteins include variants of SEQ ID NO: 159. Preferred fragments of (b) comprise an epitope from SEQ ID NO: 159. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 159 while retaining at least one epitope of SEQ ID NO: 159. Other fragments omit one or more protein domains. Immunogenic fragments of IC 125 are identified in table 1 of reference 82.

IC126

IC126 is annotated in reference 82 as a hypothetical protein. For reference purposes, the amino acid sequence of full length IC126 is SEQ ID NO: 160 herein. The use of IC126 for immunisation is reported in reference 82 (SEQ ID NO: 282 therein).

Preferred IC126 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 160; and/or (b) comprising a fragment of at least ‘n’ consecutive amino acids of SEQ ID NO: 160, wherein ‘n’ is 7 or more (e.g. 8, 10, 12, 14, 16, 18, 20, 25, 30, 35, 40, 50, 60, 70, 80, 90, 100, 150, 200, 250 or more). These IC126 proteins include variants of SEQ ID NO: 160. Preferred fragments of (b) comprise an epitope from SEQ ID NO: 160. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 160 while retaining at least one epitope of SEQ ID NO: 160. Other fragments omit one or more protein domains. Immunogenic fragments of IC 126 are identified in table 1 of reference 82.

IC127

IC127 is annotated in reference 82 as a hypothetical protein. For reference purposes, the amino acid sequence of full length IC127 is SEQ ID NO: 161 herein. In the R6 genome IC127 is spr0061 [205]. The use of IC127 for immunisation is reported in reference 82 (SEQ ID NO: 283 therein).

Preferred IC127 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 161; and/or (b) comprising a fragment of at least ‘n’ consecutive amino acids of SEQ ID NO: 161, wherein ‘n’ is 7 or more (e.g. 8, 10, 12, 14, 16, 18, 20, 25, 30, 35, 40, 50, 60, 70, 80, 90, 100, 150, 200, 250 or more). These IC127 proteins include variants of SEQ ID NO: 161. Preferred fragments of (b) comprise an epitope from SEQ ID NO: 161. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 161 while retaining at least one epitope of SEQ ID NO: 161. Other fragments omit one or more protein domains. Immunogenic fragments of IC 127 are identified in table 1 of reference 82.

IC128

IC128 is annotated in reference 82 as a hypothetical protein. For reference purposes, the amino acid sequence of full length IC128 is SEQ ID NO: 162 herein. In the R6 genome IC128 is spr0641 [205]. The use of IC128 for immunisation is reported in reference 82 (SEQ ID NO: 284 therein).

Preferred IC128 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 162; and/or (b) comprising a fragment of at least ‘n’ consecutive amino acids of SEQ ID NO: 162, wherein ‘n’ is 7 or more (e.g. 8, 10, 12, 14, 16, 18, 20, 25, 30, 35, 40, 50, 60, 70, 80, 90, 100, 150, 200, 250 or more). These IC128 proteins include variants of SEQ ID NO: 162. Preferred fragments of (b) comprise an epitope from SEQ ID NO: 162. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 162 while retaining at least one epitope of SEQ ID NO: 162. Other fragments omit one or more protein domains. Immunogenic fragments of IC 128 are identified in table 1 of reference 82.

IC129

IC129 is annotated in reference 82 as a hypothetical protein. For reference purposes, the amino acid sequence of full length IC129 is SEQ ID NO: 163 herein. In the R6 genome IC129 is spr1205 [205]. The use of IC129 for immunisation is reported in reference 82 (SEQ ID NO: 285 therein).

Preferred IC129 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 163; and/or (b) comprising a fragment of at least ‘n’ consecutive amino acids of SEQ ID NO: 163, wherein ‘n’ is 7 or more (e.g. 8, 10, 12, 14, 16, 18, 20, 25, 30, 35, 40, 50, 60, 70, 80, 90, 100, 150, 200, 250 or more). These IC129 proteins include variants of SEQ ID NO: 163. Preferred fragments of (b) comprise an epitope from SEQ ID NO: 163. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 163 while retaining at least one epitope of SEQ ID NO: 163. Other fragments omit one or more protein domains. Immunogenic fragments of IC 129 are identified in table 1 of reference 82.

IC130

IC130 is annotated in reference 82 as a hypothetical protein. For reference purposes, the amino acid sequence of full length IC130 is SEQ ID NO: 164 herein. In the R6 genome IC130 is spr1841 [205]. The use of IC130 for immunisation is reported in reference 82 (SEQ ID NO: 286 therein).

Preferred IC130 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 164; and/or (b) comprising a fragment of at least ‘n’ consecutive amino acids of SEQ ID NO: 164, wherein ‘n’ is 7 or more (e.g. 8, 10, 12, 14, 16, 18, 20, 25, 30, 35, 40, 50, 60, 70, 80, 90, 100, 150, 200, 250 or more). These IC130 proteins include variants of SEQ ID NO: 164. Preferred fragments of (b) comprise an epitope from SEQ ID NO: 164. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 164 while retaining at least one epitope of SEQ ID NO: 164. Other fragments omit one or more protein domains. Immunogenic fragments of IC130 are identified in table 1 of reference 82.

IC131

IC131 is annotated in reference 82 as a hypothetical protein. For reference purposes, the amino acid sequence of full length IC131 is SEQ ID NO: 165 herein. In the R6 genome IC131 is spr1777 [205]. The use of IC131 for immunisation is reported in reference 82 (SEQ ID NO: 287 therein).

Preferred IC131 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 165; and/or (b) comprising a fragment of at least ‘n’ consecutive amino acids of SEQ ID NO: 165, wherein ‘n’ is 7 or more (e.g. 8, 10, 12, 14, 16, 18, 20, 25, 30, 35, 40, 50, 60, 70, 80, 90, 100, 150, 200, 250 or more). These IC131 proteins include variants of SEQ ID NO: 165. Preferred fragments of (b) comprise an epitope from SEQ ID NO: 165. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 165 while retaining at least one epitope of SEQ ID NO: 165. Other fragments omit one or more protein domains. Immunogenic fragments of IC 131 are identified in table 1 of reference 82.

spr0222

The original ‘spr0222’ sequence was annotated in references 115, 116, 117, 118, 119 and 120 as ‘ABC transporter ATP-binding protein - iron transport’ (see GI:15457768). For reference purposes, the amino acid sequence of full length spr0222 as found in the R6 strain is given as SEQ ID NO: 121 herein. Its use in immunisation is suggested in reference 78.

Preferred spr0222 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 121; and/or (b) comprising a fragment of at least ‘n’ consecutive amino acids of SEQ ID NO: 121, wherein ‘n’ is 7 or more (e.g. 8, 10, 12, 14, 16, 18, 20, 25, 30, 35, 40, 50, 60, 70, 80, 90, 100, 150, 200, 250 or more). These spr022 proteins include variants of SEQ ID NO: 121. Preferred fragments of (b) comprise an epitope from SEQ ID NO: 121. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 121 while retaining at least one epitope of SEQ ID NO: 121. Other fragments omit one or more protein domains.

CibO

CbiO is annotated as a cobalt transporter ATP-binding subunit. For reference purposes, the amino acid sequence of full length CbiO is SEQ ID NO: 167 herein. In the R6 genome CbiO is spr2025 [205]. The use of CbiO for immunisation is reported in reference 79 (‘ID2’ therein).

Preferred CbiO polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 167; and/or (b) comprising a fragment of at least ‘n’ consecutive amino acids of SEQ ID NO: 167, wherein ‘n’ is 7 or more (e.g. 8, 10, 12, 14, 16, 18, 20, 25, 30, 35, 40, 50, 60, 70, 80, 90, 100, 150, 200, 250 or more). These CbiO proteins include variants of SEQ ID NO: 167. Preferred fragments of (b) comprise an epitope from SEQ ID NO: 167. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 167 while retaining at least one epitope of SEQ ID NO: 167. Other fragments omit one or more protein domains.

30S ribosomal protein S8

For reference purposes, the amino acid sequence of 30S ribosomal protein S8 is SEQ ID NO: 168 herein. In the R6 genome the S8 subunit is spr0203 [205].

Preferred S8 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 168; and/or (b) comprising a fragment of at least ‘n’ consecutive amino acids of SEQ ID NO: 168, wherein ‘n’ is 7 or more (e.g. 8, 10, 12, 14, 16, 18, 20, 25, 30, 35, 40, 50, 60, 70, 80, 90, 100, 150, 200, 250 or more). These S8 proteins include variants of SEQ ID NO: 168. Preferred fragments of (b) comprise an epitope from SEQ ID NO: 168. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 168 while retaining at least one epitope of SEQ ID NO: 168. Other fragments omit one or more protein domains.

Combinations

A composition useful for immunisation preferably comprises an RrgB epitope identified herein. In a typical embodiment, a composition useful for immunisation comprises epitopes from at least two RrgB clades, typically three RrgB clades, either as a hybrid polypeptide or as separate polypeptides. In addition, a composition may include: (i) one or more further polypeptides that elicit antibody responses against pneumococcal proteins, particularly against pneumococcal proteins other than RrgB; (ii) a capsular saccharide from pneumococcus; and/or (iii) one or more further immunogens that elicit antibody responses that recognise epitopes on non-pneumococcal organisms.

RrgB epitopes from one or more clades may be combined with one or more (i. e. 1, 2, 3, 4, 5, 6, 7, 8, 9, 10, 11, 12, or all 13) protein antigens, preferably selected from the group consisting of: (1) a spr0057 antigen; (2) a spr0565 antigen; (3) a spr1098 antigen; (4) a spr1416 antigen; (5) a spr1418 antigen; (6) a spr0867 antigen; (7) a spr1431 antigen; (8) a spr1739 antigen; (9) a spr2021 antigen; (10) a spr0096 antigen; (11) a spr1707 antigen; (12) a spr1875 antigen; and/or (13) a spr0884 antigen.

Similarly, RrgB epitopes from one or more clades may be combined with one or more (i.e. 1, 2, 3, 4, 5, 6, 7, 8, 9, 10, 11, 12, 13, 14, 15, 16, 17, 18, 19, or all 20) protein antigens selected from the group consisting of: (1) ClpP; (2) LytA; (3) PhtA; (4) PhtB; (5) PhtD; (6) PhtE; (7) ZmpB; (8) CbpD; (9) CbpG; (10) PvaA; (11) CPL1; (12) PspC; (13) PspA; (14) PsaA; (15) PrtA; (16) Sp133; (17) PiaA; (18) PiuA; (19) CbiO; and/or (20) 30S ribosomal protein S8.

These further antigens may be added as separate polypeptides. As an alternative, they may be added as hybrids e.g. a spr0057-spr0096 hybrid or a spr0096-spr2021 hybrid, a spr0565-PhtD hybrid, etc. As a further alternative, they may be fused to a RrgB epitope sequence to provide a hybrid polypeptide e.g. a RrgB-spr0057 hybrid.

For example, a chimeric RrgB polypeptide including epitopes from two or three RrgB clades may be combined with: (a) a mixture of spr0057, spr0096 and spr2021; (b) a mixture of spr0057, spr0565 and spr2021; (c) a mixture of spr0057, spr0096 and spr0565; (d) a mixture of spr0057, spr0096, spr0565 and spr2021; (e) a mixture of spr1418, spr0884 and spr0096; (f) a mixture of spr1418, spr0884 and spr2021; (g) a mixture of spr1418, spr0884, spr0096 and spr2021; (h) a mixture of spr0884, spr1416 and spr0057; (h) a mixture of spr0884, spr1416 and spr0096; (h) a mixture of spr0884, spr1416, spr0057 and spr0096; or (i) a mixture of spr1418, spr1431 and spr0565. Where these mixtures include both spr0057 and spr0096, a hybrid protein can be used e.g. comprising SEQ ID NO: 82 (see SEQ ID NO: 200 of ref. 121) or comprising SEQ ID NO: 83. Where these mixtures include both spr0096 and spr2021, a hybrid protein can be used e.g. comprising SEQ ID NO: 84 (see SEQ ID NO: 205 of ref. 121).

In a further example, a chimeric RrgB polypeptide including epitopes from two or three RrgB clades may be combined with a pneumococcal immunogen comprising an spr2021 (also referred to as SP2216) antigen, an SP1732 antigen and optionally a PsaA antigen. A suitable pneumococcal immunogen of this sort is the immunogen disclosed in reference 91 that comprises the antigens “SP2216-1” (SEQ ID NO: 1 in reference 91; SEQ ID NO: 97 herein), “SP 1732-3” (SEQ ID NO: 2 in reference 91; SEQ ID NO: 98 herein) and, optionally, PsaA (SEQ ID NO: 3 in reference 91; SEQ ID NO: 99 herein). Polypeptides comprising immunogenic fragments of these SEQ ID NOs can be used in place of the actual disclosed SEQ ID NOs e.g. comprising at least one immunogenic fragment from each of SEQ ID NOs 97 & 98. Polypeptides comprising variants of spr2021 (SP2216), SP1732 and optionally PsaA can also be used in place of the actual disclosed SEQ ID NOs e.g. comprising at least one variant from each of SEQ ID NOs 97 and 98. Examples of this combination include the combination of a pneumococcal immunogen as disclosed in reference 91 with a chimeric RrgB polypeptide comprising chimera II-I-III (e.g. SEQ ID NO: 21) or chimera III-II-I (e.g. SEQ ID NO:15) as detailed below. The further antigens may be added as separate polypeptides. As an alternative, they may be added as hybrids e.g. a spr2021-SP1732 hybrid or a spr2021-SP1732-PsaA hybrid. As a further alternative, they may be fused to a RrgB polypeptide sequence, e.g. a chimeric RrgB polypeptide, to provide a hybrid polypeptide e.g. a RrgB-spr2021-SP1732 hybrid. As detailed above, compositions of the invention comprising combinations such as these can optionally comprise one or more adjuvants.

Hybrid Polypeptides

Pneumococcal antigens used in the invention may be present in the composition as individual separate polypeptides. Where more than one antigen is used, however, they do not have to be present as separate polypeptides. Instead, at least two (e.g. 2, 3, 4, 5, or more) antigens can be expressed as a single polypeptide chain (a ‘hybrid’ polypeptide). Hybrid polypeptides offer two main advantages: first, a polypeptide that may be unstable or poorly expressed on its own can be assisted by adding a suitable hybrid partner that overcomes the problem; second, commercial manufacture is simplified as only one expression and purification need be employed in order to produce two polypeptides which are both antigenically useful. Hybrids consisting of amino acid sequences from two, three, four, five, six, seven, eight, nine, or ten pneumococcal antigens are useful. In particular, hybrids consisting of amino acid sequences from two, three, four, or five pneumococcal antigens are preferred, such as two or three pneumococcal antigens.

Different RrgB Glade epitopes used in the invention do not have to be present as separate polypeptides but can instead be expressed as a single polypeptide chain (a ‘hybrid’ polypeptide or ‘chimera’). Hybrid polypeptides offer two main advantages: first, a polypeptide that may be unstable or poorly expressed on its own can be assisted by adding a suitable hybrid partner that overcomes the problem; second, commercial manufacture is simplified as only one expression and purification need to be employed in order to produce two polypeptides which are both antigenically useful.

Hybrid polypeptides can include sequences from only RrgB antigens but in other embodiments can include non-RrgB antigens (usually pneumococcal non-RrgB antigens), such as other pilus subunits. If non-RrgB antigens are present these may be to the N-terminus of any two RrgB sequences, to the C-terminus of any two RrgB sequences, or may be between two RrgB sequences.

The hybrid polypeptide may comprise two or more polypeptide sequences from the first antigen group. The hybrid polypeptide may comprise one or more polypeptide sequences from the first antigen group and one or more polypeptide sequences from the second antigen group. The hybrid polypeptide may comprise one or more polypeptide sequences from the first antigen group and one or more polypeptide sequences from the third antigen group. The hybrid polypeptide may comprise one or more polypeptide sequences from the second antigen group and one or more polypeptide sequences from the third antigen group. The hybrid polypeptide may comprise two or more polypeptide sequences from the seventh antigen group. The hybrid polypeptide may comprise two or more polypeptide sequences from the eighth antigen group. The hybrid polypeptide may comprise two or more polypeptide sequences from the ninth antigen group. The hybrid polypeptide may comprise two or more polypeptide sequences from the tenth antigen group. Moreover, the hybrid polypeptide may comprise two or more polypeptide sequences from each of the antigens listed above, or two or more variants of the same antigen in the cases in which the sequence has partial variability across strains.

In one embodiment, a hybrid polypeptide according to the invention consists of 50 or fewer, 45 or fewer, 40 or fewer, 35 or fewer, 34, 33 or fewer amino acid residues. Different hybrid polypeptides may be mixed together in a single formulation. Hybrids may be combined with non-hybrid RrgB antigens or other non-RrgB antigens. Hybrids may be combined with non-hybrid antigens selected from the first, second or third antigen groups. Within such combinations, a pneumococcal antigen may be present in more than one hybrid polypeptide and/or as a non-hybrid polypeptide. It is preferred, however, that an antigen is present either as a hybrid or as a non-hybrid, but not as both.

The hybrid polypeptides can also be combined with conjugates or non-pneumococcal antigens as described above.

Hybrid polypeptides may be represented by the formula NH₂-A-{-X-L-}ₙ-B—COOH. Hybrid polypeptides can be represented by the formula NH₂-A-{-X-L-}ₙ-B—COOH, wherein: X is an amino acid sequence of a pneumococcal antigen, as described above; L is an optional linker amino acid sequence; A is an optional N-terminal amino acid sequence; B is an optional C-terminal amino acid sequence; n is an integer of 2 or more (e.g. 2, 3, 4, 5, 6, etc.). Usually n is 2 or 3.

If a —X— moiety has a leader peptide sequence in its wild-type form, this may be included or omitted in the hybrid protein. In some embodiments, the leader peptides will be deleted except for that of the —X— moiety located at the N-terminus of the hybrid protein i.e. the leader peptide of X₁ will be retained, but the leader peptides of X₂ . . . X. will be omitted. This is equivalent to deleting all leader peptides and using the leader peptide of X₁ as moiety -A-.

For each n instances of {—X-L-}, linker amino acid sequence -L- may be present or absent. For instance, when n=2 the hybrid may be NH₂—X₁-L₁-X₂-L₂-COOH, NH₂—X₁—X₂—COOH, NH₂—X₁-L₁-X₂—COOH, NH₂—X₁—X₂-L₂-COOH, etc. Linker amino acid sequence(s) -L- will typically be short (e.g. 20 or fewer amino acids i.e. 20, 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e. comprising Glyₙ where n=2, 3, 4, 5, 6, 7, 8, 9, 10 or more), and histidine tags (i.e. His where n=3, 4, 5, 6, 7, 8, 9, 10 or more). Other suitable linker amino acid sequences will be apparent to those skilled in the art. A useful linker is GSGGGG (SEQ ID NO: 7) or GSGSGGGG (SEQ ID NO: 8), with the Gly-Ser dipeptide being formed from a BamHI restriction site, thus aiding cloning and manipulation, and the (Gly)₄ tetrapeptide being a typical poly-glycine linker. Other suitable linkers, particularly for use as the final Lₙ are a Leu-Glu dipeptide or Gly-Ser. Linkers will usually contain at least one glycine residue to facilitate structural flexibility e.g. a -L- moiety may contain 1, 2, 3, 4, 5, 6, 7, 8, 9, 10 or more glycine residues. Such glycines may be arranged to include at least two consecutive glycines in a Gly-Gly dipeptide sequence, or a longer oligo-Gly sequence i.e. Glyₙ where n=2, 3, 4, 5, 6, 7, 8, 9, 10 or more. Other suitable linkers, particularly for use as the final Lₙ are a Leu-Glu dipeptide or SEQ ID NO: 235.

-A- is an optional N-terminal amino acid sequence. This will typically be short (e.g. 40 or fewer amino acids i.e. 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. His where n=3, 4, 5, 6, 7, 8, 9, 10 or more). Other suitable N-terminal amino acid sequences will be apparent to those skilled in the art. If X₁ lacks its own N-terminus methionine, —A- is preferably an oligopeptide (e.g. with 1, 2, 3, 4, 5, 6, 7 or 8 amino acids) which provides a N-terminus methionine e.g. Met-Ala-Ser, or a single Met residue. In a nascent polypeptide the -A- moiety can provide the polypeptide's N-terminal methionine (formyl-methionine, fMet, in bacteria). One or more amino acids may be cleaved from the N-terminus of a nascent -A- moiety, however, such that the -A- moiety in a mature polypeptide of the invention does not necessarily include a N-terminal methionine.

—B— is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. His where n=3, 4, 5, 6, 7, 8, 9, 10 or more, such as SEQ ID NO: 9), or sequences which enhance protein stability. Other suitable C-terminal amino acid sequences will be apparent to those skilled in the art, such as a glutathione-S-transferase, thioredoxin, 14 kDa fragment of S.aureus protein A, a biotinylated peptide, a maltose-binding protein, an enterokinase flag, etc.

It is preferred that -A-, —B— and -L- sequences do not include a sequence that shares 10 or more contiguous amino acids in common with a human polypeptide sequence.

In some embodiments, a -L- moiety comprises a non-RrgB antigen. In some embodiments, the -A- moiety comprises a non-RrgB antigen, and in some the —B— moiety comprises a non-RrgB antigen.

The invention also provides nucleic acid which encodes a hybrid polypeptide of the invention.

Wherein the chimeric protein comprises three clades from RrgB, the hybrid is preferably selected from the list:

RrgB I-II-III
e.g. SEQ ID NO: 246
(also referred to as RrgB123)
RrgB I-III-II
e.g. SEQ ID NO: 248
(also referred to as RrgB132)
RrgB III-II-I
e.g. SEQ ID NO: 250
(also referred to as RrgB321)
RrgB III-I-II
e.g. SEQ ID NO: 252
(also referred to as RrgB312)
RrgB II-III-I
e.g. SEQ ID NO: 254
(also referred to as RrgB231)
RrgB II-I-III
e.g. SEQ ID NO: 256
(also referred to as RrgB213)

Preferably, the RrgB hybrid is selected from RrgBI-II-III, RrgBIII-II-I, RrgBIII-I-II and RrgBII-III-I. More preferably, the RrgB hybrid is selected from RrgBI-II-III and RrgBIII-II-I. Most preferably, the RrgB hybrid is RrgBIII-II-I.

Further examples of hybrids include polypeptides that comprise an amino acid sequence selected from the group consisting of: spr2021-spr0057 (e.g. SEQ ID NO: 193); spr2021-spr0096 (e.g. SEQ ID NO: 194); spr2021-spr0565 (e.g. SEQ ID NO: 195 or SEQ ID NO: 196 or SEQ ID NO: 197); spr2021-RrgA (e.g. SEQ ID NO: 198); spr0057-spr2021 (e.g. SEQ ID NO: 199); spr0057-spr0096 (e.g. SEQ ID NO: 200); spr0057-RrgA (e.g. SEQ ID NO: 201); spr0057-spr0565 (e.g. SEQ ID NO: 202 or SEQ ID NO: 203 or SEQ ID NO: 204); spr0096-spr2021 (e.g. SEQ ID NO: 205); spr0096-spr-0057 (e.g. SEQ ID NO: 206); spr0096-RrgA (e.g. SEQ ID NO: 207); spr0096-spr0565 (e.g. SEQ ID NO: 208 or SEQ ID NO: 209 or SEQ ID NO: 210); RrgA-spr2021 (e.g. SEQ ID NO: 211); RrgA-spr0565 (e.g. SEQ ID NO: 212 or SEQ ID NO: 213 or SEQ ID NO: 214); RrgA-spr0057 (e.g. SEQ ID NO: 215); RrgA-spr0096 (e.g. SEQ ID NO: 216); spr0565-spr0057 (e.g. SEQ ID NO: 217 or SEQ ID NO: 218 or SEQ ID NO: 219); spr0565-spr0096 (e.g. SEQ ID NO: 220 or SEQ ID NO: 221 or SEQ ID NO: 222); spr0565-spr2021 (e.g. SEQ ID NO: 223 or SEQ ID NO: 224 or SEQ ID NO: 225); or spr0565-RrgA (e.g. SEQ ID NO: 226 or SEQ ID NO: 227 or SEQ ID NO: 228).

### Combinations of Pneumococcal Protein and Saccharide Antigens

In addition to a S. pneumoniae protein antigen, compositions of the invention may also include one or more pneumococcal capsular saccharide(s), which will typically be conjugated to carrier protein(s). Further information about such saccharides and conjugation is provided below.

The individual antigens identified in the antigen groups may be used as carrier proteins for pneumococcal capsular saccharides, to form a covalent conjugate. Thus the invention provides an immunogenic composition comprising a conjugate of (1) an antigen selected from the first, second, third, fourth, fifth, sixth, seventh, eighth, ninth or tenth antigen groups and (2) a pneumococcal capsular saccharide. Further characteristics of such a conjugate are described above. The use of pneumococcal proteins as carriers in conjugates is known in the art [e.g. refs. 122, 124 & 103]. These conjugates may be combined with any of the further antigens disclosed herein.

Pneumococcal protein antigens may be combined with one or more pneumococcal capsular saccharide(s), which will typically be conjugated to carrier protein(s). Thus the invention provides an immunogenic composition comprising (i) a TLR agonist; (ii) an insoluble metal salt; (iii) one or more S. pneumoniae protein antigen(s) as discussed above, preferably as a mixture or hybrid; and (iv) one or more pneumococcal capsular saccharides.

The protein antigen in component (iii) is preferably combination of at least two RrgB Glade epitopes.

A saccharide used in component (iv) of this combination is ideally present as a conjugate comprising a saccharide moiety and a carrier protein moiety. The carrier moiety in the conjugate may be e.g. a single RrgB polypeptide, a hybrid RrgB polypeptide, a non-RrgB pneumococcal polypeptide, or a non-pneumococcal polypeptide.

The saccharide is from the capsular saccharide of a pneumococcus. The saccharide may be a polysaccharide having the size that arises during purification of the saccharide from bacteria, or it may be an oligosaccharide achieved by fragmentation of such a polysaccharide. In the 7-valent PREVNAR™ product, for instance, 6 of the saccharides are presented as intact polysaccharides while one (the 18C serotype) is presented as an oligosaccharide.

A composition may include a capsular saccharide from one or more of the following pneumococcal serotypes: 1, 2, 3, 4, 5, 6A, 6B, 7F, 8, 9N, 9V, 10A, 11A, 12F, 14, 15B, 17F, 18C, 19A, 19F, 20, 22F, 23F and/or 33F. A composition may include multiple serotypes e.g. 2, 3, 4, 5, 6, 7, 8, 9, 10, 11, 12, 13, 14, 15, 16, 17, 18, 19, 20, 21, 22, 23 or more serotypes. 7-valent, 9-valent, 10-valent, 11-valent and 13-valent conjugate combinations are already known in the art, as is a 23-valent unconjugated combination.

For example, a 10-valent combination may include saccharide from serotypes 1, 4, 5, 6B, 7F, 9V, 14, 18C, 19F and 23F. An 11-valent combination may further include saccharide from serotype 3. A 12-valent combination may add to the 10-valent mixture: serotypes 6A and 19A; 6A and 22F; 19A and 22F; 6A and 15B; 19A and 15B; r 22F and 15B; A 13-valent combination may add to the 11-valent mixture: serotypes 19A and 22F; 8 and 12F; 8 and 15B; 8 and 19A; 8 and 22F; 12F and 15B; 12F and 19A; 12F and 22F; 15B and 19A; 15B and 22F. etc. One useful 13-valent combination includes capsular saccharide from serotypes 1, 3, 4, 5, 6A, 6B, 7F, 9V, 14, 18C, 19, 19F and 23F. If saccharides are enclosed then it is preferred to include 1, 2 or 3 of serotypes 1, 5 and 14.

A carrier protein in a conjugate may or may not be one of the RrgB antigens of (1). If it is not a RrgB antigen it may instead be a different pneumococcal antigen, such as spr0057, spr0096 and spr2021, etc., or pneumolysin [122] or its non-toxic derivatives [123], or pneumococcal surface protein PspA [124], In some embodiments, though, the carrier is not a pneumococcal antigen, and may be e.g. a bacterial toxin or toxoid. Typical carrier proteins are diphtheria or tetanus toxoids or mutants thereof. The CRM₁₉₇ diphtheria toxin mutant [125] is useful, and is the carrier in the PREVNAR™ product. Other suitable carrier proteins include N. meningitidis outer membrane protein complex [126], synthetic peptides [127,128], heat shock proteins [129,130], pertussis proteins [131,132], cytokines [133], lymphokines [133], hormones [133], growth factors [133], artificial proteins comprising multiple human CD4′ T cell epitopes from various pathogen-derived antigens [134] such as N19 [135], protein D from Hinfluenzae [136-138], iron-uptake proteins [139], toxin A or B from C. difficile [140], recombinant P. aeruginosa exoprotein A (rEPA) [141], etc.

Where a composition includes more than one conjugate, each conjugate may use the same carrier protein or a different carrier protein. Reference 142 describes potential advantages when using different carrier proteins in multivalent pneumococcal conjugate vaccines

In some embodiments, a single conjugate may carry saccharides from multiple serotypes [143]. Usually, however, each conjugate will include saccharide from a single serotype.

Conjugates may have excess carrier (w/w) or excess saccharide (w/w). In some embodiments, a conjugate may include equal weights of each.

The carrier molecule may be covalently conjugated to the carrier directly or via a linker. Direct linkages to the protein may be achieved by, for instance, reductive amination between the saccharide and the carrier, as described in, for example, references 144 and 145. The saccharide may first need to be activated e.g. by oxidation. Linkages via a linker group may be made using any known procedure, for example, the procedures described in references 146 and 147. A preferred type of linkage is an adipic acid linker, which may be formed by coupling a free -NH₂ group (e.g. introduced to a glucan by amination) with adipic acid (using, for example, diimide activation), and then coupling a protein to the resulting saccharide-adipic acid intermediate [148,149]. Another preferred type of linkage is a carbonyl linker, which may be formed by reaction of a free hydroxyl group of a saccharide CDI [150, 151] followed by reaction with a protein to form a carbamate linkage. Other linkers include β-propionamido [152], nitrophenyl-ethylamine [153], haloacyl halides [154], glycosidic linkages [155], 6-aminocaproic acid [156], ADH [157], C₄ to C₁₂ moieties [158], etc. Carbodiimide condensation can also be used [159].

Further Antigens

In some embodiments, a composition of the invention includes a S. pneumoniae antigen and also an antigen from a different organism e.g. from a virus (enveloped or non-enveloped), from a Gram-negative bacterium, or from another Gram-positive bacterium.

These extra antigen(s) may take various forms e.g. a whole organism, an outer-membrane vesicle, a polypeptide, a saccharide, a liposaccharide, a conjugate (e.g. of a carrier and a hapten, or of a carrier and a saccharide or liposaccharide), etc. Where the immunogen is a polypeptide, it will typically be a surface polypeptide e.g. an adhesin, a hemagglutinin, an envelope glycoprotein, a spike glycoprotein, etc.

For example, the invention can use a S. pneumoniae antigen as discussed herein, in combination (e.g. in admixture) with one or more of the following antigens:
a polypeptide from Streptococcus agalactiae.
a capsular saccharide from S.agalactiae, for example from one or more of serotypes Ia, lb, II, III, and/or V.
a polypeptide from Streptococcus pyogenes.
a polypeptide from Staphylococcus aureus. For instance, the immunogen may comprise an IsdA antigen, an IsdB antigen, a C1fA antigen, a C1fB antigen, a SdrD antigen, a Spa antigen, an EsxA antigen, an EsxB antigen, a Sta006 antigen, a hemolysin, and/or a Sta011 antigen. Suitable S. aureus immunogens and their combinations are disclosed in reference 160.
a polypeptide from Staphylococcus epidermidis.
a capsular saccharide from Neisseria meningitidis. Capsular saccharides are particularly useful for protecting against meningococcal serogroups A, C, W135 and/or Y.
a polypeptide from N. meningitidis, for example as disclosed in reference 161.
an outer membrane vesicle from N. meningitidis, for example from a serogroup B strain.
an antigen from a hepatitis virus, such as a hepatitis A virus, a hepatitis B virus, a hepatitis C virus and/or a hepatitis E virus. For instance, the antigen may be hepatitis B virus surface antigen (HBsAg). A typical amount of HBsAg per unit dose of a vaccine is between 5-20 μg, but lower doses can be used with the invention due to the antigen-sparing nature of the adjuvants.
a polypeptide antigen from a respiratory syncytial virus. Immunogens may be from a group A RSV and/or a group B RSV. Suitable immunogens may comprise the F and/or G glycoproteins or fragments thereof e.g. as disclosed in references 162 & 163.
a polypeptide antigen from a Chlamydia bacterium, including C. trachomatis and C. pneumoniae. Suitable immunogens include those disclosed in references 164-170.
a polypeptide antigen from an Escherichia coli bacterium, including extraintestinal pathogenic strains. Suitable immunogens include those disclosed in references 171-173.
a polypeptide antigen from a coronavirus, such as the human SARS coronavirus. Suitable immunogens may comprise the spike glycoprotein.
a polypeptide antigen from Helicobacter pylori bacterium. Suitable immunogens include CagA [174-177], VacA [178,179], and/or NAP [180-182].
a polypeptide antigen from a Corynebacterium diphtheriae bacterium. Suitable immunogens include diphtheria toxoid.
a polypeptide antigen from a Clostridium tetani bacterium. Suitable immunogens include tetanus toxoid.
a polypeptide antigen from a Bordetella pertussis bacterium. Pertussis antigens are either cellular (whole cell, in the form of inactivated B. pertussis cells; ‘wP’) or acellular (‘aP’). Where acellular antigens are used, one, two or (preferably) three of the following antigens are included: (1) detoxified pertussis toxin (pertussis toxoid, or TT'); (2) filamentous hemagglutinin (‘FHA’); (3) pertactin (also known as the ‘69 kiloDalton outer membrane protein’). The PT may be chemically detoxified or may be a mutant PT in which enzymatic activity has been reduced by mutagenesis [183] e.g. the 9K/129G double mutant [184]. As well as PT, FHA and pertactin, it is also possible to include fimbriae (e.g. agglutinogens 2 and 3) in an acellular pertussis antigen component.
a capsular saccharide antigen from a Haemophilus influenzae type B bacterium (“Hib”). Suitable immunogens include conjugates of the Hib capsular saccharide (“PRP”).
an inactivated poliovirus antigen. A typical composition will include three poliovirus antigens—poliovirus Type 1 (e.g. Mahoney strain), poliovirus Type 2 (e.g. MEF-1 strain), and poliovirus Type 3 (e.g. Saukett strain).
a polypeptide antigen from a cytomegalovirus (‘CMV’). For example, the immunogen may be a recombinant glycoprotein B e.g. the soluble antigen used in reference 185.
a human papillomavirus antigen. Useful immunogens are Ll capsid proteins, which can assemble to form structures known as virus-like particles (VLPs). The VLPs can be produced by recombinant expression of L1 in yeast cells (e.g. in S. cerevisiae) or in insect cells (e.g. in Spodoptera cells, such as Sfrugiperda, or in Drosophila cells). For yeast cells, plasmid vectors can carry the L1 gene(s); for insect cells, baculovirus vectors can carry the L1 gene(s). More preferably, the composition includes L1 VLPs from both HPV-16 and HPV-18 strains. This bivalent combination has been shown to be highly effective [186]. In addition to HPV-16 and HPV-18 strains, it is also possible to include Ll VLPs from HPV-6 and HPV-11 strains.
a saccharide antigen from a Candida fungus such as C. albicans. For instance, the immunogen may be a β-glucan, which may be conjugated to a carrier protein. The glucan may include β-1,3 and/or β-1,6 linkages. Suitable immunogens include those disclosed in references 187 & 188
a polypeptide antigen from a Moraxella catarrhalis bacterium.

Where the additional antigen is a saccharide, it is preferably conjugated to a carrier protein, such as a bacterial toxin (e.g. diphtheria or tetanus toxins, or toxoids or mutants thereof, including the CRM197 diphtheria toxin mutant) or other carrier, as listed above.

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. In some embodiments, however, the composition does not comprise all three of (i) a diphtheria toxoid, (ii) a tetanus toxoid and (iii) a pertussis toxoid; thus these compositions are DTP-free.

Antibodies

Antibodies against pneumococcal antigens can be used for passive immunisation [189]. Thus the invention provides an antibody that binds to polypeptide comprising one or more of the identified epitopes. Typically, the antibody binds specifically to a polypeptide of the invention. The invention further provides a combination of antibodies for simultaneous, separate or sequential administration, wherein the combination includes at least two of: (a) an antibody which recognises a first amino acid sequence as defined above; (b) an antibody which recognises a second amino acid sequence as defined above; (c) an antibody which recognises a third amino acid sequence as defined above; (d) an antibody which recognises a fourth amino acid sequence as defined above; (a) an antibody which recognises a fifth amino acid sequence as defined above; and/or (a) an antibody which recognises a sixth amino acid sequence as defined above.

The invention also provides the use of such antibodies and antibody combinations in therapy. The invention also provides the use of such antibodies and antibody combinations in the manufacture of a medicament. The invention also provides a method for treating a mammal comprising the step of administering to the mammal an effective amount of such an antibody or combination. As described above for immunogenic compositions, these methods and uses allow a mammal to be protected against pneumococcal infection.

The term “antibody” includes intact immunoglobulin molecules, as well as fragments thereof which are capable of binding an antigen. These include hybrid (chimeric) antibody molecules [190, 191]; F(ab′)2 and F(ab) fragments and Fv molecules; non-covalent heterodimers [192, 193]; single-chain Fv molecules (sFv) [194]; dimeric and trimeric antibody fragment constructs; minibodies [195, 196]; humanized antibody molecules [197-199]; and any functional fragments obtained from such molecules, as well as antibodies obtained through non-conventional processes such as phage display. Preferably, the antibodies are monoclonal antibodies. Methods of obtaining monoclonal antibodies are well known in the art. Humanised or fully-human antibodies are preferred.

Polypeptides used with the Invention

Polypeptides used with the invention can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides <40 amino acids long involves in vitro chemical synthesis [200,201]. Solid-phase peptide synthesis is particularly preferred, such as methods based on tBoc or Fmoc [202] chemistry. Enzymatic synthesis may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [204]. Where D-amino acids are included, however, it is preferred to use chemical synthesis. Polypeptides may have covalent modifications at the C-terminus and/or N-terminus. Recombinantly-expressed proteins are preferred, particularly for hybrid polypeptides.

Polypeptides can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides are preferably provided in purified or substantially purified form i.e. substantially free from other polypeptides (e.g. free from naturally-occurring polypeptides), particularly from other pneumococcal or host cell polypeptides, and are generally at least about 50% pure (by weight), and usually at least about 90% pure i.e. less than about 50%, and more preferably less than about 10% (e.g. 5% or less) of a composition is made up of other expressed polypeptides.

Polypeptides may be attached to a solid support. Polypeptides may comprise a detectable label (e.g. a radioactive or fluorescent label, or a biotin label).

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains. Polypeptides can be naturally or non-naturally glycosylated (i.e. the polypeptide has a glycosylation pattern that differs from the glycosylation pattern found in the corresponding naturally occurring polypeptide).

Strains and Variants

Many polypeptide antigens are defined above by reference to “spr” nomenclature. This nomenclature refers to the numbering used in reference 205 for unique identification of open reading frames in the

R6 strain of S. pneumoniae. The basic reference sequence for any “spr” number can easily be found in public gene databases. For instance, GenBank accession number NC_{—}003098 (GI:15902044) is the complete R6 genome sequence (2,038,615 bp), and the individual spr sequences are given as “locus_tag” entries in the genome sequence's “features” section. Thus the amino acid sequence for any given spr number, and its natural coding sequence, can be established unambiguously for strain R6. Functional annotations are also given in the databases.

The invention is not limited to sequences from the R6 strain. Genome sequences of several other strains of S. pneumoniae are available, including those of 23F [206], 670 [207] and TIGR4 [208,209,210]. Standard search and alignment techniques can be used to identify in any of these (or other) further genome sequences the homolog of any particular spr sequence from R6. Moreover, the available R6 (and other) sequences can be used to design primers for amplification of homologous sequences from other strains. Thus the invention is not limited to R6 sequences, but rather encompasses such variants and homologs from other strains of S. pneumoniae, as well as non-natural variants. In general, suitable variants of a particular SEQ ID NO include its allelic variants, its polymorphic forms, its homologs, its orthologs, its paralogs, its mutants, etc.

Thus, for instance, polypeptides used with the invention may, compared to the R6 reference sequence, include one or more (e.g. 1, 2, 3, 4, 5, 6, 7, 8, 9, etc.) amino acid substitutions, such as conservative substitutions (i.e. substitutions of one amino acid with another which has a related side chain). Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may also include one or more (e.g. 1, 2, 3, 4, 5, 6, 7, 8, 9, etc.) single amino acid deletions relative to the R6 sequences. The polypeptides may also include one or more (e.g. 1, 2, 3, 4, 5, 6, 7, 8, 9, etc.) insertions (e.g. each of 1, 2, 3, 4 or 5 amino acids) relative to the R6 sequences.

Similarly, a polypeptide used with the invention may comprise an amino acid sequence that:
(a) is identical (i.e. 100% identical) to a sequence disclosed in the sequence listing;
(b) shares sequence identity (e.g. 60%, 65%, 70%, 75%, 80%, 85%, 90%, 91%, 92%, 93%, 94%, 95%, 96%, 97%, 98%, 99%, 99.5% or more) with a sequence disclosed in the sequence listing;
(c) has 1, 2, 3, 4, 5, 6, 7, 8, 9 or 10 (or more) single amino acid alterations (deletions, insertions, substitutions), which may be at separate locations or may be contiguous, as compared to the sequences of (a) or (b); and
(d) when aligned with a particular sequence from the sequence listing using a pairwise alignment algorithm, each moving window of x amino acids from N-terminus to C-terminus (such that for an alignment that extends to p amino acids, where p>x, there are p-x+1 such windows) has at least xy identical aligned amino acids, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if xy is not an integer then it is rounded up to the nearest integer. The preferred pairwise alignment algorithm is the Needleman-Wunsch global alignment algorithm [211], using default parameters (e.g. with Gap opening penalty=10.0, and with Gap extension penalty=0.5, using the EBLOSUM62 scoring matrix). This algorithm is conveniently implemented in the needle tool in the EMBOSS package [212].

Where hybrid polypeptides are used, the individual antigens within the hybrid (i.e. individual —X— moieties) may be from one or more strains. Where n=2, for instance, X₂ may be from the same strain as X₁ or from a different strain. Where n=3, the strains might be (i) X₁═X₂═X₃ (ii) X₁═X₂≠X₃(iii) X₁≠X₂═X₃ (iv) X₁≠X₂≠X₃ or (v) X₁=X₃≠X₂, etc.

Within group (c), deletions or substitutions may be at the N-terminus and/or C-terminus, or may be between the two termini. Thus a truncation is an example of a deletion. Truncations may involve deletion of up to 40 (or more) amino acids at the N-terminus and/or C-terminus.

In general, when a polypeptide of the invention comprises a sequence that is not identical to a complete pneumococcal sequence from the sequence listing (e.g. when it comprises a sequence listing with <100% sequence identity thereto, or when it comprises a fragment thereof) it is preferred in each individual instance that the polypeptide can elicit an antibody that recognises the complete pneumococcal sequence.

Formulae (C), (D), (E) and (H)—TLR 7 agonists

The TLR agonist can be a compound according to any of formulae (C), (D), (E), and (H):

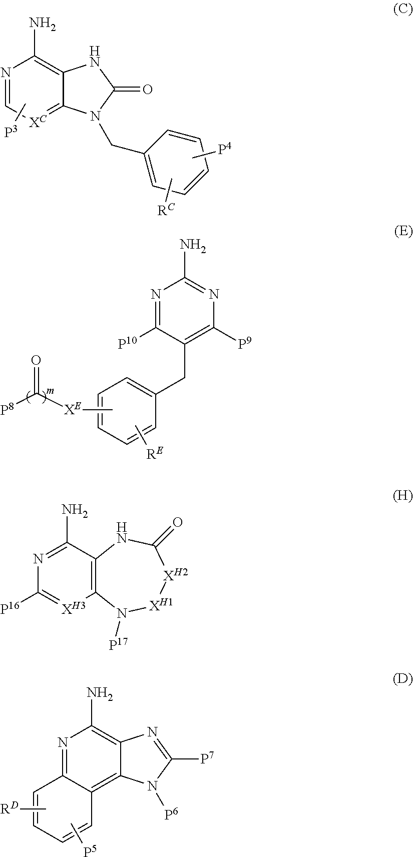

wherein:
(a) P³ is selected from H, C₁-C₆alkyl, CF₃, and —((CH₂)ₚO)_{q}(CH₂)ₚOₛ— and —Y-L-X—P(O)(OR^{X})(OR^{Y}); and P⁴ is selected from H, C₁-C₆alkyl, —C₁-C₆alkylaryl and —Y-L-X—P(O)(OR^{X})(OR^{Y}); with the proviso that at least one of P³ and P⁴ is —Y-L-X—P(O)(OR^{X})(OR^{Y}),
(b) P⁵ is selected from H, C₁-C₆alkyl, and —Y-L-X—P(O)(OR^{X})(OR^{Y}); P⁶ is selected from H, C₁-C₆alkyl each optionally substituted with 1 to 3 substituents selected from C₁-C₄alkyl and OH, and —Y-L-X—P(O)(ORNOR^{Y}); and P⁷ is selected from H, C₁-C₆alkyl, —((CH₂)ₚO)ₚ(CH₂)ₚOₛ—, —NHC₁-C₆alkyl and —Y-L-X—P(O)(OR^{X})(OR^{Y}); with the proviso that at least one of P⁵, P⁶ and P⁷ is —Y-L-X—P(O)(OR^{X})(OR^{Y});
(c) P⁸ is selected from H, C₁-C₆alkyl, C₁-C₆alkoxy, —NHC₁-C₆alkyl each optionally substituted with OH, and —Y-L-X—P(O)(OR^{X})(OR^{Y}); and P⁹ and P¹⁹ are each independently selected from H, C₁-C₆alkyl, C₁-C₆alkoxy, —NHC₁-C₆alkyl each optionally substituted with OH and C₁-C₆alkyl, and —Y-L-X—P(O)(OR^{X})(OR^{Y}); with the proviso that at least one of P^{8′} P⁹ or P¹⁹ is —Y-L-X—P(O)(OR^{X})(OR^{Y});
(d) P¹⁶ and each P¹⁸ are each independently selected from H, C₁-C₆alkyl, and —Y-L-X—P(O)(OR^{X})(OR^{Y}); P¹⁷ is selected from H, C₁-C₆alkyl, aryl, heteroaryl, C₁-C₆alkylaryl, C₁-C₆alkyl heteroaryl, C₁-C₆alkylaryl-Y-L-X—P(O)(OR^{X})(OR^{Y}) and —Y-L-X—P(O)(OR^{X})(OR^{Y}), each optionally substituted with 1 to 2 substituents selected from C₁-C₆alkyl or heterocyclyl with the proviso that at least one of P^{16′} P¹⁷ or a P¹⁸ contains a —Y-L-X—P(O)(OR^{X})(OR^{Y}) moiety;
R^{X} and R^{Y} are independently selected from H and C₁-C₆alkyl;
R^{C}, R^{D} and R^{H} are each independently selected from H and C₁-C₆alkyl;
X^{C} is selected from CH and N;
R^{E} is selected from H, C₁-C₆alkyl, C₁-C₆alkoxy, C(O)C₁-C₆alkyl, halogen and —((CH₂)ₚO)_{q}(CH₂)ₚ—;
X^{E} is selected from a covalent bond, CR^{E2}R^{E3} and NR^{E4};
R^{E2}, R^{E3} and R^{E4} are independently selected from H and C₁-C₆alkyl;
X^{H1}-V^{H2} is selected from —CR^{H2}R^{H3}—, —CR^{H2}R^{H3}—CR^{H2}R^{H3}—, —C(O)CR^{H2}R^{H3}—, —C(O)CR^{H2}R^{H3}—, —CR^{H2}R^{H3}C(O)—, —NR^{H4}C(O)—, C(O)NR^{H4}—, CR^{H2}R^{H3}S(O)₂ and —CR^{H2}=CR^{H2}—;
R^{H2}, R^{H3} and R^{H4} are each independently selected from H, C₁-C₆alkyl and P¹⁸;
X^{H3} is selected from N and CN;
X is selected from a covalent bond, O and NH;
Y is selected from a covalent bond, O, C(O), S and NH;
L is selected from, a covalent bond C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂;
m is selected from 0 or 1;
each p is independently selected from 1, 2, 3, 4, 5 and 6;
q is selected from 1, 2, 3 and 4; and
s is selected from 0 and 1.

### Formula (G)—TLR8 Agonist

The TLR agonist can be a compound according to formula (G):

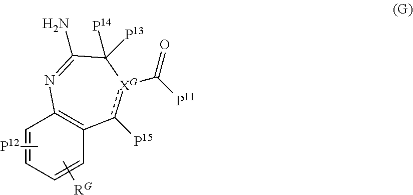

wherein:
P¹¹ is selected from H, C₁-C₆alkyl, C₁-C₆ alkoxy, NR^{V}R^{W} and —Y-L-X—P(O)(OR^{X})(OR^{Y});
P¹² is selected from H, C₁-C₆alkyl, aryl optionally substituted by —C(O)NR^{V}R^{W}, and —Y-L-X—P(O)(OR^{X})(OR^{Y});
P¹³, P¹⁴ and P¹⁵ are independently selected from H, C₁-C₆alkyl, C₁-C₆ alkoxy and —Y-L-X—P(O)(OR^{X})(OR^{Y});
with the proviso that at least one of P¹¹, P¹², P¹³, P¹⁴ or P¹⁵ is —Y-L-X—P(O)(OR^{X})(OR^{Y});
R^{V} and R^{W} are independently selected from H, C₁-C₆alkyl or together with the nitrogen atom to which they are attached form a 4 to 7 remembered heterocyclic ring;
X^{G} is selected from C, CH and N;
[symbol] represents an optional double bond, wherein X^{G} is C if [symbol] is a double bond; and
R^{G} is selected from H and C₁-C₆alkyl;
X is selected from a covalent bond, O and NH;
Y is selected from a covalent bond, O, C(O), S and NH;
L is selected from, a covalent bond C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂;
each p is independently selected from 1, 2, 3, 4, 5 and 6 and
q is selected from 1, 2, 3 and 4.

Formulae (I) and (II)—TLR 7 agonists [6]

The TLR agonist can be a compound according to formula (I) or formula (II):

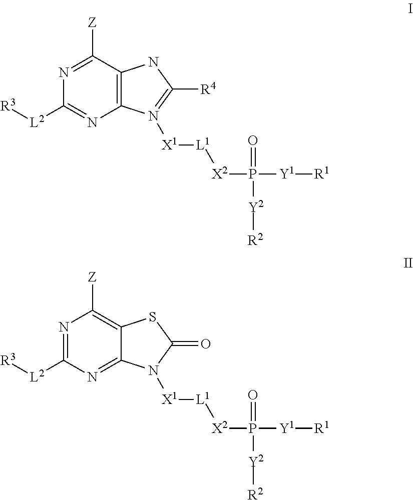

wherein:
Z is —NH₂ or —OH;
X¹ is alkylene, substituted alkylene, alkenylene, substituted alkenylene, alkynylene, substituted alkynylene, carbocyclylene, substituted carbocyclylene, heterocyclylene, or substituted heterocyclylene;
L¹ is a covalent bond, arylene, substituted arylene, heterocyclylene, substituted heterocyclylene, carbocyclylene, substituted carbocyclylene, —S—, —S(O)—, S(O)₂, —NR⁵—, or —O—
X² is a covalent bond, alkylene, or substituted alkylene;
L² is NR⁵—, —N(R⁵)C(O)—, —O—, —S—, —S(O)—, S(O)₂, or a covalent bond;
R³ is H, alkyl, substituted alkyl, heteroalkyl, substituted heteroalkyl, alkenyl, substituted alkenyl, aryl, substituted aryl, arylalkyl, substituted arylalkyl, heterocyclyl, substituted heterocyclyl, heterocyclylalkyl, or substituted heterocyclylalkyl;
Y¹ and Y² are each independently a covalent bond, —O— or —NR⁵—; or —Y¹—R¹ and —Y²—R² are each independently —O—N═C(R⁶R²);
R¹ and R² are each independently H, alkyl, substituted alkyl, carbocyclyl, substituted carbocyclyl, heterocyclyl, substituted heterocyclyl, alkenyl, substituted alkenyl, alkynyl, substituted alkynyl, arylalkyl, substituted arylalkyl, heterocyclylalkyl, substituted heterocyclylalkyl, -alkylene-C(O)—O—R⁵, (substituted alkylene)-C(O)—O—R⁵, -alkylene-O—C(O)—R⁵, —(substituted alkylene)-O—C(O)—R⁵, —alkylene-O—C(O)—O—R⁵, or -(substituted alkylene)-O—C(O)—O—R⁵
R⁴ is H, halogen, —OH, —O-alkyl, —O-alkylene-O—C(O)—O—R⁵, —O—C(O)—O—R⁵, —SH, or —NH(R⁵);
each R⁵, R⁶, and R² are independently H, alkyl, substituted alkyl, carbocyclyl, substituted carbocyclyl, heterocyclyl, substituted heterocyclyl, alkenyl, substituted alkenyl, alkynyl, substituted alkynyl, arylalkyl, substituted arylalkyl, heterocyclylalkyl, or substituted heterocyclylalkyl.

Formula (J)—TLR2 agonists [213]

The TLR agonist can be a compound according to formula (J):

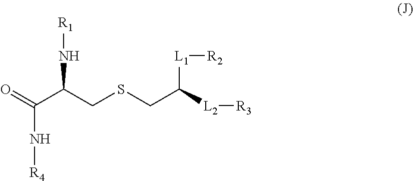

wherein:
R¹ is H, —C(O)—C₇-C₁₈alkyl or —C(O)—C₁-C₆alkyl;
R² is C₇-C₁₈alkyl ;
R³ is C₇-C₁₈alkyl;
L₁ is —CH₂OC(O)—, —CH₂O—, —CH₂NR⁷C(O)— or —CH₂OC(O)NR⁷—;
L₂ is —OC(O)—, —O—, —NR⁷C(O)— or —OC(O)NR⁷—;
R⁴ is -L₃R⁵ or -L₄R⁵;
R⁵ is —N(R⁷)₂, —P(O)(OR⁷)₂, —C(O)OR⁷, —NR⁷C(O)L₃R⁸, —NR⁷C(O)L₄R⁸, —OL₃R⁶, —C(O)NR⁷L₃R⁸, —C(O)NR⁷L₄R⁸, —S(O)₂OR⁷, —OS(O)₂OR⁷, C₁-C₆alkyl, a C₆aryl, a C₁₀aryl, a C₁₄aryl, 5 to 14 ring membered heteroaryl containing 1 to 3 heteroatoms selected from O, S and N, C₃-C₈cycloalkyl or a 5 to 6 ring membered heterocycloalkyl containing 1 to 3 heteroatoms selected from O, S and N, wherein the aryl, heteroaryl, cycloalkyl and heterocycloalkyl of R⁵ are each unsubstituted or the aryl, heteroaryl, cycloalkyl and heterocycloalkyl of R⁵ are each substituted with 1 to 3 substituents independently selected from —OR⁹, —OL₃R⁶, —OL₄R⁶, —OR⁷, and —C(O)OR⁷;
L₃ is a C₁-C₁₀alkylene, wherein the C₁-C₁₀alkylene of L₃ is unsubstituted, or the C₁-C₁₀alkylene of L₃ is substituted with 1 to 4 R⁶ groups, or the C₁-C₁ₒalkylene of L₃ is substituted with 2 C₁-C₆alkyl groups on the same carbon atom which together, along with the carbon atom they are attached to, form a C₃-C₈cycloakyl;
L₄ is —((CR⁷R¹⁰)ₚO)_{q}(CR¹⁰R¹⁰)ₚ— or —(CR¹¹R¹¹)((CR⁷R⁷)ₚO)_{q}(CR¹⁰R¹⁰)ₚ—, wherein each R¹¹ is a C₁-C₆alkyl groups which together, along with the carbon atom they are attached to, form a C₃-C₈cycloakyl;
each R⁶ is independently selected from halo, C₁-C₆alkyl, C₁-C₆alkyl substituted with 1-2 hydroxyl groups, —OR⁷, —N(R⁷)₂, —C(O)OH, —C(O)N(R⁷)₂, —P(O)(OR⁷)₂, a C₆aryl, a C₁₀aryl and a C₁₄aryl;
each R⁷ is independently selected from H and C₁-C₆alkyl;
R⁸ is selected from —SR⁷, —C(O)OH, —P(O)(OR⁷)₂, and a 5 to 6 ring membered heterocycloalkyl containing 1 to 3 heteroatoms selected from O and N;
R⁹ is phenyl;
each R¹⁹ is independently selected from H and halo;
each p is independently selected from 1, 2, 3, 4, 5 and 6, and
q is 1, 2, 3 or 4.

Preferably R⁵ is P(O)(OR⁷)₂, —NR⁷C(O)L₃-P(O)(OR⁷)₂, —NR⁷C(O)L₄-P(O)(OR⁷)₂, —OL₃-P(O)(OR⁷)₂, —C(O)NR⁷L₃-P(O)(OR⁷)₂, or —C(O)NR⁷L₄-P(O)(OR⁷)₂.

In some embodiments of (J), R₁ is H. In other embodiments of (J), R₁ is —C(O)—C₁₅alkyl;

In some embodiments of (J): (i) L₁ is —CH₂OC(O)— and L₂ is —OC(O)—, —O—, —NR⁷C(O)— or —OC(O)NR⁷—; or (ii) or L₁ is —CH₂O— and L₂ is —OC(O)—, —O—, —NR⁷C(O)— or —OC(O)NR⁷—; or (iii) L₁ is —CH₂NR⁷C(O)— and L₂ is —OC(O)—, —O—, —NR⁷C(O)— or —OC(O)NR⁷—; or (iv) L₁ is —CH₂OC(O)NR⁷— and L₂ is —OC(O)—, —O—, NR⁷C(O)— or —OC(O)NR⁷—.

In some embodiments of (J): (i) L₁ is —CH₂OC(O)— and L₂ is —OC(O)—; or (ii) L₁ is —CH₂O— and L₂ is —O—; or (iii) L₁ is —CH₂O— and L₂ is —NHC(O)—; or (iv) L₁ is —CH₂OC(O)NH— and L₂ is —OC(O)NH—.

In some embodiments of (J), (i) R² is —C₁₁alkyl and R³ is —C₁₁alkyl; or (ii) R² is —C₁₆alkyl and R³ is —C₁₆alkyl; or (iii) R² is —C₁₆alkyl and R³ is —C₁₁alkyl; or (iv) R² is -C₁₂alkyl and R³ is -C₁₂alkyl; or (v) R² is —C₇alkyl and R³ is —C₇alkyl; or (vi) R² is —C₉alkyl and R³ is —C₉alkyl; or (vii) R² is —C₁₈alkyl and R³ is —C₈alkyl; or (viii) R² is —C₁₃alkyl and R³ is —C₁₃alkyl; or (ix) R² is —C₁₂alkyl and R³ is —C₁₁alkyl; or (x) R² is —C₁₂alkyl and R³ is —C₁₂alkyl; or (xi) R² is -C₁₀alkyl and R³ is —C₁₀alkyl; or (xii) R² is —C₁₅alkyl and R³ is —C₁₅alkyl.

In some embodiments of (J), R² is —C₁₁alkyl and R³ is —C₁₁alkyl.

In some embodiments of (J), L₃ is a C₁-C₁₀alkylene, wherein the C₁-C₁₀alkylene of L₃ is unsubstituted or is substituted with 1 to 4 R⁶ groups.

In some embodiments of (J): L₄ is —((CR⁷R⁷)ₚO)_{q}(CR¹⁰ R¹⁰)ₚ—; each R¹⁰ is independently selected from H and F; and each p is independently selected from 2, 3, and 4.

In some embodiments of (J), each R⁶ is independently selected from methyl, ethyl, i-propyl, i-butyl, —CH₂OH, —OH, —F, —NH₂, —C(O)OH, —C(O)NH₂, —P(O)(OH)₂ and phenyl.

In some embodiments of (J), each R⁷ is independently selected from H, methyl and ethyl.

Formula (K) [214]

The TLR agonist can be a compound according to formula (K):

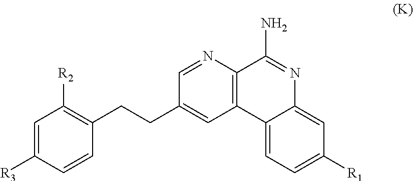

wherein:
R¹ is H, C₁-C₆alkyl, —C(R⁵)₂OH, -L¹R⁵, -L¹R⁶, -L²R⁵, -L²R⁶, —OL²R⁵, or —OL²R⁶;
L¹ is —C(O)— or —O—;
L² is C₁-C₆alkylene, C₂-C₆alkenylene, arylene, heteroarylene or —((CR⁴R⁴)ₚO)_{q}(CH₂)ₚ—, wherein the C₁-C₆alkylene and C₂-C₆alkenylene of L² are optionally substituted with 1 to 4 fluoro groups;
each L³ is independently selected from C₁-C₆alkylene and —((CR⁴R⁴)ₚO)_{q}(CH₂)ₚ—, wherein the C₁-C₆alkylene of L³ is optionally substituted with 1 to 4 fluoro groups;
L⁴ is arylene or heteroarylene;
R² is H or C₁-C₆alkyl;
R³ is selected from C₁-C₄alkyl, L³R⁵, -L¹R⁵, -L³R⁷, -L³L⁴L³R⁷, -L³L⁴R⁵, -L³L⁴L³R⁵, —OL³R⁵, —OL³R⁷, —OL³L⁴R⁷, —OL³L⁴L³R⁷, —OR⁸, —OL³L⁴R⁵, —OL³L⁴L³R⁵ and —C(R⁵)₂OH;
each R⁴ is independently selected from H and fluoro;
R⁵ is —P(O)(OR⁹)₂,
R⁶ is CF₂P(O)(OR⁹)₂ or —C(O)OR¹⁰;
R⁷ is CF₂P(O)(OR⁹)₂ or —C(O)OR¹⁰;
R⁸ is H or C₁-C₄alkyl;
each R⁹ is independently selected from H and C₁-C₆alkyl;
R¹⁰ is H or C₁-C₄alkyl;
each p is independently selected from 1, 2, 3, 4, 5 and 6, and
q is 1, 2, 3 or 4.

The compound of formula (K) is preferably of formula (K′):

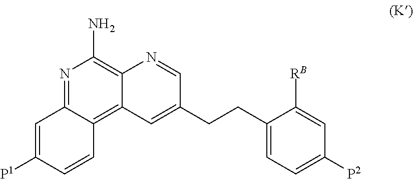

wherein:
P¹ is selected from H, C₁-C₆alkyl optionally substituted with COOH and —Y-L-X—P(O)(OR^{X})(OR^{Y});
P² is selected from H, C₁-C₆alkyl, C₁-C₆alkoxy and —Y-L-X—P(O)(OR^{X})(OR^{Y});
with the proviso that at least one of P¹ and P² is —Y-L-X—P(O)(OR^{X})(OR^{Y});
R^{B} is selected from H and C₁-C₆alkyl;
R^{X} and R^{Y} are independently selected from H and C₁-C₆alkyl;
X is selected from a covalent bond, O and NH;
Y is selected from a covalent bond, O, C(O), S and NH;
L is selected from, a covalent bond C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂;
each p is independently selected from 1, 2, 3, 4, 5 and 6; and
q is selected from 1, 2, 3 and 4.

In some embodiments of formula (K′): P¹ is selected from C₁-C₆alkyl optionally substituted with COOH and —Y-L-X—P(O)(OR^{X})(OR^{Y}); P² is selected from C₁-C₆alkoxy and —Y-L-X—P(O)(OR^{X})(OR^{Y}); R^{B} is C₁-C₆alkyl; X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; q is selected from 1 and 2.

Formula (F)—TLR 7 agonists [7]

The TLR agonist can be a compound according to formula (F):

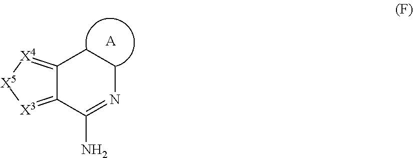

wherein:
   X³ is N;
   X⁴ is N or CR³
   X⁵ is —CR⁴═CR⁵—;
   R¹ and R² are H;
   R³ is H;
   R⁴ and R⁵ are each independently selected from H, halogen, —C(O)OR⁷, —C(O)R⁷, —C(O)N(R¹¹R¹²), —N(R¹¹R¹²), —N(R⁹)₂, —NHN(R⁹)₂, —SR⁷, —(CH₂)ₙR⁷, —(CH₂)R⁷, —LR⁸, -LR¹⁰, —OLR⁸, —OLR¹⁰, C₁-C₆alkyl, C₁-C₆heteroalkyl, C₁-C₆halo alkyl, C₂-C₈alkene, C₂-C₈alkyne, C₁-C₆alkoxy, C₁-C₆haloalkoxy, aryl, heteroaryl, C₃-C₈cycloalkyl, and C₃-C₈heterocycloalkyl, wherein the C₁-C₆alkyl, C₁-C₆heteroalkyl, C₁-C₆haloalkyl, C₂-C₈alkene, C₂-C₈alkyne, C₁-C₆alkoxy, C₁-C₆haloalkoxy, aryl, heteroaryl, C₃-C₈cycloalkyl, and C₃-C₈heterocycloalkyl groups of R⁴ and R⁵ are each optionally substituted with 1 to 3 substituents independently selected from halogen, —CN, —NO₂, —R⁷, —OR⁸, —C(O)R⁸, —OC(O)R⁸, —C(O)OR⁸, —N(R⁹)₂, —P(O)(OR⁸)₂, —OP(O)(OR⁸)₂, —P(O)(OR¹⁰)₂. —OP(O)(OR¹⁰)₂, —C(O)N(R⁹)₂, —S(O)₂R⁸, —S(O)R⁸, —S(O)₂N(R⁹)₂, and —NR⁹S(O)₂R⁸;
   or, R³ and R⁴, or R⁴ and R⁵, or R⁵ and R⁶, when present on adjacent ring atoms, can optionally be linked together to form a 5-6 membered ring, wherein the 5-6 membered ring is optionally substituted with R⁷;
   each L is independently selected from a bond, —(O(CH₂)ₘ)ₜ—, C₁-C₆alkyl, C₂-C₆alkenylene and C₂-C₆alkynylene, wherein the C₁-C₆alkyl, C₂-C₆alkenylene and C₂-C₆alkynylene of L are each optionally substituted with 1 to 4 substituents independently selected from halogen, —R⁸, —OR⁸, —N(R⁹)₂, —P(O)(OR⁸)₂, —OP(O)(OR⁸)₂, —P(O)(OR¹⁰)₂, and —OP(O)(OR¹⁰)₂;
   R⁷ is selected from H, C₁-C₆alkyl, aryl, heteroaryl, C₃-C₈cycloalkyl, C₁-C₆heteroalkyl, C₁-C₆haloalkyl, C₂-C₈alkene, C₂-C₈alkyne, C₁-C₆alkoxy, C₁-C₆haloalkoxy, and C₃-C₈heterocycloalkyl, wherein the C₁-C₆alkyl, aryl, heteroaryl, C₃-C₈cycloalkyl, C₁-C₆heteroalkyl, C₁-C₆haloalkyl, C₂-C₈alkene, C₂-C₈alkyne, C₁-C₆alkoxy, C₁-C₆haloalkoxy, and C₃-C₈heterocycloalkyl groups of R⁷ are each optionally substituted with 1 to 3 R¹³ groups, and each R¹³ is independently selected from halogen, —CN, -LR⁹, -LOR⁹, —OLR⁹, -LR¹⁰, -LOR¹⁰, —OLR¹⁰, -LR⁸, -LOR⁸, —OLR⁸, -LSR⁸, -LSR¹⁰, -LC(O)R⁸, —OLC(O)R⁸, -LC(O)OR⁸, -LC(O)R¹⁰, -LOC(O)¹⁰, -LC(O)NR⁹R¹¹, -LC(O)NR⁹R⁸, -LN(R⁹)₂, -LNR⁹R⁸, -LNR⁹R¹⁰, -LC(O)N(R⁹)₂, -LS(O)₂R⁸, -LS(O)R⁸, -LC(O)NR⁸OH, -LNR⁹C(O)R⁸, -LNR⁹C(O)OR⁸, -LS(O)₂N(R⁹)₂, —OLS(O)₂N(R⁹)₂, -LNR⁹S(O)₂R⁸, -LC(O)NR⁹LN(R⁹)₂, -LP(O)(OR⁸)₂, -LOP(O)(OR⁸)₂, -LP(O)(OR¹⁰)₂ and —OLP(O)(OR¹⁰)₂;

each R⁸ is independently selected from H, —CH(R¹⁰)₂, C₁-C₈alkyl, C₂-C₈alkene, C₂-C₈alkyne, C₁-C₆halo alkyl, C₁-C₆alkoxy, C₁-C₆hetero alkyl, C₃⁻C₈cycloalkyl, C₂-C₈heterocycloalkyl, C₁-C₆hydroxyalkyl and C₁-C₆haloalkoxy, wherein the C₁-C₈alkyl, C₂-C₈alkene, C₂-C₈alkyne, C₁-C₆heteroalkyl, C₁-C₆haloalkyl, C₁-C₆alkoxy, C₃-C₈cycloalkyl, C₂-C₈heterocycloalkyl, C₁-C₆hydroxyalkyl and C₁-C₆haloalkoxy groups of R⁸ are each optionally substituted with 1 to 3 substituents independently selected from —CN, R¹¹, —OR¹¹, —SR¹¹, —C(O)R¹¹, —OC(O)R¹¹, —C(O)N(R⁹)₂, —C(O)OR¹¹, —NR⁹C(O)R¹¹, —NR⁹R¹⁰, —NR¹¹R¹², —N(R⁹)₂, —OR⁹, —OR¹⁰, —C(O)NR¹¹R¹², —C(O)NR¹¹OH, —S(O)₂R¹¹, —S(O)R¹¹, —S(O)₂NR¹¹R¹², —NR¹¹S(O)₂R¹¹, —P(O)(OR¹¹)₂, and —OP(O)(OR¹¹)₂;

each R⁹ is independently selected from H, —C(O)R⁸, —C(O)OR⁸, —C(O)R¹⁰, —C(O)OR¹⁰, —S(O)₂R¹⁰, —C₁-C₆ alkyl, C₁-C₆ heteroalkyl and C₃-C₆ cycloalkyl, or each R⁹ is independently a C₁-C₆aIkyl that together with N they are attached to form a C₃-C₈heterocycloalkyl, wherein the C₃-C₈heterocycloalkyl ring optionally contains an additional heteroatom selected from N, O and S, and wherein the C₁-C₆ alkyl, C₁-C₆ heteroalkyl, C₃-C₆ cycloalkyl, or C₃-C₈heterocycloalkyl groups of R⁹ are each optionally substituted with 1 to 3 substituents independently selected from —CN, R¹¹, —OR¹¹, —SR¹¹, —C(O)R¹¹, OC(O)R¹¹, —C(O)OR¹¹, —NR¹¹R¹², —C(O)NR¹¹R¹², —C(O)NR¹¹OH, —S(O)₂R¹¹, —S(O)₂NR¹¹R¹², —NR¹¹S(O)₂R¹¹, —P(O)(OR¹¹)₂ and —P(O)(OR¹¹)₂;

each R¹⁰ is independently selected from aryl, C₃-C₈cycloalkyl, C₃-C₈heterocycloalkyl and heteroaryl, wherein the aryl, C₃-C₈cycloalkyl, C₃-C₈heterocycloalkyl and heteroaryl groups are optionally substituted with 1 to 3 substituents selected from halogen, —R⁸, —OR⁸, —LR⁹, -LOR⁹, —N(R⁹)₂, —NR⁹C(O)R⁸, —NR⁹CO₂R⁸. —CO₂R⁸, —C(O)R⁸ and —C(O)N(R⁹)₂;

R¹¹ and R¹² are independently selected from H, C₁-C₆alkyl, C₁-C₆heteroalkyl, C₁-C₆haloalkyl, aryl, heteroaryl, C₃-C₈cycloalkyl, and C₃-C₈heterocycloalkyl, wherein the C₁-C₆alkyl, C₁-C₆heteroalkyl, C₁-C₆haloalkyl, aryl, heteroaryl, C₃-C₈cycloalkyl, and C₃-C₈heterocycloalkyl groups of R″ and R¹² are each optionally substituted with 1 to 3 substituents independently selected from halogen, —CN, R⁸, —OR⁸, C(O)R⁸, OC(O)R⁸, —C(O)OR⁸, —N(R⁹)₂, —NR⁸C(O)R⁸, —NR⁸C(O)OR⁸, —C(O)N(R⁹)₂, C₃-C₈heterocycloalkyl, —S(O)₂R⁸, —S(O)₂N(R⁹)₂, —NR⁹S(O)₂R⁸, C₁-C₆haloalkyl and C₁-C₆haloalkoxy;
or R¹¹ and R¹² are each independently C₁-C₆alkyl and taken together with the N atom to which they are attached form an optionally substituted C₃-C₈heterocycloalkyl ring optionally containing an additional heteroatom selected from N, O and S;
ring A is an aryl or a heteroaryl, wherein the aryl and heteroaryl groups of Ring A are optionally substituted with 1 to 3 R^{A} groups, wherein each R^{A} is independently selected from —R⁸, —R⁷, —OR⁸, —OR⁸, —R¹⁰, —OR¹⁰, —SR⁸, —NO₂, —CN, —N(R⁹)₂, —NR⁹C(O)R⁸, —NR⁹C(S)R⁸, —NR⁹C(O)N(R⁹)₂, —NR⁹C(S)N(R⁹)₂, —NR⁹CO₂R⁸, —NR⁹NR⁹C(O)R⁸, —NR⁹NR⁹C(O)N(R⁹)₂, —NR⁹NR⁹CO₂R⁸, —C(O)C(O)R⁸, —C(O)CH₂C(O)R⁸, —OC₂R⁸, —(CH₂)ₙCO₂R⁸, —C(O)R⁸, —C(S)R⁸, —C(O)N(R⁹)₂, —C(S)N(R⁹)₂, —OC(O)N(R⁹)₂, —OC(O)R⁸, —C(O)N(OR⁸R⁸, —C(NOR⁸)R⁸, —S(O)₂R⁸, —S(O)₃R⁸, —SO₂N(R⁹)₂, —S(O)R⁸, —NR⁹SO₂N(R⁹)₂, —NR⁹SO₂R⁸, —P(O)(OR⁸)₂, —OP(O)(OR⁸)₂, —P(O)(OR¹⁰)₂, —OP(O)(OR¹⁰)₂, —N(OR⁸)R⁸, —CH═CHCO₂R⁸, —C(═NH)—N(R⁹)₂, and —(CH₂)ₙNHC(O)R⁸ or two adjacent R^{A} substituents on Ring A form a 5-6 membered ring that contains up to two heteroatoms as ring members;
n is, independently at each occurrence, 0, 1, 2, 3, 4, 5, 6, 7 or 8;
each m is independently selected from 1, 2, 3, 4, 5 and 6, and
t is 1, 2, 3, 4, 5, 6, 7 or 8.

Formulae (C), (D), (E), (G) and (H)

As discussed above, the TLR agonist can be of formula (C), (D), (E), (G) or (H).

The ‘parent’ compounds of formulae (C), (D), (E) and (H) are useful TLR7 agonists (see references 5-8 and 215-231) but are preferably modified herein by attachment of a phosphorus-containing moiety.

In some embodiments of formulae (C), (D) and (E) the compounds have structures according to formulae (C′), (D′) and (E′), shown below:

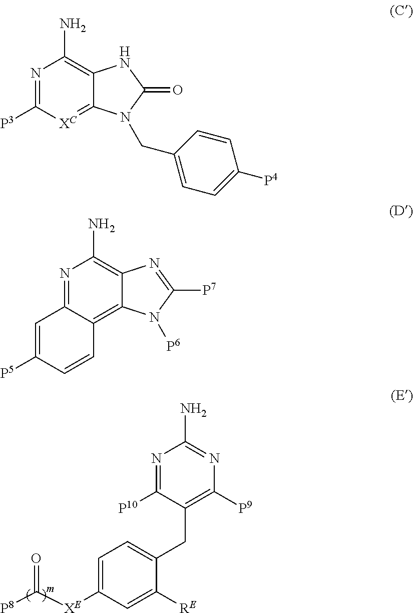

The embodiments of the invention of formulae (C), (D), (E) and (H) also apply to formulae (C′), (D′), (E′) and (H′).

In some embodiments of formulae (C), (D), (E), and (H): X is O; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

In other embodiments of formula (C): P³ is selected from C₁-C₆alkyl, CF₃, and —((CH₂)ₚO)_{q}(CH₂)ₚOₛ— and —Y-L-X—P(O)(OR^{X})(OR^{Y}); P⁴ is selected from —C₁-C₆alkylaryl and —Y-L-X—P(O)(OR^{X})(OR^{Y}); X^{C} is CH; X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; q is 1 or 2.

In other embodiments of formulae (C), (D), (E), and (H): X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂; each p is independently selected from 1, 2 and 3; and q is selected from 1 and 2.

In other embodiments of formula (C): P³ is selected from C₁-C₆alkyl, CF₃, and —((CH₂)ₚO)_{q}(CH₂)ₚOₛ— and —Y-L-X—P(O)(OR^{X})(OR^{Y}); P⁴ is selected from —C₁-C₆alkylaryl and —Y-L-X—P(O)(OR^{X})(OR^{Y}); X^{C} is N; X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; q is selected from 1 and 2.

In other embodiments of formula (D): P⁵ is selected from C₁-C₆alkyl, and —Y-L-X—P(O)(OR^{X})(OR^{Y}).

In other embodiments of formula (D): X is O; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

In other embodiments of formula (D): X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂; each p is independently selected from 1, 2 and 3; and q is selected from 1 and 2.

In other embodiments of formula (E): X is O; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

In other embodiments of formula (E): X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

In other embodiments of formula (E): X^{E} is CH₂, P⁸ is C₁-C₆alkoxy optionally substituted with —Y-L-X—P(O)(OR^{X})(OR^{Y}).

In other embodiments of formula (E): P⁹ is —NHC₁-C₆alkyl optionally substituted with OH and C₁-C₆alkyl, and —Y-L-X—P(O)(OR^{X})(OR^{Y}).

In some embodiments, a compound of formula (C) is not a compound in which P⁴ is —Y-L-X—P(O)(OR^{X})(OR^{Y}).

In some embodiments, in a compound of formula (C), P⁴ is selected from H, C₁-C₆alkyl, —C₁-C₆alkylaryl.

In some embodiments of formula (H): X^{H1}—X^{H2} is —CR^{H2}R^{H3}, R^{H2} and —R^{H3} are H, X^{H3} is N, X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂; each p is independently selected from 1, 2 and 3; and q is selected from 1 and 2.

In some embodiments of formula (H): X^{H1}—X^{H2} is —CR^{H2}R^{H3}, R^{H2} and R^{H3} are H, X^{H3} is N, X is O; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

The ‘parent’ compounds of formula (G) are useful TLR8 agonists (see references 9 & 10) but are preferably modified herein by attachment of a phosphorus-containing moiety to permit adsorption. In some embodiments of formula (G), the compounds have structures according to formula (G′);

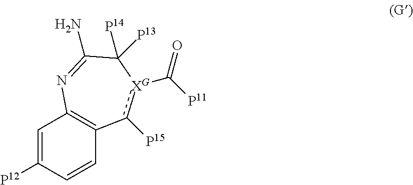

In some embodiments of formula (G) or (G′): X^{G} is [symbol] and represents a double bond.

In some embodiments of formula (G) or (G′): X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

In some embodiments of formula (G) or (G′): X is O; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

Pharmaceutical Compositions and Products

The invention provides various immunogenic compositions. These are ideally pharmaceutical compositions suitable for use in humans. Pharmaceutical compositions usually include components in addition to the TLR agonist, insoluble metal salt and/or immunogen e.g. they typically include one or more pharmaceutical carrier(s) and/or excipient(s). A thorough discussion of such components is available in reference 232.

Pharmaceutical compositions are preferably in aqueous form, particularly at the point of administration, but they can also be presented in non-aqueous liquid forms or in dried forms e.g. as gelatin capsules, or as lyophilisates, etc.

Pharmaceutical compositions may include one or more preservatives, such as thiomersal or 2-phenoxyethanol. Mercury-free compositions are preferred, and preservative-free vaccines can be prepared.

Pharmaceutical compositions can include a physiological salt, such as a sodium salt e.g. to control tonicity. Sodium chloride (NaCl) is typical, which may be present at between 1 and 20 mg/ml e.g. 10+2 mg/ml or 9 mg/ml. Other salts that may be present include potassium chloride, potassium dihydrogen phosphate, disodium phosphate dehydrate, magnesium chloride, calcium chloride, etc.

Pharmaceutical compositions can have an osmolality of between 200 mOsm/kg and 400 mOsm/kg, e.g. between 240-360 mOsm/kg, or between 290-310 mOsm/kg.

Pharmaceutical compositions may include compounds (with or without an insoluble metal salt) in plain water (e.g. w.f.i.) but will usually include one or more buffers. Typical buffers include: a phosphate buffer (except in the fifteenth aspect); a Tris buffer; a borate buffer; a succinate buffer; a histidine buffer (particularly with an aluminium hydroxide adjuvant); or a citrate buffer. Buffer salt s will typically be included in the 5-20 mM range. If a phosphate buffer is used then the concentration of phosphate ions should, in some embodiments, be <50 mM (see above) e.g. <10 mM.

Pharmaceutical compositions typically have a pH between 5.0 and 9.5 e.g. between 6.0 and 8.0.

Pharmaceutical compositions are preferably sterile.

Pharmaceutical compositions preferably non-pyrogenic e.g. containing <1 EU (endotoxin unit, a standard measure) per dose, and preferably <0.1 EU per dose.

Pharmaceutical compositions are preferably gluten free.

Pharmaceutical compositions are suitable for administration to animal (and, in particular, human) patients, and thus include both human and veterinary uses. They may be used in a method of raising an immune response in a patient, comprising the step of administering the composition to the patient. Compositions may be administered before a subject is exposed to a pathogen and/or after a subject is exposed to a pathogen.

Pharmaceutical compositions may be prepared in unit dose form. In some embodiments a unit dose may have a volume of between 0.1-1.0 ml e.g. about 0.5 ml.

The invention also provides a delivery device (e.g. syringe, nebuliser, sprayer, inhaler, dermal patch, etc.) containing a pharmaceutical composition of the invention e.g. containing a unit dose. This device can be used to administer the composition to a vertebrate subject.

The invention also provides a sterile container (e.g. a vial) containing a pharmaceutical composition of the invention e.g. containing a unit dose.

The invention also provides a unit dose of a pharmaceutical composition of the invention.

The invention also provides a hermetically sealed container containing a pharmaceutical composition of the invention. Suitable containers include e.g. a vial.

The invention also provides a kit comprising first and second kit components, wherein: (i) the first kit component comprises an insoluble metal salt and at least one S. pneumoniae antigen; and (ii) the second kit component comprises a TLR agonist. The second component ideally does not include an insoluble metal salt and/or does not include a S. pneumoniae antigen. The first and second components can be combined to provide a composition suitable for administration to a subject.

The invention also provides a kit comprising first and second kit components, wherein: (i) the first kit component comprises an insoluble metal salt and a TLR agonist; and (ii) the second kit component comprises at least one S. pneumoniae antigen. The second component ideally does not include an insoluble metal salt and/or a TLR agonist. In some embodiments, the second component is lyophilised. The first and second components can be combined to provide a pharmaceutical composition suitable for administration to a subject.

The invention also provides a kit comprising first and second kit components, wherein: (i) the first kit component comprises at least one S. pneumoniae antigen and a TLR agonist; and (ii) the second kit component comprises an insoluble metal salt. The second component ideally does not include a S. pneumoniae antigen and/or a TLR agonist. The first and second components can be combined to provide a pharmaceutical composition suitable for administration to a subject.

In some embodiments these kits comprise two vials. In other embodiments they comprise one ready-filled syringe and one vial, with the contents of the syringe being mixed with the contents of the vial prior to injection. A syringe/vial arrangement is useful where the vial's contents are lyophilised. Usually, though, the first and second kit components will both be in aqueous liquid form.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. by an inhaler, using a fine powder or a spray. The composition may be prepared as a suppository or pessary. The composition may be prepared for nasal, aural or ocular administration e.g. as a spray or drops. The composition may be in kit form, designed such that a combined composition is reconstituted just prior to administration to a patient. Such kits may comprise one or more antigens in liquid form and one or more lyophilised antigens. Injectables for intramuscular administration are typical.

Compositions comprise an effective amount of a TLR agonist i.e. an amount which, when administered to an individual, either in a single dose or as part of a series, is effective for enhancing the immune response to a co-administered S. pneumoniae antigen. This amount can vary depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. The amount will fall in a relatively broad range that can be determined through routine trials. An amount of between 1-1000 μg/dose can be used e.g. from 5-100 μg per dose or from 10-100 μg per dose, and ideally ≦300 μg per dose e.g. about 5 μg, 10 μg, 20 μg, 25 μg, 50 μg or 100 μg per dose. Thus the concentration of a TLR agonist in a composition of the invention may be from 2-2000 μg/ml e.g. from 10-200 μg/ml,or about 10, 20, 40, 50, 100 or 200 μg/ml, and ideally ≦600 μg/ml.

Methods of Treatment, and Administration of Immunogenic Compositions

The invention provides a method of raising an immune response in a subject, comprising the step of administering to the subject a composition of the invention.

The invention also provides a composition of the invention, for use in a method of raising an immune response in a subject.

The invention also provides the use of a TLR agonist, insoluble metal salt and S. pneumoniae antigen(s) in the manufacture of a medicament for raising an immune response in a subject.

The invention also provides the use of (i) a TLR agonist as defined herein and (ii) an insoluble metal salt and (iii) one or more S. pneumoniae antigens, in the manufacture of a medicament (e.g. a vaccine) for raising an immune response in a subject.

The invention is suitable for raising immune responses in human or non-human animal (in particular mammal) subjects. Compositions prepared according to the invention may be used to treat both children and adults.

The immune response stimulated by these methods and uses will generally include an antibody response, preferably a protective antibody response. Methods for assessing antibody responses after immunisation are well known in the art.

Treatment can be by a single dose schedule or a multiple dose schedule. Multiple doses may be used in a primary immunisation schedule and/or in a booster immunisation schedule. Administration of more than one dose (typically two doses) is particularly useful in immunologically nave patients. Multiple doses will typically be administered at least 1 week apart (e.g. about 2 weeks, about 3 weeks, about 4 weeks, about 6 weeks, about 8 weeks, about 10 weeks, about 12 weeks, etc.).

Chemical Groups

Unless specifically defined elsewhere, the chemical groups discussed herein have the following meaning when used in present specification:

The term “alkyl” includes saturated hydrocarbon residues including:
linear groups up to 10 atoms (C₁-C₁₀), or of up to 6 atoms (C₁-C₆), or of up to 4 atoms (C₁-C₄). Examples of such alkyl groups include, but are not limited, to C₁-methyl, C₂-ethyl, C₃-propyl and C₄-n-butyl.
branched groups of between 3 and 10 atoms (C₃-C₁₀), or of up to 7 atoms (C₃-C₇), or of up to 4 atoms (C₃-C₄). Examples of such alkyl groups include, but are not limited to, C₃- iso-propyl, C₄-sec-butyl, C₄-iso-butyl, C₄-tert-butyl and C₅-neo-pentyl.

The term “alkylene” refers to the divalent hydrocarbon radical derived from an alkyl group, and shall be construed in accordance with the definition above.

The term “alkenyl” includes monounsaturated hydrocarbon residues including:
linear groups of between 2 and 6 atoms (C₂-C₆). Examples of such alkenyl groups include, but are not limited to, C₂-vinyl, C₃-1-propenyl, C₃-allyl, C₄-2-butenyl
branched groups of between 3 and 8 atoms (C₃-C₈). Examples of such alkenyl groups include, but are not limited to, C₄-2-methyl-2-propenyl and C₆-2,3-dimethyl-2-butenyl.

The term alkenylene refers to the divalent hydrocarbon radical derived from an alkenyl group, and shall be construed in accordance with the definition above.

The term “alkoxy” includes O-linked hydrocarbon residues including:
linear groups of between 1 and 6 atoms (C₁-C₆), or of between 1 and 4 atoms (C₁-C₄). Examples of such alkoxy groups include, but are not limited to, C₁-methoxy, C₂-ethoxy, C₃-n-propoxy and C₄-n-butoxy.
branched groups of between 3 and 6 atoms (C₃-C₆) or of between 3 and 4 atoms (C₃-C₄). Examples of such alkoxy groups include, but are not limited to, C₃-iso-propoxy, and C₄-sec-butoxy and tert-butoxy.

Halo is selected from Cl, F, Br and I. Halo is preferably F.

The term “aryl” includes a single or fused aromatic ring system containing 6 or 10 carbon atoms;

wherein, unless otherwise stated, each occurrence of aryl may be optionally substituted with up to 5 substituents independently selected from (C₁-C₆)alkyl, (C₁-C₆)alkoxy, OH, halo, CN, COOR¹⁴, CF₃ and NR¹⁴R¹⁵; as defined above. Typically, aryl will be optionally substituted with 1, 2 or 3 substituents. Optional substituents are selected from those stated above. Examples of suitable aryl groups include phenyl and naphthyl (each optionally substituted as stated above). Arylene refers the divalent radical derived from an aryl group, and shall be construed in accordance with the definition above.

The term “heteroaryl” includes a 5, 6, 9 or 10 membered mono- or bi-cyclic aromatic ring, containing 1 or 2 N atoms and, optionally, an NR¹⁴ atom, or one NR¹⁴ atom and an S or an O atom, or one S atom, or one O atom; wherein, unless otherwise stated, said heteroaryl may be optionally substituted with 1, 2 or 3 substituents independently selected from (C₁-C₆)alkyl, (C₁-C₆)alkoxy, OH, halo, CN, COOR¹⁴, CF₃ and NR¹⁴R¹⁵; as defined below. Examples of suitable heteroaryl groups include thienyl, furanyl, pyrrolyl, pyrazolyl, imidazoyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, indolyl, benzimidazolyl, benzotriazolyl, quinolinyl and isoquinolinyl (optionally substituted as stated above). Heteroarylene refers the divalent radical derived from heteroaryl, and shall be construed in accordance with the definition above.

The term “heterocyclyl” is a C-linked or N-linked 3 to 10 membered non-aromatic, mono- or bi-cyclic ring, wherein said heterocycloalkyl ring contains, where possible, 1, 2 or 3 heteroatoms independently selected from N, NR¹⁴, S(O)_{q} and O; and said heterocycloalkyl ring optionally contains, where possible, 1 or 2 double bonds, and is optionally substituted on carbon with 1 or 2 substituents independently selected from (C₁-C₆)alkyl, (C₁-C₆)alkoxy, OH, CN, CF₃, halo, COOR¹⁴, NR¹⁴R¹⁵ and aryl.

In the above definitions R¹⁴ and R¹⁵ are independently selected from H and (C₁-C₆)alkyl.

When a structural formula is defined with a substituent attached to the core of the molecule by an unspecified, or “floating” bond, for example, as for the group P³ in the case of formula (C), this definition encompasses the cases where the unspecified substituent is attached to any of the atoms on the ring in which the floating bond is located, whilst complying with the allowable valence for that atom.

In the case of compounds of the invention which may exist in tautomeric forms (i.e. in keto or enol forms), for example the compounds of formula (C) or (H), reference to a particular compound optionally includes all such tautomeric forms.

General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encaphalopathies (TSEs), and in particular free from bovine spongiform encephalopathy (BSE). Overall, it is preferred to culture cells in the total absence of animal-derived materials.

Where a compound is administered to the body as part of a composition then that compound may alternatively be replaced by a suitable prodrug.

Phosphorous-containing groups employed with the invention may exist in a number of protonated and deprotonated forms depending on the pH of the surrounding environment, for example the pH of the solvent in which they are dissolved. Therefore, although a particular form may be illustrated it is intended, unless otherwise mentioned, for these illustrations to merely be representative and not limiting to a specific protonated or deprotonated form. For example, in the case of a phosphate group, this has been illustrated as —OP(O)(OH)₂ but the definition includes the protonated forms —[OP(O)(OH₂)(OH)]⁺ and —[OP(O)(OH₂)₂]²⁺ that may exist in acidic conditions and the deprotonated forms —[OP(O)(OH)(O)]⁻ and [OP(O)(O)₂]²⁻ that may exist in basic conditions.

Compounds disclosed herein can exist as pharmaceutically acceptable salts. Thus, the compounds may be used in the form of their pharmaceutically acceptable salts i.e. physiologically or toxicologically tolerable salt (which includes, when appropriate, pharmaceutically acceptable base addition salts and pharmaceutically acceptable acid addition salts).

### BRIEF DESCRIPTION OF THE DRAWINGS

FIG. 1 provides the repeating units of representative bacterial saccharides for use in the invention.

FIG. 2 shows chemical structures of S. pneumoniae polysaccharide serotypes 1, 5, 6B, 14, 19F and 23F.

FIG. 3 shows a schematic representation of the direct reductive amination reaction.

FIG. 4 shows a conjugation scheme of Pneumococcal polysaccharide serotype 5, 6B, 14, 23F to CRM197.

FIG. 5 shows a conjugation scheme of Pneumococcal polysaccharide serotype 1 to CRM197.

FIG. 6 shows a conjugation scheme of Pneumococcal polysaccharide serotype 19F to CRM197.

FIG. 7 compares the OPKA killing titers post 2 and post 3 for the 30001(14) S. pneumoniae strain. In each pair of columns, the left column represents “post 2” and the right represents “post 3”. Y-axis shows killing titer. X-axis (from left to right) corresponds to (A) PBS+Al—H; (B) PBS+Al—H/K2; (C) PCV13; (D) conjugate 1, 5, 6B, 14, 23F-CRM₁₉₇, 1 μg each antigen+Al—H; (E) conjugate 1, 5, 6B, 14, 23F-CRM₁₉₇, 0.1 μg each antigen+Al—H; (F) conjugate 1, 5, 6B, 14, 23F-CRM₁₉₇, 0.01 μg each antigen+Al—H; (G) conjugate 1, 5, 6B, 14, 23F-CRM₁₉₇, 1 μg each antigen+Al—H/K2; (H) conjugate 1, 5, 6B, 14, 23F-CRM₁₉₇, 0.1 μg each antigen+Al—H/K2; (I) conjugate 1, 5, 6B, 14, 23F-CRM₁₉₇, 0.01 μg each antigen+Al—H/K2.

FIG. 8 compares the serotype 14 antibody response to a mixture of 1-, 5-, 6B-, 14-, 23F-CRM197 conjugates adjuvanted with Al—H/K2 or Al—H. Y-axis shows mean fluorescence intensity and standard error of the mean. X-axis (from left to right) corresponds to (A) Al—H; (B) Al—H/K2; (C) PCV13; (D) conjugate 1, 5, 6B, 14, 23F-CRM₁₉₇, 1 μg each antigen+Al—H; (E) conjugate 1, 5, 6B, 14, 23F-CRM₁₉₇, 0.1 μg each antigen+Al—H; (F) conjugate 1, 5, 6B, 14, 23F-CRM₁₉₇, 0.01 μg each antigen+Al—H; (G) conjugate 1, 5, 6B, 14, 23F-CRM₁₉₇, 1 μg each antigen+Al—H/K2; (H) conjugate 1, 5, 6B, 14, 23F-CRM₁₉₇, 0.1 μg each antigen+Al—H/K2; (I) conjugate 1, 5, 6B, 14, 23F-CRM₁₉₇, 0.01 μg each antigen+Al—H/K2. *=significant difference.

FIG. 9 compares the serotype 1 antibody response to a mixture of 1-, 5-, 6B-, 14-, 23F-CRM₁₉₇ conjugates adjuvanted with Al—H/K2 or Al—H. Y-axis shows mean fluorescence intensity and standard error of the mean. X-axis (from left to right) corresponds to (A) Al—H; (B) Al—H/K2; (C) PCV13; (D) conjugate 1, 5, 6B, 14, 23F-CRM₁₉₇, 1μg each antigen+Al—H; (E) conjugate 1, 5, 6B, 14, 23F-CRM₁₉₇, 0.1 μg each antigen+Al—H; (F) conjugate 1, 5, 6B, 14, 23F-CRM₁₉₇, 0.01 μg each antigen+Al—H; (G) conjugate 1, 5, 6B, 14, 23F-CRM₁₉₇, 1 μg each antigen+Al—H/K2; (H) conjugate 1, 5, 6B, 14, 23F-CRM₁₉₇, 0.1 μg each antigen+Al—H/K2; (I) conjugate 1, 5, 6B, 14, 23F-CRM₁₉₇, 0.01 μg each antigen+Al—H/K2. *=significant difference.

FIG. 10 compares the serotype 5 antibody response to a mixture of 1-, 5-, 6B-, 14-, 23F-CRM₁₉₇ conjugates adjuvanted with Al—H/K2 or Al—H. Y-axis shows mean fluorescence intensity and standard error of the mean. X-axis (from left to right) corresponds to (A) Al—H; (B) Al—H/K2; (C) PCV13; (D) conjugate 1, 5, 6B, 14, 23F-CRM₁₉₇, 1 μg each antigen+Al—H; (E) conjugate 1, 5, 6B, 14, 23F-CRM₁₉₇, 0.1 μg each antigen+Al—H; (F) conjugate 1, 5, 6B, 14, 23F-CRM₁₉₇, 0.01 μg each antigen+Al—H; (G) conjugate 1, 5, 6B, 14, 23F-CRM₁₉₇, 1 μg each antigen+Al—H/K2; (H) conjugate 1, 5, 6B, 14, 23F-CRM₁₉₇, 0.1 μg each antigen+Al—H/K2; (I) conjugate 1, 5, 6B, 14, 23F-CRM₁₉₇, 0.01 μg each antigen+Al—H/K2; (J) serotype 5-CRM₁₉₇ , 1 μg+Al—H; (K) serotype 5-CRM₁₉₇0.1 μg+Al—H; (L) serotype 5-CRM₁₉₇ 1 μg+Al—H/K2;(M) serotype 5-CRM₁₉₇0.1 μg+Al—H/K2.

FIG. 11 provided a side-by-side comparison of all antibody titers obtained in the microsphere-based immunological assay (MIA) study. Y-axis shows fluorescence intensity log scale. X-axis (from left to right) corresponds to (A) Al—H; (B) Al—H/K2; (C) PCV13; (D) conjugate 1, 5, 6B, 14, 23F-CRM₁₉₇, 1 μg each antigen+Al—H; (E) conjugate 1, 5, 6B, 14, 23F-CRM₁₉₇, 0.1 μg each antigen+Al—H; (F) conjugate 1, 5, 6B, 14, 23F-CRM₁₉₇, 0.01 μg each antigen+Al—H; (G) conjugate 1, 5, 6B, 14, 23F-CRM₁₉₇, 1 μg each antigen+Al—H/K2; (H) conjugate 1, 5, 6B, 14, 23F-CRM₁₉₇, 0.1 μg each antigen+Al—H/K2; (I) conjugate 1, 5, 6B, 14, 23F-CRM₁₉₇, 0.01 μg each antigen+Al—H/K2; (J) serotype 5-CRM₁₉₇, 1 μg+Al—H; (K) serotype 5-CRM₁₉₇0.1 μg+Al—H; (L) serotype 5-CRM₁₉₇ 1 μg+Al—H/K2;(M) serotype 5-CRM₁₉₇0.1 μg+Al—H/K2.

FIG. 12 compares the OPKA killing titers post 2 for the SPPD (1) S. pneumoniae strain. Y-axis shows killing titer. X-axis (from left to right) corresponds to (A) PBS+Al—H; (B) PBS+Al—H/K2; (C) PCV13; (D) conjugate 1, 5, 6B, 14, 23F-CRM₁₉₇, 1 μg each antigen+Al—H; (E) conjugate 1, 5, 6B, 14, 23F-CRM₁₉₇, 0.1 μg each antigen+Al—H; (F) conjugate 1, 5, 6B, 14, 23F-CRM₁₉₇, 0.01 μg each antigen+Al—H; (G) conjugate 1, 5, 6B, 14, 23F-CRM₁₉₇, 1 μg each antigen+Al—H/K2; (H) conjugate 1, 5, 6B, 14, 23F-CRM₁₉₇, 0.1 μg each antigen+Al—H/K2; (I) conjugate 1, 5, 6B, 14, 23F-CRM₁₉₇, 0.01 μg each antigen+Al—H/K2.

FIG. 13 compares the OPKA killing titers post 2 and post 3 for the SPPD (1) S. pneumoniae strain. In each pair of columns, the left column represents “post 2” and the right represents “post 3”. Y-axis shows killing titer. X-axis (from left to right) corresponds to (A) PBS+Al—H; (B) PBS+Al—H/K2; (C) PCV13; (D) conjugate 1, 5, 6B, 14, 23F-CRM₁₉₇, 1 μg each antigen+Al—H; (E) conjugate 1, 5, 6B, 14, 23F-CRM₁₉₇, 0.1 μg each antigen+Al—H; (F) conjugate 1, 5, 6B, 14, 23F-CRM₁₉₇, 0.01 μg each antigen+Al—H; (G) conjugate 1, 5, 6B, 14, 23F-CRM₁₉₇, 1 μg each antigen+Al—H/K2; (H) conjugate 1, 5, 6B, 14, 23F-CRM₁₉₇, 0.1 μg each antigen+Al—H/K2; (I) conjugate 1, 5, 6B, 14, 23F-CRM₁₉₇, 0.01 μg each antigen+Al—H/K2.

FIG. 14 compares the OPKA killing titers post 2 for the STREP(5) S. pneumoniae strain. Y-axis shows killing titer. X-axis (from left to right) corresponds to (A) PBS+Al—H; (B) PBS+Al—H/K2; (C) PCV13; (D) conjugate 1, 5, 6B, 14, 23F-CRM₁₉₇, 1 μg each antigen+Al—H; (E) conjugate 1, 5, 6B, 14, 23F-CRM₁₉₇, 0.1 μg each antigen+Al—H; (F) conjugate 1, 5, 6B, 14, 23F-CRM₁₉₇, 0.01 μg each antigen+Al—H; (G) conjugate 1, 5, 6B, 14, 23F-CRM₁₉₇, 1 μg each antigen+Al—H/K2; (H) conjugate 1, 5, 6B, 14, 23F-CRM₁₉₇, 0.1 μg each antigen+Al—H/K2; (I) conjugate 1, 5, 6B, 14, 23F-CRM₁₉₇, 0.01 μg each antigen+Al—H/K2; (J) serotype 5-CRM₁₉₇ 1 μg+Al—H; (K) serotype 5-CRM_{197 0.1} μg+Al—H; (L) serotype 5-CRM₁₉₇ 1 μg+Al—H/K2;(M) serotype 5-CRM₁₉₇0.1 μg+Al—H/K2.

FIG. 15 compares the OPKA killing titers post 2 and post 3 for the STREP(5) S. pneumoniae strain. In each pair of columns, the left column represents “post 2” and the right represents “post 3”. Y-axis shows killing titer. X-axis (from left to right) corresponds to (A) PBS+Al—H; (B) PBS+Al—H/K2; (C) PCV13; (D) conjugate 1, 5, 6B, 14, 23F-CRM₁₉₇, lgg each antigen+Al—H; (E) conjugate 1, 5, 6B, 14, 23F-CRM₁₉₇, 0.1 μg each antigen+Al—H; (F) conjugate 1, 5, 6B, 14, 23F-CRM₁₉₇, 0.01 μg each antigen+Al—H; (G) conjugate 1, 5, 6B, 14, 23F-CRM₁₉₇, lgg each antigen+Al—H/K2; (H) conjugate 1, 5, 6B, 14, 23F-CRM₁₉₇, 0.1 μg each antigen+Al—H/K2; (I) conjugate 1, 5, 6B, 14, 23F-CRM₁₉₇, 0.01 μg each antigen+Al—H/K2; (J) serotype 5-CRM₁₉₇ 1μg+Al—H; (K) serotype 5-CRM₁₉₇ 0.1 μg+Al—H; (L) serotype 5-CRM₁₉₇ 1μg+Al—H/K2;(M) serotype 5-CRM₁₉₇ 0.1 μg+Al—H/K2.

FIG. 16 compares the OPKA killing titers post 2 for the 30001(14) S. pneumoniae strain. Y-axis shows killing titer. X-axis (from left to right) corresponds to (A) PBS+Al—H; (B) PBS+Al—H/K2; (C) PCV13; (D) conjugate 1, 5, 6B, 14, 23F-CRM₁₉₇, 1μg each antigen+Al—H; (E) conjugate 1, 5, 6B, 14, 23F-CRM₁₉₇, 0.1 μg each antigen+Al—H; (F) conjugate 1, 5, 6B, 14, 23F-CRM₁₉₇, 0.01 μg each antigen+Al—H; (G) conjugate 1, 5, 6B, 14, 23F-CRM₁₉₇, 1μg each antigen+Al—H/K2; (H) conjugate 1, 5, 6B, 14, 23F-CRM₁₉₇, 0.1 μg each antigen+Al—H/K2; (I) conjugate 1, 5, 6B, 14, 23F-CRM₁₉₇, 0.01 μg each antigen+Al—H/K22.

FIG. 17 compares the antibody response of RrgB321 adjuvanted with Al—H, Al—H/K2 or K2. Y-axis shows median mean fluorescence intensity (MFI). X-axis (from left to right) corresponds to (A) PBS; (B) PBS+RrgB321; (C) Al—H+RrgB321; (D) Al—H/K2+RrgB321; (E) K2+RrgB321.

FIG. 18 compares the OPKA killing titers for the TIGR4 S. pneumoniae strain. Y-axis shows killing titer. X-axis shows sample sera dilution. Bars correspond to (A) PBS; (B) PBS+RrgB321; (C) Al—H+RrgB321; (D) Al—H/K2+RrgB321; (E) RrgB321+K2 (100 μg/ml).

FIG. 19 compares the OPKA killing titers for the 6B SPEC S. pneumoniae strain. Y-axis shows killing titer. X-axis shows sample sera dilution. Curves correspond to (A) PBS; (B) PBS+RrgB321; (C) Al—H+RrgB321; (D) Al—H/K2+RrgB321; (E) RrgB321+K2 (100 μg/ml).

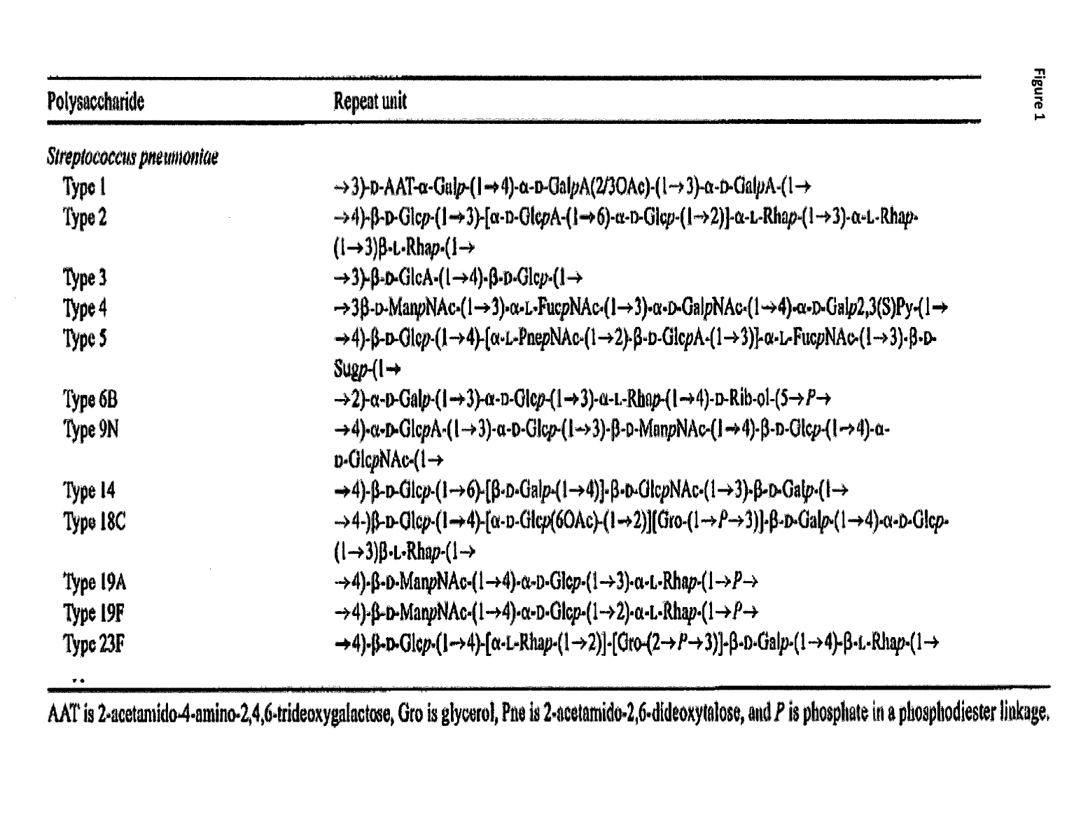

### MODES FOR CARRYING OUT THE INVENTION

### Saccharide Antigen

Preparation of S. pneumoniae Polysaccharide Conjugates

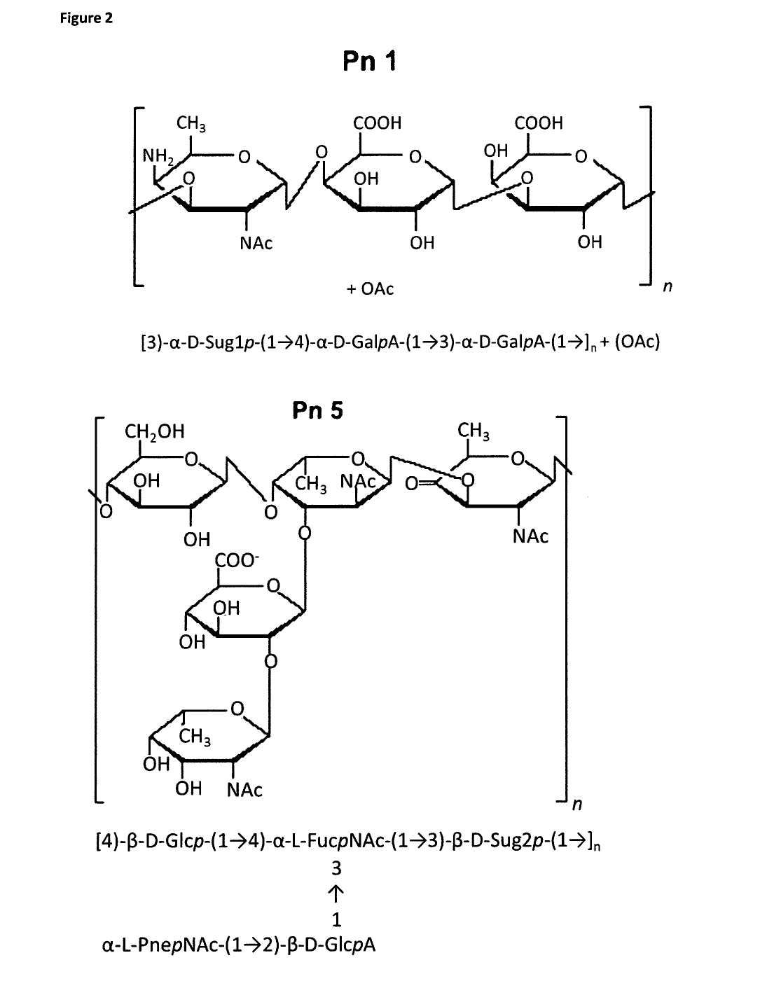

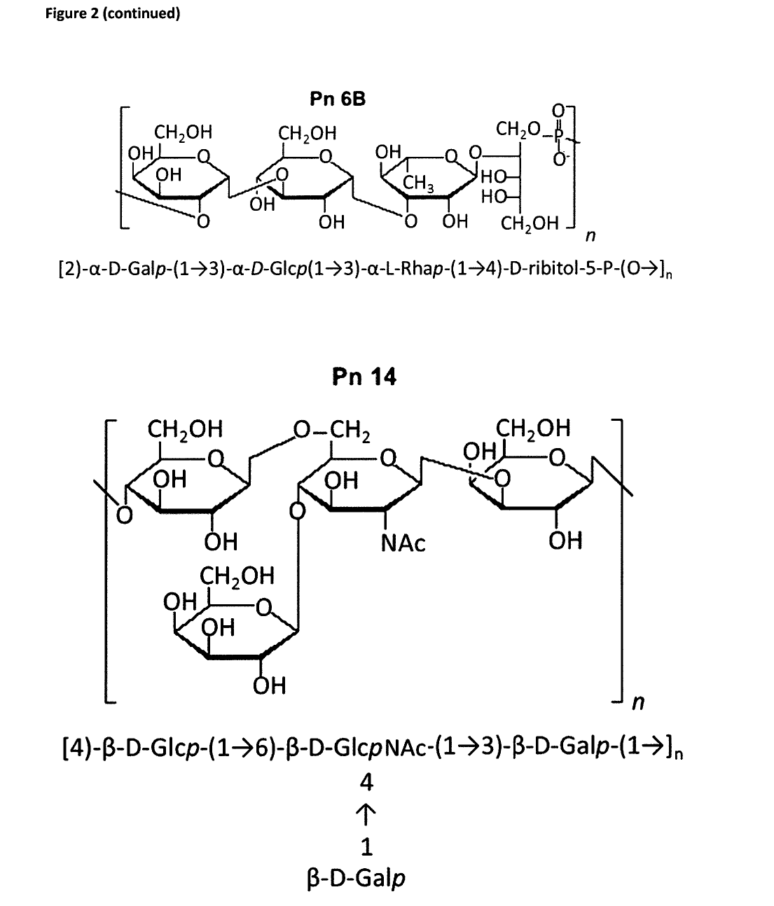

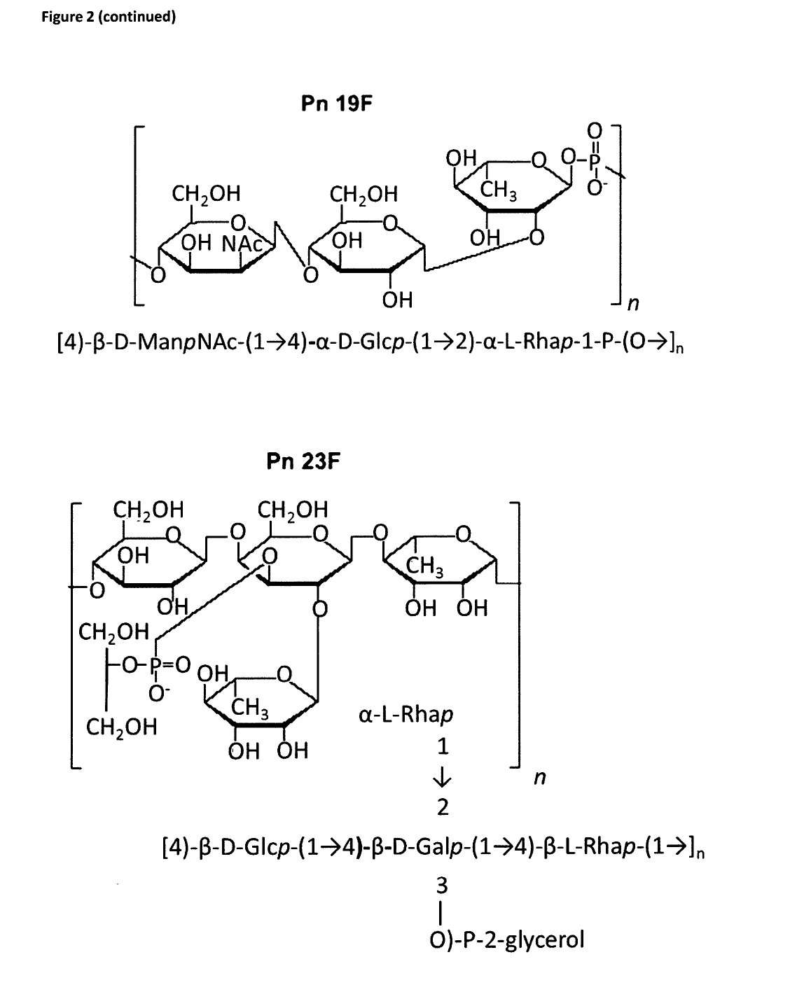

S. pneumoniae polysaccharide serotypes 1, 5, 6B, 14, 19F and 23F were purchased from ATCC or provided by internal sources, and the structural integrity of the polysaccharides was confirmed by NMR analysis. Chemical structures of S. pneumoniae polysaccharide serotypes 1, 5, 6B, 14, 19F and 23F are provided in FIG. 2.

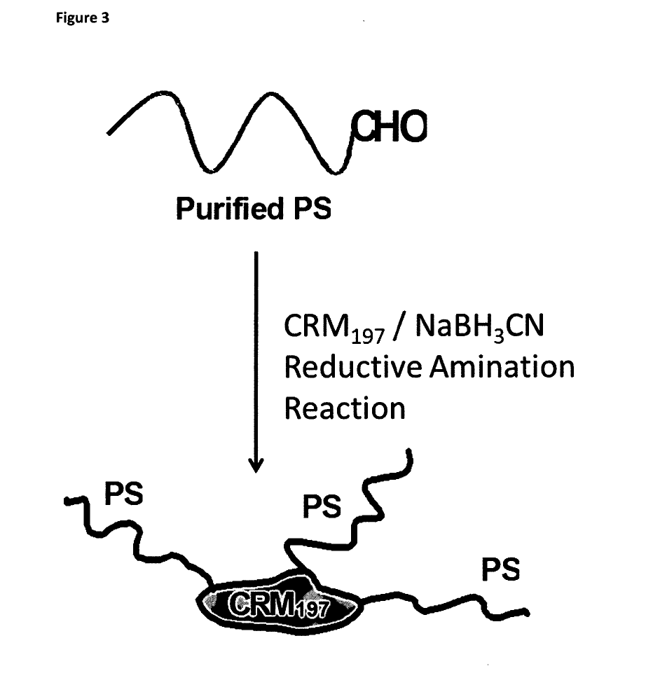

Conjugates were obtained by covalent conjugation of serotype 1, 5, 6B, 14, 19F and 23F capsular polysaccharides to the CRM₁₉₇ carrier protein via direct reductive amination reaction (FIG. 3).

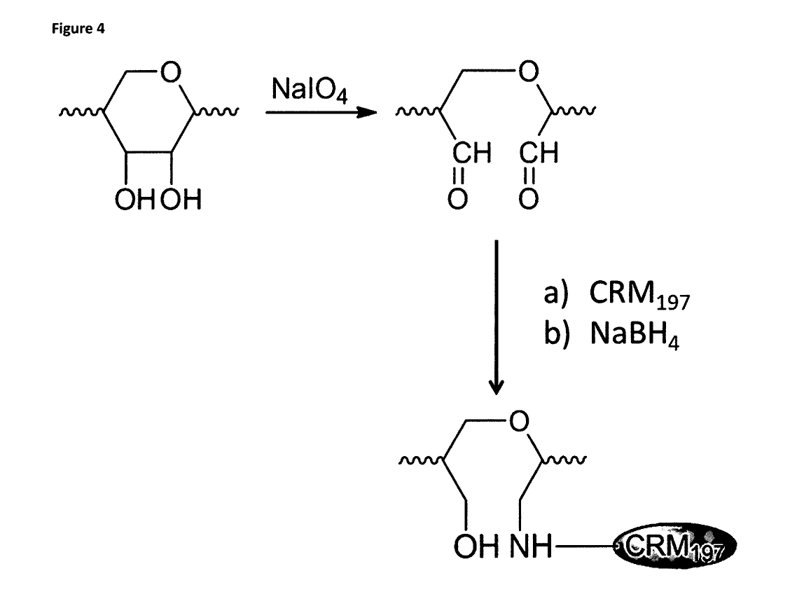

Due to differences between the chemical structures of saccharides from the different serotypes, different chemistries were required to conjugate the polysaccharides to the carrier protein. The cis-diols present on serotype 5 (α-L-PneNAc-1,2), 6B (α-D-Gal-1,3 and D-ribitol-5-P), 14 (β-D-Gal-1,4) and 23F (α-L-Rha-1,2 and β-L-Rha-1,4) were oxidized to introduce aldehyde groups, which were coupled to CRM₁₉₇ via a direct reductive amination reaction. The reductive amination reaction involves the amino groups on the side chain of a lysine in the carrier protein and the aldehyde groups introduced in the polysaccharide (see FIG. 4).

For serotype 1, the carboxylic groups present on the two a-D-GalA of the repeating unit were derivatized with a linker (aminopentandiol (APD)) in the presence of EDAC (N-Ethyl-N′-(3-dimethylaminopropyl)carbodiimide hydrochloride) as condensing reagent. The diol introduced by the linker derivatization was then oxidized to an aldehyde group and coupled to CRM₁₉₇ via direct reductive amination reaction (see FIG. 5).

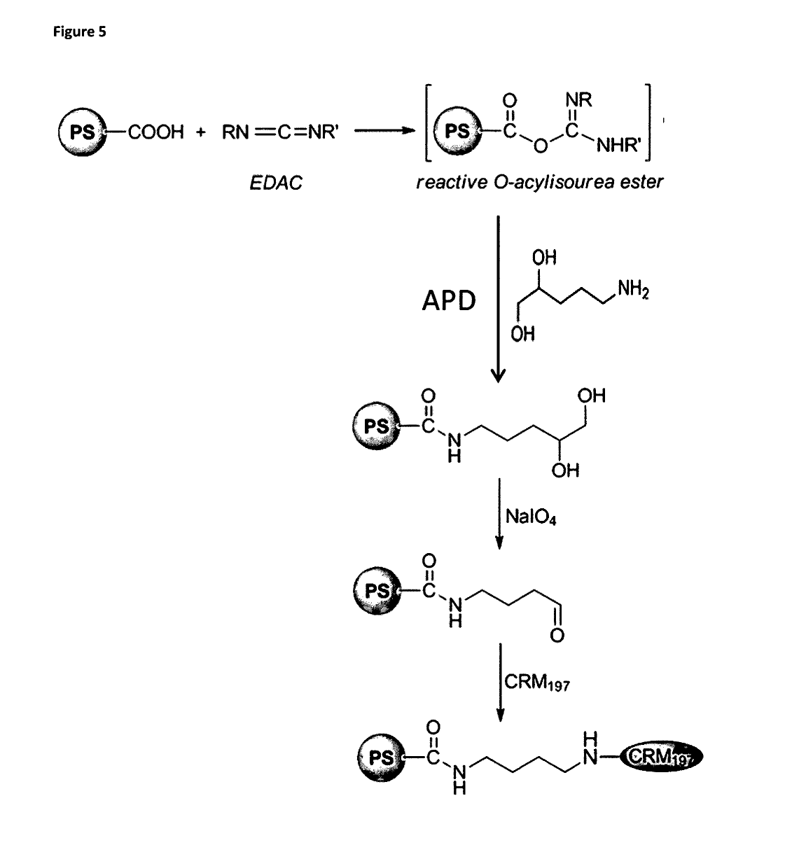

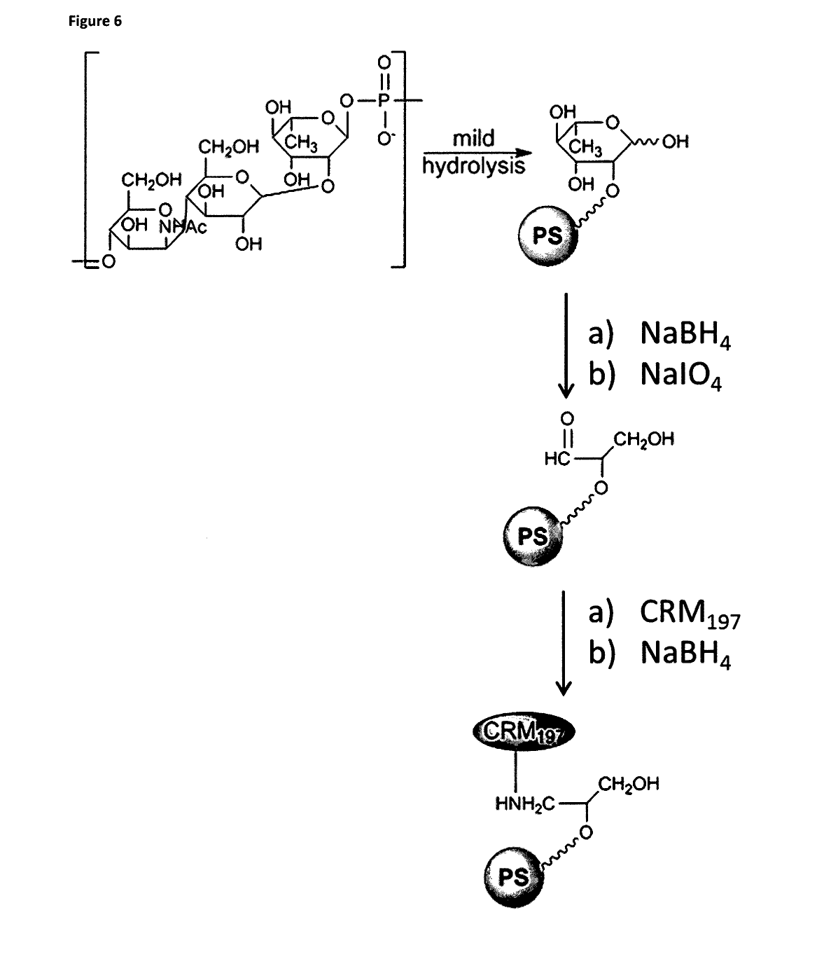

For serotype 19F, a structural modification was applied to the reducing-end. First the polysaccharide was hydrolyzed to generate a reducing-end that was reduced to introduce a cis-diol. Then, the oxidation of the diol allowed the introduction of the aldehyde groups for the conjugation to the carrier protein, CRM₁₉₇, via direct reductive amination reaction (see FIG. 6).

Oxidation of Polysaccharides

Serotype 5

The serotype 5 polysaccharide was sized by size-exclusion chromatography (SEC) with using a Sephacryl S1000 resin. The chromatographic step was performed on AktaTM system by measuring UV absorption at 215 nm.

The polysaccharide was loaded onto the Sephacryl S1000 column equilibrated in 10 mM NaPi/150M NaCl pH 7.2 buffer. The column was performed at 0.5 ml/min flow rate. The polysaccharide was collected in fractions in the first eluted single large peak. The fractions were pooled cutting out the beginning and the tail of the peak (data not shown). The pooled fractions were concentrated 3-4 fold to perform the oxidation reaction, and the target oxidation was 20% mol of polysaccharide repeating unit (MW repeating unit 896).

Sodium (meta)periodate, NaI0₄, was added to the mixture and gently mixed at room temperature overnight and in the dark. The crude reaction was then dialyzed in a membrane with a 6-8 kDa cut-off. The crude reaction was loaded into the membrane and dialyzed against distilled water (2 L of distilled water for 10 ml of crude reaction), at+4/8° C. The distilled water was changed 2-3 times. Finally, after around 16 hours, the solution was recovered. For maximum product recovery, the membrane was washed twice with distilled water, adding these washings to the solution.

The oxidized polysaccharide was characterized by reducing groups colorimetric assay (to quantify the aldehyde group introduced), and found to be 4.5% oxidised.

Serotype 6B

The reaction was performed at a polysaccharide concentration of 2 mg/ml in 500 mM NaCl buffer. The target oxidation was 40% mol of polysaccharide repeating unit (MW repeating unit 683). NaI0₄, was added and the mixture was gently mixed in the dark, overnight at room temperature. The crude reaction was then dialyzed in a membrane with a 6-8 kDa cut-off. The crude reaction was then loaded into the membrane and dialyzed against distilled water, and the product recovered, as above. The oxidized polysaccharide was characterized by reducing colorimetric assay, and found to be 23% oxidised.

Serotype 14

The serotype 14 polysaccharide was sized by SEC with Sephacryl S500 resin. The chromatographic step was performed as above.

The polysaccharide was loaded onto the Sephacryl S500 column and equilibrated as above. The column was performed at 0.3 ml/min flow rate. The polysaccharide was collected in fractions in the first eluted single large peak, and pooled as above. The pooled fractions were concentrated 2 fold to perform the oxidation reaction. The NaI0₄ step was performed as above, to obtain 0.1M as final concentration, and then the crude reaction was dialyzed in membrane with a lkDa cut-off. The crude reaction was then loaded into the membrane and dialyzed against distilled water, and the product recovered, as above. The oxidized polysaccharide was characterized by reducing colorimetric assay, and found to be 5.5% oxidised.

Serotype 23F

The reaction was performed at a polysaccharide concentration of 2 mg/ml in 500 mM NaCl buffer. The target oxidation was 40% mol of polysaccharide repeating unit (MW repeating unit 769). NaIO₄ was added and the mixture was gently mixed, in the dark, overnight at room temperature. The crude reaction was then dialyzed in membrane with a 6-8 kDa cut-off. The crude reaction was then loaded into the membrane and dialyzed against distilled water, and the product recovered, as above. The oxidized polysaccharide was characterized by reducing colorimetric assay, and found to be 21% oxidised.

Conjugation Reactions

Serotype 5

The conjugation reaction was performed with a polysaccharide serotype 5 concentration of 10 mg/mL in Na₂B₄O₇ 100 mM/NaCl 100 mM pH8.4 buffer. The PS:Protein ratio was 1:1 (weight/weight) and PS:NaBCNH₃ ratio was 1:1 (weight/weight). The carrier protein, CRM₁₉₇, was added to a solution of the serotype 5 polysaccharide in Na₂B₄O₇ 100 mM/NCl 100 mM pH8.4 buffer, and then NaBH₃CN was added. The solution was kept at 37° C. for 2 days, and then the reaction was quenched by adding NaBH₄ for 1 hour at room temperature (PS:NaBH₄ ratio was 4:1, weight/weight). The crude reaction was then purified by SEC, allowing separating the peak of unreacted protein and polysaccharide from the peak containing the conjugate.

The chromatographic step was performed on Akta™ system, and the conjugate was detected by measuring UV absorption at 215 nm, 254 nm and 280 nm. The crude conjugation reaction was loaded onto the Sephacryl S1000 column equilibrated in 10 mM NaPi/150M NaCl pH 7.2 buffer. The column was performed at 0.5 ml/min flow rate. The serotype 5-CRM₁₉₇ conjugate was collected in fractions in the first eluted peak, which appeared mainly as a single large peak, and the fractions were pooled cutting out the peak tail (data not shown).

After the purification, the conjugate solution was stored at −20° C. SDS-Page was performed to verify the covalent formation of conjugate with NuPAGE 3-8% TrisAcetate Gel, and a Western-Blot analysis was performed to verify the identity of the conjugate. The Western-blot was performed using Anti-CRM mouse serum as primary antibody (1:500) and Anti-Mouse IgG Alkaline Phosphatase serum as secondary antibody (1:5000) with Western Breeze-Chromogenic Western Blot Immunodetection Kit (data not shown).

The determination of total saccharide in the conjugate was performed by HPAEC-PAD analysis (as described in reference 233) and the determination of protein by MicroBCA assay. Table 1 shows total saccharide and protein results obtained for the serotype 5-CRM₁₉₇ conjugate.

TABLE 1
Total saccharide and protein content in serotype 5-CRM₁₉₇ conjugate

| | Saccharide | Protein | Sacc/Prot |
|---|---|---|---|
| | μg/ml | μg/ml | (w/w) |
| Pn type 5-CRM₁₉₇ | 56.00 | 218.12 | 0.26 |

Serotype 6B

The conjugation reaction was performed with a polysaccharide serotype 6B concentration of 5 mg/mL in NaPi 140 mM/NaCl 700 mM pH7.0 buffer. The PS:Protein ratio was 1:1 (weight/weight) and PS:NaBCNH₃ ratio was 1:1 (weight/weight). The carrier protein, CRM₁₉₇, was added to a solution of the serotype 6B polysaccharide in NaPi 140 mM/NaCl 700 mM pH7.0 buffer, and then NaBH₃CN was added. The solution was kept at 37° C. for 2 days, and then the reaction was quenched by adding NaBH₄ for 1 hour at room temperature (PS:NaBH₄ ratio was 6:1, weight/weight). The conjugate was purified by ammonium sulfate precipitation. This purification method allows the removal of the saccharide excess, since the saccharide does not precipitate with the conjugate and remains in solution. Ammonium sulfate (500 mg/mL) was slowly added to the crude conjugate reaction, and the mixture was kept in ice for 10-15 minutes to allow the conjugate to precipitate. The mixture was then centrifuged and the supernatant removed. The pellet, containing the conjugate, was washed 3 times with a saturated ammonium sulfate solution and then dissolved in NaPi 10 mM pH7.2 and stored at -20° C. SDS-Page was performed in order to verify the covalent formation of conjugate with NuPAGE 3-8% TrisAcetate Gel (data not shown).

The determination of total saccharide in the conjugate was performed by HPAEC-PAD analysis (using different hydrolysis conditions to those reported by reference 233: TFA 4M at 100° C. for 3 hours) and the determination of protein by MicroBCA assay. Table 2 shows total saccharide and protein results obtained for the serotype 6B-CRM₁₉₇ conjugate.

TABLE 2
Total saccharide and protein content in serotype 6B-CRM₁₉₇ conjugate

| | Saccharide | Protein | Sacc/Prot |
|---|---|---|---|
| | μg/ml | μg/ml | (w/w) |
| Pn type 6B-CRM₁₉₇ | 545.17 | 1210.00 | 0.45 |

Serotype 14

The conjugation reaction was performed at polysaccharide type 14 concentration of 8-9 mg/mL in NaPi 200 mM/NaCl 1 M pH7.2 buffer. The PS:Protein ratio was 1:1 (weight/weight) and PS:NaBCNH₃ ratio was 1:1 (weight/weight). The carrier protein, CRM₁₉₇, was added to a solution of the polysaccharide type 14 in NaPi 200 mM/NaCl 1 M pH7.2 buffer, and then NaBH₃CN was added. The solution was kept at 37° C. for 2, and then the reaction was quenched by adding NaBH₄ for 1 hour at room temperature (PS:NaBH₄ ratio was 4:1, weight/weight). The crude reaction was purified by SEC. The chromatographic step was performed on AktaTM system, and the conjugate was detected by measuring UV absorption at 215 nm, 254 nm and 280 nm. The crude conjugation reaction was loaded onto the Sephacryl 5500 column equilibrated in 10 mM NaPi/150M NaCl pH 7.2 buffer. The column was performed at 0.3 ml/min flow rate. The serotype 14-CRM₁₉₇ conjugate was collected in fractions in the first eluted peak and appeared mainly as a single large peak. The fractions were pooled cutting out the peak tail (data not shown).

After the purification, the conjugate solution was stored at −20° C. SDS-Page was performed in order to verify the covalent formation of conjugate with NuPAGE 3-8% TrisAcetate Gel and a Western-Blot was performed to verify the identity of the conjugate. The Western-blot was performed using Anti-CRM (1:500) and Anti-Pn 14 (1:1000) mouse sera as primary antibody and Anti-Mouse IgG Alkaline Phosphatase serum as secondary antibody (1:500) with Western Breeze-Chromogenic Western Blot Immunodetection Kit (data not shown).

The determination of total saccharide in the conjugate was performed by HPAEC-PAD analysis (using different hydrolysis conditions to those reported by reference 233: TFA 4M at 100° C. for 3 hours) and the determination of protein by MicroBCA assay. Table 3 shows total saccharide and protein results obtained for the serotype 14-CRM₁₉₇ conjugate.

TABLE 3
Total saccharide and protein content in serotype 14-CRM₁₉₇ conjugate

| | Saccharide | Protein | Sacc/Prot |
|---|---|---|---|
| | μg/ml | μg/ml | (w/w) |
| Pn type 14-CRM₁₉₇ | 62.16 | 145.22 | 0.43 |

Serotype 23F

The conjugation reaction was performed at polysaccharide concentration of 5 mg/mL in Na₂B₄O₇ 100 mM/NaCl 100 mM pH8.4 buffer. The PS:Protein ratio was 1:1 (weight/weight) and PS:NaBCNH₃ ratio was 1:1 (weight/weight). The carrier protein, CRM₁₉₇, was added to a solution of the polysaccharide in Na₂B₄O₇ 100 mM/NaCl 100 mM pH8.4 buffer, and then NaBH₃CN was added. The solution was kept at 37° C. for 4 days, and was then quenched by adding NaBH₄ for 1 hour at room temperature (PS:NaBH₄ ratio was 6:1, weight/weight). The conjugate was purified by ammonium sulfate precipitation. Ammonium sulfate (500 mg/mL) was slowly added to the crude conjugate reaction, and the mixture was kept in ice for 10-15 minutes to allow the conjugate precipitation, and then centrifuged and the supernatant removed. The pellet, containing the conjugate, was washed 3 times with a saturated ammonium sulfate solution and, and finally the pellet was dissolved in NaPi 10 mM pH7.2 and stored at −20° C. SDS-Page gel was performed in order to verify the covalent formation of conjugate with NuPAGE 3-8% TrisAcetate Gel (data not shown).

The determination of total saccharide in the conjugate was performed by HPAEC-PAD analysis (as described in reference 233) and the determination of protein by MicroBCA assay. Table 4 shows total saccharide and protein results obtained for the serotype 23F-CRM₁₉₇ conjugate.

TABLE 4
Total saccharide and protein content in serotype 23F-CRM₁₉₇ conjugate

| | Saccharide | Protein | Sacc/Prot |
|---|---|---|---|
| | μg/ml | μg/ml | (w/w) |
| Pn type 23F-CRM₁₉₇ | 423.74 | 2920 | 0.15 |

Serotype 1

For serotype 1, the polysaccharide was derivatised. The polysaccharide type 1 was sized by SEC with Sephacryl 51000 resin. The chromatographic step was performed on Akta™ system, and the polysaccharide was detected by measuring UV absorption at 215 nm. The polysaccharide was processed and loaded onto the Sephacryl S1000 column equilibrated in 10 mM NaPi/150M NaCl pH 7.2 buffer. The column was performed at 0.5 ml/min flow rate. The polysaccharide was collected in fractions in the first eluted single large peak, and the fractions were pooled cutting out the beginning and the tail of the peak (data not shown).

The pooled fractions were concentrated 4-5 fold and then dialyzed in membrane with a 1 kDa cut-off.

The sized polysaccharide was loaded onto the membrane and dialyzed against distilled water (2 L of distilled water for 10 ml of crude reaction), the distilled water was changed 2-3 times; the dialysis process was carried out at +4/8° C. The product was recovered as above.

The dialyzed sized polysaccharide was dried to perform the derivatization reaction with AminoPentanDiol (APD). The carboxylic groups of the polysaccharide were derivatized with APD in the presence of EDAC as condensing agent. The reaction was carried out at pH 5 where the water soluble carbodiimides have better performance. The reaction was performed at polysaccharide concentration of 6-7 mg/ml in 10 mM NaPi/200 mM NaCl buffer pH 5. EDAC, 10 equivalent relative to the moles of polysaccharide repeating unit (MW repeating unit 538), was added and the mixture was gently mixed to allow the complete solubilisation, which occurs almost immediately. Then, APD, 14 equivalent relative to the moles of polysaccharide repeating unit, was added to the solution. The reaction was gently stirred for 4 hour at 37° C., and then the crude reaction was dialyzed in a membrane with a 6-8 kDa cut-off. The crude reaction was loaded in the membrane and dialyzed and the product recovered, as above.

The APD linker introduced on the polysaccharide was oxidized to obtain an aldehyde group from the diol group. The polysaccharide oxidation was performed with a target oxidation of 40% mol of polysaccharide repeating unit (MW repeating unit 538). NaIO₄ was added and the mixture was gently mixed, at the dark, for 5 hours at room temperature.

The crude oxidized polysaccharide was characterized by formaldehyde colorimetric assay (to quantify the APD introduction) which revealed a 40% of derivatization degree. The crude reaction was purified by PD10 Desalting column, pre-packed, which contains Sephadex G-25 Medium. A PD-10 Desalting column was equilibrated with approximately 25 ml distilled water. Then the crude reaction was then loaded on the column (in a volume of 2.5 ml) and the sample to enter the packed bed completely. The flow-through was recovered and then the column was washed 7 times with 3.5 ml, and each eluate was collected. The flow-through and the eluate have been analyzed by spectrophotometry at 214 nm. The first three eluates were merged and dried. The oxidized polysaccharide was characterized by reducing colorimetric assay (to quantify the aldehyde group introduced), which revealed 6% of APD oxidation.

The conjugation reaction was performed at a polysaccharide concentration of 2.5-3.0 mg/mL in Na₂B₄O₇ 100 mM/NaCl 300 mM pH8.4 buffer. The PS:Protein ratio was 1:2.5 (weight/weight) and PS:NaBCNH₃ ratio was 1:1 (weight/weight). The carrier protein, CRM₁₉₇, was added to a solution of the polysaccharide type 1 in Na₂B₄O₇ 100 mM/NaCl 300 mM pH8.4 buffer, and then NaBH₃CN was added. The solution was kept at 37° C. for 2 days, and then the reaction was quenched by adding NaBH₄ for 1 hour at room temperature (PS:NaBH₄ ratio was 8:1, weight/weight). The crude reaction was purified by SEC. The chromatographic step was performed on AktaTM system, the conjugate was detected by measuring UV absorption at 215 nm, 254 nm and 280 nm. The crude conjugation reaction was loaded onto the Sephacryl S1000 column equilibrated in 10 mM NaPi/150M NaCl pH 7.2 buffer, and performed at 0.5 ml/min flow rate.

The serotype 1-CRM₁₉₇ conjugate was collected in fractions in the first eluted peak and appeared mainly as a single large peak. The fractions were pooled cutting out the peak tail (data not shown).

After the purification, the conjugate solution was stored at -20° C. SDS-Page was performed in order to verify the covalent formation of conjugate with NuPAGE 3-8% TrisAcetate Gel and a Western-Blot analysis to verify the identity of the conjugate. The Western-blot was performed using anti-CRM mouse serum as primary antibody (1:500) and Anti-Mouse IgG Alkaline Phosphatase serum as secondary antibody (1:5000) with Western Breeze-Chromogenic Western Blot Immunodetection Kit (data not shown).

The determination of protein in the conjugate was performed by MicroBCA assay. Instead for the saccharide concentration a theoretical value was calculated, assuming a saccharide/protein ratio of 0.25 (weight/weight), due to the technical issue with HPAEC-PAD analysis. Table 5 shows total saccharide and protein results obtained for serotype 1-CRM₁₉₇ conjugate.

TABLE 5
Total saccharide and protein content in serotype 5-CRM₁₉₇ conjugate

| | Saccharide | Protein | Sacc/Prot |
|---|---|---|---|
| | μg/ml | μg/ml | (w/w) |
| Pn type 1-CRM₁₉₇ | 100.00* | 399.27 | 0.25 |

*Theoretical value

Serotype 19F

For serotype 19F, the polysaccharide was derivatised. The reducing-end unit of the polysaccharide type 19F was modified in order to generate aldehyde groups useful for the conjugation with the protein. The polysaccharide was hydrolyzed in 5 mM AcOH at concentration of 10 mg/ml for 2 hours at 120° C. After 2 hours, the crude reaction was neutralized at pH 6.5-7.0. The structural identity of hydrolyzed polysaccharide was confirmed by ¹H and ³¹P NMR spectroscopy (data not shown). The NMR samples were prepared by dissolving the dry hydrolyzed polysaccharide in 750 ᵢal of deuterium oxide. Aliquot (750 μl) of the sample was transferred to 5-mm NMR tubes. For data acquisition and processing, all NMR experiments (¹H and ³¹P) have been recorded at 25° C. on Bruker 400 MHz spectrometers, and using 5-mm broadband probes. For data acquisition and processing TOPSPIN 2.1 software package was used. 1-D proton NMR spectra have been collected using a standard one-pulse experiment. Chemical shifts were referenced to HDO at 4.79 ppm (¹H).

The hydrolyzed polysaccharide was lyophilized to perform the reduction reaction. The reaction was performed at polysaccharide concentration of 5 mg/ml in 20 mM NaPi pH8.2 buffer and 40 mM NaBH₄ for 2 hours at room temperature. Then, the crude reaction was dialyzed in a membrane with a 1 kDa cut-off. The crude reaction was loaded in the membrane and dialyzed as above, and the product recovered as above. The reduced polysaccharide was characterized by ¹H and ³¹P NMR analysis to verify and confirm the structural identity after the reduction step (data not shown).

The reduced

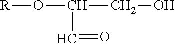

polysaccharide was lyophilized to perform the oxidation reaction. The reaction was carried out at polysaccharide concentration of 100 mg/ml in 10 mM NaPi pH 7.2 buffer. NaIO₄, 10 equivalent relative to mol of polysaccharide (MW of polysaccharide=MW repeating unit, 559, x DP) was added to obtain a final concentration of 50 mM NaIO₄, and the mixture was gently mixed, in the dark, for 4 hours at room temperature. The crude reaction was then purified by PD10 Desalting column equilibrated with approximately 25 ml distilled water. Then, the crude reaction was loaded onto the column (in a volume of 2.5 ml) and allowed the sample to enter the packed bed completely. The flow-through was recovered and then the column was washed with 3.5 ml water for 5 times, each eluted was collected. The first eluted was collected and dried. The oxidized polysaccharide was characterized by ¹H NMR analysis (data not shown). After oxidation of polysaccharide with NaI0₄ new peaks appear in the anomeric region, which can be assigned to the protons of the adduct at the reducing end terminus and the Glc residue linked in α and β conformation.

In addition, a lower intensity signal appears in the CH₃ region due to the smaller chain length obtain after the oxidation.

The conjugation reaction was performed at polysaccharide concentration of 5 mg/mL in NaPi 150 mM/NaCl 800 mM pH7.0 buffer. The PS:Protein ratio was 4:1 (weight/weight) and PS:NaBCNH₃ ratio was 2:1 (weight/weight). The carrier protein, CRM₁₉₇, was added to a solution of the polysaccharide in NaPi 150 mM/NaCl 800 mM pH7.0 buffer, and then NaBH₃CN was added. The solution was kept at 37° C. for 2 days.

The conjugate was purified by ammonium sulfate precipitation, as above. The pellet, containing the conjugate, was washed 3 times with a saturated ammonium sulfate solution and, at the end, the pellet was dissolved in Tris 10 mM pH7.2 and stored at −20° C. SDS-Page gel was performed in order to verify the covalent formation of conjugate with NuPAGE 3-8% TrisAcetate Gel (data not shown).

The determination of total saccharide in the conjugate was performed by HPAEC-PAD analysis (as described in reference 233) and the determination of protein by MicroBCA assay. Table 6 shows total saccharide and protein results obtained for serotype 19F-CRM₁₉₇ conjugate.

TABLE 6
Total saccharide and protein content in serotype 19F-CRM₁₉₇ conjugate

| | Saccharide | Protein | Sacc/Prot |
|---|---|---|---|
| | μg/ml | μg/ml | (w/w) |
| Pn type 19F-CRM₁₉₇ | 300.59 | 2157.00 | 0.14 |

Vaccine Preparation and Administration

References 214 and 234 disclose TLR7 agonists having formula (K) as discussed above. One of these compounds, 3-(5-amino-2-(2-methyl-4-(2-(2-(2-phosphonoethoxy)ethoxy)ethoxy)phenethyl)benzo[f]-[1,7]naphthyridin-8-yl)propanoic acid is referred to hereafter as compound “K2”:

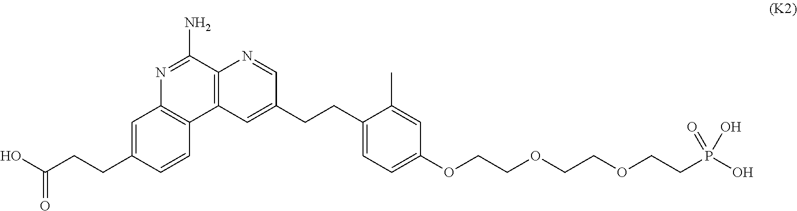 [SMILES: Cc1cc(OCCOCCOCCP(=O)(O)O)ccc1CCc1cnc2c(N)nc3cc(CCC(=O)O)ccc3c2c1]

Compound K2 is added to water at 4 mg/ml, then 1M NaOH is added to ensure full solubilisation, with stirring for 15 minutes at room temperature. This material is added to a suspension of aluminium hydroxide adjuvant (Al—H; 2 mg/ml) to give the desired final concentration. This mixture is shaken for 2 hours at ambient temperature to ensure full adsorption, and then histidine buffer components are added (10 mM histidine buffer, pH 6.5).

The compound can also be used as an arginine salt monohydrate (obtained by mixing 98 mg of the compound with 1.7 ml of 0.1M arginine in 80/20 methanol/water to give a 57 mg/mL solution, followed by addition of 7 ml ethanol to precipirate the salt) in which case it is seen that the NaOH is not required for solubilisation prior to mixing with the Al—H. 100 μg K2 was administered per dose, in a 100 μl dosage volume); the Al—H concentration is always 2 mg/ml. At all strengths >95% of compound K2 is adsorbed to the Al—H. The adsorbed adjuvant is referred to hereafter as “Al—H/K2”.

The five polysaccharide CRM conjugates (serotypes 1, 5, 6B, 14 and 23F) are mixed sequentially with Al—H/K2 to give a final concentration of 1, 0.1, or 0.01 μg per dose for each glycoconjugate. The order in which the glycoconjugates is added has little effect.

Immunisation Schemes

8 Balb/c mice were used per immunisation group. Mice received intramuscular immunisation of 1, 0.1, or 0.01 μg of polysaccharide, adjuvanted with Al—H or Al—H/K2. Adjuvant controls were included. Volume per administration was 100 μl (50 μl per leg). Positive control was the aluminium phosphate-adjuvanted 13-valent conjugate vaccine (PCV13, Prevnar), which contains serotypes 1, 3, 4, 5, 6A, 6B, 7F, 9V, 14, 18C, 19A, 19F, and 23F each conjugated to CRM₁₉₇. Each 100 μl dose of PCV13 comprises 25 μg aluminium phosphate adjuvant, ˜0.44 μg of each saccharide from serotypes 1, 3, 4, 5, 6A, 7F, 9V, 14, 18C, 19A, 19F, and 23F, and 0.88 pl of serotype 6B saccharide. Mice were immunised on days 0 (“post 1”), 14 (“post 2”) and 28 (“post 3”). Sera were obtained 2 weeks after second and third immunisations (i.e. post 2 and post 3, respectively).

TABLE 7
Immunisation scheme

| Group | Mice | Antigen Name | Ant Dose | Adjuvant |
|---|---|---|---|---|
| 1 | 1-8 | Al—H | — | Al—H |
| 2 | 9-16 | Al—H/K2 | — | Al—H/K2 |
| 3 | 17-24 | PVC13 | — | — |
| 4 | 25-32 | Conj type 1-, 5-, | 1 μg for each | A—-H |
| | | 6B-, 14-, 23F-CRM | | |
| 5 | 33-40 | Conj type 1-, 5-, | 0.1 μg for each | Al—H |
| | | 6B-, 14-, 23F-CRM | | |
| 6 | 41-48 | Conj type 1-, 5-, | 0.01 μg for each | Al—H |
| | | 6B-, 14-, 23F-CRM | | |
| 7 | 49-56 | Conj type 1-, 5- | 1 μg for each | Al—H/K2 |
| | | 6B-, 14-, 23F-CRM | | |
| 8 | 57-64 | Conj type 1-, 5- | 0.1 μg for each | Al—H/K2 |
| | | 6B-, 14-, 23F-CRM | | |
| 9 | 65-72 | Conj type 1-, 5-, | 0.01 μg for each | Al—H/K2 |
| | | 6B-, 14-, 23F-CRM | | |
| 10 | 72-80 | Conj type 5-CRM | 1 μg | Al—H |
| 11 | 81-88 | Conj type 5-CRM | 0.1 μg | Al—H |
| 12 | 89-96 | Conj type 5-CRM | 1 μg | Al—H/K2 |
| 13 | 97-104 | Conj type 5-CRM | 0.1 μg | Al—H/K2 |

Microsphere-Based Immunological Assay Results

An indirect MIA assay was performed to compare the adjuvant effects of Al—H and Al—H/K2 on the conjugates detailed in Table 7.

Statistical analyses were performed using the Mann-Whitney test (p-value) to evaluate significance between groups immunized with different adjuvant at the same dose. Titers of IgG in serum were expressed as means of individual serum samples from 8 mice+/−standard errors of the means.

Serotype 14-Antibody Titers Against Mixture of 1-, 5-, 6B-, 14-, 23F-CRM₁₉₇ Conjugates Adjuvanted with Al—H or Al—H/K2

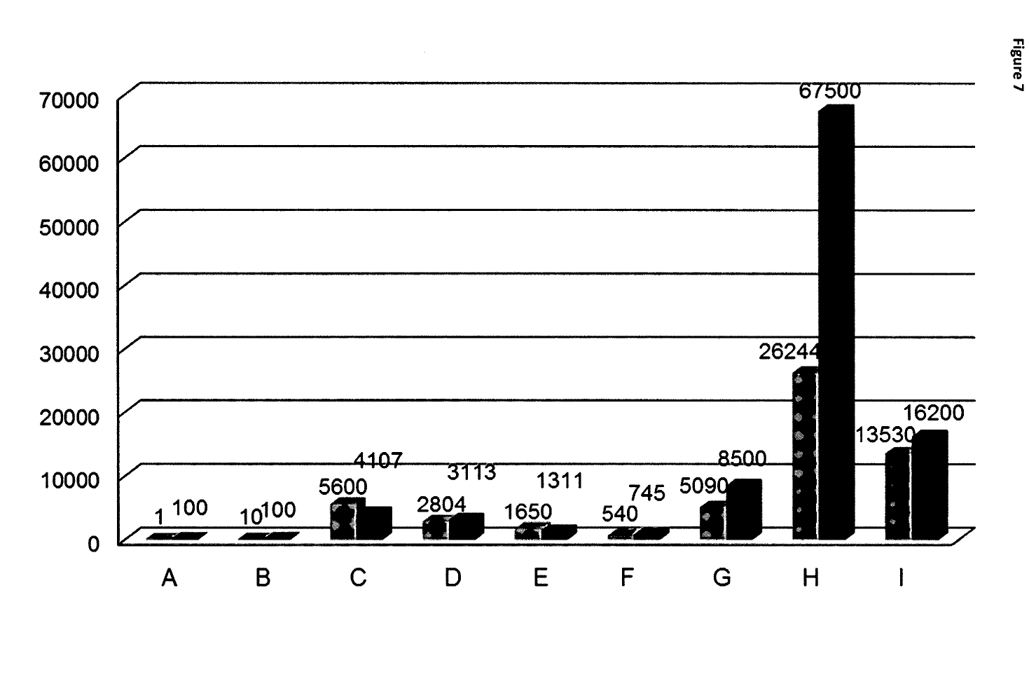

FIG. 8 compares the serotype 14 antibody response to a mixture of 1-, 5-, 6B-, 14-, 23F-CRM₁₉₇ conjugates adjuvanted with Al—H/K2 or Al—H. These data demonstrate that, at antigen concentrations 0.1 μg and 0.01 μg per antigen, adjuvanting with Al—H/K2 leads to a significantly higher anti-serotype 14 immune response than adjuvanting with Al—H.

At the highest conjugate dose tested (1 μg per antigen), there was no statistically significant difference between MFI results when adjuvanted with Al—H/K2 or Al—H. As indicated by the outlier in the “conj+Al—H” column in FIG. 8 however, one of the Al—H-adjuvanted vaccinations was unsuccessful, generating an MFI measurement similar to the Al—H-only control. By contrast, the Al—H/K2 adjuvanted vaccinations generated consistent MFI values.

Therefore, Al—H/K2 allows good immune responses when using lower aluminium concentrations, although this effect is less apparent at higher antigen doses. Moreover, compositions adjuvanted with Al—H/K2 performed at least as well as the PCV13 control, adjuvanted with aluminium phosphate.

Serotype 1-Antibody Titers Against Mixture of 1-, 5-, 6B-, 14-, 23F-CRM₁₉₇ Conjugates Adjuvanted with Al—H or Al—H/K2

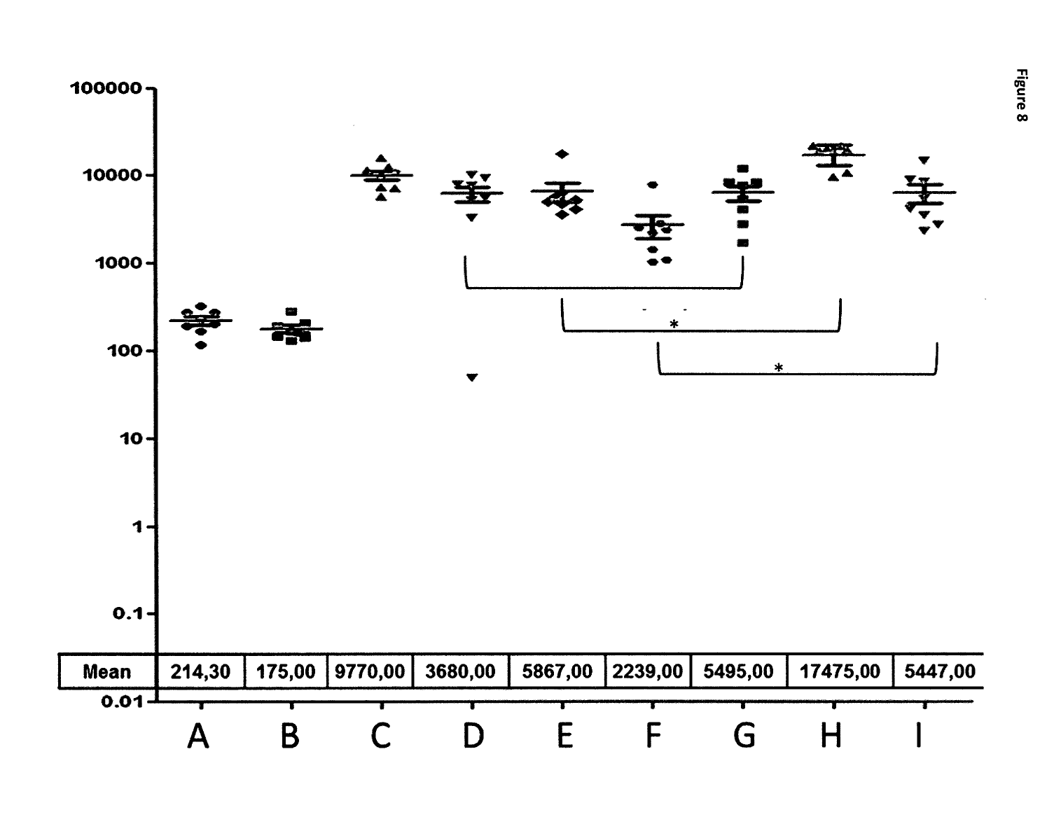

FIG. 9 compares the serotype 1 antibody response to a mixture of 1-, 5-, 6B-, 14-, 23F-CRM₁₉₇ conjugates adjuvanted with Al—H/K2 or Al—H. These data demonstrate that, at all antigen concentrations tested (0.01, 0.1 and 1 μg per antigen), adjuvanting with Al—H/K2 leads to significantly higher anti-serotype 1 immune response than Al—H.

No significant statistical difference was observed between Al—H/K2-adjuvanted groups receiving 0.1 or 1 μg dose, per antigen (p-value=0.19).

These data show that adjuvanting with Al—H/K2 allows good immune responses when using less aluminium. This effect is less apparent at higher doses but at higher doses, Al—H/K2 performs as well as Al—H. Moreover, compositions adjuvanted with Al—H/K2 performed similar to the PCV13 control, adjuvanted with aluminium phosphate.

Serotype 5-Antibody Titers Against Mixture of 1-, 5-, 6B-, 14-, 23F-CRM₁₉₇ Conjugates Adjuvanted with Al—H or Al—H/K2

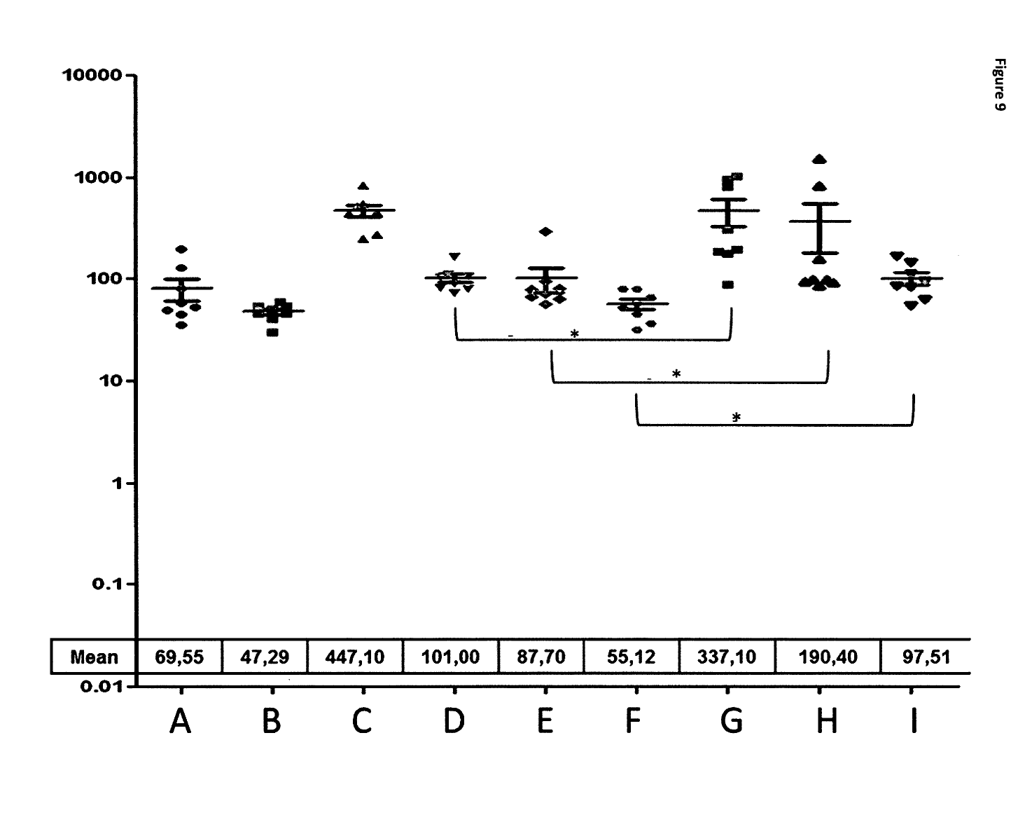

FIG. 10 compares the serotype 5 antibody response to a mixture of 1-, 5-, 6B-, 14-, 23F-CRM₁₉₇ conjugates adjuvanted with Al—H/K2 or Al—H. These data demonstrate that Al—H/K2 performs as well as Al—H at adjuvanting an anti- serotype 5 immune response. Moreover, compositions adjuvanted with Al—H/K2 performed similar to the PCV 13 control, adjuvanted with aluminium phosphate.

Summary of IgG Titers

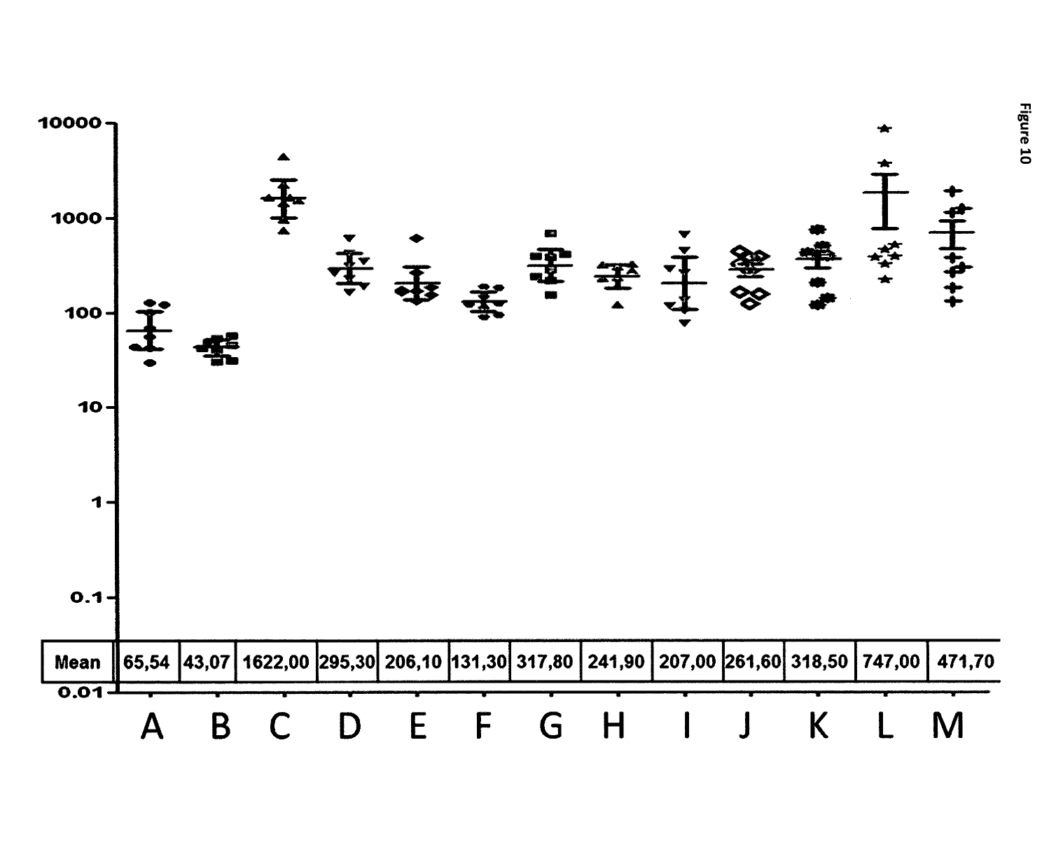

As indicated above, with Al—H/K2 represents an alternative to Al—H for adjuvanting anti-saccharide immune responses to conjugated saccharides, and in many cases, Al—H/K2 represents an improvement over Al—H. FIG. 11 provides a side-by-side comparison of all antibody titers obtained in the MIA study. These data demonstrate that immunisation with the mixture of 1-, 5-, 6B-, 14-, 23F-CRM₁₉₇ elicits an immune response to each of the saccharides tested (serotypes 1, 5 and 14). The anti-serotype 14 immune response is particularly high.

FIG. 11 also demonstrates that the anti-serotype 5 immune response following vaccination with a mixture of conjugated antigens 1-, 5-, 6B-, 14-, 23F-CRM₁₉₇ is comparable to the anti-serotype 5 immune response following vaccination with serotype 5-CRM₁₉₇ alone. This suggests that administration of conjugated saccharide antigens as a mixture does not lead to antigen interference.

Opsonophagocytosis Killing Assay (OPKA)

OPKA was performed to test in vitro the functionality of antibodies raised in mice vaccinated with the glycoconjugate vaccines described in Table 7. Sera were obtained after second and third immunisations (denoted “post 2” and “post 3”, respectively). OPKA is recognized by the World Health Organisation as a gold standard for licensing glycoconjugated vaccines for S. pneumoniae.

The OPKA methodology is well known. Briefly, mouse sera are pooled, heat inactivated for 30′ at 56° C. then diluted (3 fold serial dilutions, initial dilution 1:12). Bacteria are grown up to OD 0.5, divided in aliquots while staying on ice and stored at −80° C. Frozen stocks are used directly in the assay, ˜1200 CFUs/well. In this assay, strains SPPD (ST1), STREP5 (ST5), and 30001 (ST14) were tested. HL-60 cells (ATCC N° ^{CCL240TM}) propagated in vitro and differentiated with 0.8% Dimethylformamide (DMF) for 5 days (bacteria/cells ratio 1:400). Complement source was Baby Rabbit Complement previously screened for toxicity and activity, final concentration 12% (batch 2000, Liquid). All reaction components were incubated 1 hour at 37° C.+5% CO2. 5 μl from each well were spotted on THY agar plates which were incubated overnight at 37° C.+5% CO₂ Read Out at T60 (after 1 hr incubation). CFU/ml in test serum was compared to CFU/ml without test serum.

Internal Acceptance Criteria were B0 or input (Bacteria loaded initially in the plate): after overnight incubation we verified the number of CFU/spot (between 60-80). Bacteria viability: B1 (after 60′)/B0≧2.5 fold. Background signal in pre-immune and placebo samples are attributed to an unspecific killing (NSK). NSK %={CFU in Control B (Bacteria+HL60+active complement)/CFU in Control A (Bacteria+HL60+inactive complement)}×100. Comparative killing % in positive control (OMNI sera, anti whole bacteria antibodies produced in rabbit).

OPKA Results

SPPD (STI) Strain

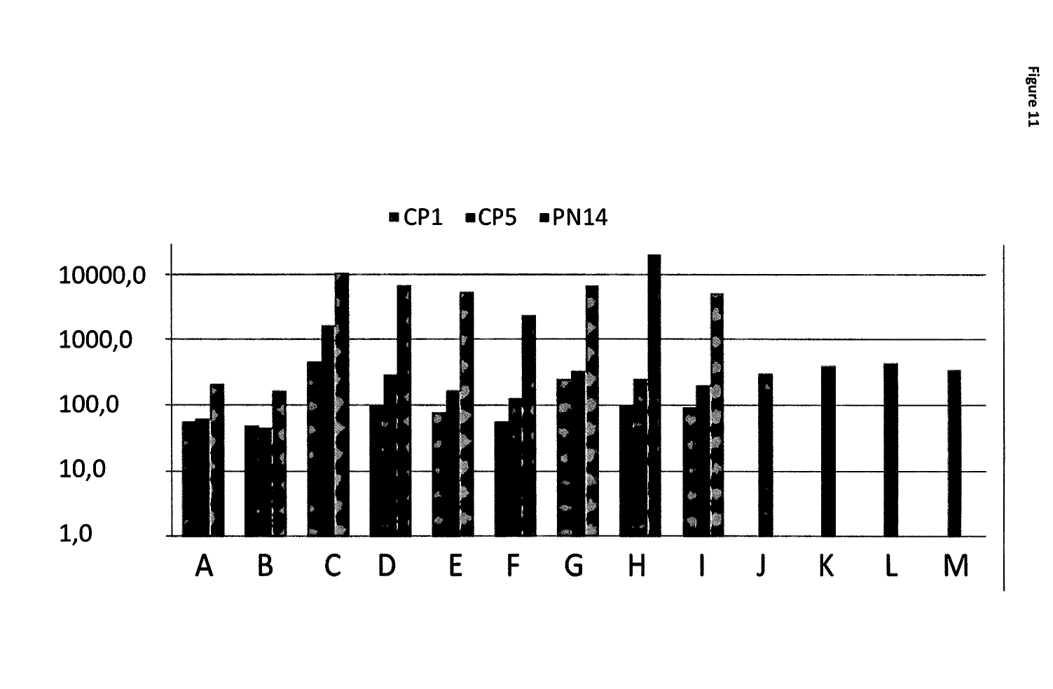

FIG. 12 compares the killing titers post 2 for the SPPD (1) S. pneumoniae strain. These data demonstrate that adjuvanting the mixture of 1-, 5-, 6B-, 14-, 23F-CRM₁₉₇ conjugates with Al—H provides kililing titers comparable to adjuvant-only controls. Al—H was found to be ineffective at all antigen concentrations tested. By contrast, adjuvanting the mixture of conjugated saccharide antigens with Al—H/K2 provided a high level killing titer that exceeded the PCV13 positive control at all concentrations rested. In particular, vaccinating with the highest antigen concentration tested (1 ug of each antigen), and adjuvanting with Al—H/K2 significantly outperformed even the positive control.

These data suggest that adjuvanting a mixture of serotype 1-, 5-, 6B-, 14-, 23F-CRM₁₉₇ conjugates with Al—H/K2 elicits high level killing titers against the SPPD (1) S. pneumoniae strain. Moreover, compositions adjuvanted with Al—H/K2 performed similar to the PCV13 control, adjuvanted with aluminium phosphate.

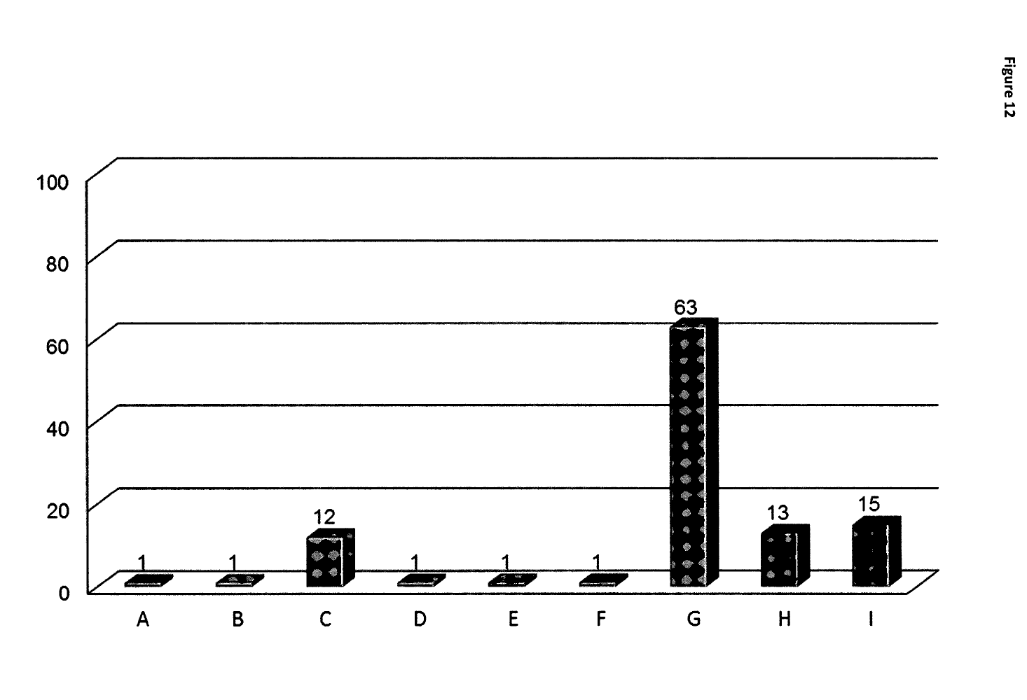

FIG. 13 compares the killing titers post 3 for the SPPD (1) S. pneumoniae strain. These data demonstrate that adjuvanting the mixture of 1-, 5-, 6B-, 14-, 23F-CRM₁₉₇ conjugates with Al—H provides kililing titers comparable to adjuvant-only controls, even after a third immunisation. By contrast, mixtures adjuvanted with Al—H/K2 elicited high level killing titers against this strain, at all antigen concentrations tested. Although killing titer levels were higher post 3 than post 2, there was not a striking difference between these titers. This contrasts the killing titers observed following vaccination with the positive control, PCV13, which required a third immunisation to achieve killing titers comparable to the Al—H/K2-adjuvanted mixture containing lug of each antigen.

Overall, Al—H/K2 was observed to be a far more effective adjuvant than Al—H at eliciting killing titers against the SPPD (1) S. pneumoniae strain, particularly at the highest antigen concentration tested. Interestingly, there were no striking differences between Al—H/K2-adjuvanted responses post 2 and post 3. Moreover, compositions adjuvanted with Al—H/K2 performed similar to the PCV13 control, adjuvanted with aluminium phosphate.

STREP(5) Strain

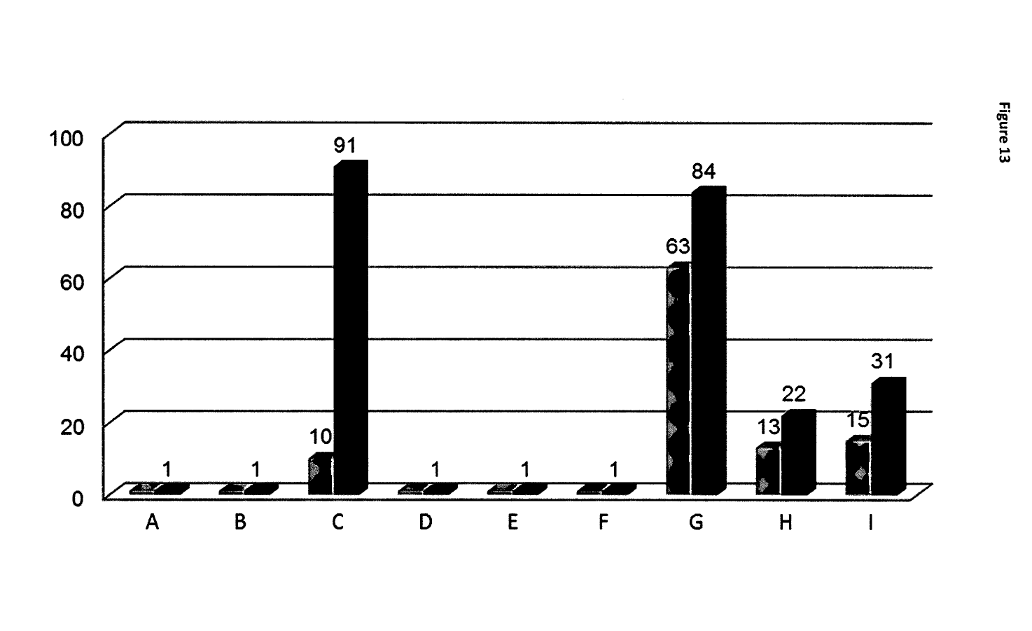

FIG. 14 compares the killing titers post 2 for the STREP(5) S. pneumoniae strain. Sera were obtained from mice vaccinated with the mixture of 1-, 5-, 6B-, 14-, 23F-CRM₁₉₇ conjugates, or only the serotype 5-CRM₁₉₇ conjugate, adjuvanted with Al—H or Al—H/K2. These data demonstrate that the positive control, PCV13 induces only a low level killing titer against this strain. Similarly, the mixture of 1-, 5-, 6B-, 14-, 23F-CRM₁₉₇ conjugates provided low level killing titer against this strain, even when adjuvanted with Al—H/K2. Similar data were observed with Al—H-adjuvanted vaccines comprising only the serotype 5-CRM₁₉₇ conjugate.

Surprisingly, these data demonstrate that vaccination with only the serotype 5-CRM₁₉₇ conjugate, adjuvanted with Al—H/K2 provides an extremely high killing titer against the STREP(5) strain that significantly exceeds the killing titer achieved by the positive control. Moreover, compositions adjuvanted with Al—H/K2 performed similar to the PCV13 control, adjuvanted with aluminium phosphate.

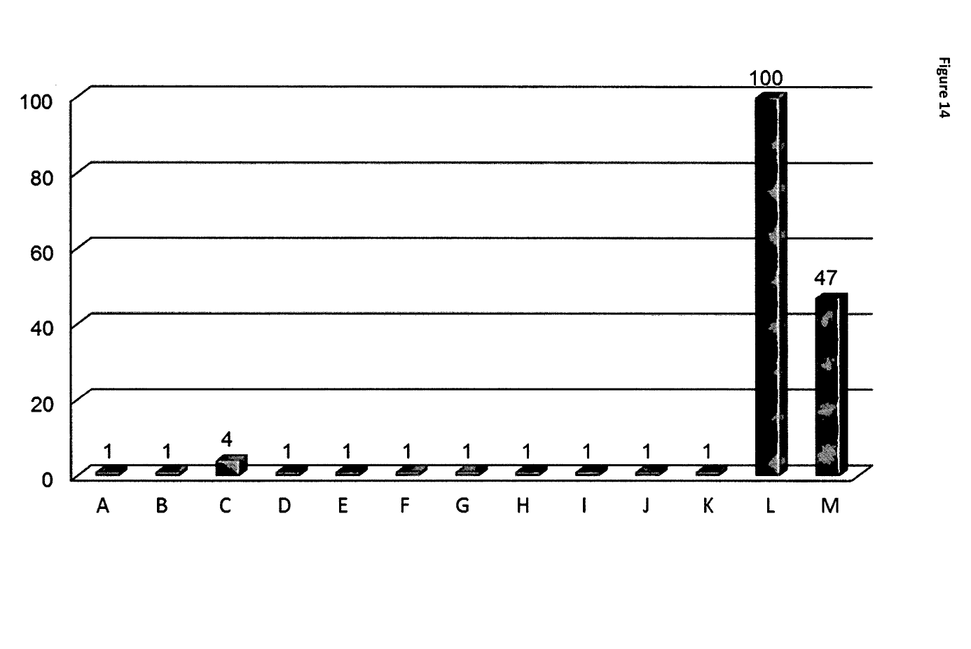

FIG. 15 compares the killing titers post 3 for the STREP(5) S. pneumoniae strain. These data demonstrate that the positive control, PCV13 induces only a low level killing titer against this strain, even after the third immunisation, and similarly low level killing titers were observed for Al—H-adjuvanted vaccines. By contrast, the killing titer for the mixture adjuvanted with Al—H/K2 (0.01 μg of each antigen) was significantly enhanced post 3, and was far superior to Al—H-adjuvanted mixture and the positive control. Similarly, killing titers following vaccination with the serotype 5-CRM₁₉₇ conjugate, adjuvanted with Al—H/K2, were found to be significantly increased post 3.

Overall, Al—H/K2 was again observed to be a far more effective adjuvant than Al—H at eliciting killing titers against the STREP(5) S. pneumoniae strain after administering the mixture of 1-, 5-, 6B-, 14-, 23F-CRM₁₉₇ conjugates (0.01 μg per antigen). Similarly high killing titers were observed using the serotype 5-CRM₁₉₇ conjugate adjuvanted with Al—H/K2. Moreover, compositions adjuvanted with Al—H/K2 significantly outperformed the PCV13 control, adjuvanted with aluminium phosphate.

There were also striking differences between Al—H/K2-adjuvanted responses post 2 and post 3, whereas Al—H-adjuvanted mixtures elicited killing titers comparable to the adjuvant-only controls.

30001 (14) Strain

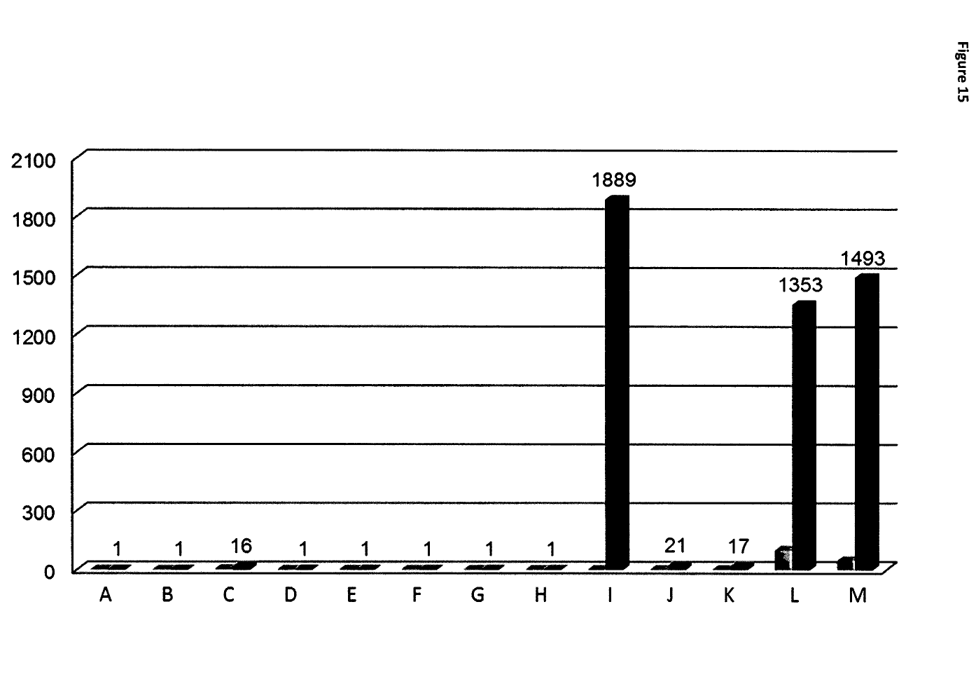

FIG. 16 compares the killing titers post 2 for the 300001(14) S. pneumoniae strain. These data demonstrate that adjuvanting the mixture of 1-, 5-, 6B-, 14-, 23F-CRM₁₉₇ conjugates with Al—H/K2 provides significantly higher kililing titers against the the 300001(14) strain than when adjuvanted with Al—H, and all antigen concentrations tested. Similarly, the mixture adjuvanted with Al—H/K2 far exceeded the killing titer against this strain achieved by vaccination with the positive control, PCV13.

Interestingly, the killing titer achieved using the Al—H/K2-adjuvanted saccharide conjugate mixture was higher following vaccination with 0.01 or 0.1 μg of each antigen than 1 μg of each antigen. Moreover, compositions adjuvanted with Al—H/K2 performed similar to the PCV13 control, adjuvanted with aluminium phosphate.

FIG. 7 compares the killing titers post 3 for the 300001(14) S. pneumoniae strain. These data demonstrate that adjuvanting the mixture of 1-, 5-, 6B-, 14-, 23F-CRM₁₉₇ conjugates with Al—H provides kililing titers worse than the positive control. Also, the killing titers for the positive control and Al—H-adjuvanted mixtures post 3 were the same or less than post 2. By contrast, the Al—H/K2-adjuvanted mixtures significantly outperformed Al—H-adjuvanted mixtures and the positive control post 3. Although killing titer levels were higher post 3 than post 2, there was not a striking difference between titers obtained using 0.01 μg or 1 μg of each antigen. Surprisingly however, killing titers observed using 0.1 μg of each antigen, adjuvanted with Al—H/K2 increased significantly post 3, compared to post 2. Moreover, compositions adjuvanted with Al—H/K2 performed at least as well as the PCV13 control, adjuvanted with aluminium phosphate.

Overall, Al—H/K2 was again observed to be a far more effective adjuvant than Al—H at eliciting killing titers against the 300001(14) S. pneumoniae strain, particularly when 0.01 μg of each antigen was used. Al—H/K2-adjuvanted responses post 2 and post 3 were surprisingly higher when 0.01 μg of each antigen was used.

### Protein Antigen

Preparation of S. pneumoniae RrgB321

The combination of RrgB321 used in this study corresponds to a fusion between RrgB sequences from (3) Taiwan-23F (2) Finland 6B-12 (1) TIGR4, and thus comprises pili of clades III, II and I. Details of RrgB321 are provided in references 235 and 236. RrgB321 was expressed with a His tag. The final RrgB321 vaccine composition contains antigen (0.4 mg/ml), compound K2 (2 mg/ml), Al—H (2 mg/ml), NaCl (9 mg/ml), histidine buffer (10 mM, pH 6.5), formulation volume: 0.05 ml (in water).

In some immunisation groups, some formulation components were omitted. In these cases the formulation process simply proceeded to the next step.#

Adsorption Studies

To determine whether the K2 remains adsorbed to Al—H, compositions were prepared as above. HPLC analyses revealed that at least 97% K2 remains adsorbed to Al—H (2 mg/ml). SDS-PAGE analyses revealed that RrgB321 consistently adsorbed to Al—H with high efficiency (>95% adsorption) in the absence of K2, and approximately 50% in the presence of K2. Therefore, the antigen is largely adsorbed in the presence of K2, while K2 is almost entirely adsorbed into Al—H. Neither antigen nor K2 showed detectable degradation patterns after formulation.

Immunisation Schemes

8 C57BL/6 mice were used per immunisation group. Mice received intramuscular immunisation of RrgB321, adjuvanted with Al—H or Al—H/K2. Each dose contained 20 μg RrgB321, 2 mg/ml Al—H and/or 100 μg K2 (as appropriate). Adjuvant controls were included. Volume per administration was 50 μl (into one leg). Mice were immunised after days 0 (priming), 14 (first boost), and 28 (second boost). Sera were obtained at day 38.

TABLE 8
Immunisation scheme

| Group | Antigen Name |
|---|---|
| 1 | Omniserum |
| 2 | RrgB321 + K2 |
| 3 | PBS |
| 4 | PBS + RrgB321 |
| 5 | Al—H + RrgB321 |
| 6 | Al—H/K2 + RrgB321 |

Adjuvant Effect on Antibody Response to RrgB

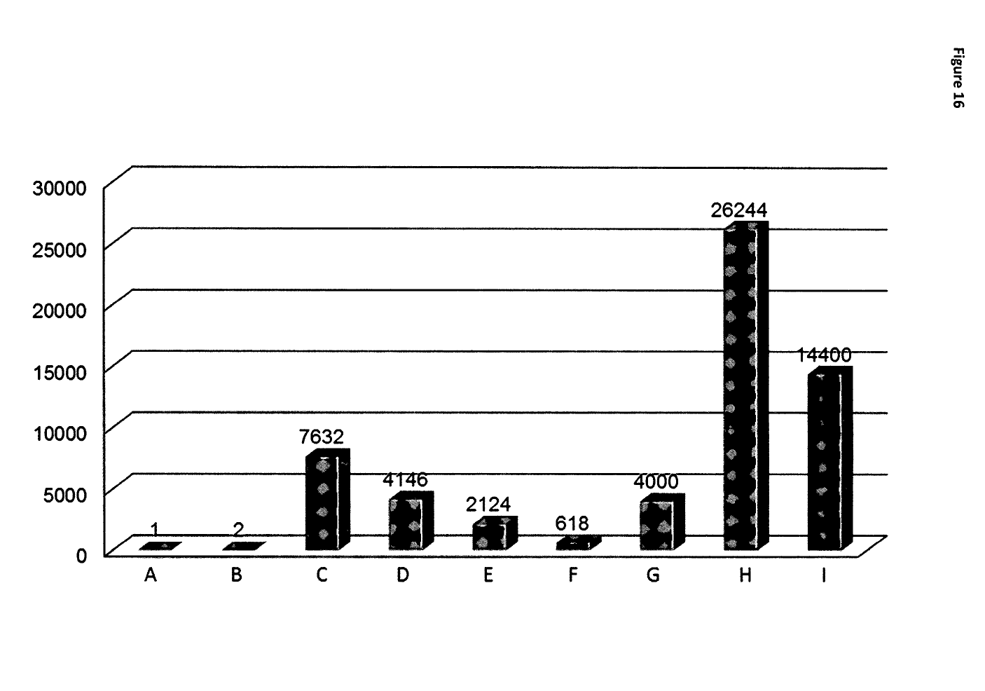

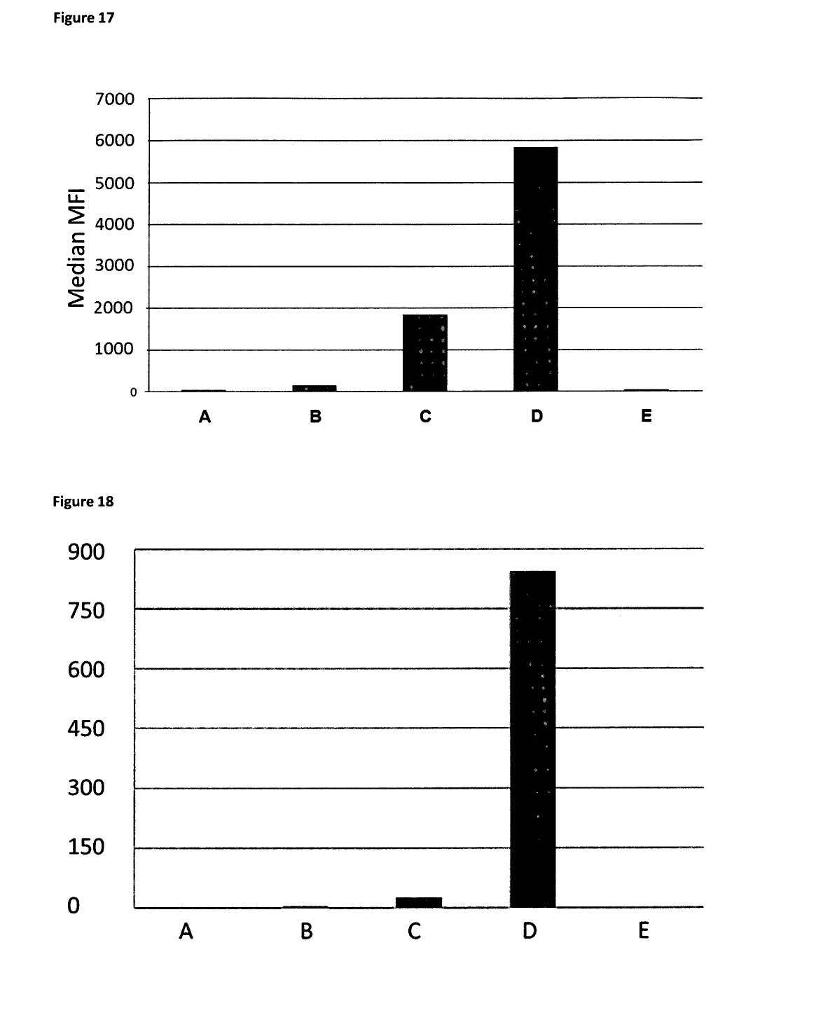

The microsphere-based immunological assay (MIA) was performed to compare the adjuvant effects of Al—H, Al—H/K2 and K2 on RrgB321 (see FIG. 17). The MIA assay (Luminex technology) is well known, and is described in e.g. reference 237.

These data, expressed in MFI, demonstrate that RrgB321 adjuvanted with Al—H/K2 elicits a significantly higher antibody response (approximately 3 times higher) than when adjuvanted with Al—H. Interestingly, administration of RrgB321 in combination with only K2 (i.e. omiting Al—H from Al—H/K2) did not elicit a detectable antibody response. This suggests that adsorption of Al—H to K2 may be important for adjuvanting an immune response, and that co-delivery of Al—H and K2 may be important for eliciting an optimal immune response.

Opsonophagocytosis Killing Assay (OPKA)

OPKA was performed to test in vitro the functionality of antibodies raised in mice vaccinated with the compositions above. Sera were obtained. A positive control rabbit polyclonal antiserum was also used, called “Omniserum”. The OPKA assay is well known, and is described in e.g. reference 237. In this assay, strains TIGR4 and 6B SPEC were tested.

OPKA Results

TIGR4

FIG. 18 compares the killing titers for the TIGR4 S. pneumoniae strain. These data demonstrate that antiserum obtained following immunisation with RrgB321 adjuvanted with Al—H/K2 proved very effective at killing TIGR4 cells in vitro, with a calculated titer of 844 (see Table 9). Adjuvanting with Al—H alone was found to be less effective at killing TIGR4 cells in vitro, with a calculated titer (the dilution of a test serum at which 50% of bacteria killing is observed) of 26.

TABLE 9

| | Titer |
|---|---|
| PBS | <12 |
| PBS + RrgB321 | <12 |
| Al—H (RrgB321 adsorbed) | 26 |
| Al—H/K2 (RrgB321 adsorbed) | 844 |
| RrgB321 + K2 (100 ul/ml) | <12 |

Immunisation with RrgB321 alone (adjuvanted with PBS) was able to induce approximately 40% killing, whereas immunisation with RrgB321 mixed with K2 alone elicited antibodies with minimal in vitro function.

6B SPEC

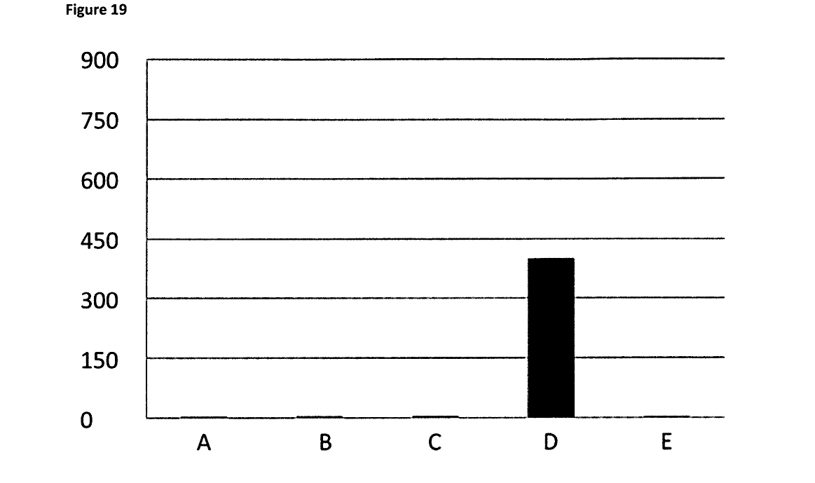

FIG. 19 compares the killing titers for the 6B SPEC S. pneumoniae strain. These data again demonstrate that antiserum obtained following immunisation with RrgB321 adjuvanted with Al—H/K2 proved very effective at killing 6B SPEC cells in vitro, with a calculated titer of 401 (see Table 10). Adjuvanting with Al—H alone was significantly less effective at killing 6B SPEC cells in vitro, with a calculated titer of <12.

TABLE 10

| | Titer |
|---|---|
| PBS | <12 |
| PBS + RrgB321 | <12 |
| Al—H (RrgB321 adsorbed) | <12 |
| Al—H/K2 (RrgB321adsorbed) | 401 |
| RrgB321 + K2 (100 ul/ml) | <12 |

Interestingly, immunisation with RrgB321 mixed with K2 alone elicited antibodies with minimal in vitro function.

These data demonstrate that adjuvanting compositions comprising S. pneumoniae protein antigen(s) with TLR agonist and an aluminium salt causes strong increase in serum opsonophagocytic activity, compared to alum salt, or TLR agonist alone. This indicates a much higher functional activity against S. pneumoniae strains of Glade I and II, and likely also strains of Glade III.

It will be understood that the invention has been described by way of example only and modifications may be made whilst remaining within the scope and spirit of the invention.

### REFERENCES

[1] WO2012/072769.

[2] WO2012/031140.

[3] Rosenberg et al. (2010) J Immunol 184:136.20.

[4] U.S. Pat. No. 4,666,886.

[5] WO2009/118296.

[6] WO2008/005555.

[7] WO2009/111337.

[8] WO2009/067081.

[9] WO2007/040840.

[10] WO2010/014913.

[11] UK patent application GB-A-2220211.

[12] Myers et al. (1990) pages 145-156 of Cellular and molecular aspects of endotoxin reactions.

[13] Ulrich (2000) Chapter 16 (pages 273-282).

[14] Johnson et al. (1999) J Med Chem 42:4640-9.

[15] Baldrick et al. (2002) Regulatory Toxicol Pharmacol 35:398-413.

[16] WO 94/21292.

[17] Vaccine Design . . . (1995) eds. Powell & Newman. ISBN: 030644867X. Plenum.

[18] Clausi et al. (2008) J Pharm Sci DOI 10.1002/jps.21390.

[19] WHO Technical Report Series No. 927, 2005. Pages 64-98.

[20] US-2008/0102498.

[21] US-2006/0228381.

[22] Watson (2000) Pediatr Infect Dis J19:331-332.

[23] Rubin (2000) Pediatr Clin North Am 47:269-285, v.

[24] Jedrzejas (2001) Microbiol Mol Biol Rev 65:187-207.

[25] Vaccines (2008) eds. Plotkin, Orenstein and Offit. ISBN 978-1-4160-3611-1.

[26] Jones (2005) An. Acad. Bras. Cienc, 77(2) 293-324.

[27] Jones (2005) J Pharm Biomed Anal 38 840-850.

[28] US-2007/0231340.

[29] US-2007/0184072.

[30] US-2006/0228380.

[31] WO2008/143709.

[32] Research Disclosure, 453077 (January 2002)

[33] EP-A-0378881.

[34] EP-A-0427347.

[35] WO93/17712

[36] WO94/03208.

[37] WO98/58668.

[38] EP-A-0471177.

[39] WO91/01146

[40] Falugi et al. (2001) Eur J Immunol 31:3816-3824.

[41] Baraldo et al. (2004) Infect Immun 72(8):4884-7.

[42] EP-A-0594610.

[43] Ruan et al. (1990) J Immunol 145:3379-3384.

[44] WO00/56360.

[45] Kuo et al. (1995) Infect Immun 63:2706-13.

[46] Michon et al. (1998) Vaccine. 16:1732-41.

[47] WO02/091998.

[48] WO01/72337

[49] WO00/61761.

[50] WO00/33882

[51] WO2007/071707

[52] WO99/42130.

[53] U.S. Pat. No. 4,761,283.

[54] U.S. Pat. No. 4,356,170.

[55] U.S. Pat. No. 4,882,317.

[56] U.S. Pat. No. 4,695,624.

[57] Mol. Immunol., 1985, 22, 907-919

[58] EP-A-0208375.

[59] Bethell G. S. et al., J. Biol. Chem., 1979, 254, 2572-4

[60] Hearn M. T. W., J. Chromatogr., 1981, 218, 509-18

[61] WO00/10599.

[62] Geyer et al., Med. Microbiol. Immunol, 165: 171-288 (1979).

[63] U.S. Pat. No. 4,057,685.

[64] U.S. Pat. Nos. 4,673,574; 4,761,283; 4,808,700.

[65] U.S. Pat. No. 4,459,286.

[66] U.S. Pat. No. 4,965,338.

[67] U.S. Pat. No. 4,663,160.

[68] WO2007/000343.

[69] Bagnoli et al. (2008) J Bacteriol. 190(15):5480-92.

[70] WO2007/116322.

[71] LeMieux et al. (2006) Infect Immun 74:2453-6.

[72] Nelson et al. (2007) Mol Microbiol 66:329-40.

[73] PCT/EP2011/071566

[74] Needleman & Wunsch (1970) J. Mol. Biol. 48, 443-453.

[75] Rice et al. (2000) Trends Genet 16:276-277.

[76] WO02/079241

[77] WO02/34773.

[78] WO00/06737.

[79] WO00/06738.

[80] WO00/58475.

[81] WO2003/082183..

[82] WO2004/092209.

[83] Kirkham et al. (2006) Infect Immun. 74(1):586-93.

[84] WO2005/108580.

[85] Berry et al. (1999) Infect Immun 67(2):981-5.

[86] U.S. Pat. No. 6,716,432.

[87] WO90/06951.

[88] WO99/03884.

[89] Baba et al. (2002) Infect Immun 70: 107-113.

[90] U.S. Pat. No. 7,217,791

[91] WO2008/061953.

[92] Cao et al. (2007) Vaccine 25(27):4996-5005.

[93] WO2005/063283.

[94] WO2003/104272.

[95] WO00/37105.

[96] Adamou et al. (2001) Infect Immun. 69(2):949-58.

[97] WO00/37105.

[98] Adamou et al. (2001) Infect Immun. 69(2):949-58.

[99] WO98/18930.

WO02/22168

Wizemann et al. (2001) Infect Immun 69:1593-8.

WO99/53940.

WO02/22167.

WO02/08426.

WO01/12219.

Briles et al. (2000) J Infect Dis 182:1694-1701.

Talkington et al. (1996) Microb Pathog. 21(1):17-22.

WO00/76540.

Bethe et al. (2001) FEMS Microbiol Lett. 205(1):99-104.

WO01/81380.

Brown et al. (2001) Infect Immun 69:6702-6.

Whalan et al. (2005) FEMS Immunol Med Microbiol 43:73-80.

Jomaa et al. (2006) Vaccine. 24(24):5133-9.

Giefing et al. (2008) J Exp Med 205:117-131.

Findeis et al., Trends Biotechnol. (1993) 11:202

Chiou et al. (1994) Gene Therapeutics: Methods And Applications Of Direct Gene Transfer. ed. Wolff

Wu et al., J. Biol. Chem. (1988) 263:621

Wu et al., J. Biol. Chem. (1994) 269:542

Zenke et al., Proc. Natl. Acad. Sci. (USA) (1990) 87:3655

Wu et al., J. Biol. Chem. (1991) 266:338

WO2009/016515

Kuo et al. (1995) Infect Immun 63:2706-13.

Michon et a/. (1998) Vaccine. 16:1732-41.

WO02/091998.

Research Disclosure, 453077 (January 2002).

EP-A-0372501.

EP-A-0378881.

EP-A-0427347.

WO93/17712.

WO94/03208.

WO98/58668.

EP-A-0471177.

WO91/01146.

Falugi et al. (2001) Eur J Immunol 31:3816-3824.

Baraldo et al. (2004) Infect Immun 72(8):4884-7.

EP-A-0594610.

Ruan et al. (1990) J Immunol 145:3379-3384.

WO00/56360.

WO01/72337.

WO00/61761.

WO00/33882

WO2007/071707

WO99/42130.

U.S. Pat. No. 4,761,283.

U.S. Pat. No. 4,356,170.

U.S. Pat. No. 4,882,317.

U.S. Pat. No. 4,695,624.

Mol. Immunol., 1985, 22, 907-919

EP-A-0208375.

Bethell G. S. et al., J. Biol. Chem., 1979, 254, 2572-4

Hearn M. T. W., J. Chromatogr., 1981, 218, 509-18

WO00/10599.

Geyer et al., Med. Microbiol. Immunol, 165 : 171-288 (1979).

U.S. Pat. No. 4,057,685.

U.S. Pat. Nos. 4,673,574; 4,761,283; 4,808,700.

U.S. Pat. No. 4,459,286.

U.S. Pat. No. 4,965,338.

U.S. Pat. No. 4,663,160.

WO2007/000343.

WO2010/119343.

Giuliani et al. (2006) Proc Natl Acad Sci USA. 103:10834-9.

WO95/27787.

WO03/010317.

WO2007/110700.

WO2006/138004.

WO2005/084306.

WO2005/002619.

WO03/049762.

WO02/02606.

WO00/37494.

WO2008/020330.

WO2006/091517.

WO2006/089264.

Covacci & Rappuoli (2000) J. Exp. Med. 19:587-592.

WO93/18150.

Covacci et al. (1993) Proc. Natl. Acad. Sci. USA 90:5791-5795.

Tummuru et al. (1994) Infect. Immun. 61:1799-1809.

Marchetti et al. (1998) Vaccine 16:33-37.

Telford et al. (1994) J. Exp. Med. 179:1653-1658.

Evans et al. (1995) Gene 153:123-127.

WO96/01272 & WO96/01273, especially SEQ ID NO:6.

WO97/25429.

Rappuoli et al. (1991) TIBTECH 9:232-238.

Nencioni et al. (1991) Infect Immun. 59(2): 625-30.

Dasarai et al. (2011) J Gen Virol PMID: 21307228.

Harper et al. (2004) Lancet 364(9447):1757-65. WO03/097091.

Cassone & Torosantucci (2006) Expert Rev Vaccines 5:859-67.

Brandt et al. (2006) J Antimicrob Chemother. 58(6):1291-4. Epub 2006 Oct 26

Winter et al., (1991) Nature 349:293-99

U.S. Pat. No. 4,816,567.

Inbar et al., (1972) Proc. Natl. Acad. Sci. U.S.A. 69:2659-62.

Ehrlich et al., (1980) Biochem 19:4091-96.

Huston et al., (1988) Proc. Natl. Acad. Sci. U.S.A. 85:5897-83.

Pack et al., (1992) Biochem 31, 1579-84.

Cumber et al., (1992) J. Immunology 149B, 120-26.

Riechmann et al., (1988) Nature 332, 323-27.

Verhoeyan et al., (1988) Science 239, 1534-36.

GB 2,276,169.

Bodanszky (1993) Principles of Peptide Synthesis (ISBN: 0387564314).

Fields et al. (1997) Meth Enzymol 289: Solid-Phase Peptide Synthesis. ISBN: 0121821900.

Chan & White (2000) Fmoc Solid Phase Peptide Synthesis. ISBN: 0199637245.

Kullmann (1987) Enzymatic Peptide Synthesis. ISBN: 0849368413.

Ibba (1996) Biotechnol Genet Eng Rev 13:197-216.

Hoskins et al. (2001) J. Bacteriol. 183:5709-5717.

GenBank NC_{—}004512.

GenBank NC_{—}003440.

GenBank NC_{—}003028.

Tettelin et al. (2001) Science 293:498-506.

WO02/077021.

Needleman & Wunsch (1970) J. Mol. Biol. 48, 443-453.

Rice et al. (2000) Trends Genet 16:276-277.

WO2011/119759

WO2011/027222.

WO2007/034917.

WO2007/034173.

WO2008/114817.

US2009-0105212.

US2009-0118263.

US2009-0143400.

US2009-0192153.

WO2007/093901.

WO2009/019553.

US2009/0221631.

WO2008/004948.

WO2008/135791.

US2009/0099216.

US2009/0202484.

WO 2013/131983 PCT/EP2013/054545

WO2008/101867.

WO2010/077613.

US2010/0143301.

Remington: The Science and Practice of Pharmacy (Gennaro, 2000; 20th edition, ISBN: 0683306472)

Talaga (2002) Vaccine 20:2474-84.

WO2011/049677.

WO2010/140119

PCT/EP2011/071566

Harfouche et al. 2011. Infection and immunity Vol 80, no. 1, pp 451-160

1. A composition selected from the group consisting of:
(A) a composition comprising (i) a TLR agonist, (ii) an insoluble metal salt, and (iii) one or more S. pneumoniae saccharide or protein agtigens, wherein the TLR agonist is an agonist of human TLR7;
(B) a composition comprising (i) a TLR agonist, (ii) an insoluble metal salt, and (ii) one or more S. pneumoniae saccharride or protein antigens, wherein the insoluble metal salt is an aluminium salt;
(C) a composition comprising (i) a TLR agonist, (ii) an insoluble metal salt, (iii) a buffer, and (iv) one or more S. pneumoniae saccharide or protein antigens;
(D) a composition comprising (i) a TLR agonist, (ii) an insoluble metal salt, and (iii) one or more S. pneumoniae saccharide or protein antigens, wherein the pH of the composition is pH 6 to pH 8;
(E) a composition comprising (i) a TLR agonist, (ii) an insoluble metal salt, and (iii) one or more S. pneumoniae saccharide antigens, wherein at least one of the one or more S. pneumoniae saccharide antigens is conjugate to CMR197;
(F) a composition comprising (i) a TLR agonist, (ii) an insoluble metal salt, and (iii) one or more S. pneumoniae saccharide antigens selected from 2-10 different S. pneumoniae saccharide antigens serotypes;
(G) a composition comprising (i) a TLR agnoist, (ii) an insoluble metal salt, and (iii), S. pneumoniae saccharide antigens consisiting of 11 different serotypes, provided that the 11 different serotypes are not all of the serotypes in the group consisting of 1, 3, 4, 5, 6B, 7F, 9V, 14, 18C, 19F and 23F;
(H) a composition comprising (i) a TLR agnoist, (ii) an insoluble metal salt, and (iii) one or more S. pneumoniae saccharide antigens, wherein at least one of the one or more S. pneumoniae saccharide antigens is conjugated directly to a carrier;
(I) a composition comprising (i) an aluminum hydroxide adjuvant, (ii) a TLR7 agonist of formula (K) 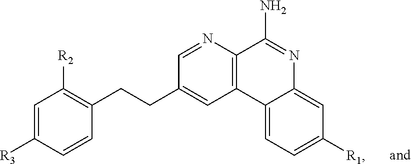
(iii) one or more S. pneumoniae saccharide antigens selected from the group consisting of serotypes 1, 5, 6B, 14, and 23F, wherein each saccharide is conjugated to CRM₁₉₇ and at least one of the TLR7 agonist or at least one of the saccharides is adsorbed to the aluminium hydroxide adjuvant;
(J) a composition comprising; (i) an aluminium hydroxide adjuvant, (ii) a TLR7 agonist of formula (K) 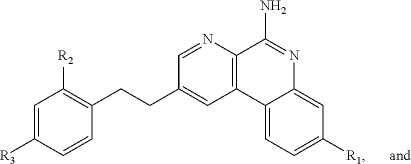
(iii) one or more S. pneumoniae saccharide antigen from only serotype 5 conjugated to CRM₁₉₇ wherein at least one of the TLR7 agonists or at least one of the saccharides is adsobred to the aluminum hydroxide adjuvant; and
(K) a composition comprising: (i) an adjuvant complex comprising a first TLR agonist adsorbed to an insoluble metal salt, (ii) an adjuvant complex comprising a second TLR agonist adsorbed to an insoluble metal salt, and (iii) one or more S. pneumoniae saccharide or protein antigens.

2-12. (canceled)

13. The composition of claim 1, wherein the one or more S. pneumoniae saccharide antigens has a serotype selected from the goup consisting of serotypes 1, 2, 3, 4, 5, 6A, 6B, 7F, 8, 9N, 9V, 10A, 11A, 12F, 14, 15B, 17F, 18C, 19A, 19F, 20, 22F, 23F, and 33F.

14. The composition of claim 13, comprising a 5-, 7-, 9-, 10-, 11-, 12-, or 13-valent combination of serotypes.

15. The composition of claim 14, wherein the 5-valent combination of S. pneumoniae saccharides serotypes is 1, 5, 6B, 14, and 23F.

16. (canceled)

17. The composition of claim 14, wherein the 5-valent combination of S. pneumoniae saccharides is serotypes 4, 6B, 9V, 14, 18C, 19F, and 23F.

18. (canceled)

19. The composition of claim 14, wherein the 9-valent combination of S. pneumoniae saccharides is serotypes 1, 4, 5, 6B, 9V, 14, 18C, 19F, and 23F.

20. (canceled)

21. The composition of claim 14, wherein the 10-valent combination of S. pneumoniae saccharides is serotypes 1, 4, 5, 6B, 7F, 9V, 14, 18C, 19F, and 23F.

22-23. (canceled)

24. The composition of claim 14, wherein the 12-valent combination of serotypes is sterotypes 1, 4, 5, 6B, 7F, 9V, 14, 18C, 19F, and 23F, further comprising two additional serotypes selected from the groups consisting of 6A and 19A; 6A and 22F; 19A and 22F; 6A and 15B; 19A and 15B; and 22F and 15B.

25. (canceled)

26. The composition of claim 14, wherein the 13-valent combination of serotypes is serotypes 1, 3, 4, 5, 6B, 7F, 9V, 14, 18C, 19F, and 23F, and two additional serotypes selected from the groups consisting of: 19A and 22F; 8 and 12F; 8 and 15B; 8 and 19A; 8 and 22F; 12F and 15B; 12F and 19A; 12F and 22F; 15B and 19A; 15B and 22F; and 6A and 19A.

27. The composition of claim 26, wherein the 13-valent combination of serotypes is serotypes 1, 3, 4, 5, 6A, 6B, 7F, 9V, 14, 18C, 19, 19F, and 23F, or serotypes 1, 3, 4, 5, 6A, 6B, 7F, 9V, 14, 18C, 19A, 19F, and 23F.

28. The composition of claim 1, comprising an S. pneumoniae saccharide antigen from a serotype selected from the group consisting of serotypes 1, 2, 3, 4, 5, 6A, 6B, 7F, 8, 9N, 9V, 10A, 11A, 12F, 14, 15B, 17F, 18C, 19A, 19F, 20, 22F, 23F, and 33F.

29. The composition of claim 28, wherein the serotype is selected from the group consisting of serotype 1, 5, 6B, 14, and 23F.

30. The composition of claim 1, wherein the one or more S. pneumoniae saccharide antigens is conjugated to a carrier protein.

31. The composition of claim 30, wherein a carrier protein is selected from the group consisting of a bacterial toxin, a bacterial toxoid, a mutant bacterial toxin, and a mutant bacterial toxoid.

32-34. (canceled)

35. The composition of claim 30, wherein the carrier is conjugated directly to the saccharide.

36-40. (canceled)

41. The composition of claim 1, wherein the composition is pH 6 to pH 8.

42. The composition of claim 1, wherein the TLR7 agonist of formula (K) is the compound K2, 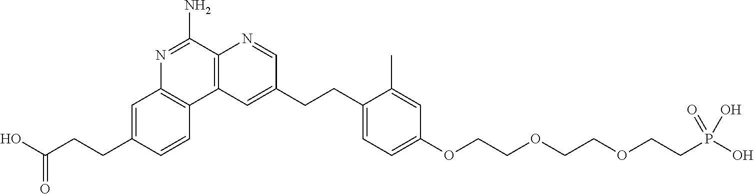 [SMILES: Cc1cc(OCCOCCOCCP(=O)(O)O)ccc1CCc1cnc2c(N)nc3cc(CCC(=O)O)ccc3c2c1]
or a pharmaceutically acceptable salt thereof.

43. The composition of claim 1, wherein the composition comprises a S. pneumoniae protein antigen.

44. A method of raising an immune response in a subject, comprising the step of administering to the subject the composition of claim 1.

45. (canceled)
